(19) United States
(12) Patent Application Publication
(10) Pub. No.: US 2026/0196300 A1
(43) Pub. Date: Jul. 9, 2026

(54) DETERMINING SPLICE SITES IN NUCLEOTIDE SEQUENCES USING CONDITIONAL PROBABILITIES GENERATED VIA A NEURAL NETWORK

(71) Applicant: Illumina, Inc., San Diego, CA (US)

(72) Inventors: Kishore Jaganathan, San Francisco, CA (US); Kai-How Farh, Hillsborough, CA (US)

(21) Appl. No.: 18/855,601

(22) PCT Filed: Sep. 25, 2024
(86) PCT No.: PCT/US2024/048475

§ 371 Date: Oct. 9, 2024

Related U.S. Application Data
(60) Provisional application No. 63/585,507, filed on Sep. 26, 2023.
(60) Provisional application No. 63/585,518, filed on Sep. 26, 2023.
(60) Provisional application No. 63/585,526, filed on Sep. 26, 2023.
(60) Provisional application No. 63/585,531, filed on Sep. 26, 2023.
(60) Provisional application No. 63/585,535, filed on Sep. 26, 2023.

(51) Int. Cl. G16B 30/00; G16B 5/20; G16B 20/20; G16B 40/20

(52) U.S. Cl. CPC G16B 30/00; G16B 5/20; G16B 20/20; G16B 40/20

(57) ABSTRACT

This disclosure describes methods, non-transitory computer-readable media, and systems that generate quantitative predictions of splice sites for nucleotide sequences. For example, in some cases, the disclosed systems use a neural network to analyze a nucleotide sequence that includes a conditioned nucleobase and flanking nucleobases. Based on the analysis, the neural network generates conditional probabilities for the flanking nucleobases, where each conditional probability indicates a likelihood that a corresponding flanking nucleobase is part of a matching splice site (e.g., a matching donor or acceptor) for the conditioned nucleobase. By generating such conditional probabilities, the disclosed system quantitatively identifies potential splice sites within the nucleotide sequence.

### CROSS-REFERENCE TO RELATED APPLICATIONS

This application claims priority to and the benefit of U.S. Provisional Patent Application No. 63/585,507, entitled “PREDICTING CONTEXT-SPECIFIC SPLICING WITHIN NUCLEOTIDE SEQUENCES THROUGH CONDITIONAL PROBABILITIES GENERATED VIA A NEURAL NETWORK,” filed on Sep. 26, 2023 (IP-2615-PRV); U.S. Provisional Patent Application No. 63/585,518, entitled “PREDICTING INTRON RETENTION WITHIN NUCLEOTIDE SEQUENCES BY USING A NEURAL NETWORK TO QUANTITATIVELY PREDICT SPLICING,” filed on Sep. 26, 2023 (IP-2636-PRV); U.S. Provisional Patent Application No. 63/585,526, entitled “A TRANSFORMER MODEL FOR PREDICTING SPLICING WITHIN NUCLEOTIDE SEQUENCES,” filed on Sep. 26, 2023 (IP-2637-PRV); U.S. Provisional Patent Application No. 63/585,531, entitled “DETERMINING SPLICE SITES IN NUCLEOTIDE SEQUENCES USING CONDITIONAL PROBABILITIES GENERATED VIA A NEURAL NETWORK,” filed on Sep. 26, 2023 (IP-2638-PRV); and U.S. Provisional Patent Application No. 63/585,535, entitled “IDENTIFYING BIOCHEMICAL-COMPOUND TREATMENTS FOR ALTERNATIVE SPLICING BASED ON NEURAL-NETWORK-BASED ANALYSES OF NUCLEOTIDE SEQUENCES,” filed on Sep. 26, 2023 (IP-2697-PRV). Each of the aforementioned applications is hereby incorporated by reference in its entirety.

### BACKGROUND

In recent years, biotechnology firms and research institutions have improved software for predicting how splicing events occur for a target nucleotide sequence, such as a precursor messenger ribonucleic acid (pre-mRNA) sequence from a genomic sample. For instance, some existing exon-junction prediction systems employ models to generate predictions that indicate which nucleobases within a nucleotide sequence correspond to a splice site (e.g., a donor or an acceptor). Such splice site predictions can aid in the identification of alternative splicing that may be caused by variants within a genomic host, which can lead to various diseases—including cancer and neurological disorders—or other unfavorable conditions. Thus, splice site predictions have been used to identify target variants in a population subset for new drugs as well as target variants that may be the subject of other therapies, such as genetic editing.

Despite these advances, existing exon-junction prediction systems continue to exhibit various drawbacks and disadvantages that impair the robustness and accuracy of their model outputs. As explained further below, such existing systems typically suffer these impairments due to their approach to the splice-site prediction task and/or the architecture of their implemented models.

For example, existing exon-junction prediction systems can apply an inflexible approach to splice site prediction by rigidly implementing a qualitative approach that confines the prediction task to a simplified binary classification problem. To illustrate, many existing systems employ models—including state-of-the-art neural-network-based models—that are limited to generating a single set of binary classification outputs that indicate whether target nucleobases from an input nucleotide sequence are part of a splice site or not part of the splice site. The set of outputs is often context-agnostic, where the output classifications generally represent splicing for any given context but also no specific context associated with the input nucleotide sequence or, at least, a context average. To illustrate, outputs of existing systems typically represent the average across multiple tissues associated with the input nucleotide sequence, but the average has no particularized relevance to a specific tissue type. Thus, while these existing systems can provide outputs that identify splice sites via binary classification, they fail to provide further granularity on the splicing of an input nucleotide sequence. Consequently, such binary and context-agnostic classifications are typically limited to determining whether nucleobases are (i) part of a reference splice site—without any or much consideration for alterative splice sites—or (ii) part of a splice site across non-specific tissue types. Nor do existing splice-site classifications separately or adequately account for the role of intron retention in how an input nucleotide sequence is spliced.

Existing exon-junction prediction systems are often limited to providing binary splice-site classification outputs in part due to their standard supervised learning methods and basic or limited training data. For instance, many existing systems train machine-learning models using training data that consists of short read data providing the locations of splice sites. These existing systems typically train machine-learning models by providing an input sequence, determining a loss using a standard loss function (e.g., a standard cross-entropy loss function) and a corresponding ground truth splicing event for a reference, and modifying the model parameters according to a loss. Further constraining such training, the training data typically is limited to (or gives no indication of) a particular tissue type or other context type and lacks sufficient volume on intron-retention events to accurately differentiate nucleotide sequences that contribute to intron retention. By using such standard loss functions and undifferentiated training data, existing exon-junction prediction systems cannot facilitate training a machine-learning model to output classifications that go beyond undifferentiated contexts or binary participation in reference splicing.

As existing exon-junction prediction systems typically treat the splice-site prediction task as a binary classification problem, these systems often fail to provide accurate, robust splice site predictions for some input nucleotide sequences. Such accuracy failures are particularly pronounced in predictions that fail to capture relatively complex, alternative splicing or fail to account for such alternative splicing events altogether. For example, while existing systems may be able to identify splice sites, they often fail to generate outputs that accurately reflect how a variant present within a nucleotide sequence affects splicing in less predictable contexts, such as exon skipping, intron retention, exon extension, or intron extension. As but one example, a failure to account for intron retention in generating model outputs tends to lead to an inaccurate representation of the splicing that can occur for an input nucleotide sequence based on intron retention. Indeed, as variants can affect the splicing that occurs-such as the use rate of certain splice sites-existing systems that provide binary classifications fail to accurately portray the impact of these variants with respect to rates of particular nucleobases contributing to reference splicing relative to alternative splicing.

The failed or inaccurate prediction of complex splicing events by existing exon-junction prediction systems has both diagnostic and therapeutic consequences. In many human genes, studies have found intron retention affects gene expression and can cause (or be associated with) complex diseases. For instance, intron retention has been found to fine tune (or otherwise affect) gene expression in thymus-derived (T) cells, smooth muscle cells, and differentiation of neuron and erythroblasts. As further examples, intron retention has been found to cause (or otherwise be associated with) apoE4-linked Alzheimer's disease, Parkinson's disease, and certain types of cancer. Beyond intron retention, exon skipping and other forms of more complex alternative splicing is associated with neurological disorders and many other diseases. Despite the diagnostic and therapeutic consequences of inaccurate splicing predictions, existing exon-junction prediction systems continue to inaccurately predict such complex splicing events based on input pre-mRNA sequences.

Notwithstanding the inaccuracies of some existing models for more complex splicing, existing exon-junction prediction systems typically implement machine-learning models with architectures not well suited for increasing accuracy for predicting complex splicing events based on pre-mRNA sequences. For instance, many existing state-of-the-art systems utilize convolutional neural networks (CNNs) to execute the splice-site prediction task, which have proven less well suited for relatively longer input nucleotide sequences (e.g., 10K bases) than other candidate machine-learning-model architectures. Indeed, CNNs can capture local motifs but sometimes fail to adequately perform analyses that rely on long-range information. As splicing may be based on long-range dependencies within a nucleotide sequence, existing systems that utilize CNNs sometimes fail to flexibly leverage or recognize these long-range dependencies. While other model types, such as transformer models, are known to outperform CNNs in recognizing long-range dependencies, their computational complexity—which scales quadratically with input sequence length—have prevented and complicated biotechnology institutions' efforts to directly apply transformer models to the task of analyzing long input nucleotide sequences (e.g., 4K or 10K base-pairs long) that incorporate such dependencies. Additional problems of instability, slow convergence, and high training loss also make transformer models a challenging fit for the task of predicting splicing events.

By predicting splice site events using CNNs that fail to adequately recognize long-range dependencies, existing exon-junction prediction systems often provide inaccurate results. In particular, by failing to recognize long-range dependencies that may play a role in the splicing of a nucleotide sequence, existing systems often fail to accurately determine whether a given nucleobase is part of a splice site. Thus, the predictions generated by such models can fail to identify splice sites or potentially falsely identify splice sites.

These, along with additional problems and issues exist in existing exon-junction prediction systems.

### SUMMARY

This disclosure describes one or more embodiments of systems, methods, and non-transitory computer readable storage media that solve one or more of the problems described above or provide other advantages over the art. For example, the disclosed systems can utilize a neural network to analyze an input nucleotide sequence (e.g., a precursor messenger ribonucleic acid sequence) and generate conditional probabilities that indicate likelihoods that nucleobases from the input nucleotide sequence are part of a splice site based on a conditioned position. To generate such conditional splice-site probabilities, the disclosed systems can provide, to a splice-site-predictor neural network, an input nucleotide sequence that includes a conditioned nucleobase at a conditioned position-such as a conditioned acceptor or a conditioned donor- and upstream and downstream flanking nucleobases. For each of the upstream or downstream flanking nucleobases, the splice-site-predictor neural network generates a conditional probability indicating a likelihood that the flanking nucleobase is part of a matching donor or a matching acceptor at the conditioned position. In some cases, for instance, the splice-site-predictor neural network generates positive-value probabilities for a few of the flanking nucleobases, but probabilities of zero (or near zero) for the remaining flanking nucleobases. Based on the conditional probabilities for the flanking nucleobases, the disclosed systems can determine that at least one flanking nucleobase is part of the matching donor or the matching acceptor at the conditioned nucleobase.

This disclosure describes different embodiments of implementing such a splice-site-predictor neural network and corresponding conditional splice-site probabilities. In some cases, for instance, the disclosed systems utilize a splice-site-predictor neural network to generate context-specific (e.g., tissue-specific, disease-specific) conditional probabilities. In particular, the disclosed systems can utilize the splice-site-predictor neural network to generate multiple sets of conditional probabilities, where each set corresponds to a different instance of a context. In addition or in the alternative to such a context-specific approach, in some instances, the disclosed systems utilize the splice-site-predictor neural network to model intron retention by generating an additional output that indicates a likelihood that an intron is retained within messenger ribonucleic acid (mRNA) corresponding to the input nucleotide sequence. In some implementations, the disclosed systems utilize, as the splice-site-predictor neural network, a transformer model having an architecture that has been adapted to analyzing relatively longer input nucleotide sequences for splice site predictions.

To train the splice-site-predictor neural network to generate its outputs, in some cases, the disclosed systems employ one or more unique training techniques. For example, to train the splice-site-predictor neural network to generate context-specific conditional probabilities, the disclosed systems can employ multi-task learning, where the splice-site-predictor neural network learns to generate both the context-specific conditional probabilities and genomic track signals from an input nucleotide sequence. Further, to train the splice-site-predictor neural network to generate outputs indicating a likelihood of intron retention, the disclosed systems can augment its training data to include data points indicative of intron retention events. In some instances, the disclosed systems also use one or more loss functions (e.g., a cross-context loss) that adapt neural-network parameters for modeling context-specific probabilities and/or intron retention.

The disclosed systems can leverage the conditional probabilities generated by the splice-site-predictor neural network for more accurate, more specific, and more therapeutic outcomes. For instance, the disclosed systems can utilize conditional probabilities to determine whether a variant is associated with (e.g., at least partially causes) alternative splicing, to determine how splicing differs within different contexts (e.g., different tissue, disease, or cell types), to identify effects of splicing within a genomic host, and/or to discover treatments for undesirable effects.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

This disclosure will describe one or more embodiments of the invention with additional specificity and detail by referencing the accompanying figures. The following paragraphs briefly describe those figures, in which:

FIG. 1 illustrates a schematic diagram of a computing system in which a splice-site prediction system can operate in accordance with one or more embodiments.

FIG. 2 illustrates an overview of the splice-site prediction system generating outputs based on analyzing a nucleotide sequence with a conditioned nucleobase in accordance with one or more embodiments.

FIG. 3 illustrates the splice-site prediction system using a neural network to generate conditional probabilities from a nucleotide sequence in accordance with one or more embodiments.

FIG. 4 illustrates different configurations of nucleotide sequences that can be provided to a neural network in accordance with one or more embodiments.

FIG. 5 illustrates the splice-site prediction system generating a conditioned position identifier for a nucleotide sequence in accordance with one or more embodiments.

FIGS. 6A-6B illustrates example forms of alternative splicing that can be identified using the conditional probabilities generated by the splice-site prediction system in accordance with one or more embodiments.

FIG. 7 illustrates a table and graphs reflecting experimental results regarding the effectiveness of the splice-site prediction system in accordance with one or more embodiments.

FIG. 8 illustrates a table reflecting additional experimental results regarding the effectiveness of the splice-site prediction system in accordance with one or more embodiments.

FIG. 9 illustrates an architecture of a transformer neural network utilized by the splice-site prediction system to analyze nucleotide sequences and generate conditional probabilities in accordance with one or more embodiments.

FIG. 10 illustrates graphs reflecting experimental results regarding the performance of different implementations of a transformer neural network that can be employed by the splice-site prediction system based on the number of residual convolutional blocks included in accordance with one or more embodiments.

FIG. 11A illustrates an architecture of an attention unit utilized by the splice-site prediction system within a transformer neural network in accordance with one or more embodiments.

FIG. 11B illustrates graphs reflecting experimental results regarding the performance of a transformer neural network having various attention unit architectures that can be implemented by the splice-site prediction system in accordance with one or more embodiments.

FIG. 12A illustrates reshaping data within an attention unit of a transformer neural network to reduce the time complexity of the transformer neural network in accordance with one or more embodiments.

FIG. 12B illustrates graphs reflecting experimental results regarding the performance of a transformer neural network that can be implemented by the splice-site prediction system having various data shapes within its attention unit(s) in accordance with one or more embodiments.

FIG. 13 illustrates the splice-site prediction system training a neural network to generate conditional probabilities for nucleotide sequences in accordance with one or more embodiments.

FIG. 14 illustrates alternative transformer neural network architectures that can be implemented by the splice-site prediction system in accordance with one or more embodiments of the present disclosure.

FIGS. 15A-15B illustrates graphs reflecting experimental results regarding the performance of different implementations of a transformer neural network that can be employed by the splice-site prediction system based on training batch size in accordance with one or more embodiments.

FIGS. 16A-16B illustrates graphs reflecting experimental results regarding the performance of different implementations of a transformer neural network that can be employed by the splice-site prediction system based on the learning rate used during training in accordance with one or more embodiments.

FIGS. 17A-17B illustrates graphs reflecting experimental results regarding the performance of different implementations of a transformer neural network that can be employed by the splice-site prediction system based on the stepwise learning rate used during training in accordance with one or more embodiments.

FIG. 18 illustrates a table and a graph reflecting yet further experimental results regarding the performance of different implementations of a transformer neural network that can be employed by the splice-site prediction system in accordance with one or more embodiments.

FIG. 19 illustrates graphs reflecting additional experimental results regarding the performance of different implementations of a transformer neural network that can be employed by the splice-site prediction system in accordance with one or more embodiments.

FIG. 20 illustrates an overview of the splice-site prediction system modeling intron retention for a nucleotide sequence in accordance with one or more embodiments.

FIG. 21 illustrates an example of how intron retention affects splicing within a nucleotide sequence and the significance of modeling intron retention when detecting splice sites in accordance with one or more embodiments.

FIG. 22 illustrates an output generated by a neural network employed by the splice-site prediction system that includes an intron-retention probability for a nucleotide sequence in accordance with one or more embodiments.

FIG. 23 illustrates the splice-site prediction system training a neural network to model intron retention in accordance with one or more embodiments.

FIGS. 24A-24B illustrate the splice-site prediction system determining exon-intron boundary counts in accordance with one or more embodiments.

FIGS. 25A-25C illustrate graphs reflecting experimental results regarding the effectiveness of the splice-site prediction system based on the threshold used in determining the exon-intron boundary counts in accordance with one or more embodiments.

FIG. 26 illustrates the splice-site prediction system performing data augmentation to modify the training data used for training a neural network to model intron retention in accordance with one or more embodiments.

FIGS. 27A-27B illustrate graphs reflecting experimental results regarding the effectiveness of the splice-site prediction system based on techniques used for training a neural network to model intron retention in accordance with one or more embodiments.

FIGS. 28A-28D illustrate graphs reflecting additional experimental results regarding the effectiveness of the splice-site prediction system based on techniques used for training a neural network to model intron retention in accordance with one or more embodiments.

FIG. 29 illustrates a graph reflecting further experimental results regarding the effectiveness of the splice-site prediction system based on techniques used for training a neural network to model intron retention in accordance with one or more embodiments.

FIG. 30 illustrates an overview of the splice-site prediction system modeling tissue-specific splicing for a nucleotide sequence in accordance with one or more embodiments.

FIG. 31 illustrates a motif used by the splice-site prediction system to generate an associated genomic track signal in accordance with one or more embodiments.

FIGS. 32A-32B illustrates graphs reflecting experimental results regarding the effectiveness of the splice-site prediction system in using motifs within a nucleotide sequence to generate tissue-specific conditional probabilities in accordance with one or more embodiments.

FIG. 33 illustrates an example of an enhanced exon and the role of modifying the motif to determine whether the motif is associated with an enhanced exon or a repressed exon in accordance with one or more embodiments.

FIG. 34 illustrates the splice-site prediction system using multi-task learning to train a neural network to generate tissue-specific conditional probabilities and genomic track signals in accordance with one or more embodiments.

FIG. 35 illustrates the splice-site prediction system organizing training data from a data set for training a neural network to generate tissue-specific conditional probabilities in accordance with one or more embodiments.

FIGS. 36A-36B illustrates graphs reflecting experimental results regarding the effectiveness of the splice-site prediction system based on pre-processing of training data in accordance with one or more embodiments.

FIG. 37 Illustrates the splice-site prediction system using logits to evaluate the performance of a neural network in generating tissue-specific conditional probabilities in accordance with one or more embodiments.

FIG. 38 illustrates the splice-site prediction system using probability differences in training a neural network in accordance with one or more embodiments.

FIGS. 39A-39B illustrates graphs reflecting experimental results regarding the effectiveness of the splice-site prediction system based on training of a neural network to generate tissue-specific conditional probabilities in accordance with one or more embodiments.

FIGS. 40A-40B illustrates graphs reflecting experimental results regarding the effectiveness of the splice-site prediction system based on training of a neural network to generate tissue-specific conditional probabilities using a soft-weighed cross-entropy loss in accordance with one or more embodiments.

FIGS. 41A-41B illustrates graphs reflecting additional experimental results regarding the effectiveness of the splice-site prediction system based on training of a neural network to generate tissue-specific conditional probabilities in accordance with one or more embodiments.

FIG. 42 illustrates a graph reflecting further experimental results regarding the effectiveness of the splice-site prediction system based on training of a neural network using a cross-tissue loss in accordance with one or more embodiments.

FIG. 43 illustrates an overview of the splice-site prediction system leveraging probabilities generated for a nucleotide sequence in accordance with one or more embodiments.

FIG. 44 illustrates examples of alternative splicing that can be identified or associated with variant nucleobases by the splice-site prediction system in accordance with one or more embodiments.

FIG. 45 illustrates an example diagram for generating variant-to-phenotype scores using a diagnostic variant model in accordance with one or more embodiments.

FIG. 46 illustrates the splice-site prediction system leveraging context-specific conditional probabilities determined for an artificial nucleotide sequence in accordance with one or more embodiments.

FIG. 47 illustrates a flowchart of a series of acts of generating conditional probabilities for nucleotide sequences indicating likelihoods of matching splice sites in accordance with one or more embodiments.

FIG. 48 illustrates a flowchart of a series of acts of utilizing a transformer neural network to generate conditional probabilities for nucleotide sequences in accordance with one or more embodiments.

FIG. 49 illustrates a flowchart of a series of acts of generating intron-retention probabilities and conditional probabilities for nucleotide sequences in accordance with one or more embodiments.

FIG. 50 illustrates a flowchart of a series of acts of generating context-specific conditional probabilities for nucleotide sequences in accordance with one or more embodiments.

FIG. 51 illustrates a flowchart of a series of acts of leveraging conditional probabilities for a nucleotide sequence to determine whether a variant nucleobase contained therein is associated is alternative splicing in accordance with one or more embodiments.

FIG. 52 illustrates a block diagram of an example computing device in accordance with one or more embodiments.

### DETAILED DESCRIPTION

This disclosure describes one or more embodiments of a splice-site prediction system that utilizes a neural network to output conditional probabilities that nucleobases within an input nucleotide sequence (e.g., a pre-messenger ribonucleic acid sequence) are part of a splice site at a conditioned position. In particular, the splice-site prediction system can utilize the neural network to generate the conditional probabilities given a conditioned (e.g., fixed) nucleobase at a conditioned position within the input nucleotide sequence. In some cases, the conditional probabilities produced by the neural network that the form of context-specific (e.g., tissue-specific, disease-specific) conditional probabilities. In addition to conditional probabilities, in some instances, the neural network further outputs an intron-retention probability indicating the likelihood that an intron from the input nucleotide sequence is retained in mRNA when the input nucleotide sequence is spliced. To facilitate the disclosed conditional splice-site probabilities and/or intron-retention probabilities, in some implementations, the splice-site prediction system utilizes a uniquely constructed transformer neural network that analyzes input nucleotide sequences.

To train the neural network to generate the disclosed outputs, in some embodiments, the splice-site prediction system uses one or more training techniques, including multi-task learning, data augmentation, and/or loss functions (e.g., cross-tissue loss or weighted losses). The following paragraphs give an overview of the splice-site prediction system implementing and training such a splice-site-predictor neural network to generate conditional splice-site probabilities.

As indicated above, in one or more embodiments, the splice-site prediction system utilizes a neural network to generate conditional probabilities from a nucleotide sequence. For instance, the splice-site prediction system can provide a nucleotide sequence, such as a pre-messenger ribonucleic acid (pre-mRNA) sequence, as input to the neural network. In some cases, the nucleotide sequence includes a conditioned nucleobase at a conditioned (i.e., fixed) position and further includes nucleobases that flank the conditioned positioned (e.g., that precede and/or follow the conditioned position). In some instances, the splice-site prediction system provides a separate input that indicates the conditioned position within the nucleotide sequence.

Based on an analysis of the input nucleotide sequence, the splice-site prediction system can generate conditional splice site probabilities. For instance, in some cases, the splice-site prediction system utilizes the neural network to generate a conditional probability for each flanking nucleobase of the nucleotide sequence. Each conditional probability can indicate a likelihood that the corresponding flanking nucleobase is part of a splice site (e.g., a donor or an acceptor). In particular, each conditional probability can indicate that, given the conditioned nucleobase at the conditioned position, the corresponding flanking nucleobase is part of a matching splice site.

By structuring and implementing a neural network to process a nucleotide sequence with a conditioned nucleobase, the splice-site prediction system utilizes the neural network to generate conditional probabilities that a flanking nucleobase constitutes a matching acceptor or a matching donor. When a conditioned nucleobase at a conditioned position is part of a donor, the neural network generates conditional probabilities that a flanking base is either (i) an upstream acceptor or (ii) a downstream acceptor matching the donor. When the conditioned nucleobase at the conditioned position is part of an acceptor, the neural network generates conditional probabilities that a flanking base is either (i) an upstream donor or (ii) a downstream donor matching the acceptor.

As mentioned above, in some implementations, the splice-site prediction system utilizes a transformer neural network to analyze a nucleotide sequence and generate corresponding conditional probabilities. In particular, the splice-site prediction system utilizes a transformer neural network that has been adapted for the task. For instance, in some cases, the transformer neural network includes one or more convolutional blocks to capture the local motifs of the nucleotide sequence. Further, in some instances the transformer neural network includes one or more transformer blocks to capture the long-range dependencies within the nucleotide sequence.

In addition to improved neural-network architecture, in one or more embodiments, the splice-site prediction system utilizes the neural network (e.g., the transformer neural network) to model intron retention. In particular, the splice-site prediction system can use the neural network to generate an output that indicates a likelihood of intron retention for a nucleotide sequence. For instance, in some cases, the splice-site prediction system configures the neural network to generate an output vector that includes an intron-retention probability and the conditional probabilities for a nucleotide sequence. The intron-retention probability and the conditional probabilities can sum to a value of one in some instances, such that the neural network can model how intron retention affects the splicing of a nucleotide sequence (e.g., how intron retention affects the likelihood that the flanking nucleobases will be part of a matching splice site).

To train the neural network to model intron retention, in some cases, the splice-site prediction system employs one or more unique training techniques. For instance, in at least one implementation, the splice-site prediction system augments the training data to include data points that account for intron retention. Indeed, in some cases, the splice-site prediction system utilizes training data that is compatible for training the neural network to learn the splice-site prediction task but does not inherently include data points that are useful for modeling intron retention. Thus, the splice-site prediction system modifies the training data to include such data points (e.g., exon-intron boundary counts and/or proxy positions for exon-intron boundaries). In some implementations, the splice-site prediction system utilizes one or more loss function engineering techniques or data transformation algorithms to improve the ability to predict intron retention events.

As suggested above, unlike the undifferentiated and generalized binary classifications of existing exon-junction prediction system, the splice-site prediction system can generate conditional probabilities specific to a tissue, disease, cell type, or other context. Accordingly, in one or more embodiments, the splice-site prediction system utilizes the neural network to generate context-specific conditional probabilities for a nucleotide sequence. For instance, the splice-site prediction system can utilize the neural network to generate multiple sets of conditional probabilities, where each set corresponds to a different instance of a context and each conditional probability from a set indicates a likelihood that a corresponding flanking nucleobase is part of a splice site that matches the nucleobase at the conditioned position for the particular context instance associated with that set.

To illustrate, in one or more embodiments, the splice-site prediction system utilizes the neural network to generate tissue-specific conditional probabilities from a nucleotide sequence. In particular, the splice-site prediction system can utilize the neural network to generate multiple sets of conditional probabilities, where each set corresponds to a different tissue. Accordingly, each conditional probability in a set of conditional probabilities can indicate a likelihood that a corresponding flanking nucleobase is part of a splice site that matches the nucleobase at the fixed position for the particular tissue associated with that set.

To accompany the tissue-specific conditional probabilities, in some cases, the splice-site prediction system further utilizes the neural network to generate one or more genomic track signals indicating RNA binding site properties, histone associations, or other properties of the input nucleotide sequence. For instance, the splice-site prediction system can utilize the neural network to generate multiple sets of tissue-specific conditional probabilities and one or more genomic track signals (e.g., simultaneously) from the same input nucleotide sequence.

To train the disclosed neural networks to generate context-specific conditional probabilities and/or genomic track signals, in one or more embodiments, the splice-site prediction system implements various training techniques. For instance, in some cases, the splice-site prediction system employs multi-task learning to train the neural network to generate context-specific conditional probabilities and genomic track signals. In some instances, the splice-site prediction system utilizes loss functions—such as a cross-context loss function (e.g., cross-tissue loss function) and/or a soft-weighted cross-entropy loss function—to learn parameters for the neural network for generating these outputs.

Having generated or identified conditional splice site probabilities and/or intron-retention probabilities from the disclosed neural networks, in some embodiments, the splice-site prediction system leverages such probabilities for various therapeutic and research purposes. For instance, in some cases, the splice-site prediction system uses the probabilities to compare a nucleotide sequence with a reference sequence and determine whether a variant included in the nucleotide sequence is associated with (e.g., at least partially causes) alternative splicing. In some cases, the splice-site prediction system can further determine whether the variant is associated with a disease or other disorder of the genomic host of the nucleotide sequence. The splice-site prediction system can also identify options for treating the disease by treating the variant or adjacent nucleobases that may be causing the alternative splicing. For instance, clinicians or researchers can further determine biochemical compounds that target the variant or associated gene affected by such alternative splicing. Additionally, or alternatively, researchers use splice site scores derived from conditional probabilities as features in a model to determine a causal or other relationship between the variant or associated gene and a target phenotype (e.g., a disease).

As indicated above, the splice-site prediction system provides several technical advantages over existing exon-junction prediction systems that, among other things, improve the types of spice-site predictions possible for machine-learning models, deliver a computationally efficient architecture for a transformer neural network, and improve the accuracy of both existing and more complex splice-site predictions. For instance, the splice-site prediction system offers a new, unconventional approach to predicting splice sites with conditional probabilities. Indeed, where existing, state-of-the art systems implement a qualitative approach that frames the task as a binary classification problem, the splice-site prediction system implements a quantitative approach that reframes the task as a conditional probability problem. Thus, the splice-site prediction system implements a neural network that generates conditional probabilities, where existing systems rely on models that generate binary classifications. To illustrate, the splice-site prediction system implements an unconventional ordered combination of steps that involves providing, to a neural network, an input that includes a nucleotide sequence and indicates a conditioned position within the sequence; analyzing the input via the neural network; and generating conditional probabilities that each indicate, given the conditioned position, a likelihood that a corresponding flanking nucleobase of the nucleotide sequence is part of a matching splice site. The splice-site prediction system can further configure the neural network to model intron retention, generate context-specific (e.g., tissue-specific) conditional probabilities, and/or generate genomic track signals-features previously unavailable for the splice-site prediction task under existing systems.

The splice-site prediction system also offers new, unconventional methods of training a neural network for the splice-site prediction task via the generation of conditional probabilities. As noted above, existing exon-junction prediction systems often rely on standard loss functions and reference-only-based ground truths to train a machine-learning model with training data that fails to represent certain splicing events. By contrast, the splice-site prediction system trains its neural network using modified training schemes that allows the neural network to account for relevant data that existing systems fail to consider. For instance, in addition to using short read data for training, the splice-site prediction system trains its neural network with long read data, allowing the neural network to (i) predict a downstream donor for a conditioned acceptor at the conditioned position or (ii) predict an upstream acceptor for a conditioned donor at the conditioned position. Further, the splice-site prediction system employs unconventional data augmentation and loss function engineering techniques to model intron retention and/or unconventionally employs multi-task learning to simultaneously train the neural network to generate tissue-specific conditional probabilities and genomic track signals for the improvement of splice site prediction.

In part due to its unique quantitative approach and training, the splice-site prediction system offers a more flexible approach to splice site prediction that generates more informative outputs when compared to existing systems. Indeed, where existing systems provide binary classifications that are limited to indicating whether a nucleobase may be used as a splice site, the splice-site prediction system flexibly generates conditional probabilities that both identify nucleobases that may be used as a splice site and indicate their usage rates (e.g., represented by the relative conditional probabilities for flanking bases) for splicing based on a conditioned position. The disclosed conditional splice-site probabilities accordingly represent not only more accurate predictions, but also a different type of prediction that accounts for conditional probabilities of flanking nucleobases within a threshold range of a conditioned nucleobase, where the conditional probabilities facilitate comparisons of splicing usage rates unattainable by existing systems. Further, the splice-site prediction system flexibly models intron retention and context-specific splicing. Indeed, the splice-site prediction system utilizes a neural network that has been trained to flexibly account for data or data patterns that provide nuances within an input nucleotide sequence indicative of intron retention or context-specific splicing, which existing models fail to weigh. Overall, by using the quantitative approach and training described herein, the splice-site prediction system can flexibly provide a more complex analysis of long nucleotide sequences than available under existing systems to incorporate the dependencies and nuances associated with long-range motifs and generate numerical outputs representative of that analysis. Indeed, in many cases, the splice-site prediction system analyzes longer input sequences than could be processed by existing systems (e.g., sequence lengths of up to thirty-two thousand nucleobases compared to the ten thousand limit of many existing systems), allowing for a more complex incorporation of dependencies of motifs that exist across large distances.

Further, the splice-site prediction system generates more accurate splice-site predictions compared to existing systems for both (i) pre-mRNA splicing previously predicted with confidence but also (ii) more complex pre-mRNA splicing that existing systems fail to accurately predict. For instance, by generating conditional probabilities rather than binary classifications, the splice-site prediction system gives a more accurate prediction of which position within a nucleotide sequence will be used as a splice site for a splicing event than existing binary classifications. By providing conditional probabilities for a long range of nucleobases flanking a conditioned nucleobase, the splice-site prediction system provides a more accurate indication of how a motif affects splicing within a nucleotide sequence than existing systems-including, but not limited to, conditional probabilities that more accurately predict exon skipping, intron retention, exon extension, or intron extension than existing systems. To illustrate, the splice-site prediction system allows for comparing the predictions of a sequence that includes a variant to predictions for a reference to determine how the usage rate of splice sites change due to the variant. Additionally, by modeling intron retention, the splice-site prediction system improves the accuracy of determining the usage rates of splice sites. Indeed, by recognizing the percentage of splicing events that result in intron retention, the splice-site prediction system more accurately determines the probability a given splice site is used during those splicing events.

Beyond improved accuracy, the splice-site prediction system also offers a new, first-of-its kind model for splice site prediction, including a new transformer neural network architecture that provides improved flexibility and accuracy when compared to models of existing systems. For example, the splice-site prediction system offers a neural network configured to generate conditional probabilities indicative of splicing, rather than binary classifications. Indeed, the splice-site prediction system implements a neural network that generates conditional probabilities that flexibly account for a conditional position within an input sequence. Further, the splice-site prediction system offers a transformer neural network that has been specifically adapted to the task of predicting splice sites based on local motifs and long-term dependencies. To illustrate, the splice-site prediction system implements a transformer neural network that includes convolutional blocks and transformer blocks, where the transformer blocks include additional layer normalization and/or reshaping features that improve upon those areas that typically prevent the practical implementation of transformers for splice site prediction, such as by improving training stability, convergence speed, training losses, and inference time complexity.

Because existing transformer neural networks are typically associated with large time and space complexities (e.g., on the order of O(n2)), in some embodiments, the disclosed transformer neural network includes unique features that improves performance during inference and/or training for relatively longer nucleotide sequences (e.g., 3K base-pairs, 4K base-pairs, or more). For instance, in some cases, the disclosed transformer neural network adds layer normalization (LayerNorm) to the linear/quadratic output generated within an attention unit (e.g., FLASH) of each transformer block, which improves the convergence speed of the network and leads to a lower valid loss. Further, the transformer neural network can reshape tensors within the attention unit to reduce the computation on attention and facilitate a deeper model (e.g., up to 80 layers) that gives lower valid loss. As explained further below, the transformer neural network can reshape or chunk data of a tensor having dimensions for batch size, sequence length, and an attention dimension according to a reshaping factor. Such data reshaping reduces the computational complexity of the attention operation within the attention unit based on a reshaping factor while preserving the data represented in the tensor. The disclosed transformer neural network can further apply the reshaping to a query tensor, a key tensor, and a value tensor.

In addition to a more computationally efficient architecture relative to other transformer neural networks, the splice-site prediction system offers a more accurate model for splice site prediction. Indeed, when compared to the models implemented by existing systems—including state-of-the-art models—the neural network utilized by the splice-site prediction system provides a more accurate prediction of the splice sites for a nucleotide sequence. The accompanying figures and descriptions below illustrate and quantify various examples of more accurate splice site predictions.

As illustrated by the foregoing discussion, the present disclosure utilizes a variety of terms to describe features and advantages of the splice-site prediction system. As used herein, for example, the term “neural network” refers to a type of machine learning model, which can be tuned (e.g., trained) based on inputs to approximate unknown functions used for generating the corresponding outputs. In particular, a neural network can include a model of interconnected artificial neurons (e.g., organized in layers) that communicate and learn to approximate complex functions and generate outputs based on a plurality of inputs provided to the model. In some instances, a neural network includes one or more machine learning algorithms. Further, in some cases, a neural network includes an algorithm (or set of algorithms) that implements deep learning techniques that utilize a set of algorithms to model high-level abstractions in data. Example neural networks include a convolutional neural network, a recurrent neural network (e.g., a long short-term memory neural network), a generative adversarial neural network, a graph neural network, or a transformer neural network. In some embodiments, a neural network includes a combination of neural networks or neural network components.

In some cases, the splice-site prediction system utilizes a splice-site-predictor neural network to generate conditional probabilities or other outputs. As used herein, the term “splice-site-predictor neural network” refers to a neural network that generates conditional probabilities indicating likelihoods that nucleobases from an input nucleotide sequence are part of a splice site based on a conditioned position. For example, in some cases, the splice-site-predictor neural network is trained to generate conditional probabilities based on training nucleotide sequences. As explained below, a splice-site-predictor neural network may also generate intron-retention probabilities and/or genomic track signals. As further set forth below, a splice-site-predictor neural network may take the form of a transformer neural network. But a splice-site-predictor neural network can also take the form of a convolutional neural network, a recurrent neural network, or other neural networks noted above.

As further used herein, the term “alternative splicing” refers to a splicing of pre-mRNA that differs from a reference or baseline splicing of pre-mRNA. For instance, alternative splicing can include a splicing of pre-mRNA that removes introns from and includes exons in processed mRNA with a different sequence from a reference or baseline mRNA that includes different exons. Consequently, alternative splicing can produce isoforms comprising an alternative amino-acid sequence that differs from a reference or baseline amino-acid sequence for a protein. In some embodiments, alternative splicing can be one or both of qualitative and quantitative alternative splicing. In quantitative alternative splicing, the pre-mRNA can be spliced at different rates such that a given acceptor or given donor within the pre-mRNA marks a splice site at a different rate in comparison to an alternative acceptor or donor within the pre-mRNA relative to reference or baseline splicing. In qualitative alternative splicing, the pre-mRNA can be spliced to produce a processed mRNA sequence (and corresponding isoform) that differs from a processed mRNA sequence (and corresponding isoform) produced by reference or baseline splicing. As indicated above, some forms of alternative splicing cause or are otherwise associated with a disease. Further, alternative splicing can be context specific in that the quantitative rate of splicing pre-mRNA at different sites or qualitative isoform produced by splicing pre-mRNA in one tissue type, cell type, cell-line type, disease type, etc. can represent reference splicing, whereas the same quantitative rate or qualitative isoform in another tissue type, cell type, cell-line type, disease type, etc. can represent alternative splicing.

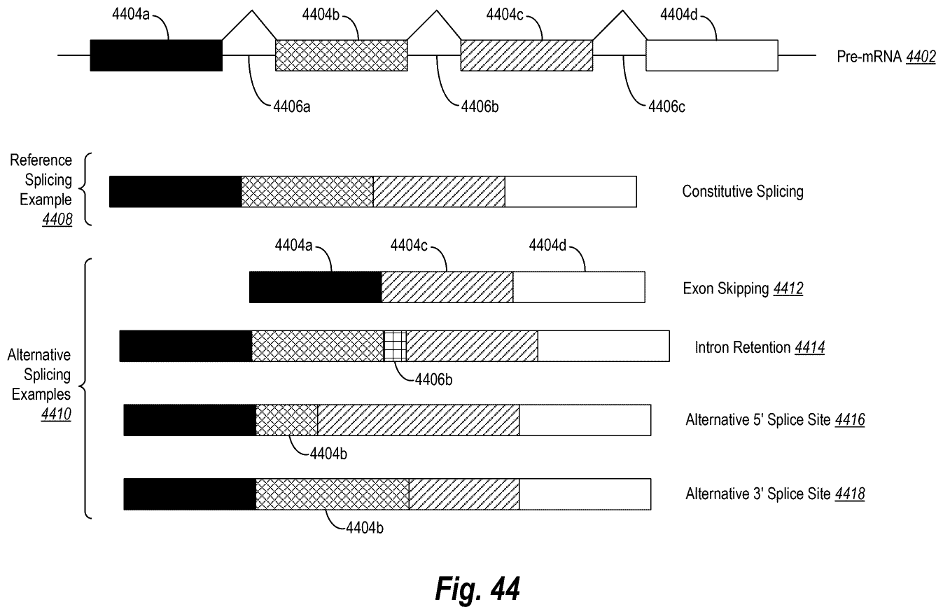

By contrast, the term “reference splicing” refers to a splicing of pre-mRNA resulting from a reference pre-mRNA sequence or refers to a standard type of splicing pre-mRNA. For instance, reference splicing can include a splicing of pre-mRNA that removes introns from and includes exons in processed mRNA consistent with data or observations of splicing a reference pre-mRNA sequence based on a reference genome (e.g., from a human transcriptome database). In some embodiments, reference splicing can be one or both of qualitative and quantitative reference splicing. In quantitative reference splicing, a reference pre-mRNA sequence can be spliced at a given acceptor or given donor at a different rate than an alternative acceptor or donor within the reference pre-mRNA sequence, as indicated by data or observations of splicing the reference pre-mRNA sequence. In qualitative reference splicing, a reference pre-mRNA sequence can be spliced to produce a processed mRNA sequence (and corresponding isoform) consistent with other processed mRNA sequences (and corresponding isoforms) observed in data for reference pre-mRNA sequence. As described below, FIG. 44 provides further examples of reference splicing and alternative splicing.

Additionally, as used herein, the term “conditioned position” refers to a fixed or designated position within a group of available positions. For instance, a conditioned position can include a designated position within various positions within a nucleotide sequence. To illustrate, a conditioned position can include a designated position within a pre-mRNA sequence, where each nucleobase within the pre-mRNA sequence is associated with a position (i.e., its position within the pre-mRNA sequence). Accordingly, as used herein, the term “conditioned nucleobase” refers to a nucleobase that is at a conditioned position within a nucleotide sequence. In some cases, a conditioned nucleobase is associated with a splice site of a nucleotide sequence. In other words, the conditioned nucleobase at the conditioned position is part of a donor or an acceptor of the nucleotide sequence. A conditioned position, however, can be associated with non-splice-site nucleobases in many instances. For instance, the conditioned nucleobase at the conditioned position can include a nucleobase in the interior of an exon or an intron, rather than at an exon-intron boundary.

As further used herein, the term “conditioned position identifier” refers to an identifier of a conditioned position within a nucleotide sequence. In particular, a conditioned position identifier can include an identifier that is associated with a nucleotide sequence and identifies the location of a conditioned position within the nucleotide sequence. For instance, where a nucleotide sequence is provided as input to a neural network, a conditioned position identifier can include additional input that flags or otherwise identifies the conditioned position of the nucleotide sequence for the neural network. To illustrate, in some cases, a conditioned position identifier includes a one hot encoding vector or a portion of a one hot encoding vector that indicates the position within a nucleotide sequence that is the conditioned position. Other examples of a conditioned position identifier include an index value where the positions of a nucleotide sequence are indexed, or a vector of distance values where each distance value in the vector is associated with a position within a nucleotide sequence and indicates a distance between the position and the conditioned position within the nucleotide sequence.

Additionally, as used herein, the term “conditioned donor” refers to a donor associated with a conditioned position within a nucleotide sequence for purposes of predicting pre-mRNA splicing. In particular, a conditioned donor can include a donor having a conditioned nucleobase at the conditioned position. To illustrate, in some cases, a conditioned donor includes a dinucleotide corresponding to a donor that includes a conditioned nucleobase at a conditioned position.

Similarly, as used herein, the term “conditioned acceptor” refers to an acceptor associated with a conditioned position within a nucleotide sequence for purposes of predicting pre-mRNA splicing. In particular, a conditioned acceptor can include an acceptor having a conditioned nucleobase at the conditioned position. To illustrate, in some cases, a conditioned acceptor includes a dinucleotide corresponding to an acceptor that includes a conditioned nucleobase at a conditioned position.

As used herein, the term “flanking nucleobase” refers to a nucleobase that flanks a conditioned nucleobase within a nucleotide sequence. In particular, a flanking nucleobase can include a nucleobase that is at a position within a nucleotide sequence other than a conditioned position. In some cases, a flanking nucleobase includes a nucleobase that is part of a particular portion of a nucleotide sequence other than a conditioned nucleobase and some other portion of the nucleotide sequence. For instance, in some cases, a nucleotide sequence includes a conditioned nucleobase, a set of flanking nucleobases, and one or more additional portions, such as a set of nucleobases that make up a context sequence. In some cases, a flanking nucleobase includes a nucleobase that is upstream or downstream from a conditioned nucleobase within a threshold number of nucleobases.

Additionally, as used herein, the term “matching donor” refers to a donor that matches a conditioned nucleobase within a nucleotide sequence for purposes of predicting pre-mRNA splicing. In particular, a matching donor can include a splice site that may be used as a donor during splicing of a nucleotide sequence given the conditioned nucleobase at the conditioned position within the nucleotide sequence. In some cases, a matching donor includes a splice site that may be used as the most proximate donor to the conditioned nucleobase during splicing based on the direction from the conditioned nucleobase under consideration (e.g., upstream or downstream). In other words, a matching donor can include a splice site that may be used as the next donor upstream from the conditioned nucleobase or the next donor downstream from the conditioned nucleobase during splicing.

Also, as used herein, the term “matching acceptor” refers to an acceptor that matches a conditioned nucleobase within a nucleotide sequence for purposes of predicting pre-mRNA splicing. In particular, a matching acceptor can include a splice site that may be used as an acceptor during splicing of a nucleotide sequence given the conditioned nucleobase at the conditioned position within the nucleotide sequence. In some cases, a matching donor includes a splice site that may be used as the most proximate acceptor to the conditioned nucleobase during splicing based on the direction from the conditioned nucleobase under consideration (e.g., upstream or downstream). In other words, a matching acceptor can include a splice site that may be used as the next acceptor upstream from the conditioned nucleobase or the next acceptor downstream from the conditioned nucleobase during splicing.

As used herein, the term “conditional probability” refers to a measure of a probability of an event based on a condition. In particular, a conditional probability can include a probability that an event has occurred or will occur based on a condition. For instance, a conditional probability can be associated with a nucleobase within a nucleotide sequence and indicate a likelihood that the nucleobase is part of a splice site based on a conditioned nucleobase at a conditioned position within the nucleotide sequence. Indeed, in some cases, a conditional probability indicates a likelihood that the associated nucleobase is part of a matching donor or a matching acceptor corresponding to a conditioned nucleobase within a nucleotide sequence. In some instances, as will be discussed in more detail below, a conditional probability corresponds to a usage rate of a splice site based on a conditioned nucleobase at a conditioned position.

A conditional probability can refer to one or both nucleobases that are part of a matching donor or a matching acceptor corresponding to a conditioned nucleobase at a conditioned position. In some embodiments, for instance, a conditional probability indicating a likelihood that a flanking nucleobase is part of a matching donor corresponding to a conditioned nucleobase represents a conditional probability that the flanking nucleobase is a guanine (G) from a guanine-uracil (GU) pair or a guanine-thymine (GT) pair of the matching donor. Alternatively, in some implementations, a conditional probability indicating a likelihood that a flanking nucleobase is part of a matching donor corresponding to a conditioned nucleobase represents a conditional probability that the flanking nucleobase is one of a G and U from a GU pair of the matching donor. Likewise, in some implementations, a conditional probability indicating a likelihood that a flanking nucleobase is part of a matching donor corresponding to a conditioned nucleobase represents a conditional probability that the flanking nucleobase is one of a G and T from a GT pair of the matching donor.

By contrast, in some embodiments, a conditional probability indicating a likelihood that a flanking nucleobase is part of a matching acceptor corresponding to a conditioned nucleobase represents a conditional probability that the flanking nucleobase is a guanine (G) from an adenine-guanine (AG) pair of the matching acceptor. Alternatively, in some implementations, a conditional probability indicating a likelihood that a flanking nucleobase is part of a matching acceptor corresponding to a conditioned nucleobase represents a conditional probability that the flanking nucleobase is one of a G and A from an AG pair of the matching acceptor.

As used herein, the term “feature vector” refers to a set of numerical values representing features of an input to a neural network. To illustrate, a feature vector can include a set of values corresponding to latent and/or patent attributes and characteristics of an input to a neural network (e.g., an input nucleotide sequence). In some cases, a neural network generates a feature vector based on an analysis of the input or based on an analysis of a previously generated feature vector. Indeed, in some cases, a neural network generates a feature vector internally and utilizes the feature vector for further processing (e.g., to generate a neural network output or to generate a subsequent feature vector). As used herein, the term “convolutional feature vector” refers to a feature vector generated by one or more convolutional blocks or layers of a neural network. Similarly, as used herein, the term “attention-based feature vector” refers to a feature vector generated by one or more attention blocks or layers of a neural network. For instance, an attention-based feature vector can include a feature vector generated by a transformer block of a neural network.

Additionally, as used herein, the term “reshaping factor” refers to a factor for reorganizing or changing dimensions of a collection of values generated within a neural network. In particular, a reshaping factor can include a factor for reorganizing data within a collection of data, such as a feature tensor. For instance, a reshaping factor can include a factor for reorganizing data from one dimension of a feature tensor under another dimension of the feature tensor.

Further, as used herein, the term “intron-retention probability” refers to a probability that indicates a likelihood that a nucleotide sequence retains an intron. In particular, an intron-retention probability can indicate a likelihood that an intron is retained in messenger ribonucleic acid (mRNA) corresponding to the nucleotide sequence. Indeed, in some cases, a nucleotide sequence includes a pre-messenger ribonucleic acid (pre-mRNA) sequence, and an intron-retention probability indicates a likelihood that an intron within the pre-mRNA sequence is retained after splicing (i.e., is still present within the resulting mRNA after splicing).

As further used herein, the term “exon-intron boundary count” includes a value corresponding to a number of nucleotide reads for a nucleotide sequence that represent intron retention. In particular, an exon-intron boundary count can include a value corresponding to a number of nucleotide reads for a nucleotide sequence that extend into an intron of the nucleotide sequence. Indeed, in some cases, where the nucleotide reads for a nucleotide sequence correspond to spliced mRNA reads, an exon-intron boundary count corresponds to how many of the nucleotide reads contain at least a portion of the intron included in the corresponding pre-mRNA. In some cases, an exon-intron boundary count represents an estimation of the number of nucleotide reads within the read data of a training dataset that represent intron retention. The term “exon-intron boundary” can include a boundary between an exon and a downstream intron as well as a boundary between an intron and a downstream exon.

Additionally, as used herein, the term “exon-intron boundary proxy” refers to a position within a nucleotide sequence that does not correspond to a boundary between an exon and an intron but is treated as a proxy for such a boundary for some purpose. In other words, an exon-intron boundary proxy can act as a stand-in for a boundary between an exon and an intron despite its positioning away from the boundary. For instance, an exon-intron boundary proxy can be positioned within the interior of an exon or intron of a nucleotide sequence. As will be discussed in more detail below, however, the splice-site prediction system can treat an exon-intron boundary proxy as corresponding to a boundary between an exon and an intron for one or more purposes related to training a neural network to model intron retention.

As used herein, the term “context” refers to an attribute or a classification of attributes associated with (i) a genomic sample for which a nucleotide sequence is extracted or synthesized or (ii) a biological assay or test. In particular, a context can refer to an attribute class having multiple distinguishable instances or class members. To illustrate, example contexts can include, but are not limited to, tissues, sub-tissues, diseases, disease categories, disease severities, cell-types, cell-lines, and/or assay types. In some instances, a context refers more specifically to a member or instance of an attribute class, such as a particular tissue, sub-tissue, disease, disease category, disease severity, cell-type, cell-line, or assay type.

As further used herein, the term “context instance” refers to an attribute within an attribute class associated with (i) a genomic sample for which a nucleotide sequence is extracted or synthesized or (ii) a biological assay or test. In particular, a context instance can refer to a member of an attribute class that share similarities to other members of the attribute class but is distinguishable from those other members. To illustrate, example context instances can include, but are not limited to, different tissues, sub-tissues, diseases, disease categories, disease severities, cell-types, cell-lines, and/or assay types.

Relatedly, as used herein, the term “context-specific conditional probability” refers to a conditional probability generated with respect to a particular context or instance of a context. In particular, a context-specific conditional probability can include a conditional probability that is associated with a nucleobase within a nucleotide sequence and indicates a likelihood that the nucleobase is part of a splice site with respect to a particular context or context instance associated with the nucleotide sequence. Indeed, as will be described in more detail below, the splice-site prediction system can utilize a neural network to generate multiple sets of context-specific conditional probabilities from a nucleotide sequence where each set of context-specific conditional probabilities corresponds to a different instance of a context and includes conditional probabilities with respect to that context instance. Example contexts can include, but are not limited to, tissues, sub-tissues, diseases, disease categories, disease severities, cell-types, cell-lines, and/or assay types. Thus, context instances can include, but are not limited to different tissues, sub-tissues, diseases, disease categories, disease severities, cell-types, cell-lines, and/or assay types. To illustrate, the splice-site prediction system can generate multiple sets of context-specific conditional probabilities for multiple tissues, sub-tissues, diseases, disease categories, disease severities, cell-types, cell-lines, or assay types where each set of context-specific conditional probabilities can represent, respectively, a different tissue, sub-tissue, disease, disease category, disease severity, cell-type, cell-line, or assay type.

As a context can include tissues, a context-specific conditional probability can include a tissue-specific conditional probability. As used herein, the term “tissue-specific conditional probability” refers to a conditional probability generated with respect to a particular tissue. In particular, a tissue-specific conditional probability can include a conditional probability that is associated with a nucleobase within a nucleotide sequence and indicates a likelihood that the nucleobase is part of a splice site with respect to a particular tissue associated with the nucleotide sequence. Indeed, as will be described in more detail below, the splice-site prediction system can utilize a neural network to generate multiple sets of tissue-specific conditional probabilities from a nucleotide sequence where each set of tissue-specific conditional probabilities corresponds to a different tissue and includes conditional probabilities with respect to that tissue.

Additionally, as used herein, the term “tissue-specific spliced isoform” refers to a tissue-specific splicing result. For instance, as will be discussed in more detail below, a nucleotide sequence, such as a pre-mRNA sequence can experience splicing differently within different tissues. As a consequence, the mRNA sequence that results from the splicing can differ across different tissues. Thus, a tissue-specific spliced isoform can include the mRNA sequence that results from splicing with respect to a particular tissue.

Further, as used herein, the term “genomic track signal” includes a value or set of values that indicate one or more properties of a nucleotide sequence, including epigenetic level signals. In particular, a genomic track signal can include a value or set of values that represent one or more latent and/or patent characteristics of a nucleotide sequence, such as an epigenetic characteristic of the nucleotide sequence. A genomic track signal can include a set of continuous values or a set of discrete values. A genomic track signal can correspond to various properties or various combination of properties of a nucleotide sequence. But some examples of genomic track signals include ribonucleic acid (RNA) binding protein signals that indicate probabilities that positions within a nucleotide sequence correspond to a binding site for an RNA binding protein or histone mark signals that indicate probabilities that positions within the nucleotide sequence correspond to a histone mark. As further examples, genomic track signals can include other epigenetic signals, such as indicators or probabilities that positions within a nucleotide sequence correspond to one or more of DNase I-hypersensitive sites (DHSs), sites transcription factor (TF) bindings, levels of histone modification (HM) marks, DNA methylation marks.

As further used herein, the term “variant” refers to a nucleobase or multiple nucleobases that differs or varies from a corresponding nucleobase (or nucleobases) in a reference sequence or a reference genome. For example, a variant includes a SNP, an indel, or a structural variant that indicates nucleobases in a nucleotide sequence that differs from nucleobases in corresponding genomic coordinates of a reference sequence.

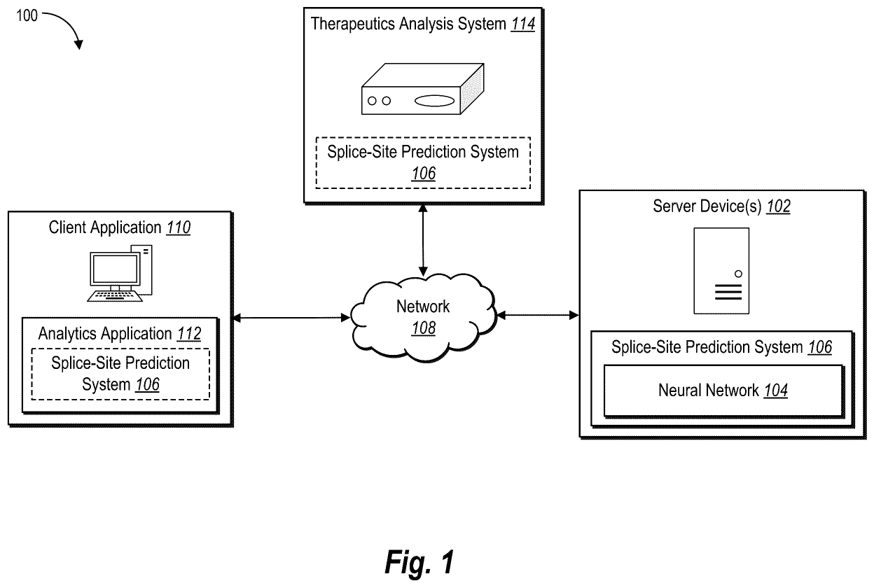

The following paragraphs describe the splice-site prediction system with respect to illustrative figures that portray example embodiments and implementations. FIG. 1 illustrates a schematic diagram of a computing system 100 in which a splice-site prediction system 106 operates in accordance with one or more embodiments. As illustrated, the computing system 100 includes one or more server device(s) 102 connected to a client device 110 and therapeutics analysis device(s) 114 via a network 108. While FIG. 1 shows an embodiment of the splice-site prediction system 106, this disclosure describes alternative embodiments and configurations below.

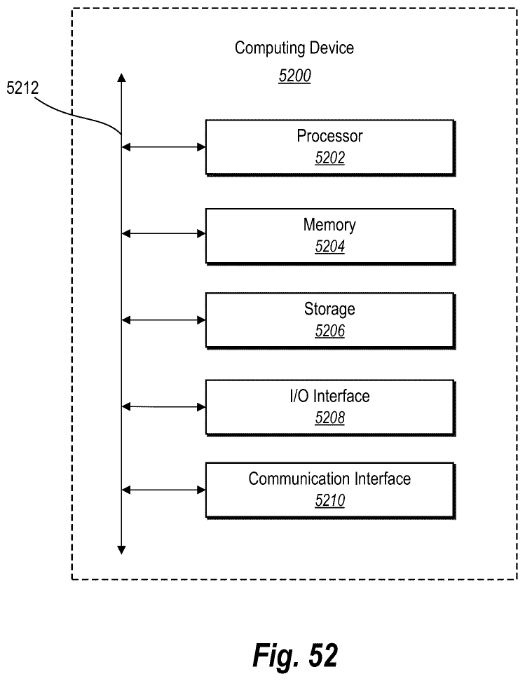

As shown in FIG. 1, the server device(s) 102, the client device 110, and the therapeutics analysis device(s) 114 are connected via the network 108. Accordingly, each of the components of the computing system 100 can communicate via the network 108. The network 108 comprises any suitable network over which computing devices can communicate. Example networks are discussed in additional detail below with respect to FIG. 52.

As indicated by FIG. 1, the therapeutics analysis device(s) 114 comprises a device for analyzing (and identifying candidate therapeutics for) amino-acid sequences corresponding to proteins and/or nucleotide sequences representing coding and non-coding genomic regions. In some embodiments, the therapeutics analysis device(s) 114 analyzes a set of amino-acid sequences or a set of nucleotide sequences from a database comprising samples exhibiting genetic diversity. From among the analyzed set of amino-acid sequences and/or analyzed set of nucleotide sequences, the therapeutics analysis device(s) 114 can identify subsets of amino-acid sequences and/or nucleotide sequences exhibiting common variant amino acids or variant nucleotides. In combination with or separate from such variant identification, the therapeutics analysis device(s) 114 can execute machine-learning models (or other models) that identify coding or non-coding genomic regions that are intolerant to variation and for which variants can cause loss or change in biological functions. For identified subsets of amino-acid sequences and/or nucleotide sequences, in some cases, the therapeutics analysis device(s) 114 identifies candidate biologics, drugs, or gene-editing protocols for treatment.

In addition, or in the alternative to communicating across the network 108, in some embodiments, the therapeutics analysis device(s) 114 bypasses the network 108 and communicates directly with the server device(s) 102 or the client device 110. Additionally, as shown in FIG. 1, in one or more embodiments, the therapeutics analysis device(s) 114 includes the splice-site prediction system 106.

As further indicated by FIG. 1, the server device(s) 102 may generate, receive, analyze, store, and transmit digital data, such as data for amino-acid sequences or nucleotide sequences. As shown in FIG. 1, the therapeutics analysis device(s) 114 may send (and the server device(s) 102 may receive) various data from the therapeutics analysis device(s) 114, including data representing amino-acid sequences or nucleotide sequences. The server device(s) 102 may also communicate with the client device 110. In particular, the server device(s) 102 can send data representing amino-acid sequences or nucleotide sequences (or variants thereof), conditional probabilities (including tissue-specific conditional probabilities), genomic track signals, or intron-retention probabilities to the client device 110.

Additionally, as shown in FIG. 1, the server device(s) 102 can include the splice-site prediction system 106. In one or more embodiments, as explained further below, the splice-site prediction system 106 analyzes a nucleotide sequence having a conditioned nucleobase at a conditioned position and generates conditional probabilities for flanking nucleobases of the nucleotide sequence that each indicate a likelihood that an associated flanking nucleobase is part of a matching splice site for the conditioned nucleobase. In particular, in some embodiments, the splice-site prediction system 106 employs a neural network 104 to analyze the nucleotide sequence and generate the conditional probabilities. For instance, in some cases, the splice-site prediction system 106 employs a transformer neural network as the neural network 104, such as a transformer neural network having an architecture that has been adapted to the task of generating conditional probabilities from a nucleotide sequence. In some implementations, the splice-site prediction system 106 trains the neural network 104 to also model intron retention by generating an intron-retention probability for the nucleotide sequence. Further, in some instances, the splice-site prediction system 106 trains the neural network 104 to model differences in splicing for different tissues by generating tissue-specific conditional probabilities for the nucleotide sequence. For instance, the splice-site prediction system 106 can train the neural network 104 via multi-task learning to generate tissue-specific conditional probabilities and genomic track signals from the same nucleotide sequence.

In some embodiments, the server device(s) 102 comprise a distributed collection of servers where the server device(s) 102 include a number of server devices distributed across the network 108 and located in the same or different physical locations. Further, the server device(s) 102 can comprise a content server, an application server, a communication server, a web-hosting server, or another type of server.

In some cases, the server device(s) 102 is located at or near a same physical location of the therapeutics analysis device(s) 114 or remotely from the therapeutics analysis device(s) 114. Indeed, in some embodiments, the server device(s) 102 and the therapeutics analysis device(s) 114 are integrated into a same computing device. The server device(s) 102 may run software on the therapeutics analysis device(s) 114 or the splice-site prediction system 106 to generate, receive, analyze, store, and transmit digital data, such as by sending or receiving data representing amino-acid sequences or nucleotide sequences (or variants thereof), conditional probabilities (including tissue-specific conditional probabilities), genomic track signals, or intron-retention probabilities. Additionally, or alternatively, in some embodiments, the therapeutics analysis device(s) 114 or the splice-site prediction system 106 store and access a database or table of conditional probabilities, genomic track signals, and/or intron-retention probabilities corresponding to particular proteins and/or protein positions.

As further illustrated and indicated in FIG. 1, the client device 110 can generate, store, receive, and send digital data. In particular, the client device 110 can receive data for amino-acid sequences or nucleotide sequences (or variants thereof), conditional probabilities (including tissue-specific conditional probabilities), genomic track signals, or intron-retention probabilities from the server device(s) 102 and/or the therapeutics analysis device(s) 114. The client device 110 can accordingly present data concerning conditional probabilities (including tissue-specific conditional probabilities), genomic track signals, or intron-retention probabilities within a graphical user interface to a user associated with the client device 110.

The client device 110 illustrated in FIG. 1 may comprise various types of client devices. For example, in some embodiments, the client device 110 includes non-mobile devices, such as desktop computers or servers, or other types of client devices. In yet other embodiments, the client device 110 includes mobile devices, such as laptops, tablets, mobile telephones, or smartphones. Additional details with regard to the client device 110 are discussed below with respect to FIG. 52.

As further illustrated in FIG. 1, the client device 110 includes an analytics application 112. The analytics application 112 may be a web application or a native application stored and executed on the client device 110 (e.g., a mobile application, desktop application). The analytics application 112 can include instructions that (when executed) cause the client device 110 to receive data from the splice-site prediction system 106 and present data from the therapeutics analysis device(s) 114 and/or the server device(s) 102. Furthermore, the analytics application 112 can instruct the client device 110 to display data for conditional probabilities (including tissue-specific conditional probabilities), genomic track signals, or intron-retention probabilities, such as data for an intron-retention indicator that a gene of a genomic sample associated with a nucleotide sequence comprises a variant associated with the mRNA retaining an intron after splicing.

As further illustrated in FIG. 1, the splice-site prediction system 106 may be located on the client device 110 as part of the analytics application 112 or on the therapeutics analysis device(s) 114. Accordingly, in some embodiments, the splice-site prediction system 106 is implemented by (e.g., located entirely or in part) on the client device 110. As mentioned, in yet other embodiments, the splice-site prediction system 106 is implemented by one or more other components of the computing system 100, such as the therapeutics analysis device(s) 114. In particular, the splice-site prediction system 106 can be implemented in a variety of different ways across the server device(s) 102, the network 108, the client device 110, and the therapeutics analysis device(s) 114.

Though FIG. 1 illustrates the components of the computing system 100 communicating via the network 108, in certain implementations, the components of computing system 100 can also communicate directly with each other, bypassing the network 108. For instance, and as previously mentioned, in some implementations, the client device 110 communicates directly with the therapeutics analysis device(s) 114. Additionally, in some embodiments, the client device 110 communicates directly with the splice-site prediction system 106. Moreover, the splice-site prediction system 106 can access one or more databases housed on or accessed by the server device(s) 102 or elsewhere in the computing system 100.

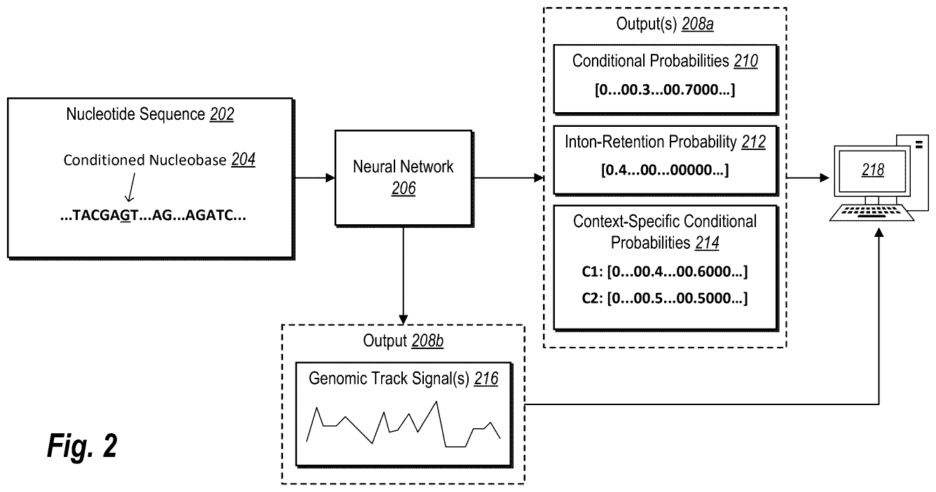

As discussed above, the splice-site prediction system 106 can analyze an input nucleotide sequence having a conditioned nucleobase at a conditioned position and generate outputs based on the analysis. FIG. 2 illustrates an overview of the splice-site prediction system 106 generating outputs based on analyzing a nucleotide sequence with a conditioned nucleobase in accordance with one or more embodiments.

As shown in FIG. 2, the splice-site prediction system 106 analyzes a nucleotide sequence 202. In particular, as shown, the nucleotide sequence 202 includes a conditioned nucleobase 204 at a conditioned position. In one or more embodiments, the conditioned nucleobase 204 includes a nucleobase upon which analysis of the nucleotide sequence 202 is based. For instance, the splice-site prediction system 106 can analyze the nucleotide sequence 202 to detect one or more matching splice sites (e.g., matching donors or matching acceptors) that correspond to the conditioned nucleobase 204.

As further shown in FIG. 2, the splice-site prediction system 106 analyzes the nucleotide sequence 202 utilizing a neural network 206. As will be discussed in more detail below, the splice-site prediction system 106 can configure the neural network 206 for the task of generating outputs based on an analysis of the nucleotide sequence 202. For instance, as mentioned, the splice-site prediction system 106 utilizes, as the neural network 206, a transformer neural network that has been adapted to the task of generating outputs from nucleotide sequences in some embodiments. In particular, the splice-site prediction system 106 can utilize a transformer neural network having architectural components and features that allow for its practical implementation in the task of splice site detection.

As illustrated in FIG. 2, based on the analysis of the nucleotide sequence 202, the splice-site prediction system 106 generates one or more of the output(s) 208a and/or the output 208b. In particular, the splice-site prediction system 106 can utilize the neural network 206 to generate one or more of the output(s) 208a and/or the output 208b.

As shown, the output(s) 208a include conditional probabilities 210, an intron-retention probability 212, and/or context-specific conditional probabilities 214. FIG. 2 represents the output(s) 208a as vectors containing corresponding values. Indeed, as will be explained in more detail below, in some cases, the splice-site prediction system 106 utilizes the neural network 206 to generate one or more output vectors populated with values. In the case of the conditional probabilities 210 and the context-specific conditional probabilities 214, the values can indicate a likelihood that flanking nucleobases within the nucleotide sequence 202 are part of a matching donor or a matching acceptor for the conditioned nucleobase 204. In the case of the intron-retention probability 212, the values can indicate a likelihood that an intron is retained as the nucleotide sequence 202 (e.g., a pre-mRNA sequence corresponding to the nucleotide sequence 202) is spliced.

Though FIG. 2 illustrates the output(s) 208a as vectors, it should be understood that the splice-site prediction system 106 can generate the output(s) 208a in other formats. For instance, in some cases, the splice-site prediction system 106 generates the output(s) 208a as single values with associated labels or other textual information that indicates an associated relevance (e.g., “intron-retention probability=0.3”). In some cases, the splice-site prediction system 106 uses the neural network 206 to generate the output(s) 208a in a vector format and then generates one or more outputs in another format utilizing the vector generated by the neural network 206. In some cases, the splice-site prediction system 106 configures the neural network 206 to directly generate the output(s) 208a in a format other than the vector format shown in FIG. 2.

In one or more embodiments, the splice-site prediction system 106 uses the neural network 206 to generate one of the output(s) 208a in isolation or to generate a combination of the output(s) 208a. For example, as will be discussed further below, the splice-site prediction system 106 generates a single output vector that includes the conditional probabilities 210 and the intron-retention probability 212 in some embodiments.

As further shown, the output 208b includes genomic track signal(s) 216. Indeed, in one or more embodiments, the splice-site prediction system 106 utilizes the neural network 206 to generate one or more genomic track signals based on its analysis of the nucleotide sequence 202. In some cases, as will be explained further below, the splice-site prediction system 106 uses the neural network 206 to generate the genomic track signal(s) 216 with one of the output(s) 208a. For instance, in some embodiments, the splice-site prediction system 106 uses the neural network 206 to generate the genomic track signal(s) 216 along with generating the context-specific conditional probabilities 214 based on its analysis of the nucleotide sequence 202. Thus, in some instances, the splice-site prediction system 106 utilizes the neural network 206 to extract various types of information from the nucleotide sequence 202 and use the extracted information to generate multiple output types.

As illustrated by FIG. 2, the splice-site prediction system 106 provides the output(s) 208a and/or the output 208b to a computing device 218. For instance, the splice-site prediction system 106 can provide the output(s) 208a and/or the output 208b for display within a graphical user interface of a client device. In some cases, the splice-site prediction system 106 provides the output(s) 208a and/or the output 208b for display as textual or graphical elements. In some cases, the splice-site prediction system 106 utilizes the output(s) 208a and/or the output 208b to determine that the nucleotide sequence 202 (or a genomic sample associated with the nucleotide sequence 202) includes a variant and provides an indicator of the variant to the computing device 218.

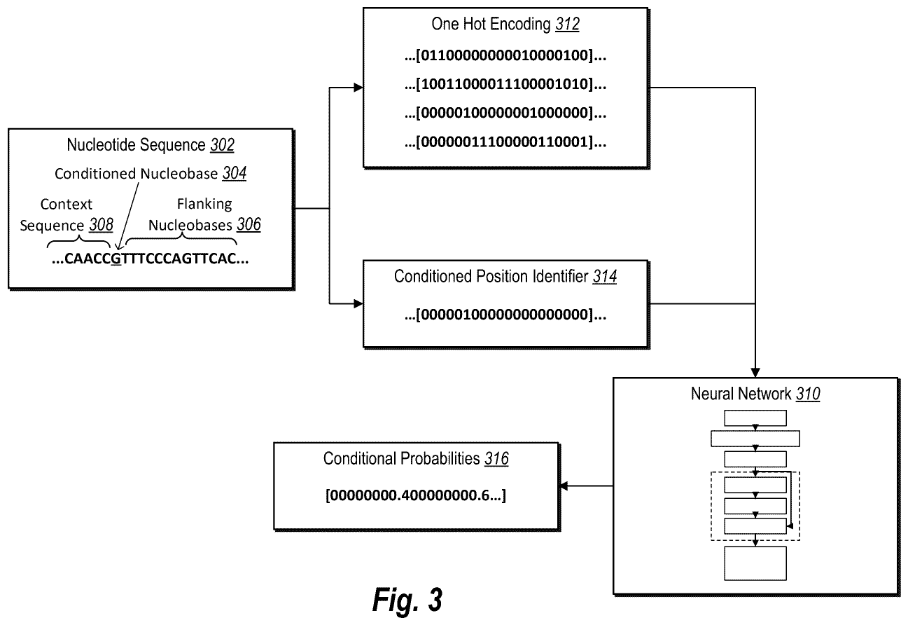

As just described, the splice-site prediction system 106 can utilize a neural network to analyze a nucleotide sequence and generate conditional probabilities based on the analysis. FIG. 3 illustrates the splice-site prediction system 106 using a neural network to generate conditional probabilities from a nucleotide sequence in accordance with one or more embodiments.

Indeed, as shown in FIG. 3, the splice-site prediction system 106 accesses a nucleotide sequence 302. In one or more embodiments, the nucleotide sequence 302 is associated with a genomic sample, such as a genomic sample extracted from a host. The nucleotide sequence 302 can include a deoxyribonucleic acid (DNA) sequence or a ribonucleic acid (RNA) sequence, such as a pre-messenger RNA (pre-mRNA sequence).

As illustrated in FIG. 3, the nucleotide sequence 302 includes a conditioned nucleobase 304 at a conditioned position. Additionally, the nucleotide sequence 302 includes flanking nucleobases 306. FIG. 3 shows the flanking nucleobases 306 of the nucleotide sequence 302 positioned downstream from the conditioned nucleobase 304. In some cases, however, the flanking nucleobases 306 can be positioned upstream from the conditioned nucleobase 304. Further, in some implementations, the flanking nucleobases 306 are positioned on both sides of the conditioned nucleobase 304. Thus, the nucleotide sequence 302 can have various configurations in various embodiments.

In some embodiments, the flanking nucleobases 306 include those nucleobases within the nucleotide sequence 302 that are within a threshold number of nucleobases from the conditioned nucleobase 304 (e.g., a threshold number of upstream nucleobases and/or a threshold number of downstream nucleobases). Indeed, as indicated by FIG. 3, in some cases, the flanking nucleobases 306 include a sequence of nucleobases that is adjacent to the conditioned nucleobase 304 within the nucleotide sequence and contains a threshold number of nucleobases. In some implementations, the splice-site prediction system 106 establishes a threshold (e.g., 4K nucleobases). In one or more embodiments, the splice-site prediction system 106 uses a default threshold. In some instances, the splice-site prediction system 106 uses a user-defined threshold received as input via a computing device. In some embodiments, the splice-site prediction system 106 determines the threshold based on hardware limitations, such as by establishing a threshold that allows for meeting certain performance standards. Thus, the flanking nucleobases 306 include the nucleobases within that distance from the conditioned nucleobase 304 (e.g., upstream and/or downstream).

Further, as illustrated in FIG. 3, the nucleotide sequence 302 includes a context sequence 308. As used herein, the term “context sequence” refers to a portion of a nucleotide sequence that provides context to a neural network with respect to the nucleotide sequence. In particular, a context sequence can provide, to a neural network, information about a nucleotide sequence in addition to the conditioned nucleobase and flanking nucleobases included therein to assist the neural network in its analysis of the conditioned nucleobase and the flanking nucleobases. For instance, in some cases, a context sequence indicates a location of the nucleotide sequence within a DNA or RNA strand. In some cases, the splice-site prediction system 106 does not search a context sequence for a donor or acceptor that matches a conditioned nucleobase but uses the context provided by the context sequence in its analysis. Though FIG. 3 illustrates the context sequence 308 upstream from the conditioned nucleobase 304 and flanking nucleobases 306, a nucleotide sequence can include a context sequence downstream from the conditioned nucleobase 304 and flanking nucleobases 306 or on both sides in some implementations. A context sequence can include thousands or tens of thousands (e.g., 32K nucleobases) of nucleobases in different embodiments.

As shown in FIG. 3, the splice-site prediction system 106 provides the nucleotide sequence 302 to a neural network 310. In particular, the splice-site prediction system 106 generates inputs for the nucleotide sequence 302 and provides the inputs to the neural network 310 (e.g., via corresponding input channels). Indeed, as shown, the splice-site prediction system 106 generates a one hot encoding input 312 and a conditioned position identifier input 314 for the nucleotide sequence 302.

In one or more embodiments, the splice-site prediction system 106 generates the one hot encoding input 312 by generating multiple one hot encoding vectors that collectively represent the arrangement of nucleobases within the nucleotide sequence 302. For instance, in some embodiments, the splice-site prediction system 106 generates a one hot encoding vector for each nucleobase type possibly represented within the nucleotide sequence 302 (e.g., adenine, cytosine, guanine, thymine, or uracil, depending on whether the nucleotide sequence 302 corresponds to DNA or RNA). To illustrate, the splice-site prediction system 106 can generate a one hot encoding vector for a nucleobase type where the one hot encoding vector has a length that is equal to the length of the nucleotide sequence 302. The splice-site prediction system 106 can further populate the one hot encoding vector with a value of one in positions where the nucleotide sequence 302 includes a nucleobase of that nucleobase type and a value of zero in the other positions.

In one or more embodiments, the splice-site prediction system 106 generates the conditioned position identifier input 314 by generating a conditioned position identifier that indicates the location of the conditioned position within the nucleotide sequence 302. In other words, the splice-site prediction system 106 generates the conditioned position identifier to flag the nucleobase that is located at the conditioned position within the nucleotide sequence 302 as the conditioned nucleobase 304. Generating conditioned position identifiers used by the splice-site prediction system 106 in various embodiments will be discussed in more detail below.

As illustrated in FIG. 3, the splice-site prediction system 106 utilizes the neural network 310 to generate conditional probabilities 316 from the nucleotide sequence 302. In particular, the splice-site prediction system 106 utilizes the neural network 310 to generate the conditional probabilities 316 from the one hot encoding input 312 and the conditioned position identifier input 314. As shown, the neural network 310 generates the conditional probabilities 316 by generating an output vector that includes the conditional probabilities 316. As mentioned above, however, the neural network 310 can provide its output in other formats in various embodiments.

In one or more embodiments, each conditional probability from the conditional probabilities 316 corresponds to a flanking nucleobase from the flanking nucleobases 306 of the nucleotide sequence 302. Further, in some cases, each conditional probability indicates a likelihood that its represented flanking nucleobase is part of a matching donor or a matching acceptor corresponding to the conditioned nucleobase 304. Said differently, in some instances, each conditional probability indicates a usage rate of its corresponding flanking nucleobase as part of a matching donor or a matching acceptor. Indeed, in some implementations, the splice-site prediction system 106 utilizes the neural network 310 to identify one or more potential splice sites for the conditioned nucleobase 304 and predict the probability with which each potential splice site is used during splicing, suggesting that each potential splice site is used with a frequency that corresponds to its predicted conditional probability.

As shown in FIG. 3, in some instances, most conditional probabilities generated by the neural network 310 have a value of zero, indicating that those nucleobases have a zero percent likelihood of being used as part of a splice site for the conditioned nucleobase 304. Further, as shown in FIG. 3, the non-zero conditional probabilities sum to a value of one. Thus, in some cases, the splice-site prediction system 106 utilizes the neural network 310 to generate normalized conditional probabilities. In some implementations, however, the nucleotide sequence 302 does not include any potential splice sites. Thus, in some instances, the conditional probabilities generated by the neural network 310 include all zero values, indicating that there are no matching splice sites within the nucleotide sequence 302.

By generating conditional probabilities that indicate likelihoods that flanking nucleobases are part of a matching splice site, the splice-site prediction system 106 operates more flexibly when compared to existing systems. Indeed, while existing systems are typically limited to providing binary classifications that fail to provide more than an indication of whether a nucleobase could be part of a splice site, the splice-site prediction system 106 flexibly provides more detailed outputs that both (i) identify nucleobases that could be part of a splice site and (ii) indicate their probability (or frequency) of being used. Thus, the splice-site prediction system 106 does more than analyze the possibility of being used as a splice site during splicing (which is performed by some existing systems), but operates to further determine the probability of being used as well (which is not available under existing systems in general or in terms of a per-nucleobase probability of being a splice site).

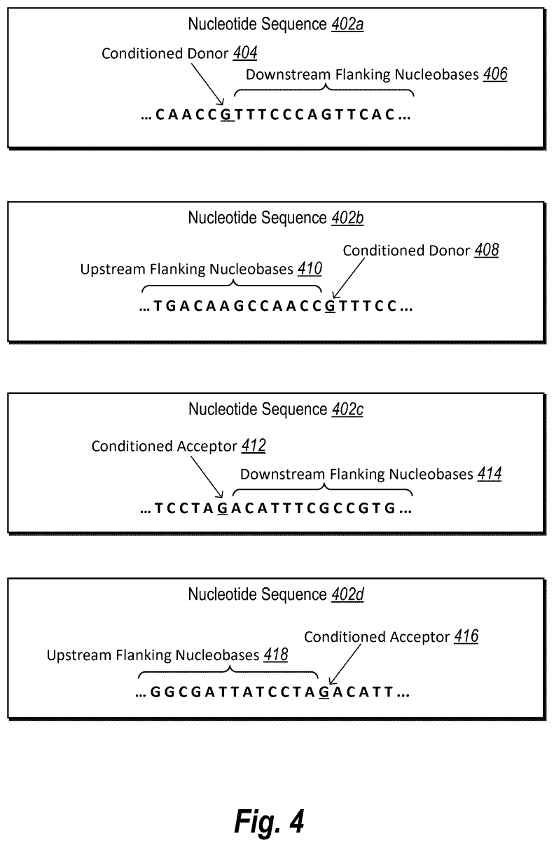

As indicated above, the splice-site prediction system 106 can provide a nucleotide sequence having different configurations to a neural network for analysis. FIG. 4 illustrates different configurations of nucleotide sequences that can be provided to a neural network in accordance with one or more embodiments.

For instance, as shown in FIG. 4, a nucleotide sequence 402a includes a conditioned donor 404. In particular, the nucleotide sequence 402a includes a conditioned nucleobase that is part of a donor (i.e., part of the conditioned donor 404). As shown, the nucleotide sequence 402a further includes downstream flanking nucleobases 406 (i.e., flanking nucleobases that are downstream from the conditioned donor 404). Additionally, FIG. 4 illustrates a nucleotide sequence 402b having a conditioned donor 408 and upstream flanking nucleobases 410 (i.e., flanking nucleobases that are upstream from the conditioned donor 408).

FIG. 4 also illustrates a nucleotide sequence 402c having a conditioned acceptor 412. In particular, the nucleotide sequence 402c includes a conditioned nucleobase that is part of an acceptor (i.e., part of the conditioned acceptor 412). FIG. 4 further illustrates the nucleotide sequence 402c having downstream flanking nucleobases 414 (i.e., flanking nucleobases that are downstream from the conditioned acceptor 412). FIG. also illustrates a nucleotide sequence 402d having a conditioned acceptor 416 and upstream flanking nucleobases 418 (i.e., flanking nucleobases that are upstream from the conditioned acceptor 416).

Thus, in some embodiments, a nucleotide sequence can include one of the four sequence configurations illustrated in FIG. 4: (i) a conditioned donor with downstream flanking nucleobases; (ii) a conditioned donor with upstream flanking nucleobases; (iii) a conditioned acceptor with downstream flanking nucleobases; or (iv) a conditioned acceptor with upstream flanking nucleobases. As such, in some instances, the splice-site prediction system 106 generates conditional probabilities that indicate one of the following: (i) likelihoods that the flanking nucleobases are part of a downstream matching acceptor that corresponds to a conditioned donor; (ii) likelihoods that the flanking nucleobases are part of an upstream matching acceptor that corresponds to a conditioned donor; (iii) likelihoods that the flanking nucleobases are part of a downstream matching donor that corresponds to a conditioned acceptor; or (iv) likelihoods that the flanking nucleobases are part of an upstream matching donor that corresponds to a conditioned acceptor.

As previously mentioned, however, the conditioned nucleobase of a nucleotide sequence can include a nucleobase that is not part of a donor or acceptor in some cases, so that the nucleotide sequence does not include a conditioned donor or a conditioned acceptor. Accordingly, in more general terms, a nucleotide sequence can include a conditioned nucleobase with upstream or downstream flanking nucleobases. Thus, in more general terms, the splice-site prediction system 106 can generate conditional probabilities that indicate likelihoods that the flanking nucleobases are part of a matching splice site that is upstream or downstream, respectively, from the conditioned nucleobase. For instance, the splice-site prediction system 106 can generate conditional probabilities that indicate one of the following: (i) likelihoods that the flanking nucleobases are part of a downstream matching acceptor that corresponds to a conditioned nucleobase; (ii) likelihoods that the flanking nucleobases are part of an upstream matching acceptor that corresponds to a conditioned nucleobase; (iii) likelihoods that the flanking nucleobases are part of a downstream matching donor that corresponds to a conditioned nucleobase; or (iv) likelihoods that the flanking nucleobases are part of an upstream matching donor that corresponds to a conditioned nucleobase.

To be clear, in some cases, the indications of the conditional probabilities generated by the splice-site prediction system 106 is based on the configuration of the nucleotide sequence that is analyzed. To illustrate, where the flanking nucleobases of the nucleotide sequence are downstream from the conditioned nucleobase, the splice-site prediction system 106 generates conditional probabilities that indicate likelihoods that the flanking nucleobases are part of a matching donor or a matching acceptor that is downstream from the conditioned nucleobase. On the other hand, where the nucleotide sequence includes flanking nucleobases that are upstream from the conditioned nucleobase, the splice-site prediction system 106 generates conditional probabilities that indicate likelihoods that the flanking nucleobases are part of a matching donor or a matching acceptor that is upstream from the conditioned nucleobase.

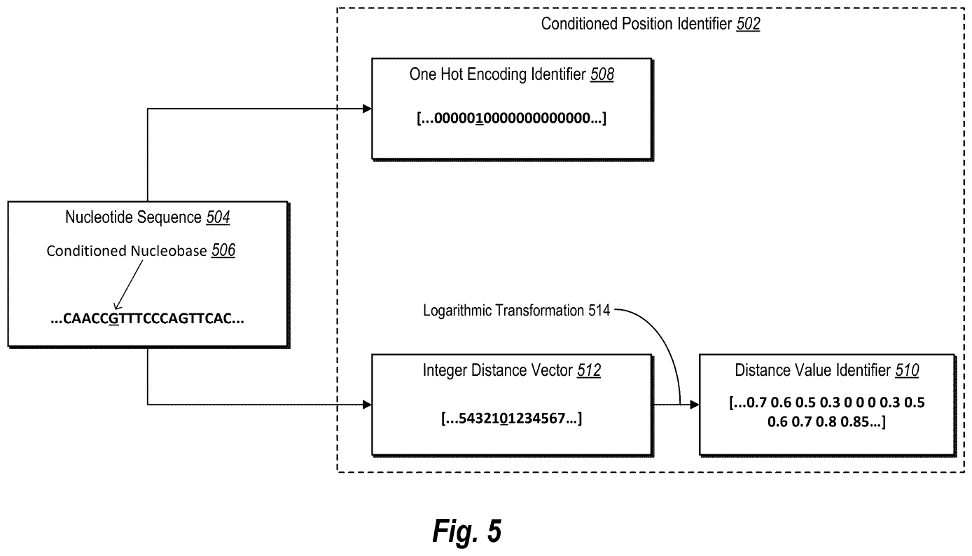

As previously mentioned, the splice-site prediction system 106 can generate a conditioned position identifier for a nucleotide sequence to indicate the location of the conditioned position within the nucleotide sequence. Further, the splice-site prediction system 106 can provide the conditioned position identifier as input to the neural network used to analyze the nucleotide sequence. FIG. 5 illustrates the splice-site prediction system 106 generating a conditioned position identifier for a nucleotide sequence in accordance with one or more embodiments.

Indeed, as shown in FIG. 5, the splice-site prediction system 106 generates a conditioned position identifier 502 for a nucleotide sequence 504 having a conditioned nucleobase 506 at a conditioned position. In particular, the splice-site prediction system 106 generates the conditioned position identifier 502 to identify, for the neural network analyzing the nucleotide sequence 504, the location of the conditioned position within the nucleotide sequence 504 or otherwise flag the nucleobase at the conditioned position as the conditioned nucleobase 506.

As shown in FIG. 5, in some embodiments, the splice-site prediction system 106 generates the conditioned position identifier 502 by generating a one hot encoding identifier 508. As further shown, in some cases, the splice-site prediction system 106 generates the one hot encoding identifier 508 by generating a one hot encoding vector. In particular, the splice-site prediction system 106 can generate a one hot encoding vector having a length that equals the length of the nucleotide sequence 504. Further, the splice-site prediction system 106 can populate the one hot encoding vector with a value of one at a position that corresponds to the conditioned position of the conditioned nucleobase 506 and a value of zero at every other position. Thus, the splice-site prediction system 106 can utilize the one hot encoding identifier 508 to directly indicate the location of the conditioned position.

As further shown in FIG. 5, in some embodiments, the splice-site prediction system 106 generates the conditioned position identifier 502 by generating a distance value identifier 510. As shown, in some cases, the splice-site prediction system 106 generates the distance value identifier 510 by generating a vector having a length that equals the length of the nucleotide sequence. In some cases, the splice-site prediction system 106 generates a vector having a length that is less than the length of the nucleotide sequence, such as a vector having a length that is equal to the number of flanking nucleobases within the nucleotide sequence (e.g., a length that excludes the conditioned nucleobase and context sequence). Further, the splice-site prediction system 106 can populate the vector with distance values where each distance value corresponds to a distance between the corresponding position of the nucleotide sequence 504 and the conditioned position of the conditioned nucleobase 506. In other words, each distance value can correspond to a number of nucleobases between the position populated with the distance value and the conditioned position. To illustrate, for a position within the vector that corresponds to a nucleobase that is directly adjacent to the conditioned position, the splice-site prediction system adds a distance value that indicates that the position is directly adjacent to conditioned position (e.g., a value of one indicating the position is one position away from the conditioned position or another value derived from the value of one and representing the adjacency).

As illustrated in FIG. 5, in one or more embodiments, the splice-site prediction system 106 generates the distance value identifier 510 by generating an integer distance vector 512. In particular, the splice-site prediction system 106 can generate the integer distance vector 512 by generating a vector having the same length as the nucleotide sequence 504 (or a length equal to the number of flanking nucleobases). The splice-site prediction system 106 can populate the integer distance vector 512 with integer values where each integer value indicates a distance (in number of positions) from its corresponding position to the conditioned position of the nucleotide sequence 504. To illustrate, for a position within the vector that corresponds to a nucleobase that is directly adjacent to the conditioned position, the splice-site prediction system adds an integer value of one.

As further illustrated in FIG. 5, the splice-site prediction system 106 generates the distance value identifier 510 from the integer distance vector 512 using a logarithmic transform 514. In particular, in some embodiments, the splice-site prediction system 106 applies the logarithmic transform 514 to each of the integer values of the integer distance vector 512 to produce the distance values of the distance value identifier 510. Indeed, in some embodiments, the neural network utilized by the splice-site prediction system 106 to analyze nucleotide sequences operates more reliably when analyzing with smaller numbers. As nucleotide sequences having a length of up to tens of thousands can be used, the splice-site prediction system 106 reduces the size of the values input to the neural network by transforming the integer values into the distance values via the logarithmic transform 514.

In some embodiments, the splice-site prediction system 106 utilizes the distance value identifier 510 as the conditioned position identifier 502 to achieve faster convergence for the neural network used to analyze nucleotide sequences. Indeed, in some implementations, the splice-site prediction system 106 employs a neural network to converges faster with distance values when compared to a one hot encoding.

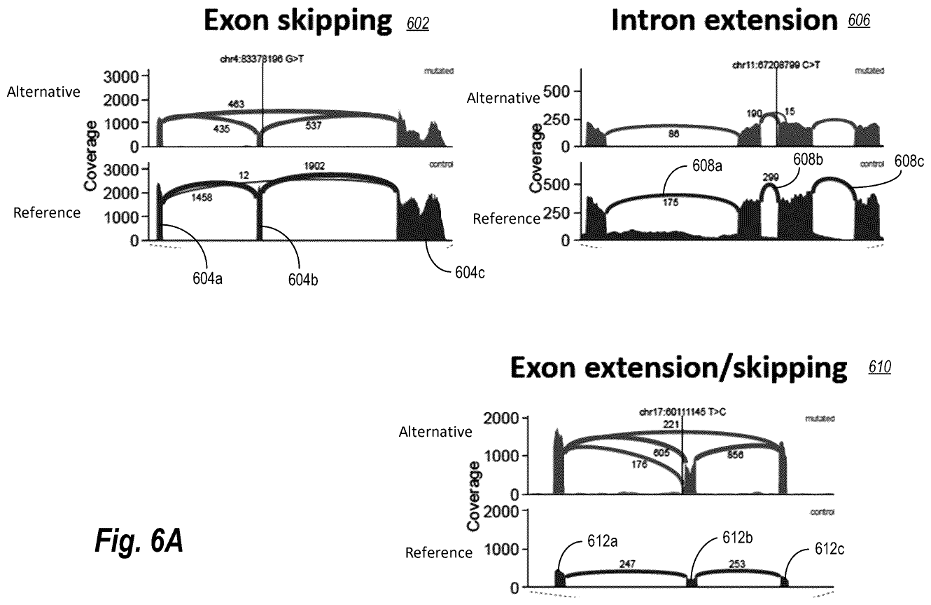

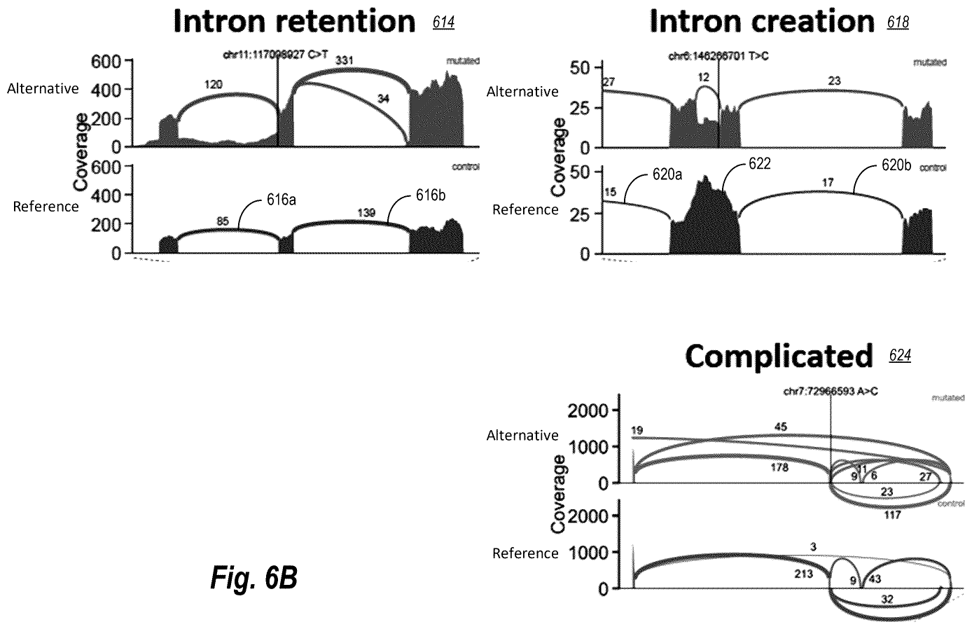

As mentioned above, the splice-site prediction system 106 utilizes a neural network to generate conditional probabilities that each indicate a likelihood that a flanking nucleobase of a nucleotide sequence is part of a splice site. In some cases, the splice-site prediction system 106 utilizes the conditional probabilities to determine whether or not some form of alternative splicing may occur for the nucleotide sequence (or, more generally, for the genomic sample associated with the nucleotide sequence). For instance, the splice-site prediction system 106 can utilize the conditional probabilities to determine how splicing for a nucleotide sequence is predicted to be different than splicing for a reference sequence. FIGS. 6A-6B illustrates example forms of alternative splicing that can be identified using the conditional probabilities generated by the splice-site prediction system 106 in accordance with one or more embodiments.

In particular, FIGS. 6A-6B illustrate coverage plots showing the coverage of a pre-splicing nucleotide sequence (e.g., a pre-mRNA sequence) provided by reads (i.e., nucleotide reads) of a spliced nucleotide sequence (e.g., a mRNA sequence). FIGS. 6A-6B show the coverage plots in pairs where the bottom coverage plot of each pair shows coverage for a nucleotide sequence experiencing normal splicing and the top coverage plot of each pair shows coverage for the nucleotide sequence due to a form of alternative splicing.

For instance, FIG. 6A illustrates a pair of coverage plots associated with an exon skipping example 602. As shown, the nucleotide sequence represented by the coverage plots for the exon skipping example 602 includes exons 604a, 604b, and 604c as well as introns between the exons 604a, 604b, and 604c. As shown by the bottom coverage plot, under normal conditions, the exons 604a-604c are typically used as splice sites. In other words, the spliced nucleotide sequence splices out the introns positioned between the exons 604a-604c but retains the exons 604a-604c themselves.

As shown by the top coverage plot for the exon skipping example 602, exon skipping occurs when an exon is skipped during the splicing process. In particular, an exon is spliced out of the nucleotide sequence (e.g., along with the adjacent introns). Indeed, as shown by the top coverage plot, a significant portion of the reads for the represented nucleotide sequence (463 reads) do not include the exon 604b. Thus, the top coverage plot indicates that the exon 604b was skipped during the splicing process; or in other words, was spliced out.

FIG. 6A also illustrates a pair of coverage plots associated with an intron extension example 606. As shown, the nucleotide sequence represented by the coverage plots for the intron extension example 606 includes introns 608a, 608b, and 608c as well as exons adjacent to and in between the introns 608a, 608b, and 608c. As shown by the bottom coverage plot, under normal conditions, the boundaries between introns 608a-608c and the exons are used as splice sites. Accordingly, the introns 608a-608c and only the introns 608a-608c are spliced out. As shown by the top coverage plot, however, intron extension occurs when splicing extends into an exon. In other words, a portion of an exon is spliced out along with the adjacent intron (as if the adjacent intron had an extended length). Indeed, as illustrated by the top coverage plot, several reads (15 reads) show the intron 608b extending into the adjacent exon, indicating that the portion of the adjacent exon was spliced out along with the intron 608b.

Additionally, FIG. 6A illustrates a pair of coverage plots associated with an exon extension/skipping example 610. As shown, the nucleotide sequence represented by the coverage plots for the exon extension/skipping example 610 includes exons 612a, 612b, and 612c as well as introns between the exons 612a, 612b, and 612c. As shown by the bottom coverage plot, under normal conditions, the boundaries between the exons 612a-612c and the introns are used as splice sites. Accordingly, the full length of the introns between the exons 612a-612c are spliced out. As shown by the top coverage plot, however, exon extension occurs when a portion of an intron is retained after splicing. In other words, less than the full length of an intron is spliced out (as if the adjacent exon had an extended length). Indeed, as illustrated by the top coverage plot, a significant number of reads (176 reads) indicate that the exon 612b was extended during splicing so that less than the entirety of the preceding intron was spliced out.

FIG. 6B illustrates a pair of coverage plots associated with an intron retention example 614. As shown, the nucleotide sequence represented by the coverage plots for the intron retention example 614 includes introns 616a and 616b as well as exons adjacent to and in between the introns 616a and 616b. As shown by the bottom coverage plot, under normal conditions, the introns 616a-616b are both spliced out. As shown by the top coverage plot, however, intron retention occurs when an intron is retained (e.g., fully retained) after splicing. Indeed, as shown in the top coverage plots, a number of reads (120 reads) show the intron 616a being spliced out. However, the top coverage plot also illustrates a number of reads indicating that the intron 616a was not spliced out (i.e., the reads associated with the peaks located under the arc for the intron 616a).

FIG. 6B also illustrates a pair of coverage plots associated with an intron creation example 618. As shown, the nucleotide sequence represented by the coverage plots for the intron creation example 618 includes introns 620a-620b as well as exons adjacent to and in between the introns 620a-620b (including the exon 622). As shown by the bottom coverage plot, under normal conditions, the introns 620a-620b, and only the introns 620a-620b are spliced out. In other words, the exons are not spliced out. As shown by the top coverage plot, however, intron creation occurs when a portion of an exon that does not adjoin either of the adjacent introns is spliced out. In other words, an inner portion of an exon is spliced out (as if it were a new intron). Indeed, as shown in the top coverage plot, several reads (12 reads) indicate that a portion of the exon 622 was spliced out as if it were separate from the intron 620a and the intron 620b.

Additionally, FIG. 6B illustrates a pair of coverage plots associated with a complicated alternative splicing example 624. In particular, the pair of coverage plots associated with the complicated alternative splicing example 624 illustrate an alternative form of splicing that may result from a combination of the alternative forms described above (or from a combination that includes one or more alternative forms of splicing not described).

Thus, in some embodiments, the splice-site prediction system 106 generates conditional probabilities that indicate alternative splicing may occur for a nucleotide sequence. Indeed, by generating the conditional probabilities, the splice-site prediction system 106 can indicate the splice sites that may be used and their associated probability of being used. Differences between the splice sites identified by the splice-site prediction system 106 or the conditional probabilities generated by the splice-site prediction system 106 and a reference that shows the use of splice sites under normal conditions can indicate that alternative splicing may be present. In some cases, based on these differences, the splice-site prediction system 106 can determine that a variant is present within the genomic sample associated with the analyzed nucleotide sequence that is a cause of the alternative form of splicing.

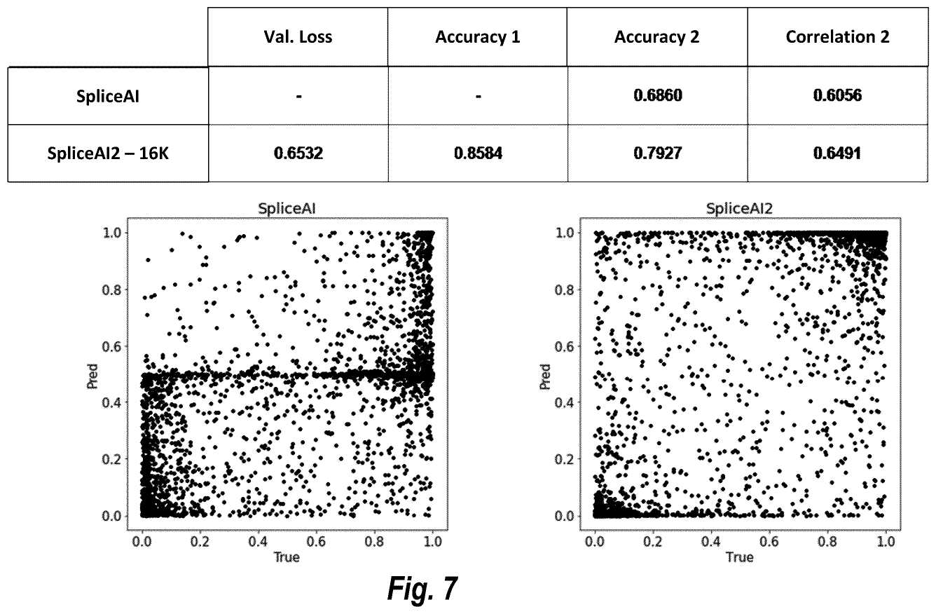

As discussed above, the splice-site prediction system 106 operates more accurately than existing systems. In particular, the splice-site prediction system 106 more accurately detects splice sites that will be used within a nucleotide sequence. Researchers have conducted studies to determine the accuracy of the splice-site prediction system 106 compared to conventional systems. FIG. 7 illustrates a table and a pair of graphs that reflect the results of these studies.

The table of FIG. 7 illustrate the performance of at least one embodiment of the splice-site prediction system 106 that analyzes nucleotide sequences that are 16,000 nucleobases long (labeled “SpliceAI2-16K”). In particular, as shown, the table of FIG. 7 shows the performance of the splice-site prediction system 106 with respect to a validation loss metric, an accuracy one metric, and accuracy two metric, and a correlation two metric. The table of FIG. 7 further indicates the performance of an existing state-of-the-art system (labeled “SpliceAI”) using the accuracy two metric and the correlation two metric. The SpliceAI system is described by Kishore Jaganathan et al., Predicting Splicing from Primary Sequence with Deep Learning, Cell, 2019 January; 176 (3): 535-548. Thus, the table of FIG. 7 compares the performance of the splice-site prediction system 106 to the performance of the existing state-of-the-art system using these last two metrics. It should be noted that the existing state-of-the-art system represented in FIG. 7 employs a model trained to generate binary classifications and adapted to provide probabilities.

To measure the performance via the accuracy one metric, the researchers focused on splice sites that are not associated with alternative splicing. Thus, when fixing a splice site (e.g., selecting a conditioned nucleobase that is part of that splice site), there is typically one position that is used as a matching splicing site. The researchers used the accuracy one metric to measure how accurate the splice-site prediction system 106 performing in assigning the highest score (e.g., the highest conditional probability) to that position. As shown in FIG. 7, the splice-site prediction system 106 operates with high accuracy.

To measure the performance via the accuracy two metric, the researchers used nucleotide sequences where competing matching splice sites would be present for a fixed splice site (e.g., a conditioned nucleobase). The researchers used the accuracy two metric to measure how accurate the models performed in assigning the highest conditional probability to the splice site that was actually used most frequently. In such a scenario, where there are two competing matching splice sites, a randomly guessing model would be expected to perform with fifty percent accuracy. As shown by the table of FIG. 7, not only did the splice-site prediction system 106 perform with an accuracy well above fifty percent, but it also outperformed the existing state-of-the-art system, showing an accuracy increase of almost eleven percent.

The researchers used the correlation two metric to measure how accurate the models were with their probability predictions. In particular, the researchers used the correlation two metric to measure how close the probability predictions generated by the models (e.g., the conditional probabilities generated by the splice-site prediction system 106) were to the actual probabilities of splice sites being used. In particular, where a nucleotide sequence has multiple competing matching splice sites where each splice site was used during splicing with a particular probability, how close did the models come to predicting those probabilities. A higher correlation value (e.g., a value closer to one) indicates better accuracy in predicting the correct probabilities. As shown by the table of FIG. 7, the splice-site prediction system 106 outperformed the existing state-of-the-art system, showing an accuracy increase of about five percent.

The scatter plots of FIG. 7 illustrate the correlations presented in the table for the splice-site prediction system 106 (right) and the existing state-of-the-art system (left). In the scatter plots, the x-axis represents the true probability of a splice site being used, and the y-axis represents the probability for the splice site predicted by the corresponding model. The scatter plots illustrate the improved performance of the splice-site prediction system 106 with respect to correlation when compared to the existing state-of-the-art system. For instance, the scatter plot associated with the existing state-of-the-art system includes a defined bar of data points extending horizontally across the plot, indicating that the existing state-of-the-art system incorrectly predicted probabilities for a large portion of the splice sites where the true probability for those splice sites was either higher or lower (sometimes much higher or lower).

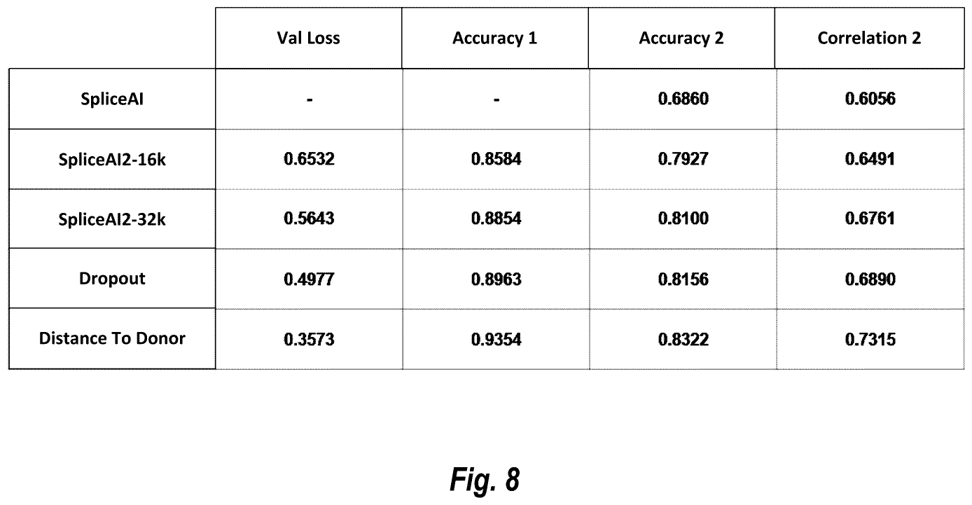

FIG. 8 illustrates an additional table reflecting additional experimental results from studies performed by the researchers. In particular, the table of FIG. 8 compares the performance of the existing state-of-the-art system discussed above with reference to FIG. 7 and multiple implementations of the splice-site prediction system 106. In particular, the table includes performance metrics for the SpliceAI2-16K implementation discussed above with reference to FIG. 7. Additionally, the table includes performance metrics for an implementation of the splice-site prediction system 106 that analyzes nucleotide sequences that are 32,000 nucleobases long (labeled “SpliceAI2-32K”). The table also includes performance metrics for an implementation of the splice-site prediction system 106 that incorporated a dropout layer into convolutional blocks of the neural network (labeled “Dropout”). Further, the table includes performance metrics for an implementation of the splice-site prediction system 106 that incorporates an input channel to the neural network for accepting a conditioned position identifier, such as a distance value identifier discussed above with reference to FIG. 5 (labeled “Distance to Donor”).

As illustrated by the table of FIG. 8, all shown implementations of the splice-site prediction system 106 outperformed the existing state-of-the-art system with regards to the accuracy two and correlation two metrics. Comparing the performance metrics for the SpliceAI2-16K and SpliceAI2-32K implementations shows that analyzing longer inputs sequences typically leads to better results. Further, as shown by the table, while incorporating a dropout layer into the neural network architecture provides some improvement to performance, incorporating the additional input channel that receives distance value identifier greatly increase accuracy and correlation while significantly reducing the validation loss.

Thus, as shown by FIGS. 7-8, the splice-site prediction system 106 provides improved performance in terms of its accuracy in detecting splice sites within nucleotide sequences compared to existing systems. Accordingly, the splice-site prediction system 106 provides a more accurate indication of how variants affect the splicing of a nucleotide sequence.

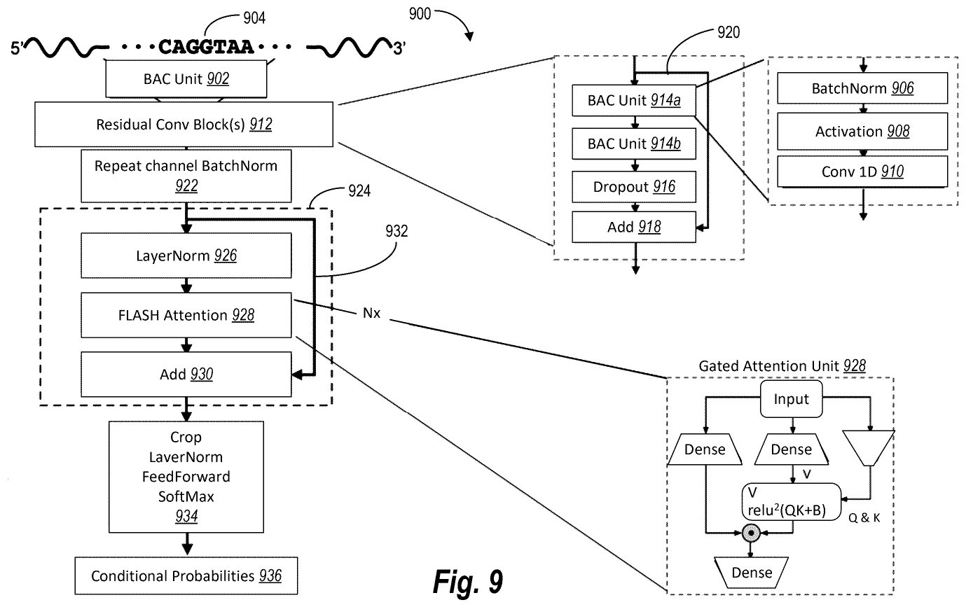

As previously discussed, the splice-site prediction system 106 can utilize a transformer neural network as the neural network implemented to analyze nucleotide sequences and generate conditional probabilities. In particular, in some cases, the splice-site prediction system 106 utilizes a transformer neural network that includes architectural components and features that allow for the practical implementation of the transformer neural network to the task of splice site detection. FIGS. 9-19 illustrate transformer neural networks that can be employed by the splice-site prediction system 106 with components and features that aid in execution of the splice-site prediction task in accordance with one or more embodiments.

FIG. 9 illustrates an architecture of a transformer neural network utilized by the splice-site prediction system 106 to analyze nucleotide sequences and generate conditional probabilities in accordance with one or more embodiments. As shown in FIG. 9, a transformer neural network 900 implemented by the splice-site prediction system 106 includes an initial BAC unit 902. As further shown, a BAC unit includes a batch normalization layer 906, an activation layer 908, and a convolutional layer 910. As indicated in FIG. 9, the transformer neural network 900 utilizes the initial BAC unit 902 to receive an input 904 to the transformer neural network 900. For instance, in some implementations, the transformer neural network 900 utilizes the initial BAC unit 902 to receive a one hot encoding input and a conditioned position identifier input as discussed above with reference to FIG. 3.

In some implementations, the input 904 to the transformer neural network 900 corresponds to a long nucleotide sequence. As an example, the input 904 can correspond to a nucleotide sequence having a sequence length between six thousand and thirty-two thousand nucleobases. As discussed above with reference to FIG. 8, performance of the splice-site prediction system 106 in detecting splice sites can improve with longer nucleotide sequences in some cases. In particular, the splice-site prediction system can analyze motifs or other data patterns that are positioned far away (e.g., hundreds to thousands of positions) from a conditioned nucleobase within a nucleotide sequence in detecting splice sites. While positioned one thousand or multiple thousands of positions away, these motifs or other patterns may be indicative of which nucleobases are part of a matching splice site for a conditioned nucleobase. Accordingly, by analyzing longer nucleotide sequences, the splice-site prediction system 106 is able to better identify splice sites using these long-range motifs or patterns.

As further shown in FIG. 9, the transformer neural network 900 further includes one or more residual convolutional blocks 912. The transformer neural network 900 can include various numbers of residual convolutional blocks in various embodiments. For example, in some implementations, the transformer neural network 900 includes three or four residual convolutional blocks. In one or more embodiments, the transformer neural network 900 utilizes the one or more residual convolutional blocks 912 to capture local motifs present within the input 904. As shown in FIG. 9, a residual convolutional block includes a first BAC unit 914a, a second BAC unit 914b, a dropout layer 916, and an addition layer 918 (e.g., a concatenation layer). Further, as shown, a residual convolutional block includes a link 920 from the input to the convolutional residual block to the addition layer 918.

In one or more embodiments, the one or more residual convolutional blocks 912 operates at a higher dimensionality of data than is associated with the input 904 to the transformer neural network 900. For instance, as mentioned, the transformer neural network 900 can receive the input 904 via five channels (e.g., four one hot encoding input channels and one conditioned position identifier input channel) while the one or more residual convolutional blocks 912 operate at a much higher dimensionality of data (e.g., thirty-two dimensions or channels). Accordingly, in some instances, the transformer neural network 900 utilizes the initial BAC unit 902 to transform the data from the lower dimensionality associated with the input 904 to the higher dimensionality used by the one or more residual convolutional blocks 912.

Additionally, as shown in FIG. 9, the transformer neural network 900 includes a repeat channel batch normalization layer 922 followed by a transformer block 924. In one or more embodiments, the transformer block 924 operates at a higher dimensionality of data than the one or more residual convolutional blocks 912. For instance, as mentioned above, the one or more residual convolutional blocks 912 operates on thirty-two dimensions, in some instances, while the transformer block 924 can operate at a much higher dimensionality (e.g., five hundred twelve dimensions). Accordingly, in some instances, the transformer neural network 900 utilizes the repeat channel batch normalization layer 922 to transform the data from the lower dimensionality of the one or more residual convolutional blocks 912 to the higher dimensionality of the transformer block 924.

As shown in FIG. 9, in some implementations, the transformer block 924 includes a fast-linear-attention-with-a-single-head (FLASH) block. In particular, the transformer block 924 includes a transformer block that incorporates FLASH attention. Indeed, as shown in FIG. 9, the transformer block 924 includes a normalization layer 926, an attention unit 928 (e.g., a FLASH-based attention unit), and an addition layer 930 (e.g., a concatenation layer). As further shown, the transformer block 924 includes a link 932 from the input to the transformer block 924 to the addition layer 930. FIG. 9 also shows the architectural composition of the attention unit 928. More detail describing the attention unit 928 and its components will be provided below.

In one or more embodiments, the transformer neural network 900 utilizes the transformer block 924 to capture long-distance interactions within the input 904. Indeed, as previously mentioned, transformer components are more suited to recognizing long-distance patterns or dependencies when compared to convolutional layers. Thus, the transformer neural network 900 uses the transformer block 924 to capture this long-distance information. In some cases, however, the presence of the one or more residual convolutional blocks 912 improves the performance of the transformer block 924 as the one or more residual convolutional blocks 912 capture local motifs that then feed into the attention of the transformer block 924, allowing for the recognition of interactions between motifs.

It should be noted that, while FIG. 9 illustrates the transformer neural network 900 incorporating FLASH-based attention, other attention architectures can be used in various embodiments. For instance, in some implementations, the transformer neural network 900 incorporates performer-based attention. In other words, in some cases, the transformer block 924 includes a performer block with a performer-based attention unit.

As indicated by FIG. 9, the transformer neural network 900 can include N transformer blocks, or the transformer block 924 shown can be repeated N times (Nx) within the neural network architecture. Indeed, in some cases, the transformer neural network 900 includes a series of transformer blocks where the output of one transformer block is provided as input to the following transformer block.

As further shown in FIG. 9, the transformer neural network 900 includes an output block 934. The output block 934 can include one or more output layers. For instance, as shown, the output block 934 includes a crop layer, a normalization layer, a feed forward layer, and a softmax layer. In one or more embodiments, the transformer neural network 900 uses the output block 934 to process the output of the transformer block 924 (or the output of the final transformer block in a series of transformer blocks) and generate an output accordingly. In particular, the transformer neural network 900 uses the output block 934 to generate conditional probabilities 936 corresponding to the input 904 to the transformer neural network 900.

Thus, the splice-site prediction system 106 can utilize the transformer neural network 900 to analyze a nucleotide sequence and generate corresponding conditional probabilities. To illustrate, in one or more embodiments, the transformer neural network 900 uses the one or more residual convolutional blocks 912 to generate a convolutional feature vector representing the nucleotide sequence. Further, the transformer neural network 900 uses the transformer block 924 to generate an attention-based feature vector from the convolutional feature vector. Where multiple transformer blocks are involved, the transformer neural network 900 can use the first transformer block to generate a first attention-based feature vector, provide the first attention-based feature vector as input to the second transformer block, use the second transformer block to generate a second attention-based feature vector from the first attention-based feature vector, and so on. Finally, the transformer neural network 900 can use the output block 934 (e.g., the one or more output layers of the output block 934) to generate the conditional probabilities 936 from the attention-based feature vector (e.g., the attention-based feature vector generated by the last transformer block).

By utilizing a transformer neural network to generate conditional probabilities, the splice-site prediction system 106 utilizes a more flexible neural network architecture when compared to existing systems. Indeed, while many existing systems were limited to convolutional networks that could recognize local motifs but were unsuited to recognize long-range dependencies, the splice-site prediction system 106 flexibly implements a transformer neural network that could capture those long-range dependencies. In particular, in implementing a transformer neural network with convolutional residual blocks as described above, the splice-site prediction system 106 implements a flexible transformer neural network that can leverage both local motifs and long-range dependencies in its analysis. In many cases, as the transformer neural network can analyze longer input sequences than could be processed by existing systems, the long-range dependencies recognized and leveraged by the splice-site prediction system 106 can span distances that would render them unrecognizable by existing systems. As such, the splice-site prediction system 106 further implements a more accurate neural network architecture that can perform splice site detection based on both local motifs and long-range dependencies. In particular, the splice-site prediction system 106 implements a transformer neural network that can more accurately determine whether a nucleobase is part of a splice site when compared to the models of existing systems.

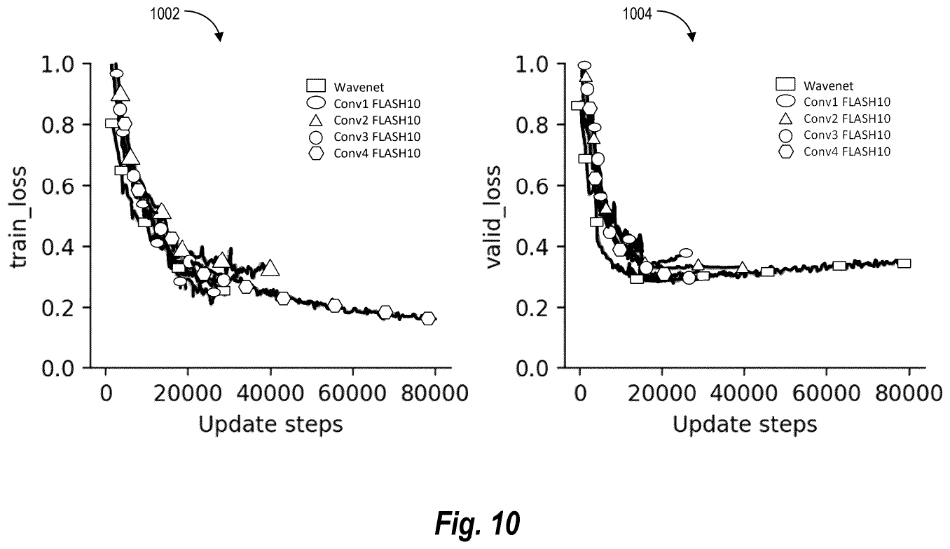

As previously mentioned, the splice-site prediction system 106 can utilize a transformer neural network having one or more residual convolutional blocks. FIG. 10 illustrates graphs reflecting experimental results regarding the performance of different implementations of a transformer neural network that can be employed by the splice-site prediction system 106 based on the number of residual convolutional blocks included in accordance with one or more embodiments.

In particular, FIG. 10 illustrates a first graph 1002 and a second graph 1004, each comparing the performance of the transformer neural network implementations with a Wavenet model described by Aaron van den Oord et al., Wavenet: A Generative Model for Raw Audio, 2016, arxiv: 1609.03499. Some variation of the Wavenet model is implemented by some existing state-of-the-art systems, including the SpliceAI system discussed above with reference to FIGS. 7-8 (though the SpliceAI system discussed above offers some additional and/or alternative features), and has been adapted to generating conditional probabilities. The transformer neural network implementations each include ten transformer blocks (e.g., ten FLASH blocks) and differ in their number of included residual convolutional blocks. In particular, the graphs show the performance for a first implementation having one residual convolutional block, a second implementation having two residual convolutional blocks, a third implementation having three residual convolutional blocks and a fourth implementation having four residual convolutional blocks.

The first graph 1002 shows the performance of the tested models with respect to a training loss metric (the x-axis). The second graph 1004 shows the performance of the tested models with respect to a validation loss metric (the x-axis). Both graphs illustrate the performance across update steps (the y-axis). As from the graphs, those iterations of the transformer neural network with three or four residual convolutional blocks decrease and stabilize better than those iterations with one or four residual convolutional blocks with respect to both metrics. Thus, the graphs indicate that the inclusion of the residual convolutional blocks prevent problems, such as overfitting, that may otherwise be present. Further, the graphs show that those iterations with three or four residual convolutional blocks approach the performance of the Wavenet model, which is a comparatively less complex model. Accordingly, the graphs indicate the including the residual convolutional blocks (e.g., three or four blocks) into the architecture of the transformer neural network provides improvements for its execution of the splice-site prediction task.

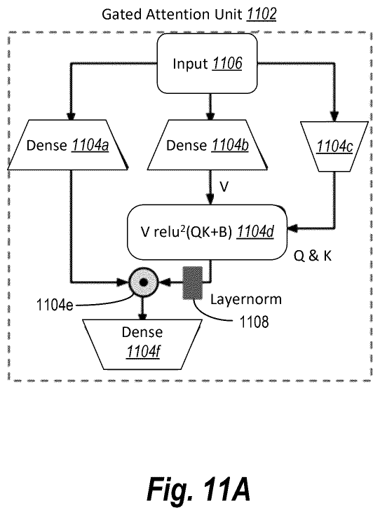

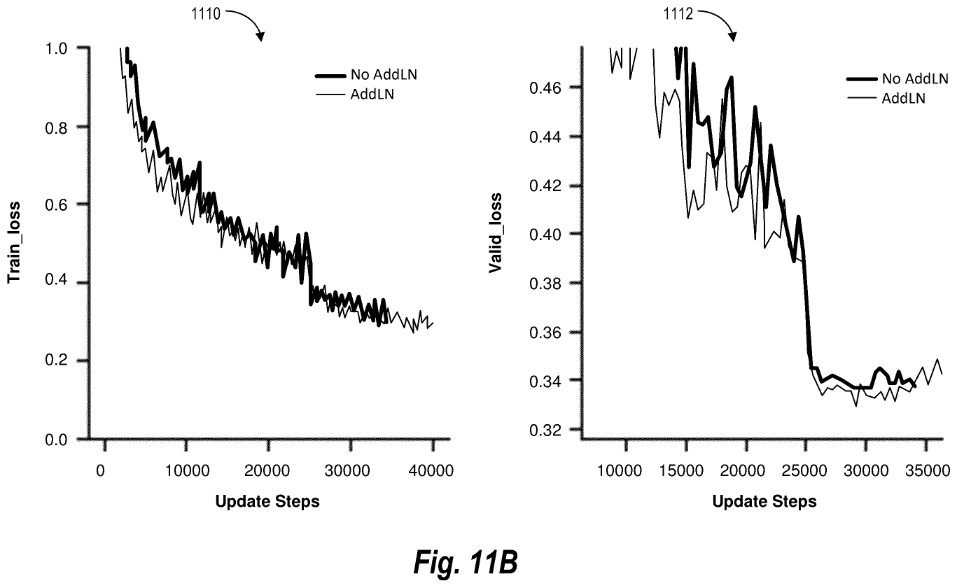

As mentioned, in one or more embodiments, the splice-site prediction system 106 utilizes a transformer neural network with one or more attention units, such as one or more FLASH-based attention units. In some instances, the splice-site prediction system 106 configures the attention unit(s) to improve the suitability of the transformer neural network for the task of splice site detection. FIGS. 11A-11B illustrate an attention unit utilized by the splice-site prediction system 106 in accordance with one or more embodiments.

In particular, FIG. 11A illustrates an architecture of an attention unit 1102 utilized by the splice-site prediction system 106 within a transformer neural network in accordance with one or more embodiments. Indeed, as shown in FIG. 11A, the attention unit 1102 includes components 1104a-1104f used to process an input 1106. Each of the components 1104a-1104f can include one or more internal layers or other components of the attention unit 1102. For example, as indicated, the component 1104a, the component 1104b, and the component 1104f include one or more dense layers. Additionally, the component 1104d includes one or more rectified linear units (ReLUs). Further, the component 1104e includes an operation, such as an element-wise multiplication operation. The attention unit 1102 with the components 1104a-1104g is similar in architecture to the attention unit 928 discussed above with reference to FIG. 9.

As indicated by FIG. 11A, the component 1104b outputs a feature tensor, such as a value tensor V. Further, the component 1104c provides additional feature tensors, such as a query tensor Q and a key tensor K. Further, as shown, the component 1104d processes the feature tensors to output its own values. In particular, in some embodiments, the component 1104d processes the feature tensors to generate a quadratic-attention output and a linear-attention output. Indeed, in one or more embodiments, the attention unit 1102 performs one or more computations that are linear in complexity and one or more additional operations that are quadratic in complexity. In some cases, the data resulting from these operations is of the same shape (e.g., the same dimensionality). In one or more embodiments, the component 1104d generates the quadratic-attention output and the linear-attention output as follows:

$${\hat{V}}_{g}^{quad} = {relu}^{2} ( {Q}_{g}^{quad} {K}_{g}^{{quad}^{T}} + b ) {V}_{g} \qquad ( 1 )$$

$${\hat{V}}_{g}^{lin} = {Q}_{g}^{lin} ( {\sum }_{h = 1}^{G} {K}_{g}^{{lin}^{T}} {V}_{h} ) \qquad ( 2 )$$

In some cases, the component 1104d generates the quadratic-attention output and the linear-attention output as shown in functions 1 and 2, respectively, based on the input 1106 to the attention unit 1102. In one or more embodiments, the attention unit 1102 utilizes the quadratic-attention output and the linear-attention output to generate an output of the attention unit 1102 as follows:

$${O}_{g} = [ {U}_{g} ⊙ ( {\hat{V}}_{g}^{quad} + {\hat{V}}_{g}^{lin} ) ] {W}_{o} \qquad ( 3 )$$

In function 3, Wₒϵ [symbol] ^{e×d} where d denotes model size and e denotes an expanded intermediate size. Further, in function 3, U_{g}ϵ [symbol] ^{T×e} where T represents the tokens. In one or more embodiments, the attention unit 1102 with the components 1104a-1104f operates as described by Weizhe Hua et al., Transformer Quality in Linear Time, International Conference on Machine Learning, 2022, arxiv: 2202.10447, which is incorporated herein by reference in its entirety.

As further shown in FIG. 11A, the splice-site prediction system 106 adds a normalization layer 1108 to the attention unit 1102 in some embodiments. Indeed, while the splice-site prediction system 106 can utilize the attention unit 1102 without the normalization layer 1108 (i.e., with just the components 1104a-1104f) as described above, the splice-site prediction system 106 incorporates the normalization layer 1108 in some implementations. As will be discussed below, incorporating the normalization layer 1108 improves the suitability of the transformer neural network for the splice-site prediction task in many cases.

As indicated in FIG. 11A, the splice-site prediction system 106 positions the normalization layer 1108 within the attention unit 1102 to accept at least one of the quadratic-attention output or the linear-attention output determined in accordance with functions 1 and 2, respectively. In particular, the splice-site prediction system 106 positions the normalization layer 1108 after the component 1104d. Thus, the splice-site prediction system 106 utilizes the normalization layer 1108 to generate the following:

$${\hat{V}}_{g}^{quad} = LayerNorm ( {\hat{V}}_{g}^{quad} ) \qquad ( 4 )$$

$${\hat{V}}_{g}^{l i n} = LayerNorm ( {\hat{V}}_{g}^{l i n} ) \qquad ( 5 )$$

As shown by functions 4 and 5, the splice-site prediction system 106 utilizes the normalization layer 1108 to perform a normalization on the quadratic-attention output and the linear-attention output, respectively. Thus, in one or more embodiments, the attention unit 1102 utilizes the normalized quadratic-attention output and the normalized linear-attention output represented by functions 4 and 5, respectively, to generate an attention output of the attention unit 1102 in accordance with function 3.

In one or more embodiments, the splice-site prediction system 106 uses the normalization layer 1108 to facilitate the analysis of longer input sequences. For instance, in some cases, as input nucleotide sequences get longer, the vectors derived from those sequences (e.g., the vectors generated by internal layers of the neural network) become large and can have detrimental effects in terms of the numerical optimization, such as the gradient flow. As such, the neural network can have difficulty optimizing during the training process. By adding the normalization layer 1108, the splice-site prediction system 106 configures the data to be in a more suitable range so that the neural network converges more quickly during training.

FIG. 11B illustrates graphs reflecting experimental results regarding the performance of a transformer neural network having various attention unit architectures that can be implemented by the splice-site prediction system 106 in accordance with one or more embodiments. In particular, FIG. 11B illustrates graphs that compare an implementation of a transformer neural network having one or more attention units without an added normalization layer (e.g., without the normalization layer 1108 shown in FIG. 11A) to an implementation having one or more attention units with the added normalization layer. The first graph 1110 shows the performance of each model with respect to a training loss metric. The second graph 1112 shows the performance of each model with respect to a validation loss metric.

As shown by the graphs in FIG. 11B, the addition of a normalization layer to the attention unit(s) improves the performance of the transformer neural network. For instance, as shown by the first graph 1110, the transformer neural network converges more quickly when its attention unit(s) include the added normalization layer. Further, as shown by the second graph 1112, the transformer neural network has a lower validation loss when its attention unit(s) include the added normalization layer.

Thus, by using one or more attention units with an added normalization layer, the splice-site prediction system 106 implements a transformer neural network that is better suited to the task of splice site detection. Indeed, as previously mentioned, existing systems avoid using transformer neural networks at least partly because their slow convergence speed complicated its practical application to the splice-site prediction task. By reducing this convergence speed via the added normalization layer(s), the splice-site prediction system 106 configures the transformer neural network to be more practically employed for detecting splice sites within nucleotide sequences. In particular, the splice-site prediction system 106 improves the training of the transformer neural network via the faster convergence to learn parameters that enable use of the transformer neural network in detecting splice sites in a timely manner Thus, the splice-site prediction system 106 can practically (e.g., efficiently) train and implement the transformer neural network where, traditionally, the detriments of the slow convergence speed outweighed the benefits of the transformer neural network.

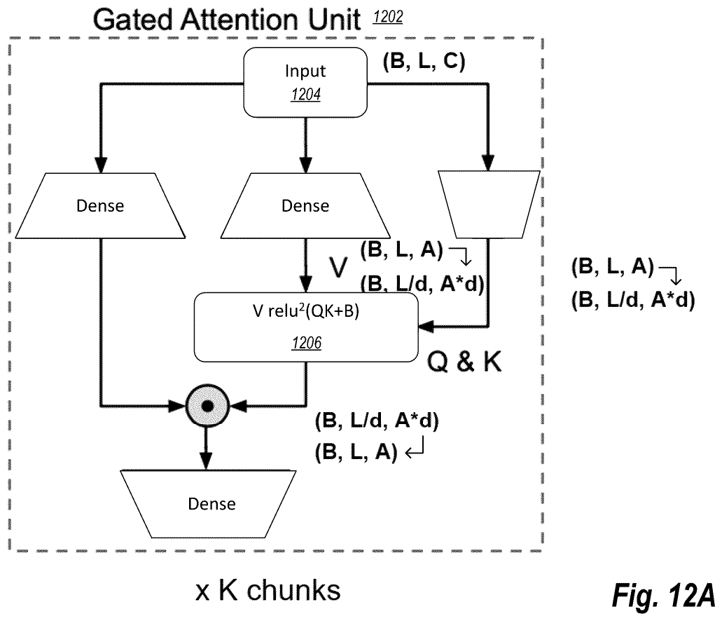

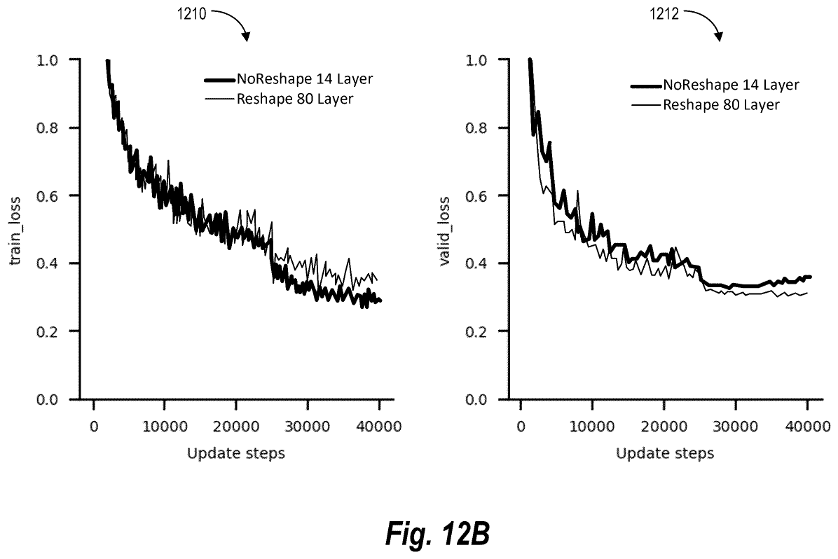

In one or more embodiments, the splice-site prediction system 106 configures the attention unit(s) to improve the suitability of the transformer neural network for the task of splice site detection by reshaping data within the attention unit(s). FIGS. 12A-12B illustrates the splice-site prediction system 106 reshaping data within an attention unit of a transformer neural network in accordance with one or more embodiments.

In particular, FIG. 12A illustrates reshaping data within an attention unit 1202 of a transformer neural network to reduce the time complexity of the transformer neural network in accordance with one or more embodiments. Indeed, FIG. 12A shows the attention unit 1202 having a configuration similar to the attention unit 928 discussed above with reference to FIG. 9 or the attention unit 1102 discussed above with reference to FIG. 11A (but without the added normalization layer). As shown in FIG. 12A, and as previously discussed with reference to FIG. 11A, the attention unit 1202 generates or otherwise operates on one or more feature tensors, such as a value tensor V, a query tensor Q, and a key tensor K. As further indicated, each of the value tensor V, the query tensor Q, and the key tensor K has dimensions (B, L, A) where B represents a batch size, L represents sequence length, and A represents an attention dimension.

In particular, as indicated by FIG. 12A, the attention unit 1202 processes data in K chunks. Indeed, in one or more embodiments, the attention unit 1202 processes the input 1204 as chunks. Thus, in some cases, the attention unit 1202 processes input of a certain size as chunks, where the number of chunks is represented as K. Thus, in one or more embodiments, the L dimension represents the sequence length per chunk. To illustrate, in some implementations, the attention unit 1202 performs the quadratic attention via chunks and merges the processed chunks at the end. For instance, in some cases, the splice-site prediction system 106 merges the processed quadratic chunks with the processed linear data (e.g., at the end of the attention unit 1202 or at the end of the neural network). As an example, where an input sequence has a length of 8K, the splice-site prediction system 106 can split the data into two 4K chunks (based on given parameters), perform the quadratic attention on each chunk, and then merge the processed data to obtain data that represents the 8K input in its entirety.

As shown in FIG. 12A, the attention unit 1202 reshapes one or more of the value tensor V, the query tensor Q, or the key tensor K. In particular, the attention unit 1202 reshapes one or more of the feature tensors before providing the feature tensors to the component 1206 (i.e., the ReLU layer). The attention unit 1202 reshapes the one or more value tensors to reorganize the data contained therein.

To illustrate, as indicated by FIG. 12A, the attention unit 1202 utilizes a reshaping factor d in reshaping the feature tensor(s). In particular, as shown, the attention unit 1202 determines groups of data of size d from the sequence length dimension L and concatenates those groups of data along the attention dimension A. Thus, the attention unit 1202 effectively divides the sequence length dimension L by the reshaping factor d but expands the attention dimension A by the same factor. Thus, the attention unit 1202 changes the organization of data elements within each reshaped feature tensor without changing the number of elements that are represented therein.

As indicated by FIG. 12A, the attention unit 1202 utilizes the component 1206 to process the reshaped feature tensor(s). In some cases, after processing by the component 1206, the attention unit 1202 returns the data to its (B, L, A) dimensionality. In one or more embodiments, where the transformer neural network includes multiple transformer blocks, each with its own attention unit, the splice-site prediction system 106 configures each attention unit to reshape the data therein as described above.

In one or more embodiments, by having the attention unit 1202 reshape one or more of the feature tensors, the splice-site prediction system 106 reduces the computational complexity within the attention unit 1202. In particular, the splice-site prediction system 106 reduces the computational complexity of the attention operation performed by the component 1206 by an amount equal to the reshaping factor d. Thus, by having the attention unit 1202 reshape one or more of the feature tensors, the splice-site prediction system 106 improves the suitability of the transformer neural network for the task of splice site detection. Indeed, as previously mentioned, transformer neural networks are typically associated with a high computational complexity (i.e., time and space complexity) that scales quadratically to input sequence length-represented as O(n²). By having the attention unit 1202 reshape one or more of the feature tensors, however, the splice-site prediction system 106 reduces the computational complexity of the transformer neural network as follows, where K represents the chunk number:

$$O ( {KL}^{2} A ) \to O ( {K ( L / d )}^{2} A d ) = O ( \frac{{KL}^{2} A}{d} ) \qquad ( 6 )$$

Indeed, the attention operation can be complex, leading to the quadratically scaling computational complexity. In particular, the complexity of the attention operation can depend on the number of positions being analyzed. Thus, by reshaping one or more of the feature tensors, the splice-site prediction system 106 increases the number of features associated with each position in one dimension (i.e., the attention dimension A) but reduces the number of positions in another dimension (i.e., the sequence length dimension L). Though the above describes the reshaping feature strictly in terms of reducing the sequence length dimension L and expanding the attention dimension A, it should be understood that the splice-site prediction system 106 can reduce or expand other dimensions of the feature tensors in various embodiments.

Thus, by reshaping the data within the attention units, the splice-site prediction system 106 can flexibly implement transformer neural networks for the task of predicting splice sites, where existing systems were typically limited to CNNs. Indeed, by configuring the attention units to reshape the data therein, the splice-site prediction system 106 reduces the computational complexity that prevented the practical application of transformer neural networks to the splice-site prediction task. Thus, the splice-site prediction system 106 can flexibly utilize a transformer neural network that is better suited to handling long input sequences when compared to the CNNs employed by conventional systems.

FIG. 12B illustrates graphs reflecting experimental results regarding the performance of a transformer neural network that can be implemented by the splice-site prediction system 106 having various data shapes within its attention unit(s) in accordance with one or more embodiments. In particular, FIG. 12B illustrates graphs that compare an implementation of a transformer neural network having one or more attention units that operate without reshaping the data therein to an implementation having one or more attention units that do reshape the data therein. Notably, the implementation of the transformer neural network having the attention units that does reshape the data therein is shown as an eighty-layer model, whereas the transformer neural network that does not reshape the data is only shown as a fourteen-layer model. Indeed, by reshaping the data within the attention units, the splice-site prediction system 106 can implement a much deeper neural network with additional layers and input sequence length than would otherwise be practical without the reshaping.

The first graph 1210 shows the performance of each model with respect to a training loss metric. The second graph 1212 shows the performance of each model with respect to a validation loss metric. As shown by the graphs, the transformer neural network that reshapes data within its attention units achieves a much lower validation loss when compared to the transformer neural network that does not reshape its data. Further, while the training loss for the reshaping transformer neural network is comparatively higher, the gap between the training loss and the validation loss for that model is smaller, indicating that the model is performing better overall.

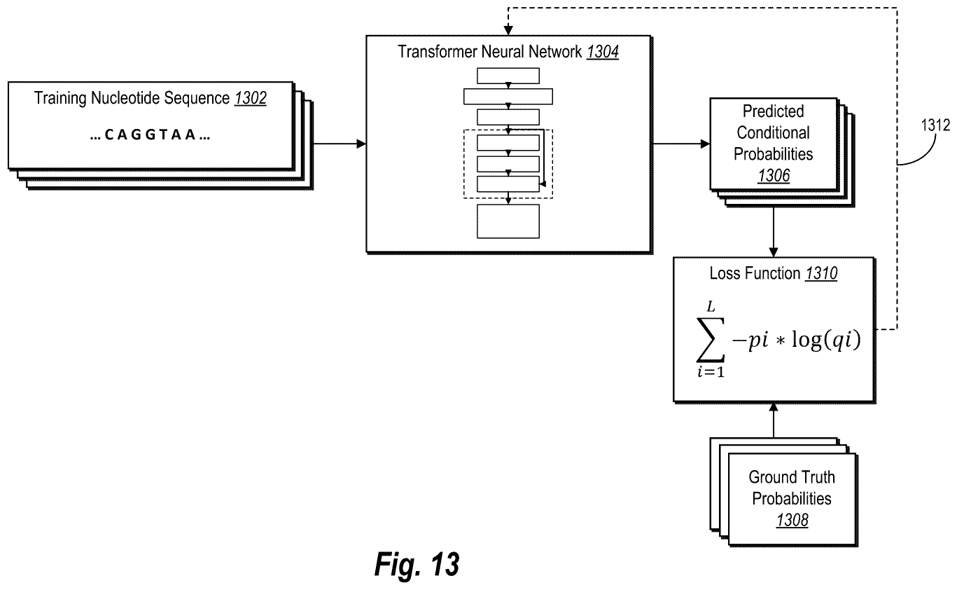

In one or more embodiments, the splice-site prediction system 106 trains a neural network, such as a transformer neural network for the task of predicting splice sites. In particular, the splice-site prediction system 106 trains the neural network to analyze input nucleotide sequences and generate corresponding conditional probabilities. FIG. 13 illustrates the splice-site prediction system 106 training a neural network to generate conditional probabilities for nucleotide sequences in accordance with one or more embodiments.

Indeed, as shown in FIG. 13, the splice-site prediction system 106 utilizes a training nucleotide sequence 1302 to train a transformer neural network 1304. In some cases, the training nucleotide sequence 1302 includes a conditioned nucleobase at a conditioned position and a plurality of flanking nucleobases within a threshold number of nucleobases from the conditioned nucleobase. In some instances, the training nucleotide sequence 1302 also includes a context sequence on one or both sides of the conditioned nucleobase and flanking nucleobases.

As shown in FIG. 13, the splice-site prediction system 106 provides the training nucleotide sequence 1302 to the transformer neural network 1304. Further, the splice-site prediction system 106 utilizes the transformer neural network 1304 to generate predicted conditional probabilities 1306 for the training nucleotide sequence 1302. In some embodiments, each predicted conditional probability from the predicted conditional probabilities 1306 corresponds to a flanking nucleobase within the training nucleotide sequence 1302 and indicates a likelihood that the flanking nucleobase is part of a matching donor or a matching acceptor for the conditioned nucleobase.

As further shown in FIG. 13, the splice-site prediction system 106 compares the predicted conditional probabilities 1306 to ground truth probabilities 1308 via a loss function 1310. In one or more embodiments, the splice-site prediction system 106 derives the ground truth probabilities 1308 that correspond to the training nucleotide sequence 1302 using read data (e.g., RNA sequencing data) for the training nucleotide sequence 1302. In some cases, the splice-site prediction system 106 can derive the ground truth probabilities 1308 using short nucleotide reads (e.g., reads of 150 nucleobases or less) and/or long nucleotide reads (e.g., reads covering more than 150 nucleobases).

In some cases, the utility of using short nucleotide reads in determining ground truth probabilities comes from the formulation of the splice-site prediction task as a conditional probability problem. In general, determining how often a position is used as a splice site is difficult in many cases because there is uneven coverage of the read data. Indeed, coverage across short nucleotide reads can systematically decrease across nucleotide sequences (e.g., from the 3′ (end) to the 5′ (start)) in read data stored in even some of the most widely used databases, such as the Genotype-Tissue Expression (GTEx) database resulting from the GTEx project as described by The GTEx Consortium, The Genotype-Tissue Expression (GTEx) Project, Nat Gent, 2013 June; 45 (6): 580-585, which is incorporated herein by reference in its entirety. This inconsistency in coverage can cause difficulty in accurately determining the use of each position as a splice site.

By formulating the splice-site prediction task as a conditional probability problem, however, the splice-site prediction system 106 can mitigate the issue of variable coverage. Indeed, read data typically covers spliced nucleotide sequences. Accordingly, many of the splice sites covered by short nucleotide reads are in neighboring positions as the nucleobases (e.g., introns) that are initially positioned between those splice sites are typically (at least partially) spliced out. Thus, this formulation allows for the determination of how often some splice sites are used given other splice sites with good accuracy.

In some instances, however, short nucleotide reads are useful for determining ground truth probabilities for some of the targeted conditional probabilities but not others. In particular, short nucleotide reads can be useful in determining ground truth probabilities that identify a downstream acceptor that matches a conditioned donor or to identify an upstream donor that matches a conditioned acceptor. This is because those splice site pairs cover the ends of an intron. Thus, splicing will typically place those splice site pairs in neighboring (e.g., adjacent) positions, and the short nucleotide read coverage will be about even for those splice site pairs (e.g., if a short nucleotide read covers a donor, it will likely also cover the matching downstream acceptor).

As used herein, the term “splice site pair” refers to a pair of corresponding (e.g., adjacent) splice sites. In particular, a splice site pair can include a pair consisting of a donor and an acceptor, where the donor and acceptor are adjacent splice sites. For instance, a splice site pair can include a first splice site (e.g., a donor or acceptor) and an adjacent downstream or upstream splice site (e.g., an acceptor or donor, respectively).

For conditional probabilities related to a donor that matches an upstream acceptor or an acceptor that matches a downstream decoder, short nucleotide reads can be unhelpful. Indeed, these splice site pairs cover the ends of an exon. Often, an exon will be larger than a short nucleotide read, such that a short nucleotide read may cover one end of the exon but not the other. Thus, short nucleotide reads often do not allow for the determination of, for example, which position will be used as a matching acceptor that is upstream from a donor. Thus, in some embodiments, the splice-site prediction system 106 utilizes long nucleotide reads to determine ground truth probabilities. Indeed, as long nucleotide reads are typically long enough to cover the span of an exon, the splice-site prediction system 106 can utilize this read data to determine ground truth probabilities for splicing pairs at the ends of exons. In some cases, the splice-site prediction system 106 also utilizes long nucleotide reads to determine ground truth probabilities for splicing pairs positioned at the ends of introns as well (e.g., in addition to using the short nucleotide reads).

Generally speaking, in one or more embodiments, the splice-site prediction system 106 determines the ground truth probability for a particular splice site within a training nucleotide sequence based on a number of reads (a count) for the splice site and the total number of splicing events represented for the training nucleotide sequence in the read data. In particular, the splice-site prediction system 106 can divide the count for the splice site by the total number of splicing events to determine the corresponding ground truth probability. In some cases, the splice-site prediction system 106 only includes reads that cover matching splice sites (i.e., junction counts) in the total number of splicing events. In some implementations, however, the splice-site prediction system 106 includes all reads, even if they do not cover a matching splice site.

In one or more embodiments, the splice-site prediction system 106 utilizes a cross-entropy loss function as the loss function 1310 to compare the predicted conditional probabilities 1306 and ground truth probabilities 1308. In particular, in some cases, the splice-site prediction system 106 utilizes the following cross-entropy loss function where pᵢ represents the ground truth probabilities 1308 and qᵢ represents the predicted conditional probabilities 1306:

$$Loss = {\sum }_{i = 1}^{L} - {p}_{i} * log ( {q}_{i} ) \qquad ( 7 )$$

As shown in FIG. 13, the splice-site prediction system 106 back propagates the loss determined via the loss function 1310 to the transformer neural network 1304 (as shown by the dashed line 1312) to update the parameters of the transformer neural network 1304. In particular, the splice-site prediction system 106 updates the parameters to minimize the error of the transformer neural network 1304 in generating conditional probabilities for nucleotide sequences.

Though FIG. 13 illustrates training the transformer neural network 1304 using one refinement iteration, it should be noted that, in some embodiments, the splice-site prediction system 106 trains the transformer neural network 1304 using multiple refinement iterations. In one or more embodiments, with each iteration of refinement, the splice-site prediction system 106 gradually improves the accuracy with which the transformer neural network 1304 generates conditional probabilities. Thus, the splice-site prediction system 106 learns parameters for the transformer neural network 1304 that facilitate its performance in the splice-site prediction task.

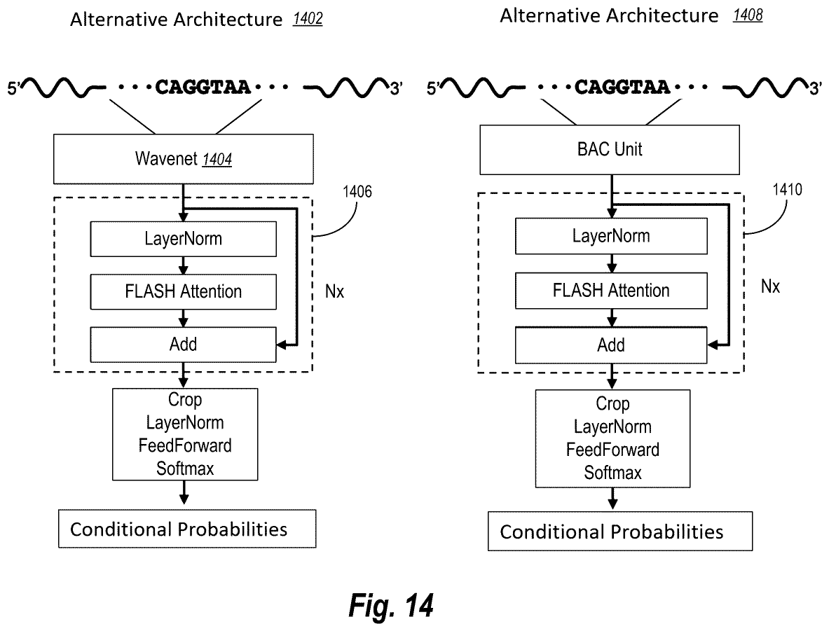

While the above describes a particular architecture of a transformer neural network, the splice-site prediction system 106 can utilize various transformer neural network architectures in various embodiments. FIG. 14 illustrates alternative transformer neural network architectures implemented by the splice-site prediction system 106 in accordance with one or more embodiments.

In particular, FIG. 14 shows a first alternative architecture 1402 for a transformer neural network that could be implemented by the splice-site prediction system 106. As shown, the first alternative architecture 1402 includes a Wavenet model 1404 that is followed by a transformer block 1406. The Wavenet model 1404 is a convolutional-based model. Accordingly, the first alternative architecture 1402 swaps the residual convolutional blocks discussed above for a Wavenet convolutional component. FIG. 14 also shows a second alternative architecture 1408. As shown, the second alternative architecture omits the convolutional components and consists largely of the transformer block 1410. Thus, the splice-site prediction system 106 can be flexible in the architecture that is trained and implemented for the splice-site prediction task.

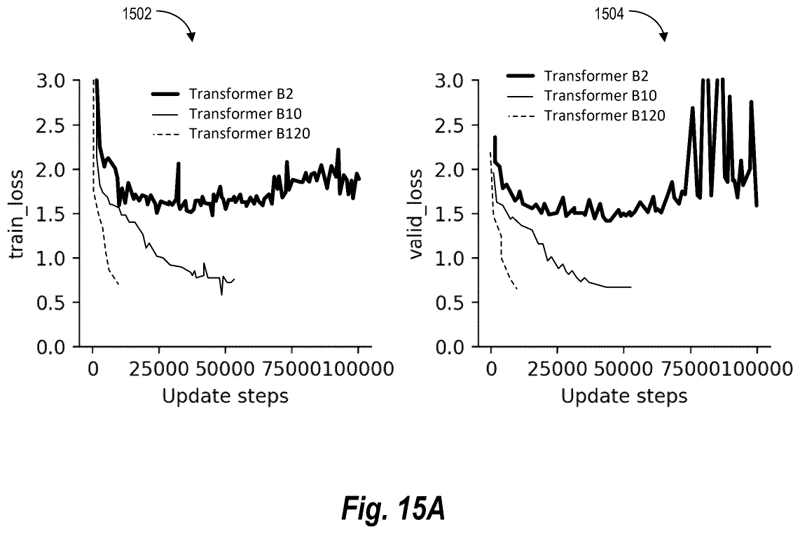

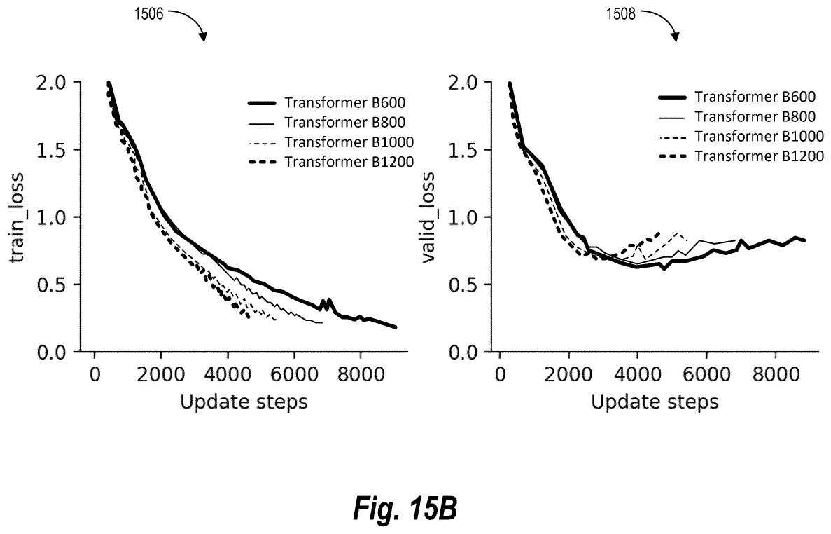

As suggested above, the splice-site prediction system 106 trains a transformer neural network for the task of splice site detection utilizing training data. In some cases, the splice-site prediction system 106 uses the training data in batches to train the transformer neural network. FIGS. 15A-15B illustrates graphs reflecting experimental results regarding the performance of different implementations of a transformer neural network that can be employed by the splice-site prediction system 106 based on training batch size in accordance with one or more embodiments.

In particular, FIG. 15A illustrates a first graph 1502 and a second graph 1504 reflecting the performance of different implementations of a transformer neural network when trained using a batch size of two, a batch size of ten, and a batch size of one hundred twenty. Each batch size corresponds to the number of training samples included in the batch. The first graph 1502 shows the performance of the tested models with respect to a training loss metric. The second graph 1504 shows the performance of the tested models with respect to a validation loss metric. FIG. 15B illustrates a third graph 1506 and a fourth graph 1508 reflecting the performance of different implementations of a transformer neural network when trained using a batch size of six hundred, a batch size of eight hundred, a batch size of one thousand, and a batch size of twelve hundred. The third graph 1506 shows the performance of the tested models with respect to a training loss metric. The fourth graph 1508 shows the performance of the tested models with respect to a validation loss metric.

As shown by FIGS. 15A-15B, batch size can have an impact on the performance of a transformer neural network. For instance, as shown by the graphs of FIG. 15A, too small a batch size can prevent the transformer neural network from converging. Rather, the transformer neural network can get stuck in a high loss without improving. Accordingly, training the transformer neural network with too small of a batch size can prevent learning. As indicated in FIG. 15A, however, a batch size of ten can be used as a minimum batch size to train a transformer neural network to generate the conditional probabilities disclosed herein. Additionally, as shown by the graphs of FIG. 15B, too large a batch size can result in poor performance. In particular, while the third graph 1506 shows a low training loss, the fourth graph 1508 shows a validation loss that fails to fall below a certain level. Further, the validation loss appears to being increasing after a certain point. Thus, in some cases, the splice-site prediction system 106 trains a transformer neural network using a batch size that allows for convergence while avoiding poor performance during inference.

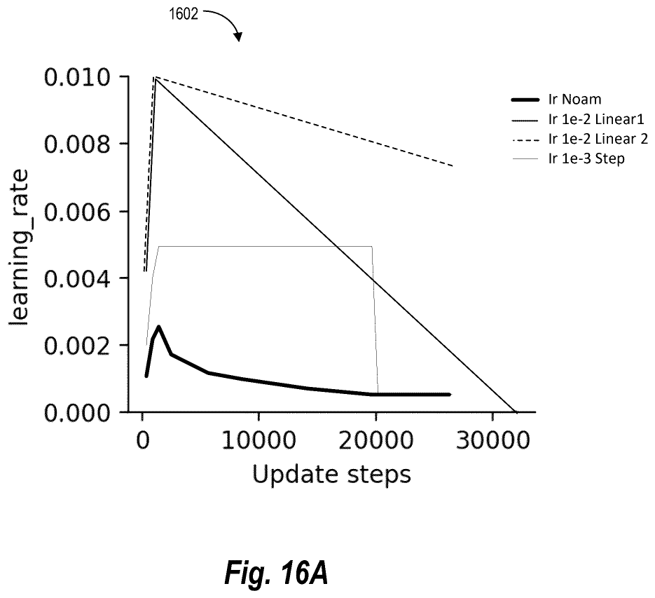

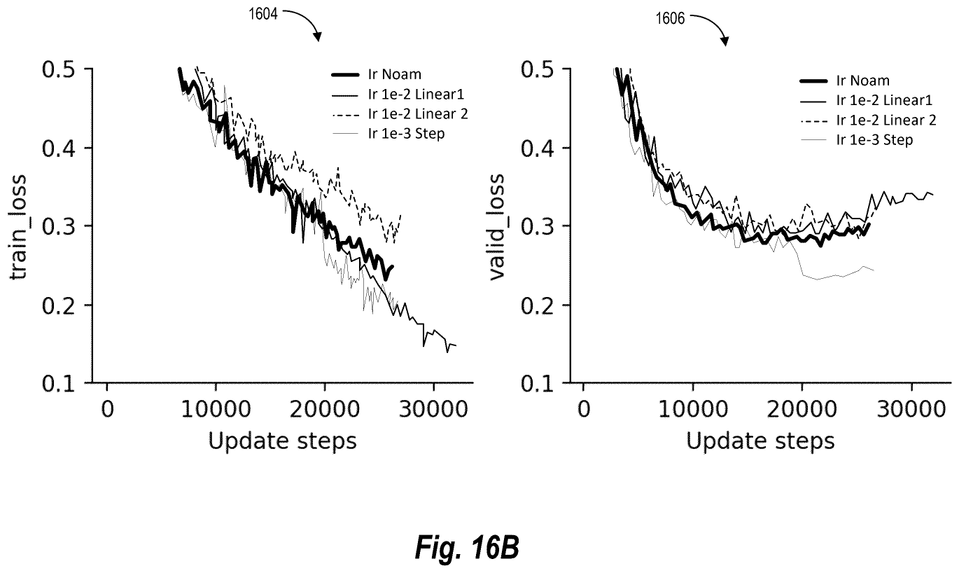

The splice-site prediction system 106 can train the transformer neural network using various learning rates in various embodiments. FIGS. 16A-16B illustrates graphs reflecting experimental results regarding the performance of different implementations of a transformer neural network that can be employed by the splice-site prediction system 106 based on the learning rate used during training in accordance with one or more embodiments.

In particular, FIG. 16A illustrates a first graph 1602 showing different learning rate configurations used to train different implementations of a transformer neural network. The learning rate configurations differ in aspects, such as initial learning rate, peak learning rate, and the speed the learning rate decays. In particular, the first graph 1602 shows a stepwise learning rate that increase, flattens out, and then drops significantly over a short period of time. In one or more embodiments, the splice-site prediction system 106 employs a stepwise learning rate, such as the one shown in the first graph 1602.

FIG. 16B illustrates a second graph 1604 and a third graph 1606 reflecting the performance of different implementations of a transformer neural network when trained using the learning rates illustrated in the first graph 1602 of FIG. 16A. The second graph 1604 shows the performance of the tested models with respect to a training loss metric. The third graph 1606 shows the performance of the tested models with respect to a validation loss metric. As shown by the graphs of FIG. 16B, the stepwise learning rate used by the splice-site prediction system 106 in some embodiments appears to provide the best performance especially in view of the validation loss metric in that the loss is lower when compared to the losses associated with the other learning rates. Accordingly, in some embodiments, the splice-site prediction system 106 utilizes a stepwise learning rate with a stepwise learning rate decay to train a transformer neural network for the task of splice site detection. Other learning rates, however, can be used in various embodiments.

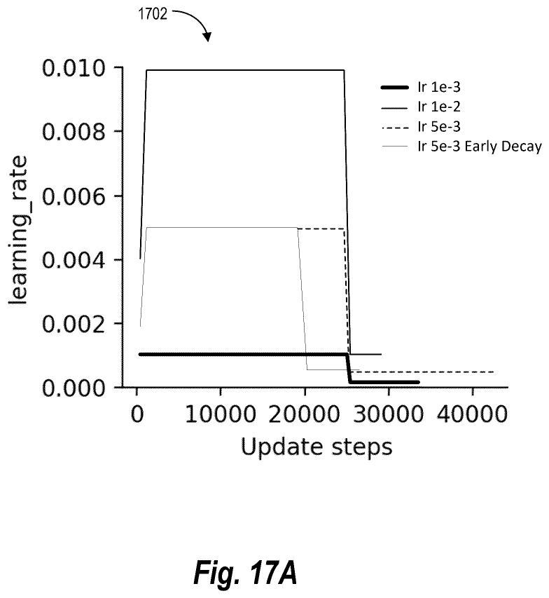

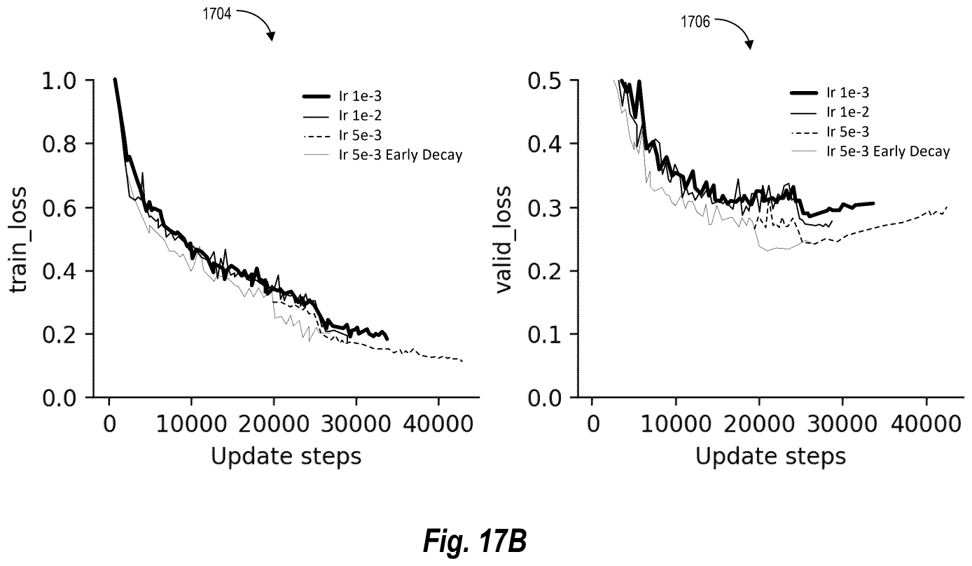

FIGS. 17A-17B illustrates graphs reflecting experimental results regarding the performance of different implementations of a transformer neural network that can be employed by the splice-site prediction system 106 based on a stepwise learning rate used during training in accordance with one or more embodiments. In particular, FIG. 17A illustrates a first graph 1702 showing different stepwise learning rates used to train different implementations of a transformer neural network. The stepwise learning rates shown differ in their initial learning rate, the learning rate at which they flatten out, when the learning rate begins to decay, and the resulting final learning rate.

FIG. 17B illustrates a second graph 1704 and a third graph 1706 reflecting the performance of different implementations of a transformer neural network when trained using the stepwise learning rates illustrated in the first graph 1702 of FIG. 17A. The second graph 1704 shows the performance of the tested models with respect to a training loss metric. The third graph 1706 shows the performance of the tested models with respect to a validation loss metric. As shown by the graphs of FIG. 17B, the scheduling of a learning rate (e.g., how the learning rate is configured in terms of its initial rate, its flattened learning rate, its timing of decay, and its final learning rate) can have a significant impact on the performance of the resulting model. In particular, the graphs suggest that models trained using the stepwise learning rate labeled “1r 5e-3” perform significantly better than those trained using the other learning rates in that they achieve lower loss. The model trained using the stepwise learning rate labeled “1r 5e-3 Early Decay” appears to provide an even better performance when compared to the model trained using the “1r 5e-3” stepwise learning rate without the early decay.

Thus, in some cases, the splice-site prediction system 106 configures a stepwise learning rate that improves the training of a transformer neural network in accordance with the results shown in FIGS. 17A-17B. To provide at least one example, the splice-site prediction system 106 can employ a schedule that involves an incrementally increasing learning rate up to between 1e-4 and 5e-3; a first fixed learning rate of between 1e-4 and 5e-3 during a first set of update steps that ends after a threshold number of update steps (e.g., a pre-configured number of update steps) between one thousand update steps and two thousand update steps; and a second fixed learning rate that is lower than 1e-4 during a second set of update steps that begins after the threshold number of update steps.

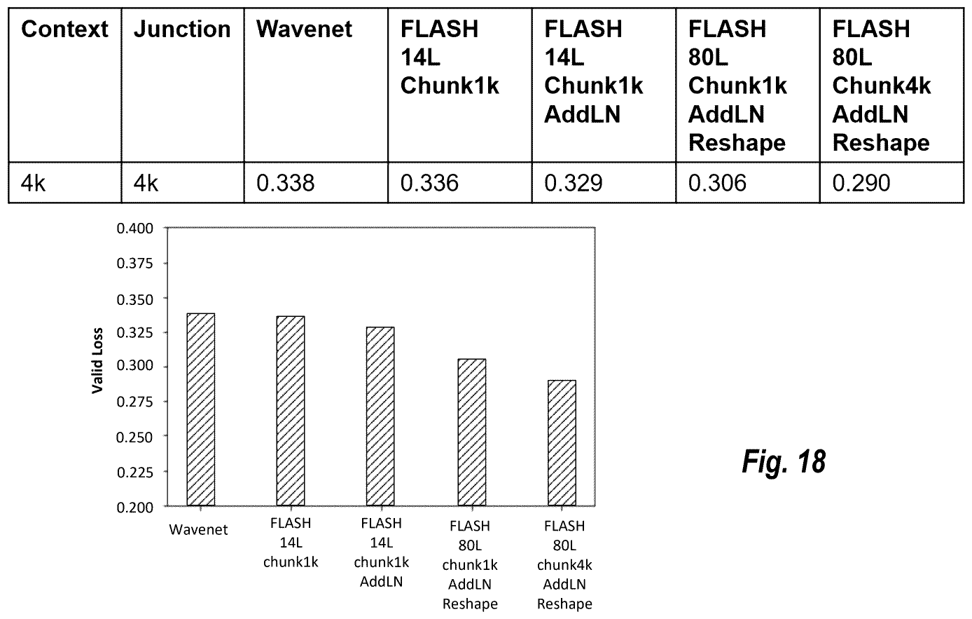

FIG. 18 illustrates a table and a graph reflecting yet further experimental results regarding the performance of different implementations of a transformer neural network that can be employed by the splice-site prediction system 106 in accordance with one or more embodiments. In particular, the table and graph compare the performance of the different implementations of the transformer neural network to the Wavenet model described above. The performance of the tested models was measured using a validation loss metric. Further, as shown in the table, the nucleotide sequences that were analyzed by each tested model included a context sequence of four thousand nucleobases and a junction of four thousand flanking nucleobases. The graph provides a visual representation of the numbers included in the table.

The various implementations of a transformer neural network represented within FIG. 18 are all FLASH-based transformer neural networks and include (i) a model that has fourteen layers and analyzes input data in chunks of one thousand; (ii) a model that has fourteen layers, analyzes input data in chunks of one thousand, and further includes an added normalization layer in the attention units; (iii) a model that has eighty layers, analyzes input data in chunks of one thousand, includes an added normalization layer in the attention units, and performs data reshaping in its attention units; and (iv) a model that has eighty layers, analyzes input data in chunks of four thousand, includes an added normalization layer in the attention units, and performs data reshaping in its attention units.

As shown in FIG. 18, all tested implementations of the transformer neural network outperformed the Wavenet model. As further shown, the transformer neural networks with the added normalization layer in the attention units outperformed the similar model without the added normalization layer. Further, the transformer neural networks with the larger number of layers and the reshaping feature significantly improved on the validation loss shown. The final transformer neural network that analyzed the data in chunks of four thousand provides the best performance out of those shown in this figure.

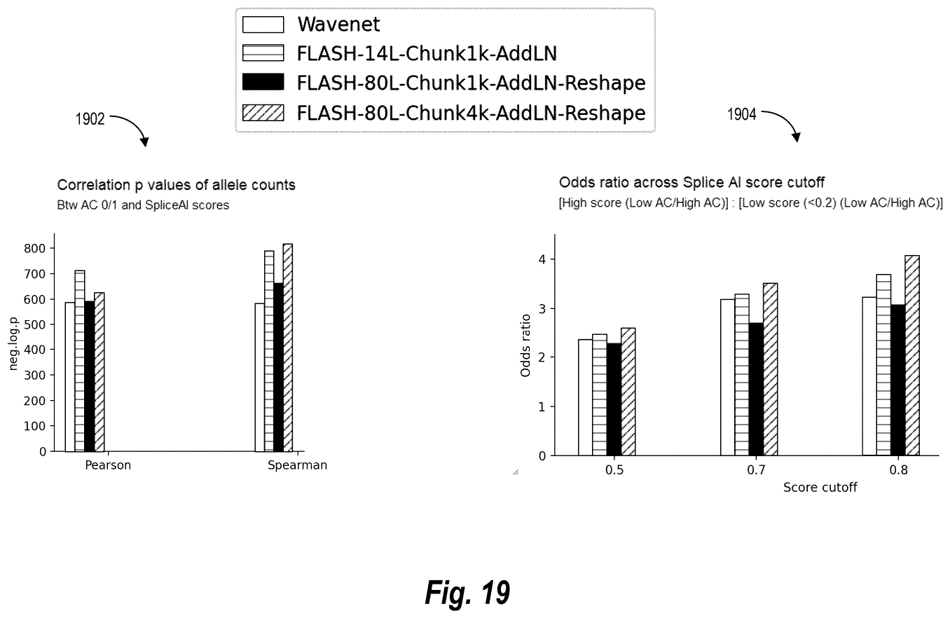

FIG. 19 illustrates graphs reflecting additional experimental results regarding the performance of different implementations of a transformer neural network that can be employed by the splice-site prediction system 106 in accordance with one or more embodiments. In particular, the graphs of FIG. 19 compare the performance of the Wavenet model to models (ii)-(iv) discussed above with reference to FIG. 18. The first graph 1902 of FIG. 19 shows the performance of the tested models with respect to a negative log probability metric indicating the Pearson and Spearman correlations. As shown by the first graph 1902, the implementations of the transformer neural network achieved much better performance compared to the Wavenet model in terms of both correlation measures. The second graph 1904 of FIG. 19 shows the performance of the tested models with respect to the odds ratio test. As indicated, the implementations of the transformer neural network generally outperformed the Wavenet model.

Thus, the splice-site prediction system 106 implements a more flexible model for the task of splice site detection when compared to existing systems. In particular, by configuring a new, first-of-its-kind transformer neural network that addresses many of the issues that have prevented transformer neural networks from being applied to the task by existing systems, the splice-site prediction system 106 implements a flexible architecture that can perform splice site detection while realizing the analytical benefits of a transformer neural network. Indeed, by improving upon the computational complexity, training stability, convergence speed, and training losses typically experienced by transformer neural networks, the splice-site prediction system 106 implements a unique transformer neural network that can be practically used for detecting splice sites. Using a transformer neural network, the splice-site prediction system 106 can analyze longer nucleotide sequences than was possible under existing systems, leading to more accurate determination of splice sites and their associated probabilities.

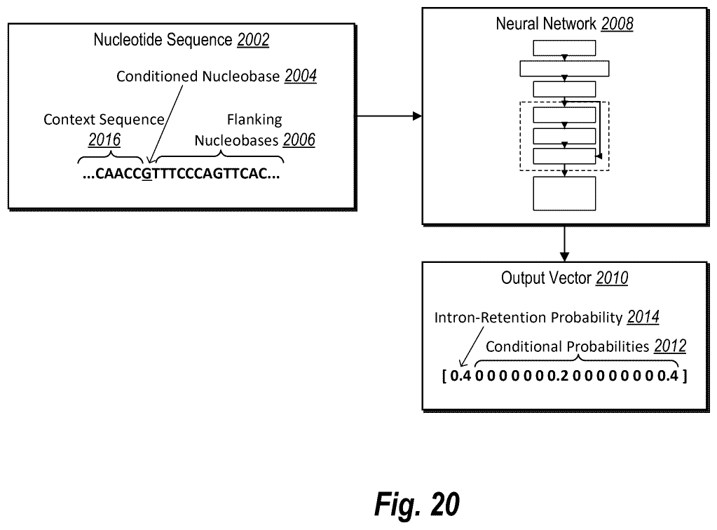

As previously mentioned, the splice-site prediction system 106 can configure the neural network (e.g., the transformer neural network) employed for splice site detection to also model intron retention. In particular, in some embodiments, the splice-site prediction system 106 configures the neural network to generate an intron-retention probability that indicates a likelihood that an intron is retained upon splicing of the nucleotide sequence. FIGS. 20-29 illustrate the splice-site prediction system 106 configuring and using a neural network to model intron retention in accordance with one or more embodiments.

FIG. 20 illustrates an overview of the splice-site prediction system 106 modeling intron retention for a nucleotide sequence in accordance with one or more embodiments. As shown in FIG. 20, the splice-site prediction system 106 provides a nucleotide sequence 2002 having a conditioned nucleobase 2004 at a conditioned position, flanking nucleobases 2006, and a context sequence 2016 to a neural network 2008. In some cases, nucleobases within the nucleotide sequence 2002 are part of an intron. For instance, one or more portions of the nucleobases preceding or following the conditioned nucleobase 2004 (e.g., one or more portions of the flanking nucleobases 2006 and/or the nucleobases that are included in the context sequence 2016) can be part of an intron. To illustrate, where the conditioned nucleobase 2004 corresponds to a donor, one or more portions of the flanking nucleobases 2006 can represent an intron as they are positioned downstream from the conditioned nucleobase 2004.

As shown, the splice-site prediction system 106 uses the neural network 2008 to generate an output vector 2010. The output vector 2010 includes conditional probabilities 2012 where each conditional probability corresponds to a flanking nucleobase from the nucleotide sequence 2002 and indicates a likelihood that the flanking nucleobase is part of a matching donor or a matching acceptor for the conditioned nucleobase 2004. The output vector 2010 further includes an intron-retention probability 2014 that indicates a likelihood that an intron is retained as the nucleotide sequence 2002 is spliced (e.g., retained in the mRNA that corresponding to the nucleotide sequence 2002 that results from splicing). Thus, in some embodiments, in addition to using the neural network 2008 to generate conditional probabilities that identify splice sites, the splice-site prediction system 106 can use the neural network 2008 to model intron retention by generating intron-retention probabilities for nucleotide sequences.

It should be noted that the values of the output vector 2010 sum to a value of one. In particular, the values of the conditional probabilities 2012 and the intron-retention probability 2014 sum to a value of one. The significance of this result will be described in more detail below.

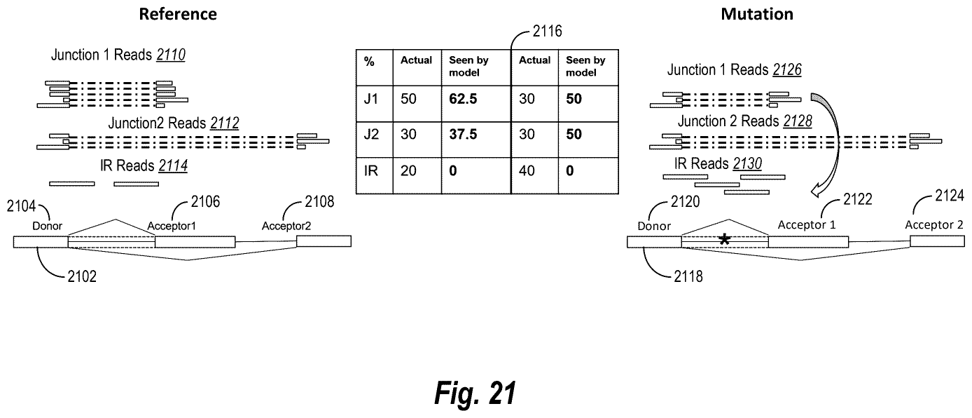

FIG. 21 illustrates an example of how intron retention affects splicing within a nucleotide sequence and the significance of modeling intron retention when detecting splice sites in accordance with one or more embodiments. FIG. 21 illustrates a reference nucleotide sequence 2102 that includes a donor 2104, a first acceptor 2106, and a second acceptor 2108. The first acceptor 2106 and the second acceptor 2108 represent potential matching acceptors for the donor 2104. FIG. 21 also illustrates an example set of reads for the reference nucleotide sequence 2102 that includes first junction reads 2110, second junction reads 2112, and intron retention reads 2114. The first junction reads 2110 includes those reads that cover the donor 2104 and the first acceptor 2106. The second junction reads 2112 includes those reads that cover the donor 2104 and the second acceptor 2108. Accordingly, the first junction reads 2110 and the second junction reads 2112 indicate that splicing has occurred in those instances. The intron retention reads 2114, on the other hand, include those reads that at least partially cover an intron within the reference nucleotide sequence 2102. Accordingly, the intron retention reads 2114 indicate that intron retention has occurred in those instances. Indeed, as previously indicated, reads typically cover a nucleotide sequence after splicing has occurred. Thus, where a read covers an intron, it can be concluded that the intron has been retained during splicing.

FIG. 21 also shows a table 2116 that includes read counts and percentages for splicing events. In particular, the percentages shown in the table 2116 include percentages of splicing events as seen by a model (e.g., SpliceAI V1) that does not model intron retention, such as one of the models used in many existing systems. In other words, the percentages shown in the table reflect the percentages of splicing events when intron retention is ignored and only accounts for reads the first junction and the second junction.

The left side of the table 2116 corresponds to the reference nucleotide sequence 2102. As shown, while the read counts reveal that intron retention accounts for twenty splicing events for the reference nucleotide sequence 2102, the model does not detect these occurrences; rather, the model only recognizes the eighty splicing events associated with the first and second junctions. Thus, instead of attributing twenty percent of the splicing events to intron retention—which is what actually occurred—the model inflates the percentage of splicing events that involve the first and second junctions. As such, not only does the model fail to detect intron retention for the reference nucleotide sequence 2102, the model also fails to accurately determine the frequency of splicing events that involve the first and second junctions. Indeed, the model provides an inaccurately high estimation of how often the first and second junctions are involved.

FIG. 21 further illustrates a mutated nucleotide sequence 2118 that corresponds to the reference nucleotide sequence 2102. For instance, the mutated nucleotide sequence 2118 can include one or more variants when compared to the reference nucleotide sequence 2102. In some instances, the one or more variants affect how the mutated nucleotide sequence 2118 experiences splicing. In other words, the one or more variants can affect the splicing events (or the frequency of splicing events) that can occur for the mutated nucleotide sequence 2118.

Like the reference nucleotide sequence 2102, the mutated nucleotide sequence 2118 includes a donor 2120, a first acceptor 2122, and a second acceptor 2124. FIG. 21 also illustrates an example set of reads for the mutated nucleotide sequence 2118 that includes first junction reads 2126, second junction reads 2128, and intron retention reads 2130.

The right side of the table 2116 corresponds to the mutated nucleotide sequence 2118. As shown, the mutation occurring within the mutated nucleotide sequence 2118 (i.e., the one or more variants included therein) has led to a change in the occurrence of the represented splicing events. In particular, the splicing events for the mutated nucleotide sequence 2118 involving the first and second junctions are shown as even, where the splicing events involving the first junction were significantly higher than those involving the second junction for the reference nucleotide sequence 2102. Further, the splicing events that involve intron retention have doubled for the mutated nucleotide sequence 2118 compared to the reference nucleotide sequence 2102.

As shown by the table 2116, however, as the model fails to detect intron retention, the percentages associated with each splicing event misrepresents the actual occurrence of those splicing events and further misrepresents how the one or more variants of the mutated nucleotide sequence 2118 affects the changes in splicing. For instance, similar to the reference nucleotide sequence 2102, the model fails to attribute any splicing events for the mutated nucleotide sequence 2118 to intron retention. Further, the model attributes fifty percent to splicing events involving each of the first and second junctions. Notably, while the number of splicing events involving the second junction has remained the same when comparing the reference nucleotide sequence 2102 to the mutated nucleotide sequence 2118, the model attributes an increased percentage of splicing events involving the second junction to the mutated nucleotide sequence 2118.

Accordingly, the failure of the model (e.g., SpliceAI V1) represented in the table 2116 to account for intron retention has led to a failure in accurately representing how the one or more variants of the mutated nucleotide sequence 2118 affected splicing. Indeed, as just mentioned, the model artificially increased the percentage attributed to the splicing events involving the second junction where there was no increase in those splicing events. Rather, the one or more variants caused a decrease in the splicing events involving the first junction and an increase in the splicing events involving intron retention. In other words, the splicing events involving intron retention take away from the splicing events involving the first junction. As intron retention is not detected by the model, however, the model falsely attributes the drop in splicing involving the first junction to an increase in splicing events that involve the second junction.

As such, FIG. 21 illustrates the impact that modeling intron retention can have in determining the frequency with which splice sites are used. In particular, modeling intron retention allows for a more accurate determination of the probability a splice site will be used in splicing as a fraction of the total number of splicing events that can occur for a nucleotide sequence. FIG. 21 also illustrates the impact that modeling intron retention can have in determining how variants within a nucleotide sequence can affect splicing.

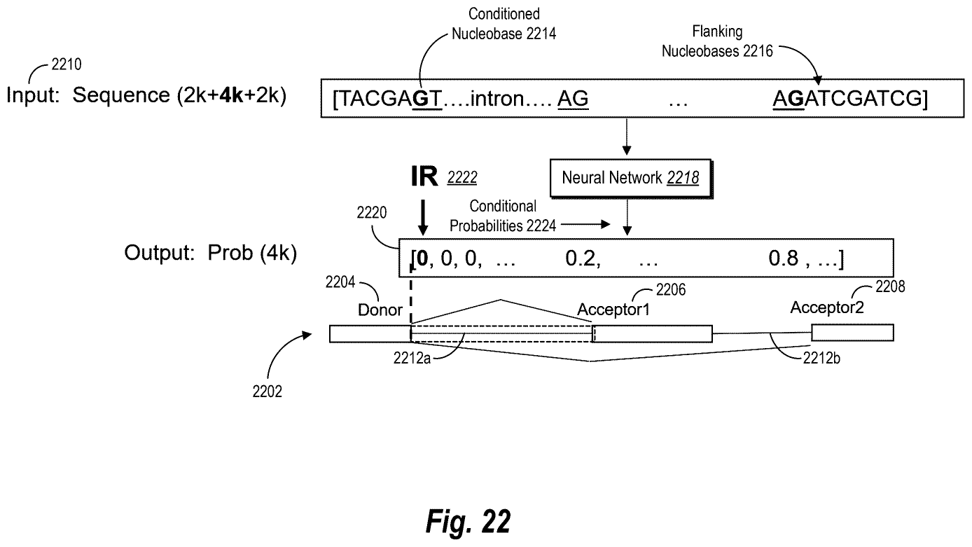

As mentioned, the splice-site prediction system 106 can configure a neural network to output an intron-retention probability that indicates a likelihood that a nucleotide sequence retains an intron after splicing. FIG. 22 illustrates an output generated by a neural network employed by the splice-site prediction system 106 that includes an intron-retention probability for a nucleotide sequence in accordance with one or more embodiments.

FIG. 22 shows a nucleotide sequence 2202 that includes a donor 2204, a first acceptor 2206, a second acceptor 2208, a first intron 2212a, and a second intron 2212b. FIG. 22 also illustrates an input sequence 2210 that corresponds to the nucleotide sequence 2202. As shown, the input sequence 2210 includes a junction of four thousand nucleobases with a context sequence of two thousand nucleobases on either side. The input sequence 2210 includes a conditioned nucleobase 2214 at a conditioned position and flanking nucleobases 2216 (i.e., the junction of four thousand nucleobases) downstream from the conditioned nucleobase 2214. As shown in FIG. 22, the splice-site prediction system 106 uses the neural network 2218 to generate an output vector 2220 that includes an intron-retention probability 2222 and conditional probabilities 2224.

As indicated by FIG. 22, the splice-site prediction system 106 can configure the neural network 2218 to output the intron-retention probability 2222 at a particular position within the output vector 2220. For instance, as shown, the splice-site prediction system 106 can configure the neural network 2218 to provide the intron-retention probability 2222 within the first position of the output vector 2220. In particular, the intron-retention probability 2222 is within the position corresponding to the flanking nucleobase that immediately follows the conditioned nucleobase 2214 within the input sequence 2210.

To explain differently, the above discussion has described that, in some cases, the splice-site prediction system 106 uses a neural network to generate an output vector of conditional probabilities where each conditional probability corresponds to a flanking nucleobase of the input sequence. As such, in some embodiments, the splice-site prediction system 106 generates an output vector having the same size as the number of flanking nucleobases within the input sequence so that each position within the output vector provides a conditional probability for a different flanking nucleobase. FIG. 22 shows, however, the splice-site prediction system 106 configuring the neural network 2218 to use one of the positions within the output vector 2220 to provide the intron-retention probability 2222 rather than a conditional probability for the corresponding nucleobase. Specifically, FIG. 22 shows the neural network 2218 using the position corresponding to the flanking nucleobase immediately following the conditioned nucleobase 2214 for providing the intron-retention probability 2222.

The splice-site prediction system 106 can use this position within the output vector 2220 to provide the intron-retention probability 2222 as the probability that the flanking nucleobase immediately following the conditioned nucleobase 2214 is a matching splice site is zero. Indeed, because the conditioned nucleobase 2214 is part of a splice site, the flanking nucleobase that immediately follows the conditioned nucleobase 2214 within the input sequence 2210 would not be part of a matching splice site. Rather, that flanking nucleobase is either part of an intron or an exon (depending on whether the conditioned nucleobase 2214 is part of a donor or an acceptor). As such, the splice-site prediction system 106 can use that position within the output vector 2220 to provide the intron-retention probability 2222 without the risk of omitting a relevant conditional probability that corresponds to a matching splice site.

More generally, in some cases, the splice-site prediction system 106 uses a position that corresponds to a flanking nucleobase within a threshold number of flanking nucleobases from the conditioned nucleobase 2214 to provide the intron-retention probability 2222. Indeed, in many cases, there are various positional options that are suitable for providing the intron-retention probability 2222. For instance, most introns and exons are over thirty nucleobases in length. Accordingly, the splice-site prediction system 106 can use any position that corresponds to the first thirty flanking nucleobases following the conditioned nucleobase 2214 for providing the intron-retention probability 2222.

It should be understood that the above describes this configuration in the context of the input sequence 2210 in which the flanking nucleobases 2216 are downstream from the conditioned nucleobase 2214. The same principal applies when an input sequence includes flanking nucleobases that are upstream from the conditioned nucleobase. In such a case, the splice-site prediction system 106 can use the position corresponding to the flanking nucleobase that immediately precedes the conditioned nucleobase to provide an intron-retention probability. More generally, the splice-site prediction system 106 can use a position that corresponds to a flanking nucleobase within a threshold number of flanking nucleobases that precede the conditioned nucleobase.

In some cases, rather than having an intron-retention probability occupying a position corresponding to a flanking nucleobase within the input sequence, the splice-site prediction system 106 configures the neural network 2218 to generate an additional output. For instance, in some cases, the splice-site prediction system 106 uses the neural network 2218 to generate an output vector corresponding to the flanking nucleobases and a separate output that includes the intron-retention probability. The splice-site prediction system 106 can provide the outputs separately or, in some cases, append the intron-retention probability to the front or back of the output vector having the conditional probabilities.

As shown in FIG. 22, the intron-retention probability 2222 has a value of zero. Indeed, in some cases, the splice-site prediction system 106 predicts that intron retention will not occur for the nucleotide sequence being analyzed. Accordingly, the splice-site prediction system 106 can generate an intron-retention probability having a value of zero to indicate an intron of the nucleotide sequence will not be retained. Where the splice-site prediction system 106 determines that there is a possibility of intron retention, the splice-site prediction system 106 can generate an intron-retention probability having a non-zero value that reflects the likelihood of intron retention.

Further, the values of the output vector 2220 (i.e., the values of the intron-retention probability 2222 and the conditional probabilities 2224) sum to a value of one. Indeed, as indicated above with reference to FIG. 21, intron retention affects the use of matching splice sites for a conditioned nucleobase in splicing events when it does occur. Accordingly, in some implementations, the splice-site prediction system 106 accounts for intron retention as part of the total number of splicing events associated with a nucleotide sequence and outputs probabilities that total to a value of one so that each probability represents a fraction of the total possible splicing events predicted for the nucleotide sequence.

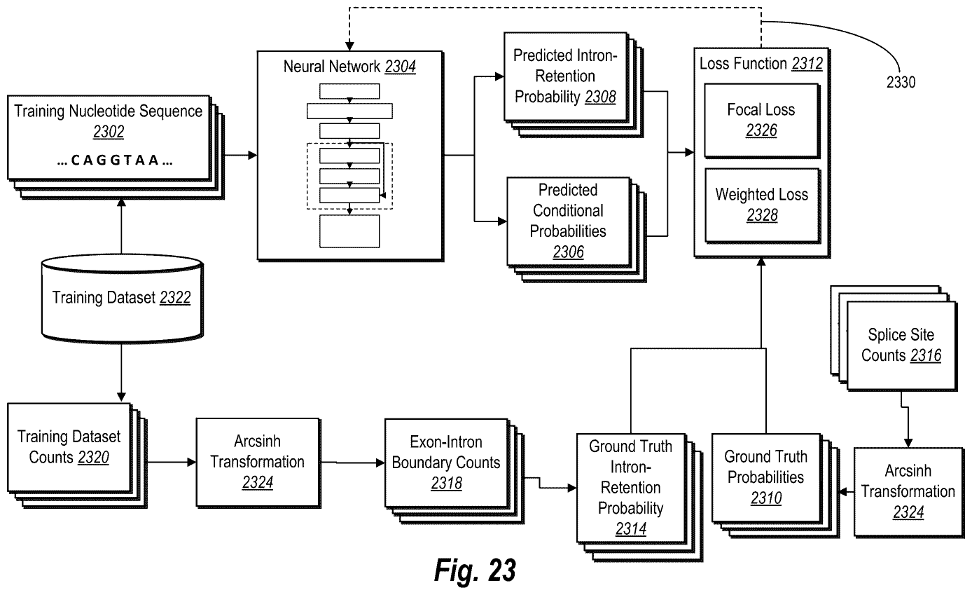

In one or more embodiments, the splice-site prediction system 106 configures a neural network to model intron retention via the training process. In particular, the splice-site prediction system 106 trains the neural network to generate conditional probabilities for flanking nucleobases of a nucleotide sequence as well as to generate an intron-retention probability. FIG. 23 illustrates the splice-site prediction system 106 training a neural network to model intron retention in accordance with one or more embodiments.

Indeed, as shown in FIG. 23, the splice-site prediction system 106 utilizes a training nucleotide sequence 2302 to train a neural network 2304. In some cases, the training nucleotide sequence 2302 includes a conditioned nucleobase at a conditioned position and a plurality of flanking nucleobases. In some instances, nucleobases within the training nucleotide sequence 2302 are part of an intron.

As shown in FIG. 23, the splice-site prediction system 106 provides the training nucleotide sequence 2302 to the neural network 2304. Further, the splice-site prediction system 106 utilizes the neural network 2304 to generate predicted conditional probabilities 2306 and a predicted intron-retention probability 2308 for the training nucleotide sequence 2302. In one or more embodiments, the splice-site prediction system 106 generates the predicted conditional probabilities 2306 and the predicted intron-retention probability 2308 within a single output vector or as a combination of separate outputs as previously described.

As further shown in FIG. 23, the splice-site prediction system 106 compares, via a loss function 2312, the predicted conditional probabilities 2306 to ground truth probabilities 2310, which are ground truth indicators for a matching splice site corresponding to the conditioned nucleobase of the training nucleotide sequence 2302. Further, the splice-site prediction system 106 compares, via the loss function 2312, the predicted intron-retention probability 2308 to a ground truth intron-retention probability, which is a ground truth indicator of intron retention.

As shown in FIG. 23, the splice-site prediction system 106 derives the ground truth probabilities 2310 from splice site counts 2316 associated with the training nucleotide sequence 2302. Indeed, as discussed above with reference to FIG. 13, the splice-site prediction system 106 can utilize read data associated with the training nucleotide sequence 2302 to determine a probability of use for matching splice sites given the conditioned nucleobase within the training nucleotide sequence 2302. For instance, the splice-site prediction system 106 can determine the ground truth probability for a particular splice site by dividing the count for the splice site by the total number of splicing events represented in the read data. As touched on in FIGS. 21-22, to model intron retention, the splice-site prediction system 106 includes read data that does not cover matching splice sites in the total number of splicing events. For instance, the splice-site prediction system 106 includes read data accounting for intron retention in the total number of splicing events.

As further shown in FIG. 23, the splice-site prediction system 106 derives the ground truth intron-retention probability 2314 from an exon-intron boundary count 2318 associated with the training nucleotide sequence 2302. In one or more embodiments, the splice-site prediction system 106 uses the exon-intron boundary count 2318 as an estimation of the number of intron retention events represented by the read data. As shown, the splice-site prediction system 106 can determine training dataset counts 2320 from a training dataset 2322 that includes the read data (and the training nucleotide sequences) and determines the exon-intron boundary count 2318 based on the training dataset counts 2320. In some cases, the training dataset counts 2320 include values determined by the splice-site prediction system 106 rather than values already included as part of the training dataset 2322.

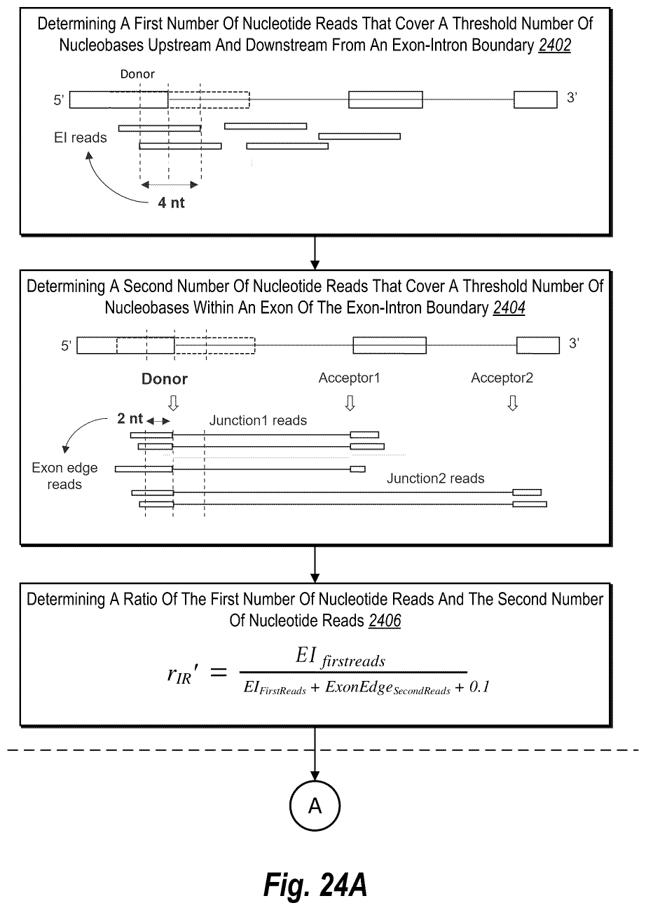

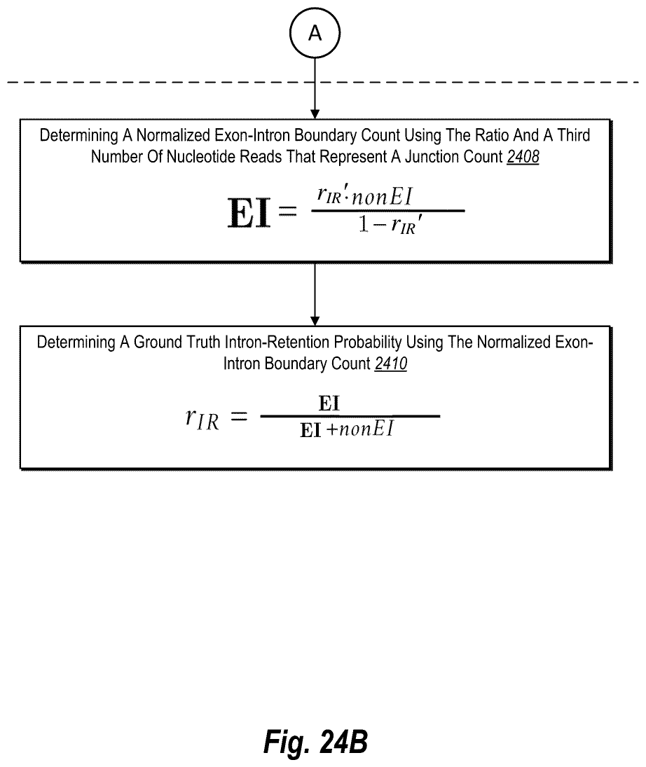

Indeed, in some cases, the training dataset 2322 used by the splice-site prediction system 106 to train the neural network 2304—such as the GTEx database—does not inherently include read data that provides a good indication of how many intron retention events are represented for a training nucleotide sequence. For instance, using nucleotide reads that cover the entire intron may be desirable to accurately determine intron retention has occurred, but the read data may not provide such coverage, which can cause biases in the read data. Accordingly, in some embodiments, the splice-site prediction system 106 estimates the number of splicing events related to intron retention by analyzing the training dataset 2322. In particular, the splice-site prediction system 106 can analyze the training dataset 2322 to determine the training dataset counts 2320 and then determine the exon-intron boundary count 2318 based on the training dataset counts 2320. More detail regarding the determination of the training dataset counts 2320 and the exon-intron boundary count 2318 will be provided below with reference to FIG. 24.

As shown in FIG. 23, the splice-site prediction system 106 applies an arcsinh transformation 2324 to the splice site counts 2316 and the exon-intron boundary count 2318. In particular, the splice-site prediction system 106 treats the splice site counts 2316 and the exon-intron boundary count 2318 as raw counts and generates transformed counts via the arcsinh transformation 2324. By applying the arcsinh transformation 2324 to the raw counts, the splice-site prediction system 106 can increase the weight of low-count events when training the neural network 2304. For instance, in some cases, intron retention is not a highly likely event for a training nucleotide sequence, so the read data may not have many reads indicating intron retention. Thus, the neural network 2304 may not learn parameters that adequately recognize the probability for intron retention when using raw counts. Accordingly, the splice-site prediction system 106 can use the arcsinh transformation 2324 to increase the weight of low count events to allow the neural network 2304 to adequately learn its parameters. Thus, the splice-site prediction system 106 can determine a ground truth probability for a particular event as follows:

$${P}_{i} = \frac{arc sinh ( {Count}_{i} )}{{\sum }_{i}^{n} arc sinh ( {Count}_{i} )} \qquad ( 8 )$$

In function 9, Countᵢ can refer to an intron retention count that indicates intron retention has occurred or a junction count that indicates the intron has been spliced out. Accordingly, the sum of Countᵢ in the denominator represents the total number of splicing events.

As shown in FIG. 23, the splice-site prediction system 106 can utilize one or more loss functions as the loss function 2312. For instance, FIG. 23 illustrates the loss function 2312 including a focal loss function 2326 and a weighted loss function 2328. In some cases, the splice-site prediction system 106 utilizes the focal loss function 2326 and/or the weighted loss function 2328 as part of a loss function engineering effort to improve training for the prediction of intron retention within nucleotide sequences. Indeed, as previously mentioned, the read data representative of intron retention can be low in many instances, particularly where the likelihood of intron retention for a given training nucleotide sequence is low. Thus, the splice-site prediction system 106 can perform loss function engineering to improve learning for intron retention detection. For instance, the splice-site prediction system 106 can utilize the focal loss function 2326 to modify a weight for nucleobase positions based on an associated probability. To illustrate, in some cases, the splice-site prediction system 106 uses the focal loss function 2326 to reduce the weight for sites with high probability. Further, the splice-site prediction system 106 can use the weighted loss function 2328 to increase the weight of exon-intron boundary counts. The splice-site prediction system 106 can implement the focal loss function 2326 as follows:

$$FL ( {p}_{t} ) = - {( 1 - {p}_{t} )}^{λ} log ( {p}_{t} ) \qquad ( 8 )$$

In function 8, the term log(pₜ) is similar to the term for cross-entropy loss shown above in function 7. For the front term, if the probability is higher (i.e., closer to one), the term will be smaller and contribute less to the final loss. If your probability is lower (i.e., closer to zero), the term will be larger and contribute more to the final loss. As read data may have less coverage indicating intron retention events, using the focal loss function 2326 allows the neural network 2304 to better learn parameters based on those events.

The splice-site prediction system 106 can implement the weighted loss function 2328 as follows:

$$CE = - {\sum }_{i}^{C} {t}_{i} log ( {f ( s )}_{i} ) * {w}_{i} \qquad ( 9 )$$

Using function 9, the splice-site prediction system 106 can control the weights of positions of a training nucleotide sequence covered by nucleotide reads. In particular, the splice-site prediction system 106 can increase the weight for those positions that would indicate intron retention has occurred (e.g., positions at the boundary between and intron and exon) and decrease the weight for other positions (e.g., positions in the exon). Thus, the splice-site prediction system 106 can use function 9 to allow read data indicative of intron retention become more significant for the neural network 2304 in learning its parameters.

As shown in FIG. 23, the splice-site prediction system 106 back propagates the loss determined via the loss function 2312 to the neural network 2304 (as shown by the dashed line 2330) to update the parameters of the neural network 2304. In particular, the splice-site prediction system 106 updates the parameters to minimize the error of the neural network 2304 in generating conditional probabilities and intron-retention probabilities for nucleotide sequences.

Though FIG. 23 illustrates training the neural network 2304 using one refinement iteration, it should be noted that, in some embodiments, the splice-site prediction system 106 trains the neural network 2304 using multiple refinement iterations. In one or more embodiments, with each iteration of refinement, the splice-site prediction system 106 gradually improves the accuracy with which the neural network 2304 generates conditional probabilities and intron-retention probabilities. Thus, the splice-site prediction system 106 learns parameters for the neural network 2304 that facilitate its performance in the splice-site prediction task and in modeling intron retention.

Further, it should be noted that FIG. 23 illustrates a training process implemented by the splice-site prediction system 106 in at least one embodiment, but not all embodiments. Indeed, some of the operations shown in FIG. 23 are optional and are omitted in various embodiments. For instance, the arcsinh transformation 2324 is omitted in some implementations. Further, in some cases, the focal loss function 2326 and/or the weighted loss function 2328 are omitted. Rather, in some embodiments, the splice-site prediction system 106 uses one or more alternative loss functions. Additionally, FIG. 23 indicates that the ground truth intron-retention probability 2314 and the ground truth probabilities 2310 are determined separately; in some instances, however, the splice-site prediction system 106 determines the ground truth intron-retention probability 2314 and the ground truth probabilities 2310 together. For instance, in some cases, the splice-site prediction system 106 determines the ground truth intron-retention probability 2314 and the ground truth probabilities 2310 together to determine probabilities having a sum that is equal to one.

As just mentioned, in one or more embodiments, the splice-site prediction system 106 determines exon-intron boundary counts for use in training a neural network to generate intron-retention probabilities (e.g., for use in determining the ground truth intron-retention probabilities). In particular, the splice-site prediction system 106 determines training dataset counts from a training dataset that includes the read data and determines the exon-intron boundary counts from the training dataset counts. FIGS. 24A-24B illustrate the splice-site prediction system 106 determining exon-intron boundary counts in accordance with one or more embodiments.

As mentioned above, in one or more embodiments, the splice-site prediction system 106 determines an exon-intron boundary count to estimate the number of nucleotide reads within the read data of a training dataset that represent intron retention events for a training nucleotide sequence. In some embodiments, the splice-site prediction system 106 determines the estimation (e.g., the exon-intron boundary count) by determining a number of nucleotide reads that cover a boundary of an intron in the training nucleotide sequence. The splice-site prediction system 106 can use the estimation to determine a ground truth intron-retention probability for the training nucleotide sequence as follows:

$${r}_{IR} = \frac{EI}{EI + nonEI} \qquad ( 10 )$$

In function 10, EI represents the exon-intron boundary count—the number of nucleotide reads that cover a boundary of an intron (i.e., an exon-intron boundary), indicating intron retention. Further, nonEI represents a number of nucleotide reads that do not cover the boundary of an intron. In other words, nonEI represents a number of nucleotide reads that do not indicate intron retention. For example, nonEI can represent junction reads that cover matching splice sites in neighboring positions indicating that the intron between the splice sites has been removed. Thus, the term EI+nonEI can represent the total number of splicing events represented for the training nucleotide sequence within the read data. In some cases, nonEI represents a number of nucleotide reads that covers an edge of an exon (e.g., a threshold number of nucleobases at the edge of the exon) but does not extend into the adjacent intron.

As shown in FIG. 24A, the splice-site prediction system 106 performs an act 2402 of determining a first number of nucleotide reads that cover a threshold number of nucleobases upstream and downstream from an exon-intron boundary. In particular, the splice-site prediction system 106 determines a first number of nucleotide reads that cover both a portion of the donor and a portion of the intron within a threshold number of nucleobases from the exon-intron boundary. In some cases, the splice-site prediction system 106 utilizes a threshold of two nucleobases. Accordingly, the splice-site prediction system 106 can use, for the first number of nucleotide reads, nucleotide reads that have a coverage of at least four nucleotides long, centered at the boundary between the intron and the exon. The splice-site prediction system 106, however, can use different threshold lengths in various embodiments.

As further shown in FIG. 24, the splice-site prediction system 106 performs an act 2404 of determining a second number of nucleotide reads that cover a threshold number of nucleobases within an exon of the exon-intron boundary. In other words, the splice-site prediction system 106 uses, as the second number or nucleotide reads, nucleotide reads that cover the exon edge of an exon-intron boundary. The splice-site prediction system 106 can use the same exon-intron boundary as used for the first number of nucleotide reads so that the same exon is represented in both sets. Further, in some cases, the splice-site prediction system 106 uses the same threshold number of nucleobases for the first and second nucleotide read sets. To illustrate, when using a threshold of two nucleobases to determine the first number of nucleotide reads, the splice-site prediction system 106 can also use the threshold of two nucleobases for determining the second number of nucleotide reads. In some cases, the second number of nucleotide reads more specifically includes those nucleotide reads that cover the exon edge of the exon-intron boundary but do not extend into the intron.

In one or more embodiments, the first number of nucleotide reads and the second number of nucleotide reads correspond to the training dataset counts 2320 discussed above with reference to FIG. 23. Indeed, the splice-site prediction system 106 determines the first number of nucleotide reads and the second number of nucleotide reads from read data included in a training dataset used for training a neural network. In some cases, the splice-site prediction system 106 employs a counting tool to determine the first number of nucleotide reads and the second number of nucleotide reads. For instance, in some implementations, the splice-site prediction system 106 uses the featureCounts tool described by Yang Liao et al., “featureCounts: An Efficient General Purpose Program for Assigning Sequence Reads to Genomic Features,” Bioinformatics, Volume 30, Issue 7, April 2014, Pages 923-930, which is incorporated herein by reference in its entirety.

Additionally, as shown in FIG. 24, the splice-site prediction system 106 performs an act 2406 of determining a ratio of the first number of nucleotide reads and the second number of nucleotide reads. In particular, as shown, the splice-site prediction system 106 determines a ratio of the first number of nucleotide reads and a sum of the first number and second number of nucleotide reads. In one or more embodiments, the splice-site prediction system 106 uses the ratio as a simplified version of the ratio provided in function 10. Thus, the splice-site prediction system 106 can determine the ratio of the first number of nucleotide reads and the second number of nucleotide reads as follows:

$${r}_{IR}^{′} = \frac{{EI}_{FirstReads}}{{EI}_{FirstReads} + {ExonEdge}_{SecondReads} + 0.1} \qquad ( 11 )$$

The splice-site prediction system 106 uses the 0.1 added to the denominator of function 11 to avoid problems that would arise if the denominator would otherwise have a value of zero. As mentioned, the splice-site prediction system 106 can use the ratio of function 11 as a simplified version of function 10. In other words, function 10 represents the ideal theoretical ratio and function 11 represents a ratio used by the splice-site prediction system 106 (in addition to function 12 shown below) to arrive at (or approximate) function 10. In some cases, the splice-site prediction system 106 uses other methods to determine or approximate function 10. For instance, in some instances, the splice-site prediction system 106 uses a third-party tool to determine the ratio shown in function 10.

As further mentioned, in some cases, the term nonEI in function 10 represents those nucleotide reads that cover an edge of an exon but do not extend into the intron. Thus, in some cases, the term nonEI is conceptually similar to (e.g., represents the same number as) the term ExonEdge_{SecondReads} in function 11. Indeed, in some cases, both terms represent nucleotide reads that cover an edge of an exon (e.g., a threshold number of nucleobases at the edge of the exon) but do not extend into the intron (e.g., at least, do not extend a threshold number of nucleobases into the intron).

As shown in FIG. 24B, the splice-site prediction system 106 also performs an act 2408 of determining a normalized exon-intron boundary count using the ratio of function 11 and a third number of nucleotide reads that represent a junction count. In particular, the splice-site prediction system 106 can determine the normalized exon-intron boundary count using the ratio and the nonEI term from function 10. For instance, in some cases, the splice-site prediction system 106 solves functions 10-11 for EI to determine the following:

$$EI = \frac{{r}_{IR}^{′} nonEI}{1 - {r}_{IR}^{′}} \qquad ( 12 )$$

It should be noted that nonEI≠0 in most instances. In other words, the splice-site prediction system 106 uses read data that includes nucleotide reads covering a junction. Accordingly, the splice-site prediction system 106 can use function 12 to determine a non-zero value for the normalized exon-intron boundary count.

Additionally, as shown in FIG. 24B, the splice-site prediction system 106 performs an act 2410 of determining a ground truth intron-retention probability using the normalized exon-intron boundary count. Indeed, the splice-site prediction system 106 can use the normalized count determined via function 12 as the corresponding term in function 10 and determine the ground truth intron-retention probability that corresponds to a training nucleotide sequence being used to train a neural network. Thus, the splice-site prediction system 106 can train a neural network to model intron retention even where the read data of the training dataset does not inherently provide a good indication of intron retention associated with training nucleotide sequences, such as by failing to provide ground truth intron-retention probabilities for the training nucleotide sequences or by otherwise failing to provide indications of which training nucleotide sequences are associated with intron retention.

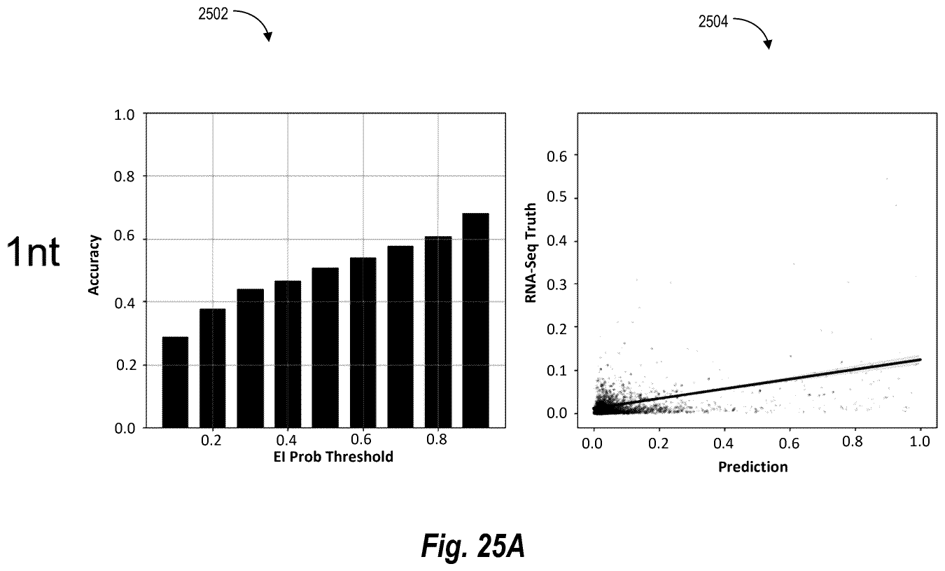

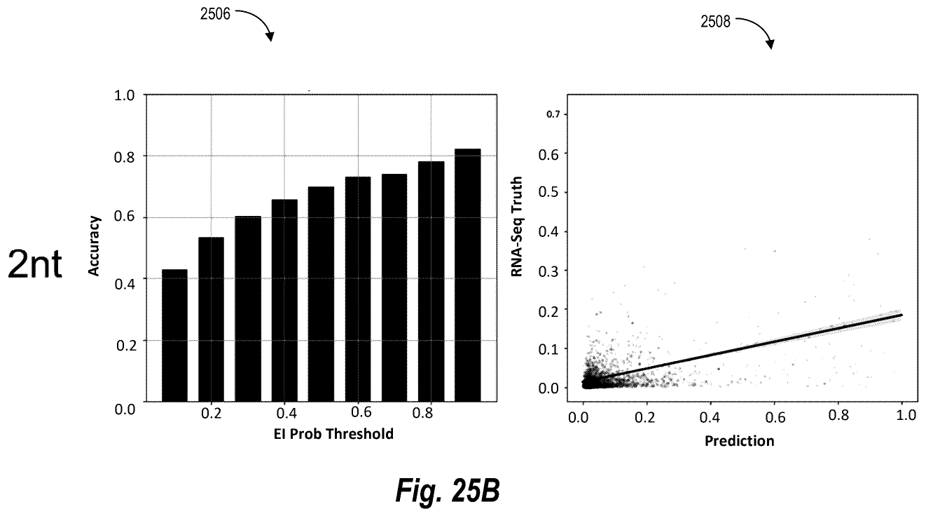

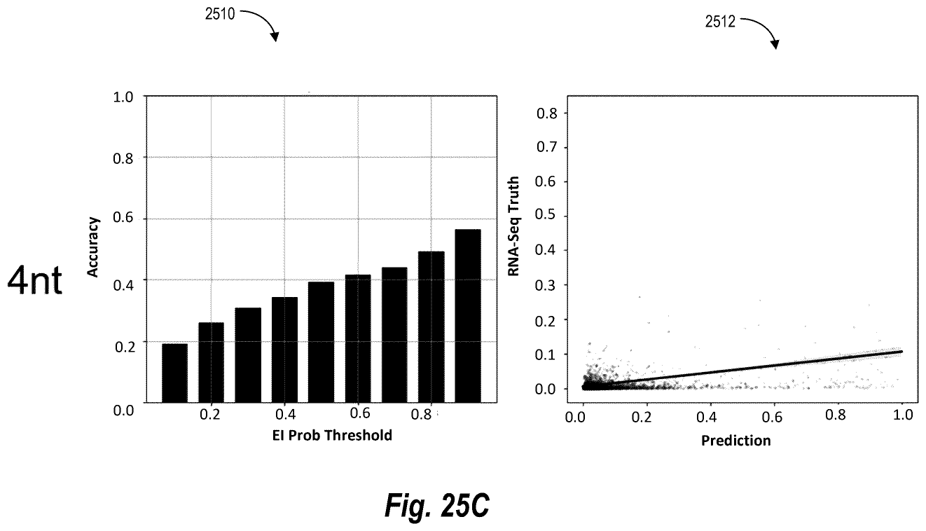

As mentioned, the splice-site prediction system 106 uses a threshold number of nucleobases in determining training dataset counts used to determine exon-intron boundary counts for training nucleotide sequences used to train a neural network to model intron retention. As further mentioned, the splice-site prediction system 106 can use various thresholds in various embodiments. FIGS. 25A-25C illustrate graphs reflecting experimental results regarding the effectiveness of the splice-site prediction system 106 based on the threshold used in determining the exon-intron boundary counts in accordance with one or more embodiments.

In particular, FIG. 25A illustrates a first graph 2502 providing an accuracy of an implementation of the splice-site prediction system 106 determining exon-intron boundary counts using a threshold of one nucleobase, and a second graph 2504 providing a correlation of the implementation. FIG. 25B illustrates a first graph 2506 providing an accuracy of an implementation of the splice-site prediction system 106 using a threshold of two nucleobases for determining exon-intron boundary counts, and a second graph 2508 providing a correlation of the implementation. Further, FIG. 25C illustrates a first graph 2510 providing an accuracy of an implementation of the splice-site prediction system 106 using a threshold of four nucleobases for determining exon-intron boundary counts, and a second graph 2512 providing a correlation of the implementation.

As shown by the graphs of FIG. 25A-25C, the splice-site prediction system 106 performs best when using a threshold of two nucleobases when compared to the other thresholds shown. In particular, the splice-site prediction system 106 using the threshold of two nucleobases achieves the best accuracy and the best correlation. Thus, as discussed above, the splice-site prediction system 106 uses a threshold of two nucleobases when determining the exon-intron boundary counts in some embodiments, though other thresholds can be used in other implementations.

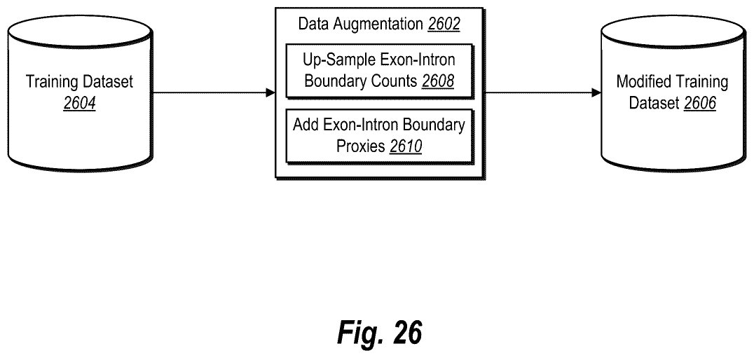

As previously mentioned, splice-site prediction system 106 can perform loss function engineering (e.g., via arcsinh transformations, focal loss functions, and/or weighted loss functions) to improve the training of a neural network to generate intron-retention probabilities. In some embodiments, the splice-site prediction system 106 employs one or more data augmentation techniques to modify the training data used to train a neural network to model intron retention. FIG. 26 illustrates the splice-site prediction system 106 performing data augmentation to modify the training data used for training a neural network to model intron retention in accordance with one or more embodiments.

As shown in FIG. 26, the splice-site prediction system 106 performs one or more data augmentation techniques 2602 to a training dataset 2604 that will be used to train a neural network to generate intron-retention probabilities for nucleotide sequences. In particular, the training dataset 2604 can include read data corresponding to the training nucleotide sequences (as well as the training nucleotide sequences themselves). As a result of the one or more data augmentation techniques 2602, the splice-site prediction system 106 generates a modified training dataset 2606.

Indeed, as previously indicated, the read data representative of intron retention can be low in many instances. In particular, even where read data indicates intron retention for a particular training nucleotide sequence, the number of nucleotide reads that indicate intron retention for the training nucleotide sequence can be low. Accordingly, training a neural network to model intron retention using such read data can lead to poor results as the neural network fails to see enough examples that allow for generalizing the task for modeling intron retention. In other words, the parameters of the neural network will be poorly suited for the task. The splice-site prediction system 106 can perform data augmentation to increase the representation of intron retention within the read data, which allows the neural network to better learn parameters for modeling intron retention.

As shown in FIG. 26, the splice-site prediction system 106 can perform data augmentation on the training dataset 2604 by up-sampling 2608 the exon-intron boundary counts determined for training nucleotide sequences. In particular, the splice-site prediction system 106 can up-sample the exon-intron boundary counts to increase the exon-intron boundary counts. Thus, the splice-site prediction system 106 can increase the representation of intron retention for the training nucleotide sequences within the read data using the representation of intron retention that is already inherent to the read data.

As further shown in FIG. 26, the splice-site prediction system 106 can perform data augmentation on the training dataset 2604 by adding 2610 exon-intron boundary proxies to the read data. To illustrate, in some cases, the splice-site prediction system 106 selects arbitrary positions within one or more of the training nucleotide sequences and treats the selected positions as if they were at an exon-intron boundary. For instance, the splice-site prediction system 106 can select a position within an exon (e.g., in the interior of the exon rather than at the boundary between the exon and an intron) of a training nucleotide sequence for an exon-intron boundary proxy. Thus, when determining exon-intron boundary counts for the training nucleotide sequences, the splice-site prediction system 106 can include the exon-intron boundary proxies as part of the counts.

The splice-site prediction system 106 can use exon-intron boundary proxies as they are positioned in areas within the training nucleotide sequences that typically do not experience splicing. For instance, a nucleobase positioned within the interior of an exon rather at its boundary typically will not have nucleotide reads indicating that the nucleobase has been spliced out. The splice-site prediction system 106 can therefore use the patterns of nucleobases surrounding the exon-intron boundary proxies to facilitate learning of patterns that result in the retention of a portion of a nucleotide sequence.

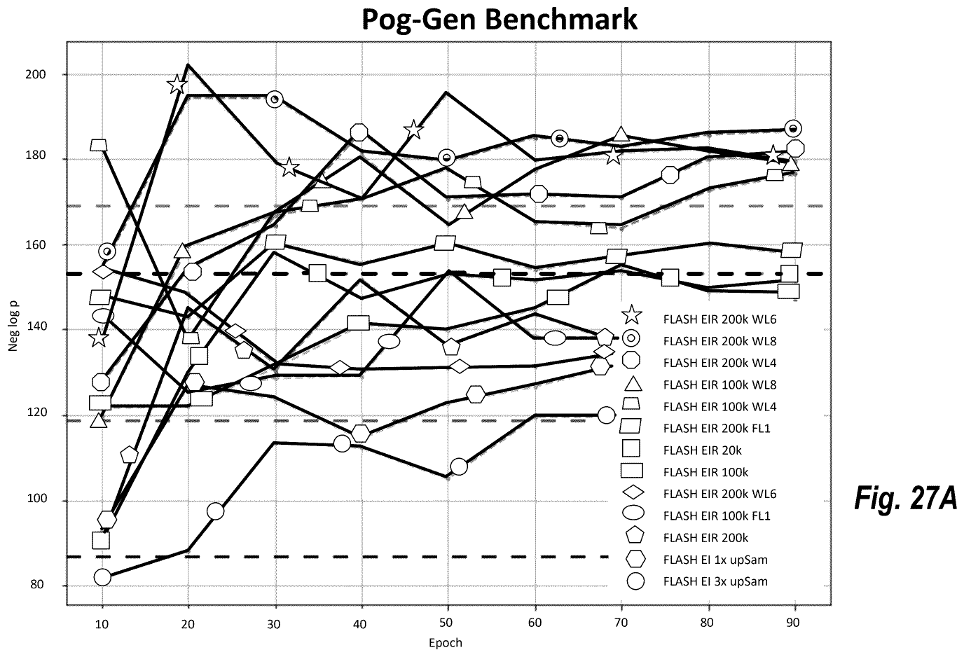

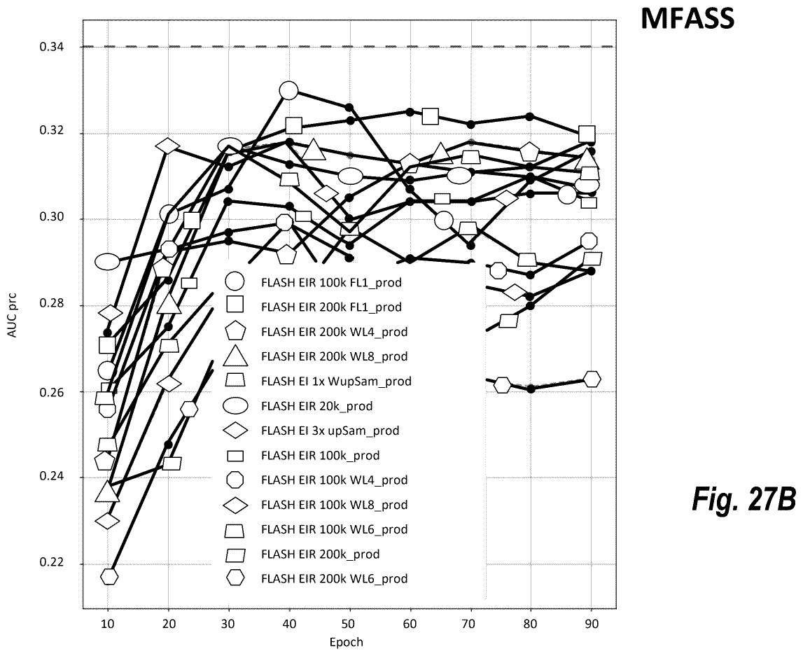

FIGS. 27A-27B illustrate graphs reflecting experimental results regarding the effectiveness of the splice-site prediction system 106 based on techniques used for training a neural network to model intron retention in accordance with one or more embodiments. As shown, all implementations of the splice-site prediction system 106 represented within the graph use a FLASH-based transformer neural network, such as the transformer neural network 900 described above with reference to FIG. 9. Further, the represented implementations use various combinations of the techniques described above for training a neural network to model intron retention.

For example, where the term “EIR” is shown in the legend of one of the graphs, the corresponding implementation of the splice-site prediction system 106 trains the neural network using exon-intron boundary proxies that have been added to the training dataset, the number of which follows the “EIR” label. Additionally, the terms “FL” and “WL” represents where the corresponding implementations employ a focal loss function or a weighted loss function, respectively, with the weighted value following the label. Further, the term “upSample” represents where the corresponding implementations up-sample the exon-intron boundary counts within the training dataset used to train the neural network.

The graph of FIG. 27A measures the performance of the tested models with respect to a negative log probability metric, while the graph of FIG. 27B measures the performance of the tested models with respect to an area-under-the-curve metric based on a precision recall curve. As shown in FIG. 27A, the implementations of the splice-site prediction system 106 that use the exon-intron boundary proxies with the weighted loss (those implementations represented at the top of the legend) performed better than the other tested systems with respect to the negative log probability metric. As shown in FIG. 27B, the implementations of the splice-site prediction system 106 that used focal loss (those implementations represented at the top of the legend) performed better than the other tested models with respect to the area-under the curve metric.

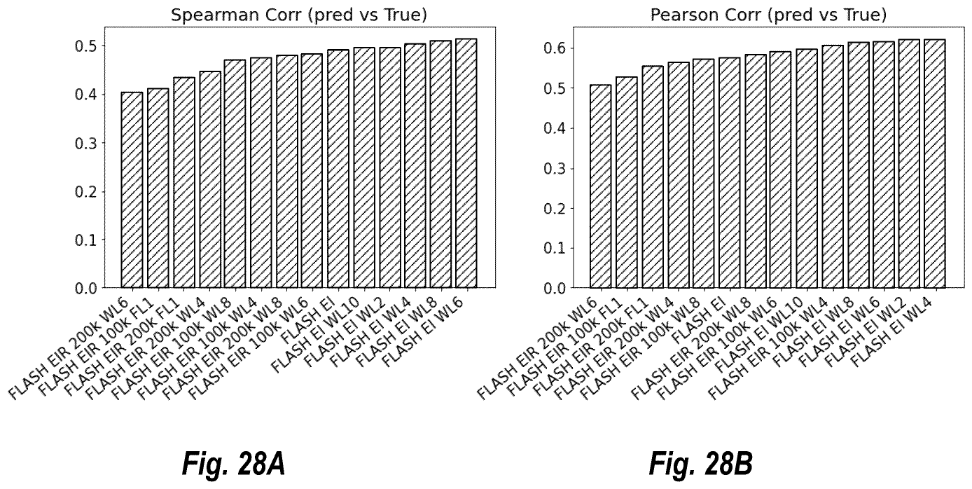

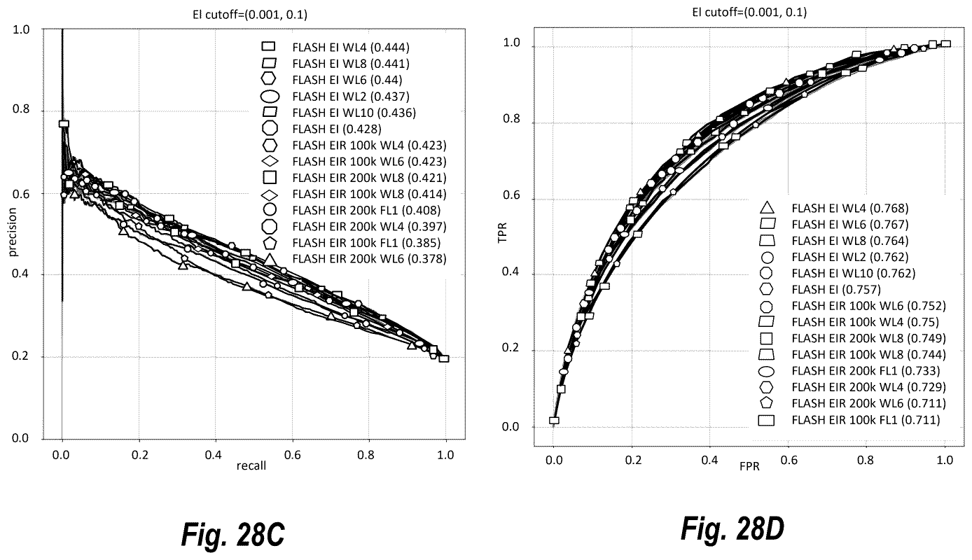

FIGS. 28A-28D illustrate graphs reflecting additional experimental results regarding the effectiveness of the splice-site prediction system 106 based on techniques used for training a neural network to model intron retention in accordance with one or more embodiments. The graphs shown in FIGS. 28A-28D compare the performance of implementations of the splice-site prediction system 106 using various combinations of training techniques to an implementation that does not use these techniques (labeled “FLASH EI”). In other words, the FLASH EI implementation uses training techniques similar to those discussed above with respect to FIG. 13 while further accounting for intron retention during training.

The graph of FIG. 28A measures the performance of the tested models with respect to Spearman correlation while the graph of FIG. 28B measures the performance with respect to Pearson correlation. The graph of FIG. 28C shows the precision of each tested model with respect to recall, and the graph of FIG. 28D shows the true positive rate (TPR) of each tested model with respect to false positive rate (FPR). As shown by the graphs of FIGS. 28A-28D, there are several implementations of the splice-site prediction system 106 using combinations of the training techniques described above that outperform the base FLASH EI implementation (e.g., an implementation of a FLASH-based transformer neural network that determines ground truth intron-retention probabilities as described above but omits the data augmentation and loss function engineering techniques described above). Thus, the graphs indicate that the described training techniques improve the ability of the splice-site prediction system 106 to model intron retention in many instances.

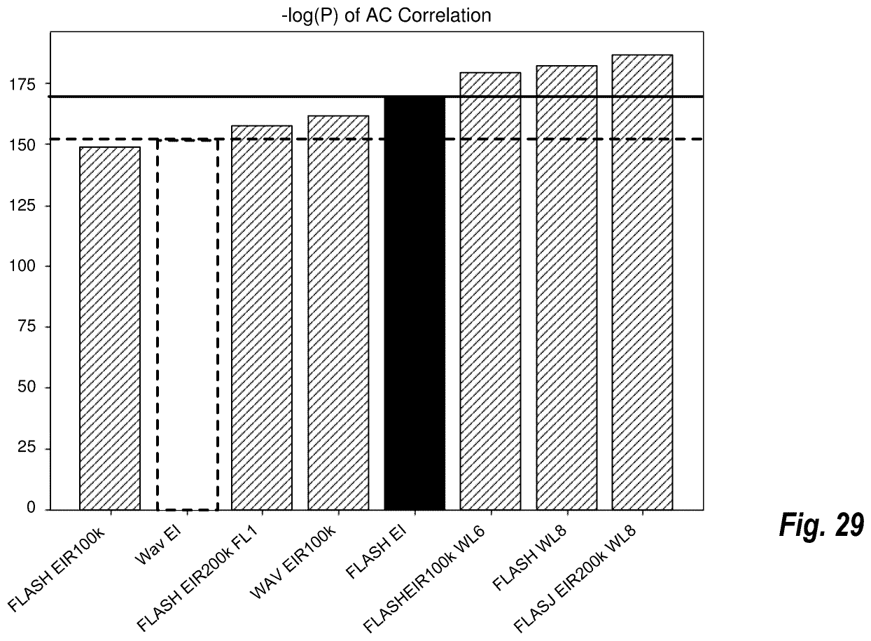

FIG. 29 illustrates a graph reflecting further experimental results regarding the effectiveness of the splice-site prediction system 106 based on techniques used for training a neural network to model intron retention in accordance with one or more embodiments. The graph of FIG. 29 compares the performance of implementations of the splice-site prediction system 106 using various combinations of training techniques to various implementations of a Wavenet model (labeled “Wav EI” and “WAV EIR 100K”). The graph measures the performance of each tested model with respect to a negative log probability metric. As shown by the graph, several implementations of the splice-site prediction system 106 outperform the implementations of the Wavenet model. The FLASH EI implementation outperforms both Wavenet model implementations, and the implementation of the splice-site prediction system 106 using a combination of two hundred thousand exon-intron boundary proxies and a weighted loss function to train its neural network provides the best results.

Thus, the splice-site prediction system 106 operates more flexibly and offers a more flexible neural network when compared to existing systems. Indeed, the splice-site prediction system 106 uses a neural network to flexibly model intron retention in addition to generating conditional probabilities for splice sites where existing systems typically limit themselves to splice site detection. By implementing loss function engineering and/or data augmentation techniques, the splice-site prediction system 106 flexibly employs new training methods that allow a neural network to effectively learn parameters for predicting intron retention for nucleotide sequences and assigning an intron retention probability to those nucleotide sequences. Further, by effectively modeling intron retention, the splice-site prediction system 106 more accurately performs the task of splice site detection. In particular, the splice-site prediction system 106 more accurately generates conditional probabilities for splice sites. Indeed, the splice-site prediction system 106 recognizes how intron retention affects the use rate of potential splice sites and adjusts its outputs to reflect that effect. Accordingly, the outputs of the splice-site prediction system 106 also accurately reflect how a variant present within a nucleotide sequence can affect the probability of intron retention for that nucleotide sequence as well as the probability of use for each potential splice site.

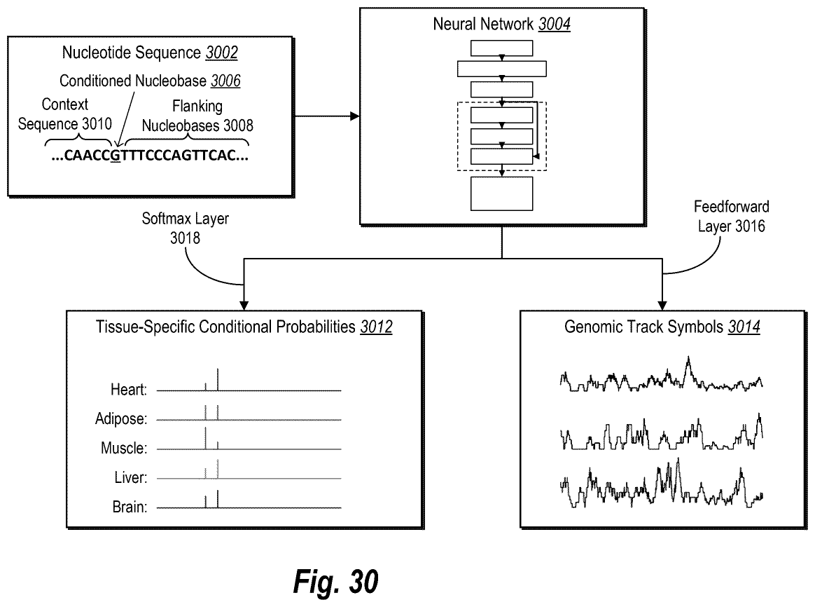

As previously mentioned, in one or more embodiments, the splice-site prediction system 106 can model context-specific splicing for nucleotide sequences. For example, the splice-site prediction system 106 can generate context-specific conditional probabilities for a nucleotide sequence that indicate splicing for the nucleotide sequence within a given context. FIGS. 30-42 illustrate the splice-site prediction system 106 configuring and using a neural network to model context-specific splicing in accordance with one or more embodiments.

The discussion surrounding FIGS. 30-42 particularly discusses modeling splicing with respect to the context of tissues. In particular, the following discusses generating multiple sets of tissue-specific conditional probabilities for a nucleotide sequence where each set of tissue-specific conditional probabilities corresponds to a different tissue. As suggested above, however, the splice-site prediction system 106 can more generally model splicing for various contexts. Accordingly, for a given context, the splice-site prediction system 106 can generate multiple sets of context-specific conditional probabilities for a nucleotide sequence where each set of context-specific conditional probabilities corresponds to a different instance of the given context. Thus, the splice-site prediction system 106 can train and implement a neural network to recognize motifs within nucleotide sequences that are relevant to splicing within a given context. Indeed, in some cases, the sets of context-specific conditional probabilities generated for a nucleotide sequence indicate differences in splicing within the given context caused by those motifs.

It should be understood that the terms “context” and “context instance” (or “instance of a context”) may be used interchangeably in some instances herein. Indeed, while the term “context” may be used more generally to refer to a contextual category (e.g., tissues, diseases, etc.), the term can also be used to refer to a more specific subject matter within that category (e.g., a particular tissue, a particular disease, etc.). The term “context instance,” on the other hand, more specifically refers to subject matter fitting within a contextual category (e.g., the particular tissue, the particular disease, etc.). Thus, there is some overlap between these terms.

FIG. 30 illustrates an overview of the splice-site prediction system 106 modeling tissue-specific splicing for a nucleotide sequence in accordance with one or more embodiments. As shown in FIG. 30, the splice-site prediction system 106 provides a nucleotide sequence 3002 to a neural network 3004 (e.g., a transformer neural network). As shown, the nucleotide sequence 3002 includes a conditioned nucleobase 3006 at a conditioned position, flanking nucleobases 3008, and a context sequence 3010.

As shown, the splice-site prediction system 106 uses the neural network 3004 to analyze the nucleotide sequence 3002 and generate tissue-specific conditional probabilities 3012 and genomic track signals 3014 based on the analysis. In particular, the splice-site prediction system 106 uses a feedforward layer 3016 of the neural network 3004 to generate the genomic track signals 3014 and a softmax layer 3018 of the neural network 3004 to generate the tissue-specific conditional probabilities 3012. Indeed, in one or more embodiments, the splice-site prediction system 106 configures the output block of the neural network 3004 differently than described above so that a layer other than the final layer of the neural network 3004 (e.g., the feedforward layer 3016) provides outputs, such as the genomic track signals 3014.

Notably, as indicated by FIG. 30, the splice-site prediction system 106 uses the neural network 3004 to generate the tissue-specific conditional probabilities 3012 and the genomic track signals 3014 from a single nucleotide sequence. Thus, the splice-site prediction system 106 can configure the neural network 3004 to generate multiple output types and multiple outputs of each output type from a single nucleotide sequence.

Though FIG. 30 indicates the splice-site prediction system 106 generating a particular number of sets of tissue-specific conditional probabilities with specific tissues represented, the splice-site prediction system 106 can generate various numbers of sets representing various tissues in various embodiments. Further, while the FIG. 30 shows the splice-site prediction system 106 generating multiple genomic track signals, the splice-site prediction system 106 can generate various numbers of genomic track signals in various embodiments. Indeed, in general, the splice-site prediction system 106 can generate one or more genomic track signals from a single nucleotide sequence.

In one or more embodiments, the splice-site prediction system 106 generates the genomic track signals 3014 to facilitate the generation of the tissue-specific conditional probabilities 3012. Indeed, in some cases, by generating the genomic track signals 3014, the splice-site prediction system 106 improves its ability to generate the tissue-specific conditional probabilities 3012. For instance, the splice-site prediction system 106 can generate the tissue-specific conditional probabilities 3012 more accurately, as will be discussed more below.

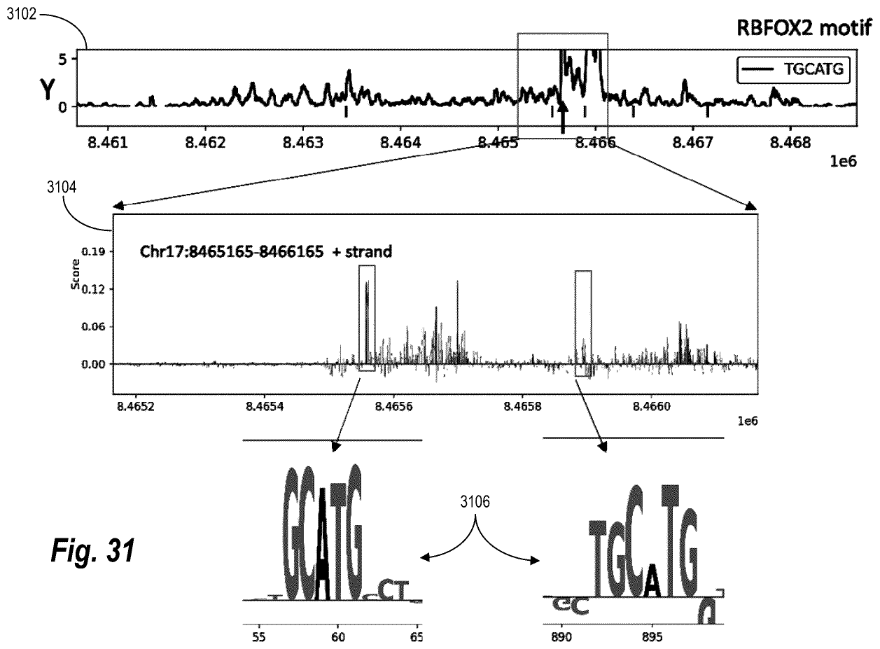

In some embodiments, the splice-site prediction system 106 can use a neural network to generate a genomic track signal for a nucleotide sequence based on one or more motifs associated with genomic track signal. FIG. 31 illustrates a motif used by the splice-site prediction system 106 to generate an associated genomic track signal in accordance with one or more embodiments.

In particular, FIG. 31 illustrates a genomic track signal 3102 for the RBFOX2 protein coding gene. FIG. 31 further illustrates a zoomed-in view 3104 of a portion of the genomic track signal 3102. As shown by the zoomed-in view 3104, the genomic track signal 3102 corresponds to a nucleotide sequence (as the zoomed-in view shows a sequence of nucleobases). Further, in FIG. 31, a motif 3106 is shown in at least two instances of the portion of the genomic track signal 3102 represented within the zoomed-in view 3104. As shown, in both instances, the motif 3106 corresponds to a peak within the genomic track signal 3102.

Thus, as indicated by FIG. 31, genomic track signals are associated with particular motifs within nucleotide sequences. The motifs may include patterns (e.g., subsequences) of nucleobases determined to be relevant to one or more genomic track signals-such as patterns whose inclusion, exclusion, or frequency, affect the strength (e.g., amplitude) of a genomic track signal. Thus, the splice-site prediction system 106 can train a neural network to learn the motifs that are associated with a particular genomic track signal. The splice-site prediction system 106 can further utilize the neural network to generate genomic tracks signals for nucleotide sequences by recognizing the motifs included therein.

In one or more embodiments, the splice-site prediction system 106 confirms the use of motifs within nucleotide sequences for generating genomic tracks signals via an in-silico mutagenesis process. To illustrate, in some cases, the splice-site prediction system 106 selects a position within a nucleotide sequence and changes the nucleobase represented therein from the reference representation. For instance, if the reference nucleotide sequence includes a G-base at that position, the splice-site prediction system 106 can change the nucleobase to an A-base, a T-base, or a C-base. The splice-site prediction system 106 can then determine the difference caused by the change to the amplitude of the genomic track signal as follows:

$$Score = {Y}_{ref} - \frac{1}{4} ( {Y}_{A} + {Y}_{G} + {Y}_{T} + {Y}_{C} ) \qquad ( 13 )$$

In function 13, Y represents the amplitude of the genomic track signal. If, after switching the reference nucleobase with another nucleobase, the score decreases, the splice-site prediction system 106 can determine that the nucleobase is significant to the amplitude in that the nucleobase affects (e.g., increases or decreases) the amplitude. In other words, the splice-site prediction system 106 can determine that the nucleobase is part of a motif that is relevant to the genomic track signal.

In some cases, the splice-site prediction system 106 iteratively traverses through the positions of the nucleotide sequence or the portion of the nucleotide sequence to be analyzed. At each position, the splice-site prediction system 106 changes the nucleobase represented by the reference, determines the score, and determines whether the score changed due to the change of the nucleobase. If the score decreases, the splice-site prediction system 106 can determine that the nucleobase at the position is relevant to the genomic track signal. Thus, in some cases, the splice-site prediction system 106 can perform the in-silico mutagenesis process as a form of manual double-checking. For instance, in some cases, the splice-site prediction system 106 performs the in-silico mutagenesis process to confirm that the motif 3106 is relevant to the genomic track signal 3102 for the RBFOX2 protein coding gene.

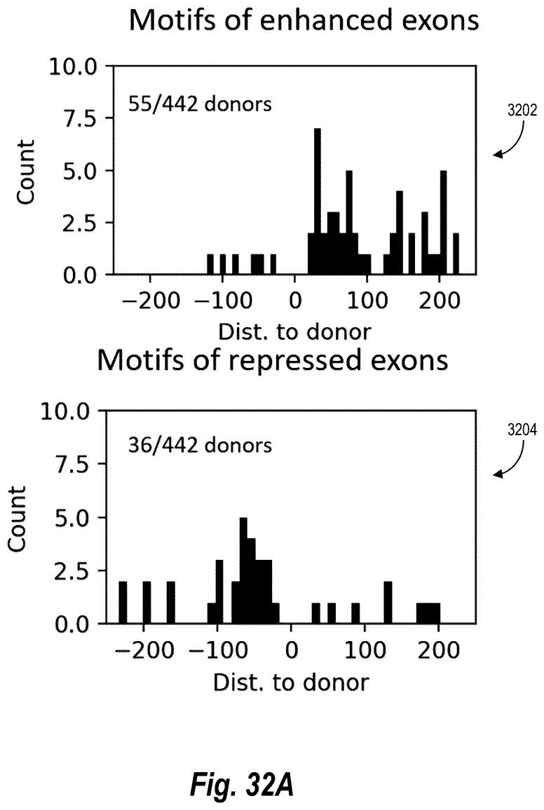

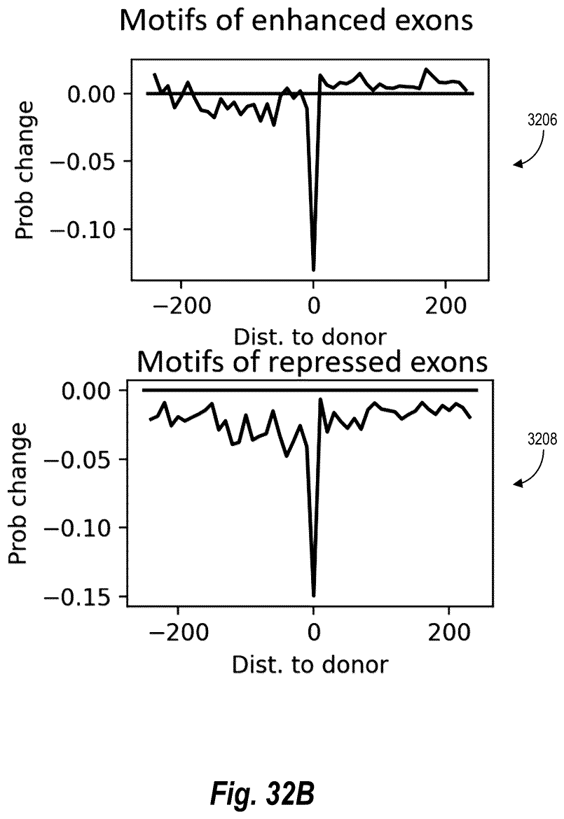

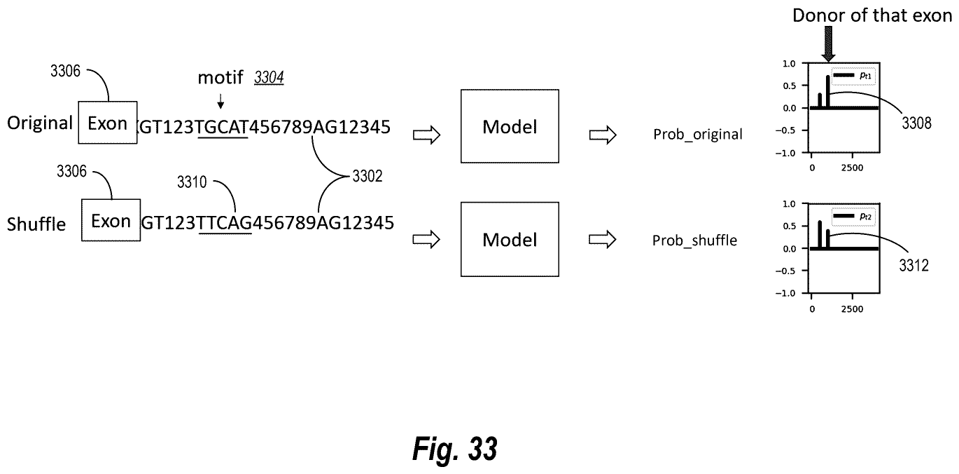

Further, in one or more embodiments, the splice-site prediction system 106 can use a neural network to generate tissue-specific conditional probabilities for a nucleotide sequence based on one or more motifs associated with tissue-specific conditional probabilities. FIGS. 32A-32B illustrates graphs reflecting experimental results regarding the effectiveness of the splice-site prediction system 106 in using motifs within a nucleotide sequence to generate tissue-specific conditional probabilities in accordance with one or more embodiments.

Researchers conducted a study that led to the experimental results using nucleotide sequences associated with potential splicing that resulted in exon skipping. For the study, the researchers selected the acceptor that would be used upon skipping of the exons as the conditioned nucleobase and provided the nucleotide sequences to a neural network used by the splice-site prediction system 106 to determine predictions for an upstream matching donor. In particular, the study focused on whether the splice-site prediction system 106 would predict that the donor of the potentially skipped exons would be used as a splice site. The researchers further located a motif (e.g., a RBFOX1 or RBFOX2 motif) that was positioned in or near the exons that would potentially be skipped and slid the motif to different positions within the nucleotide sequence to determine whether the probability of those exons being used changed. In other words, the study was configured to determine whether the splice-site prediction system 106 predicted the inclusion of the potentially skipped exons based on the motifs within the nucleotide sequences.

FIG. 32A illustrates a first graph 3202 and a second graph 3204. The first graph 3202 corresponds to predictions of the model that the potentially skipped exons would be included during splicing. The second graph 3204 corresponds to prediction of the model that the potentially skipped exons would indeed be skipped during splicing. As indicated by the first graph 3202, the splice-site prediction system 106 typically predicted that the exons would be included when the motifs were positioned within introns downstream from the exons. On the other hand, the second graph 3204 shows that the splice-site prediction system 106 typically predicted that the exons would be skipped when the motifs were positioned within the exons themselves.

FIG. 32B illustrates a third graph 3206 and a fourth graph 3208 illustrating the change in the probability determined for the exons by the splice-site prediction system 106 as the motifs are slide into different positions within their nucleotide sequences. As indicated by the third graph 3206, a motif that typically leads to a prediction of exon inclusion when the motif is positioned within a downstream intron causes the probability of exon inclusion to drop as the motif is slid into the exon itself. Additionally, as indicated by the fourth graph 3208, a motif that typically leads to a prediction of exon skipping when the motif is positioned within the exon, the probability is still poor when the motif is moved into the downstream intron.

Thus, the graphs of FIGS. 32A-32B illustrate the splice-site prediction system 106 uses a neural network to generated tissue-specific conditional probabilities for nucleotide sequences based on the motifs included therein. Indeed, the graphs show that the splice-site prediction system 106 uses the neural network to generate conditional probabilities in general based on motifs.

Motifs and their effects on the inclusion of exons during splicing can be discussed in terms of enhanced exons and suppressed exons. Indeed, FIGS. 32A-32B label their graphs as corresponding to “Motifs of Enhanced Exons” or “Motifs of Repressed Exons.” FIG. 33 illustrates an example of an enhanced exon and the role of modifying the motif to determine whether the motif is associated with an enhanced exon or a repressed exon in accordance with one or more embodiments.

In particular, FIG. 33 illustrates a nucleotide sequence 3302 with a motif 3304 downstream from an exon 3306 and a conditional probability 3308 generated for the donor associated with the boundary of the exon 3306 based on the motif 3304. FIG. 33 also illustrates the nucleotide sequence 3302 with a shuffled motif 3310 and a conditional probability 3312 generated for the donor associated with the boundary of the exon 3306 based on the shuffled motif 3310. As shown by the change in probability, the exon 3306 is enhanced by the motif 3304. In other words, the presence of the motif 3304 at its position within the nucleotide sequence 3302 increases the probability that the exon 3306 is included, rather than skipped, during splicing. On the other hand, where the presence of a motif within a nucleotide sequence causes a decrease in the probability of an exon being included, the exon is determined to be repressed by the motif. Indeed, if the motif is shuffled and the resulting probability for the exon being included increases, the exon is repressed by the motif.

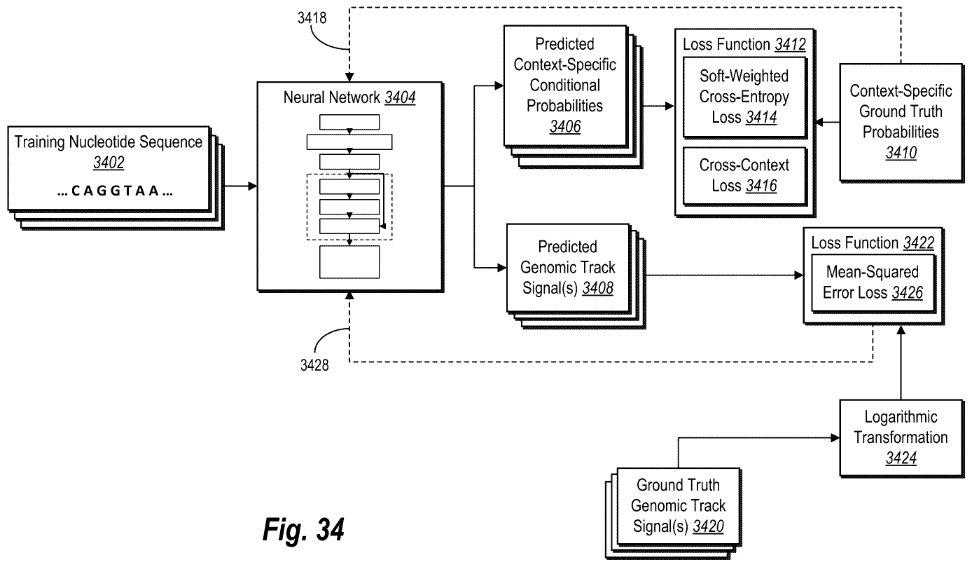

As indicated above, in one or more embodiments, the splice-site prediction system 106 can train and utilize a neural network to generate tissue-specific conditional probabilities and/or other context-specific conditional probabilities for nucleotide sequences. In particular, in some cases, the splice-site prediction system 106 employs multi-task learning to train a neural network to generate context-specific conditional probabilities and genomic track signals for nucleotide sequences. FIG. 34 illustrates the splice-site prediction system 106 using multi-task learning to train a neural network to generate context-specific conditional probabilities (e.g., tissue-specific conditional probabilities, disease-specific conditional probabilities, cell-type-specific conditional probabilities) and genomic track signals in accordance with one or more embodiments.

Indeed, as shown in FIG. 34, the splice-site prediction system 106 utilizes a training nucleotide sequence 3402 to train a neural network 3404 (e.g., a transformer neural network). In some cases, the training nucleotide sequence 3402 includes a conditioned nucleobase at a conditioned position and a plurality of flanking nucleobases within a threshold number of nucleobases from the conditioned nucleobase. In some instances, the training nucleotide sequence 3402 also includes a context sequence on one or both sides of the conditioned nucleobase and flanking nucleobases.

As shown in FIG. 34, the splice-site prediction system 106 provides the training nucleotide sequence 3402 to the neural network 3404. Further, the splice-site prediction system 106 utilizes the neural network 3404 to generate predicted context-specific conditional probabilities 3406 and one or more predicted genomic track signal(s) 3408 for the training nucleotide sequence 3402. In some embodiments, each predicted conditional probability from the predicted conditional probabilities 1306 corresponds to a flanking nucleobase within the training nucleotide sequence 1302 and indicates a likelihood that the flanking nucleobase is part of a matching donor or a matching acceptor for the conditioned nucleobase.

As indicated above, the neural network 3404 can be trained to generate different types of context-specific conditional probabilities by using naturally occurring or artificial training nucleotide sequences from (or corresponding to) genomic samples in different contexts. As part of the training depicted in FIG. 34, in some embodiments, the splice-site prediction system 106 utilizes the neural network 3404 to generate the predicted context-specific conditional probabilities 3406 in the form of tissue-specific conditional probabilities for different target tissues, disease-specific conditional probabilities for different target diseases, cell-type-specific conditional probabilities for different target cell types, cell-line-specific conditional probabilities for different target cell lines, or assay-specific conditional probabilities for different target assay types.

As further indicated above and exemplified below, the neural network 3404 can generate different types of genomic track signals. For instance, the neural network 3404 can generate the one or more predicted genomic track signal(s) 3408 in the form of (i) RNA binding protein signals that indicate probabilities that positions within a nucleotide sequence correspond to a binding site for an RNA binding protein, (ii) histone mark signals that indicate probabilities that positions within the nucleotide sequence correspond to a histone mark, (iii) DNase I-hypersensitivity signals indicating probabilities that positions within the nucleotide sequence correspond to DNase I-hypersensitive sites (DHSs), (iv) transcription-factor signals that indicate probabilities that positions within the nucleotide sequence correspond to transcription factor (TF) binding sites, (v) histone-modification-level signals indicating levels of histone modification (HM) marks for positions within the nucleotide sequence, and/or (vi) methylation-level signals indicating DNA methylation marks or levels for positions within the nucleotide sequence.

As further illustrated in FIG. 34, the splice-site prediction system 106 compares the predicted context-specific conditional probabilities 3406 to context-specific ground truth probabilities 3410 via a loss function 3412. In one or more embodiments, the context-specific ground truth probabilities 3410 provide context-specific conditional probabilities of matching splice sites corresponding to conditioned nucleobases. Indeed, in some cases, the splice-site prediction system 106 uses context-specific training data in training the neural network 3404. In some cases, the splice sites are the same for a given training nucleotide sequence across the different contexts but the conditional probabilities associated with those splice sites will differ across the contexts.

For example, in one or more embodiments, the splice-site prediction system 106 utilizes a soft-weighted cross-entropy loss function 3414 as the loss function 3412 as follows:

$$Loss = {\sum }_{t = 1}^{T} {\sum }_{i = 1}^{N} - scale * {p}_{it} * log ( {q}_{it} ) \qquad ( 14 )$$

In function 14, T represents the number of contexts that are represented and N represents the number of flanking nucleobases that are analyzes. For instance, in some embodiments, T represents a number of different tissues, a number of different diseases, a number of different cell types, a number of different cell lines, or a number of different assays. In one or more embodiments, the splice-site prediction system 106 utilizes the soft-weighted cross-entropy loss function 3414 to improve accuracy of the neural network 3404 across the sequence dimension of the data. In some embodiments, the splice-site prediction system 106 utilizes a cross-context loss function 3416 as the loss function 3412 as follows:

$$Loss = K {\sum }_{{t}_{1} = 1} {\sum }_{{t}_{2} = 1} {‖ {e}_{t 1 , t 2} - {\hat{e}}_{t 1 , t 2} ‖}_{2}^{2} \qquad ( 15 )$$

In function 15, e_{t1,t2}=pₜ₁−pₜ₂, which is the difference in ground truth probabilities for a pair of contexts. Further, ê_{t1,t2}=qₜ₁−qₜ₂, which is the difference in predicted conditional probabilities for the pair of contexts. As suggested above, in some embodiments, the pair of contexts used for the cross-context loss function 3416 constitute a pair of different tissues, a pair of different diseases, a pair of different cell types, a pair of different cell lines, or a pair of different assays. In one or more embodiments, the splice-site prediction system 106 utilizes the cross-context loss function 3416 to improve the accuracy of prediction across the context dimension. In some cases, the splice-site prediction system 106 uses a combination of the soft-weighted cross-entropy loss function 3414 and the cross-context loss function 3416 as the loss function 3412 as follows:

$$Loss = {\sum }_{t = 1}^{T} {\sum }_{i = 1}^{N} - scale * {p}_{it} * log ( {q}_{it} ) + K {\sum }_{{t}_{1} = 1} {\sum }_{{t}_{2} = 1} {‖ {e}_{t 1 , t 2} - {\hat{e}}_{t 1 , t 2} ‖}_{2}^{2} \qquad ( 16 )$$

As shown in FIG. 34, the splice-site prediction system 106 back propagates the loss determined via the loss function 3412 to the neural network 3404 (as shown by the dashed line 3418) to update the parameters of the neural network 3404. In particular, the splice-site prediction system 106 updates the parameters to minimize the error of the neural network 3404 in generating context-specific conditional probabilities for nucleotide sequences.

As further illustrated in FIG. 34, the splice-site prediction system 106 compares the one or more predicted genomic track signal(s) 3408 to one or more ground truth genomic track signals 3420 via a loss function 3422. In particular, as shown in FIG. 34, the splice-site prediction system 106 transforms the one or more ground truth genomic track signals 3420 via a logarithmic transformation 3424 before comparison via the loss function 3422. For instance, the splice-site prediction system 106 can perform the logarithmic transformation 3424 by utilizing a log(1+x) transformation, where x represents the one or more ground truth genomic track signals 3420 in another transformation. Thus, in some embodiments, the splice-site prediction system 106 can generate one or more transformed ground truth genomic track signals and compare the one or more transformed ground truth genomic track signals with the one or more predicted genomic track signal(s) 3408 via the loss function 3422 using the transformed values.

In one or more embodiments, the splice-site prediction system 106 uses the logarithmic transformation 3424 to stabilize the gradient update. Indeed, in some cases, the losses can fail to stabilize without the use of the logarithmic transformation 3424 (e.g., the losses fail to decrease or converge). In some implementations, the splice-site prediction system 106 uses the logarithmic transformation 3424 for all genomic track signals accounted for in training. By contrast, in some implementations, the splice-site prediction system 106 uses the logarithmic transformation 3424 for a subset of the signals.

As further indicated in FIG. 34, the splice-site prediction system 106 can use, as the loss function 3422, a mean-squared error loss function 3426. Indeed, in some cases, the splice-site prediction system 106 uses the following function:

$$Loss = \frac{1}{N} {\sum }_{i = 1}^{N} {( {y}_{i} - {\hat{y}}_{i} )}^{2} \qquad ( 17 )$$

In function 17, yᵢ represents a ground truth genomic track signal and ŷᵢ represents a predicted genomic track signal. The splice-site prediction system 106 can use different instances of the mean-squared error loss function 3426 in the same training iteration to compare different types of genomic track signals. In a given training iteration, for instance, yᵢ represents a ground truth histone mark signal and y; represents a predicted histone mark signal in one instance of the mean-squared error loss function 3426. In the same given training iteration, yᵢ represents a ground truth RNA binding protein signal and ŷᵢ represents a predicted RNA binding protein signal in another instance of the mean-squared error loss function 3426. In the same training iteration, the splice-site prediction system 106 may use an instance of the mean-squared error loss function 3426 to compare alternative or additional genomic track signals, such as by comparing ground truth and predicted DNase I-hypersensitivity signals, transcription-factor signals, histone-modification-level signals, or methylation-level signals.

As further shown in FIG. 34, the splice-site prediction system 106 back propagates the loss determined via the loss function 3422 to the neural network 3404 (as shown by the dashed line 3428) to update the parameters of the neural network 3404. In particular, the splice-site prediction system 106 updates the parameters to minimize an error of neural network 3404 in generating genomic tracks signals for nucleotide sequences in further iterations.

Though FIG. 34 illustrates training the neural network 3404 using one refinement or training iteration, it should be noted that, in some embodiments, the splice-site prediction system 106 trains the neural network 3404 using multiple refinement or training iterations. In one or more embodiments, with each iteration of refinement, the splice-site prediction system 106 gradually improves the accuracy with which the neural network 3404 generates context-specific conditional probabilities and genomic track signals.

Thus, the splice-site prediction system 106 learns parameters for the neural network 3404 that facilitate its performance in the splice-site prediction task. Further, the splice-site prediction system 106 learns parameters that facilitate the generation of genomic track signals. Indeed, the splice-site prediction system 106 unconventionally employs multi-task learning to simultaneously train the neural network to generate context-specific conditional probabilities and genomic track signals for the improvement of splice site prediction.

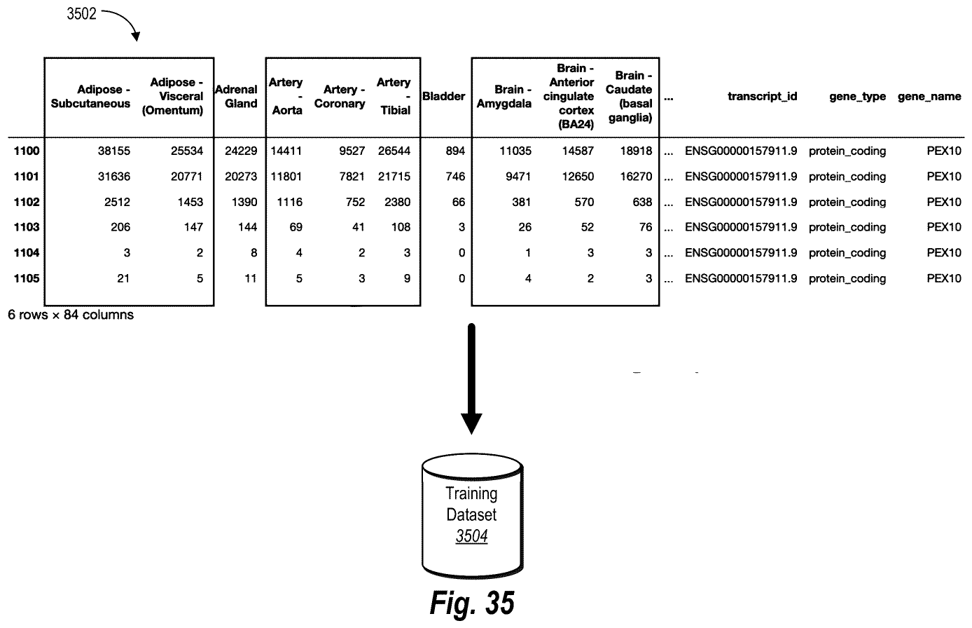

As mentioned, in one or more embodiments, the splice-site prediction system 106 utilizes training data that represents splicing for multiple contexts to train a neural network. For instance, the splice-site prediction system 106 can use sets of read data that each correspond to a nucleotide sequence in a different tissue. In some cases, the splice-site prediction system 106 determines tissue-specific ground truth probabilities from the sets of read data. FIG. 35 illustrates the splice-site prediction system 106 organizing training data from a data set for training a neural network to generate tissue-specific conditional probabilities in accordance with one or more embodiments.

FIG. 35 illustrates a table 3502 of tissue-specific data. In some cases, the splice-site prediction system 106 collects the tissue-specific data from a database maintaining the tissue-specific data, such as the GTEx database. As shown in the table 3502, the tissue-specific data includes read data specific to multiple tissues (e.g., brain tissue, artery tissue, adipose tissue, etc.). As further shown, the tissue-specific data includes read data specific to multiple tissues (e.g., aorta tissue, coronary tissue, and tibial tissue, which are sub-tissues for the artery tissue). In some cases, each row corresponds to a nucleotide sequence represented within each of the tissues.

As shown in FIG. 35, the splice-site prediction system 106 uses the table 3502 to create a training dataset 3504 for training a neural network to generate tissue-specific conditional probabilities. For instance, in some cases, the splice-site prediction system 106 adds the sub-tissue read data together to get the collective read data for a tissue. For example, the splice-site prediction system 106 can add the sub-tissue read data for each nucleotide sequence represented in the table 3502 to get read data for that nucleotide sequence that corresponds to the tissue. Thus, the splice-site prediction system 106 gathers tissue-specific read data that facilitates the training of a neural network to generate tissue-specific conditional probabilities for nucleotide sequences.

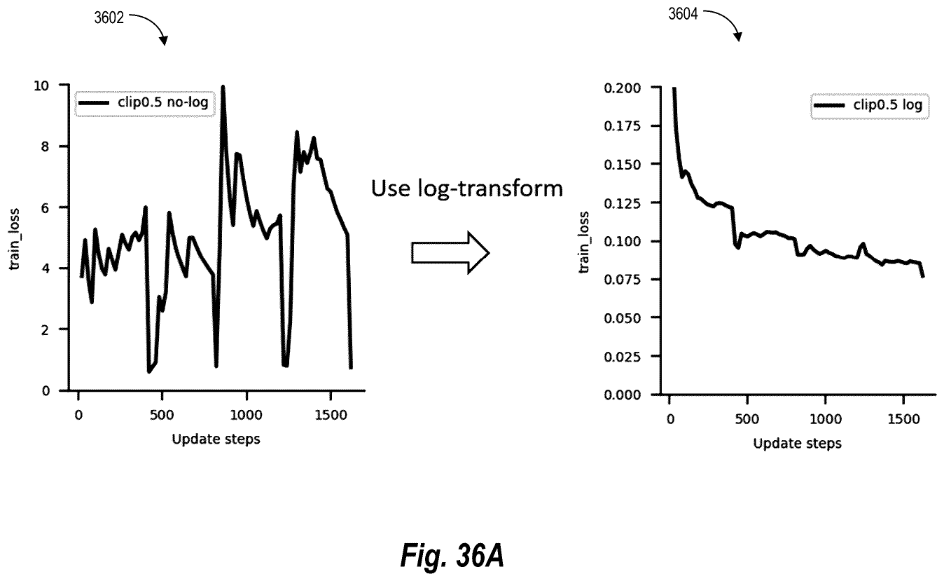

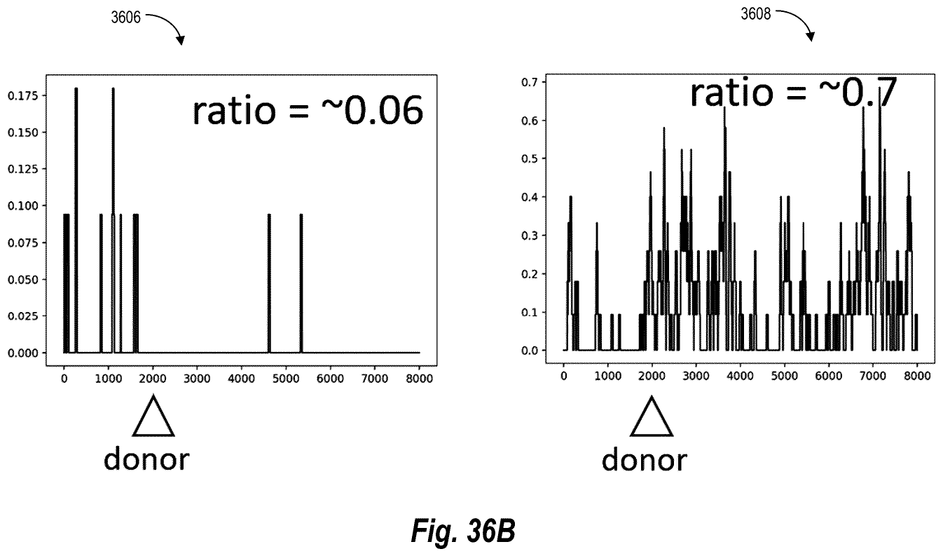

As suggested above, in some cases, the splice-site prediction system 106 pre-processes training data for using in training a neural network. In particular, the splice-site prediction system 106 can pre-process the training data to improve learning for the neural network. FIGS. 36A-36B illustrates graphs reflecting experimental results regarding the effectiveness of the splice-site prediction system 106 based on pre-processing of training data in accordance with one or more embodiments.

FIG. 36A illustrates pre-processing training data using a logarithmic transformation as discussed above with reference to FIG. 34 in accordance with one or more embodiments. In particular, FIG. 36A illustrates a first graph 3602 showing a training loss for a neural network as its trained to generate tissue-specific conditional probabilities and a genomic track signal. In particular, the first graph 3602 shows the training loss for the neural network as it learns to generate RNA binding protein signals without use of a logarithmic transformation. As shown by the first graph 3602 and as indicated above, the training loss fails to converge. Instead, the training loss is shown in a pattern of significant increases followed by significant decreases.

FIG. 36A also illustrates a second graph 3604 showing a training loss for a neural network as it learns to generate RNA binding protein signals with the use of a logarithmic transformation. In particular, the splice-site prediction system 106 applied a logarithmic transformation to the ground truth genomic track signal and uses the transformed genomic track signal to train the neural network. As shown by the second graph 3604, the training loss decreases consistently over the course of the training and approaches convergence. Accordingly, application of the logarithmic transformation provides improvements to training by stabilizing the gradient update, allowing the neural network to properly learn to generate genomic track signals.

FIG. 36B illustrates pre-processing training data using an expression level filter in accordance with one or more embodiments. Indeed, in some cases, the splice-site prediction system 106 uses expression level filtering to allow the neural network to focus on the segments of the ground truth genomic track signals that have sufficient expression. For instance, FIG. 36B illustrates a third graph 3606 showing genomic track signal amplitudes at various positions within a nucleotide sequence. As shown in the third graph 3606, very few positions within the nucleotide sequence are associated with noticeable amplitude. The amplitude spikes shown in some positions are likely due to noise. Accordingly, the splice-site prediction system 106 uses an expression level filter to establish an expression level threshold to facilitate the removal of cases of noise that could otherwise contaminate the training.

In one or more embodiments, the splice-site prediction system 106 implements an expression level filter by filtering out segments of a genomic track signal that fail to have a ratio of positions with a non-zero amplitude above a certain threshold. Segments that fail to satisfy the ratio threshold may be indicative of too much noise. For instance, the third graph 3606 indicates that only about six percent of the signal segment represented therein has a non-zero amplitude. Accordingly, in some instances, the splice-site prediction system 106 filters out that segment of the genomic track signal so that it is not used to train the neural network. The fourth graph 3608 shown in FIG. 36B, however, shows that about seventy percent of the signal segment represented therein has a non-zero amplitude. Accordingly, the splice-site prediction system 106 can determine to use the signal segment for training the neural network.

In some embodiments, the splice-site prediction system 106 implements an expression level filter by setting a threshold for the mean amplitude. In particular, the splice-site prediction system 106 can establish a threshold by which the mean amplitude of the signal segment should meet to be used in training the neural network. In some cases, the splice-site prediction system 106 compares the mean amplitude threshold against a portion or a percentile range of a signal segment. For instance, in some cases, if a percentile range of a signal segment (e.g., the 50-70 percentile) satisfies the mean amplitude threshold, the splice-site prediction system 106 determines to use the signal segment for training.

Though the splice-site prediction system 106 can use various threshold levels in various embodiments, too low of a threshold can prevent learning. For example, setting a threshold too low can allow too much noise into the training data. Further, setting a threshold too low can create biases as there may be too much low-amplitude data. Accordingly, the splice-site prediction system 106 targets setting the threshold high enough to filter out unwanted data. In some cases, the splice-site prediction system 106 experiments with various threshold levels to determine which threshold level to use.

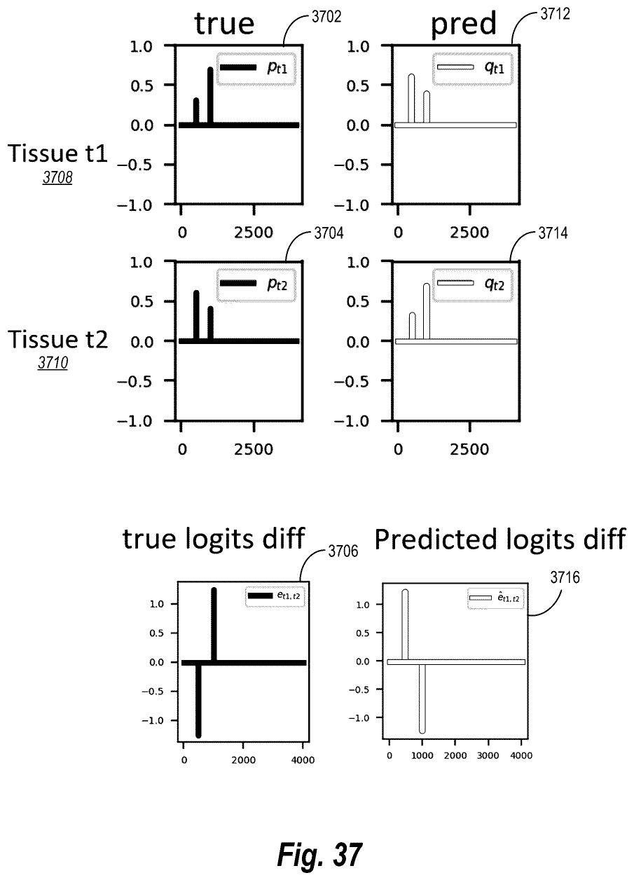

In one or more embodiments, the splice-site prediction system 106 utilizes logits in evaluating a neural network with regard to generating tissue-specific conditional probabilities. In some cases, the splice-site prediction system 106 uses logit differences. FIG. 37 Illustrates the splice-site prediction system 106 using logits to evaluate the performance of a neural network in generating tissue-specific conditional probabilities in accordance with one or more embodiments.

The splice-site prediction system 106 can use logits in evaluating a neural network to capture the significance in the differences in probabilities generated by the neural network (e.g., to capture the degree to which the probabilities differ beyond what is indicated by the numerical difference). For example, a difference in probability can have a small absolute value but still have significance. To illustrate, a change in probability from 0.1 to 0.2 is only a difference of 0.1 in terms of its absolute value, but the splice-site prediction system 106 can use logits to capture the significance of this change as the probability doubled.

As shown in FIG. 37, the splice-site prediction system 106 determines ground truth logits 3702 for a first tissue 3708 and ground truth logits 3704 for a second tissue 3710. In some cases, the splice-site prediction system 106 determines each set of ground truth logits based on corresponding ground truth probabilities, where each set of ground truth probabilities can correspond to the same nucleotide sequence and indicate conditional probabilities for the use of each position represented therein as a splice site.

As shown, the splice-site prediction system 106 can determine ground truth logit differences 3706 by comparing the ground truth logits 3702 for the first tissue 3708 and the ground truth logits 3704 for the second tissue 3710. For instance, in some cases, the splice-site prediction system 106 subtracts the ground truth logits 3704 for the second tissue 3710 from the ground truth logits 3702 for the first tissue 3708. Thus, the splice-site prediction system 106 can determine the difference of the probabilities for the first tissue 3708 and the second tissue 3710.

Similarly, as shown, the splice-site prediction system 106 determines predicted logits 3712 for the first tissue 3708 and predicted logits 3714 for the second tissue 3710. The splice-site prediction system 106 can determine each set of predicted logits based on predicted conditional probabilities generated by the neural network. The splice-site prediction system 106 can further determine predicted logit differences 3716 by comparing the logits 3712 for the first tissue 3708 and the logits 3714 for the second tissue 3710. Thus, the splice-site prediction system 106 can determine the differences in the probabilities generated by the neural network for the first tissue 3708 and the second tissue 3710.

In one or more embodiments, the splice-site prediction system 106 can compare ground truth logit differences 3706 and the predicted logit differences 3716 to determine a performance of the neural network. As an illustration, comparing the ground truth logit differences 3706 and the predicted logit differences 3716 shown in FIG. 37 reveals that the neural network made a significant error in its predictions. Thus, the splice-site prediction system 106 can determine that more training is required. Indeed, in some cases, the splice-site prediction system 106 establishes a logit differences threshold and determines that the neural network requires more training if a comparison of the predicted logit differences determined from the outputs of the neural network to the ground truth logit differences fails to satisfy the threshold.

In some embodiments, when using logits to evaluate a neural network, the splice-site prediction system 106 considers probabilities generated for all alternative splice sites. In some cases, the splice-site prediction system 106 establishes a threshold to qualify positions within a nucleotide sequence as alternative splice sites. For example, in some instances, the splice-site prediction system 106 considers positions as alternative splice sites if they have an associated probability greater than some threshold in either tissue. In some embodiments, the splice-site prediction system 106 only evaluates those splice sites associated with a logit difference that is greater than some threshold.

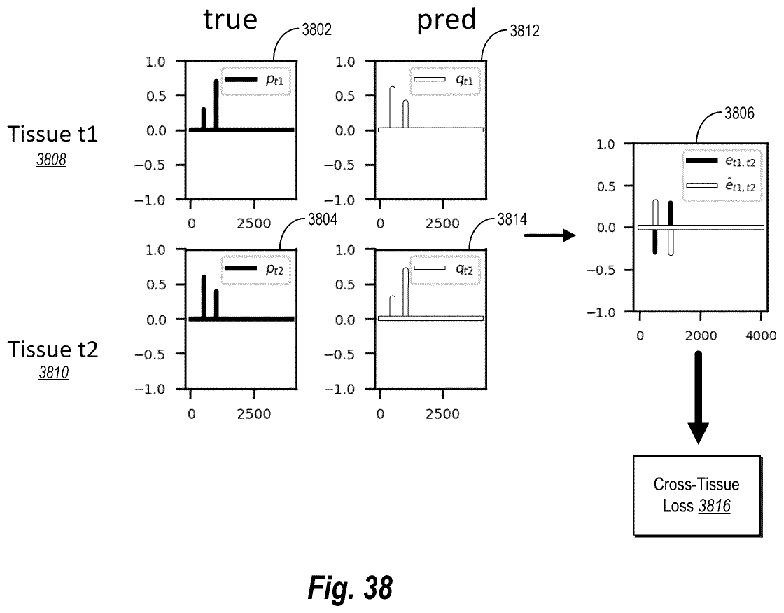

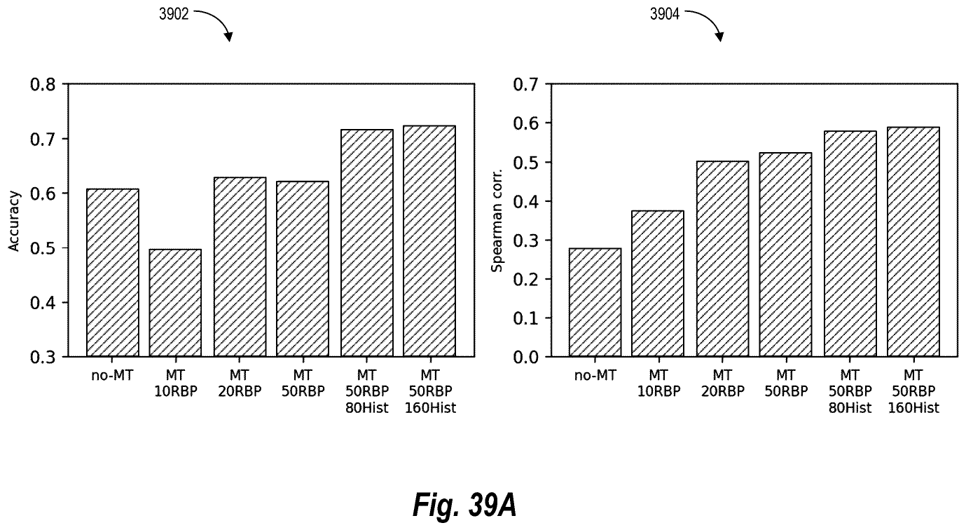

As previously mentioned, in some implementations, the splice-site prediction system 106 uses logits in evaluating a neural network to generate tissue-specific conditional probabilities. FIG. 38 illustrates the splice-site prediction system 106 using a predicted probability difference between a pair of tissues in training a neural network in accordance with one or more embodiments. As shown in FIG. 38 and explained below, the splice-site prediction system 106 uses the predicted probability difference between the terms e_{t1,t2} and ê_{t1,t2} to determine a cross-tissue loss from a cross-tissue loss function 3816.

Indeed, similar to the discussion above with reference to FIG. 37, the splice-site prediction system 106 determines ground truth logits 3802 for a first tissue 3808, ground truth logits 3804 for a second tissue 3810, and ground truth probability differences (shown in the probability differences 3806). Similarly, the splice-site prediction system 106 determines predicted logits 3812 for the first tissue 3808, predicted logits 3814 for the second tissue 3810, and predicted logit differences (shown in the logit differences).

As indicated in FIG. 38, the splice-site prediction system 106 uses the probability differences 3806 as part of the cross-tissue loss function 3816 used in training the neural network. For instance, as indicated by FIG. 38, the splice-site prediction system 106 uses the ground truth probability differences as the term e_{t1,t2} in functions 15 and 16 discussed above. Similarly, the splice-site prediction system 106 uses the predicted probability differences as the term ê_{t1,t2} in those functions. Thus, in some cases, using the cross-tissue loss from the cross-tissue loss function 3816 facilitates learning to generate tissue-specific conditional probabilities.

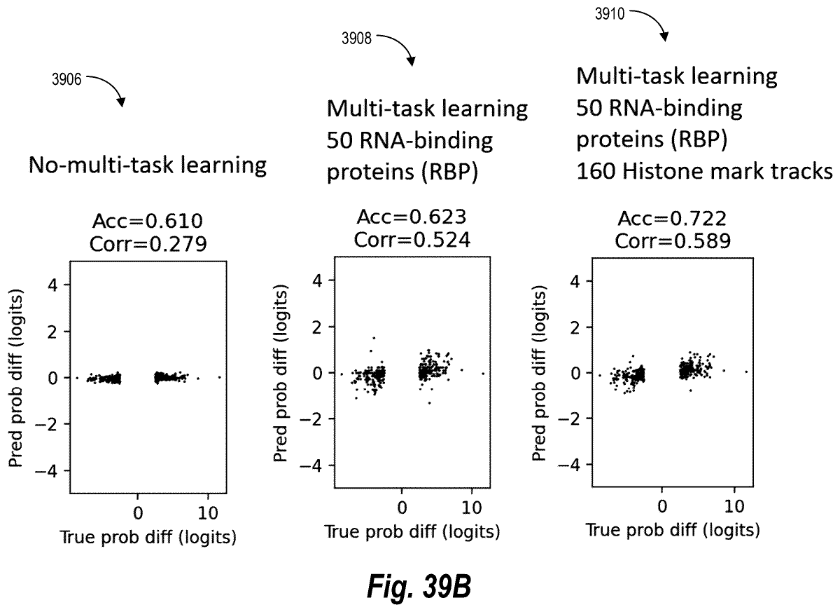

As mentioned above, in some embodiments, the splice-site prediction system 106 employs multi-task learning to train a neural network to generate tissue-specific conditional probabilities and genomic track signals. In particular, in some cases, the splice-site prediction system 106 employs the multi-task learning to improve the neural network's learning for tissue-specific splicing detection. FIGS. 39A-39B illustrates graphs reflecting experimental results regarding the effectiveness of the splice-site prediction system 106 based on training of a neural network to generate tissue-specific conditional probabilities in accordance with one or more embodiments.

FIG. 39A illustrates a first graph 3902 and a second graph 3904. Each graph compares multiple implementations of the splice-site prediction system 106 that employ multi-task learning against an implementation of the splice-site prediction system 106 that does not employ multi-task learning (labeled “no-MT”). The represented implementations that do employ multi-task learning train the neural network to generate tissue-specific conditional probabilities and one or more genomic track signals. For instance, some of the implementations train the neural network to learn RNA binding proteins (labeled “RBP”), and some of the implementations train the neural network to learn histone mark signals (labeled “Hist”). The number that follows each of these designations represent the number of RNA binding proteins or histone mark signals that are learned.

The first graph 3902 shows the performance of the tested models with respect to an accuracy metric, and the second graph 3904 shows the performance of the tested models with respect to the Spearman correlation metric. As shown by the graphs, using multi-task learning to train a neural network generally improves the ability of the splice-site prediction system 106 to determine tissue-specific conditional probabilities—and significantly so when it comes to Spearman correlation. As further indicated by the graphs, increasing the number and variety of genomic track signals that are learned provides further improved performance.

FIG. 39B illustrates a third graph 3906, a fourth graph 3908, and a fifth graph 3910. Each of the graphs illustrate the correlation of one of the tested models represented in FIG. 39A. In particular, the graphs illustrate the correlation in terms of the logit differences associated with tissue pairs. As shown by the graphs, the use of multi-task learning improves the ability of the splice-site prediction system 106 to determine the splicing differences between tissue pairs. Further, the graphs show that increasing the number and variety of genomic track signals that are learned can offer further improvement.

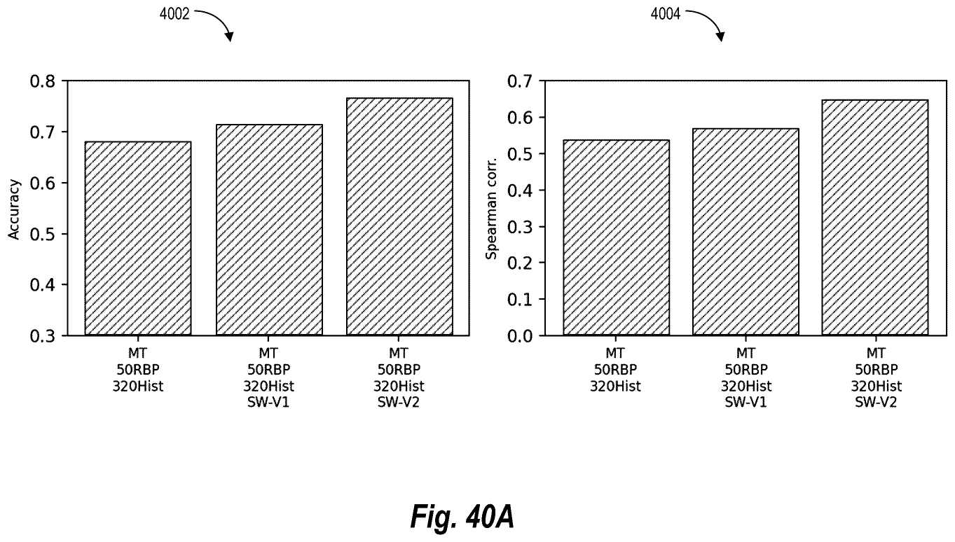

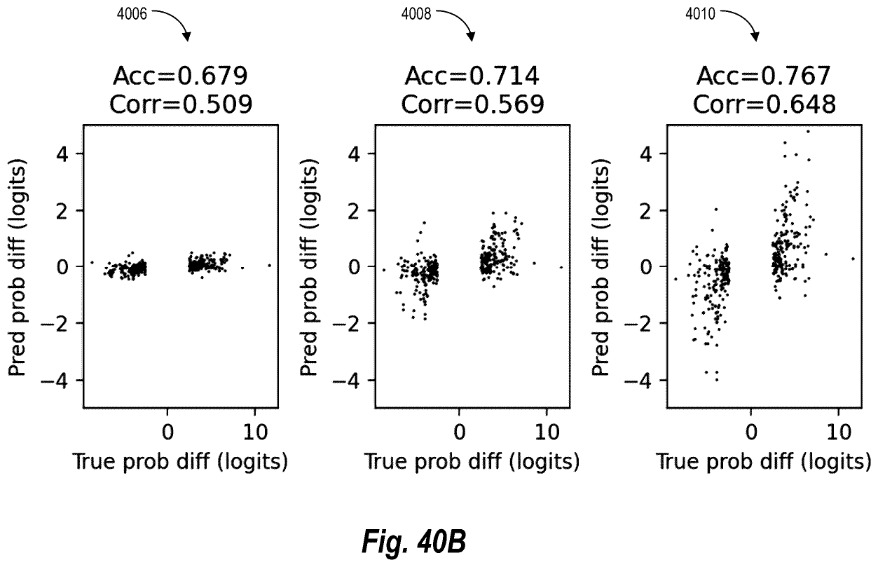

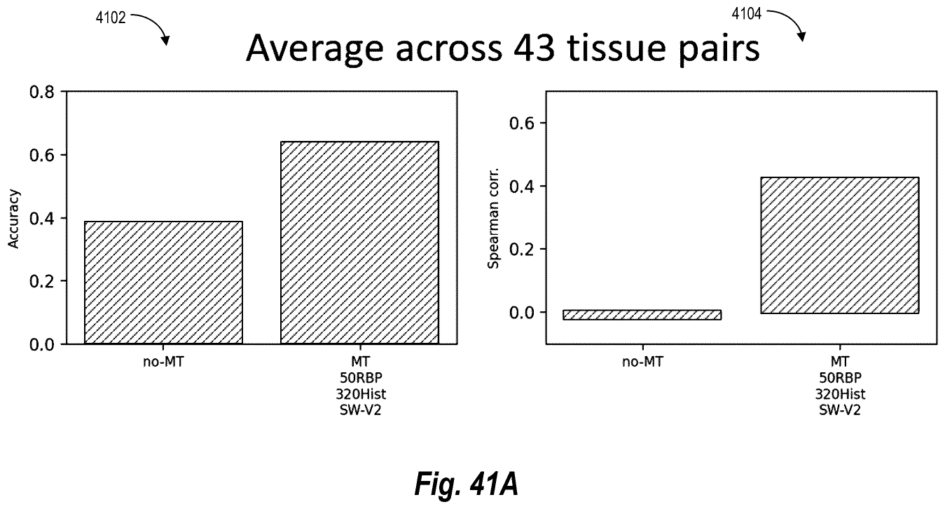

As further mentioned, in some embodiments, the splice-site prediction system 106 uses a soft-weighted cross-entropy loss when training a neural network to generate tissue-specific conditional probabilities. FIGS. 40A-40B illustrates graphs reflecting experimental results regarding the effectiveness of the splice-site prediction system 106 based on training of a neural network to generate tissue-specific conditional probabilities using a soft-weighed cross-entropy loss in accordance with one or more embodiments.

FIG. 40A illustrates a first graph 4002 and a second graph 4004. Each graph compares implementations of the splice-site prediction system 106 that employ a soft-weighted cross-entropy loss function (labeled “SW-V1” or “SW-V2”) against an implementation of the splice-site prediction system 106 that does not employ soft-weighted cross-entropy loss. Indeed, as shown in the graphs, the splice-site prediction system 106 can implement various versions of a soft-weighted cross-entropy loss function in various embodiments. The general soft-weighted cross-entropy loss function is provided above in function 14. In some cases, the splice-site prediction system 106 implements one of the following variations:

$$scale ( V 1 ) = 1 + S {\sum }_{t = 1}^{T} {\sum }_{i = 1}^{N} \vert {p}_{it} - {\overline{p}}_{i} \vert \qquad ( 18 )$$

$$scale ( V 2 ) = 1 + S {\sum }_{i = 1}^{N} {\vert {p}_{it} - {\overline{p}}_{i} \vert}^{2} \qquad ( 19 )$$

Function 18 weighs each tissue equally and includes dimensions (Batch, 1,1). Function 19 weights each tissue separately and includes dimensions (Batch, 1, Tissue).

The first graph 4002 shows the performance of the tested models with respect to an accuracy metric, and the second graph 4004 shows the performance of the tested models with respect to the Spearman correlation metric. As shown by the graphs, utilizing a soft-weighted cross-entropy loss function to train a neural network generally improves the ability of the splice-site prediction system 106 to generate tissue-specific conditional probabilities. As further shown, the second version of the soft-weighted cross-entropy loss function as shown in function 19 provides further improved performance.

FIG. 40B illustrates a third graph 4006, a fourth graph 4008, and a fifth graph 4010. Each of the graphs illustrate the correlation of one of the tested models represented in FIG. 40A. In particular, the graphs illustrate the correlation in terms of the logit differences associated with tissue pairs. As shown by the graphs, the use of a soft-weighted cross-entropy loss function improves the ability of the splice-site prediction system 106 to determine the splicing differences between tissue pairs. Further, the graphs show that use of the second version of the soft-weighted cross-entropy loss function as shown in function 19 can offer further improvement.

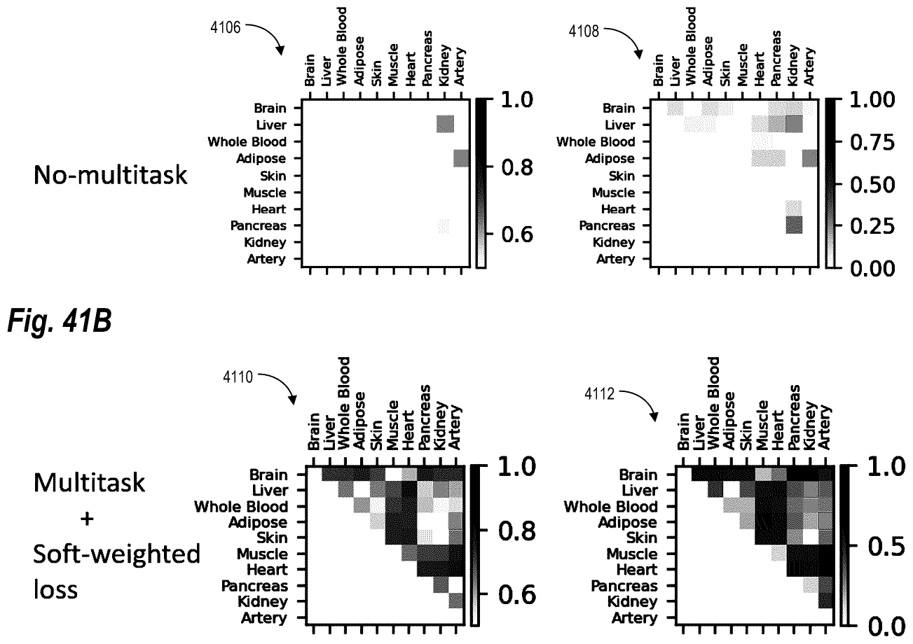

FIGS. 41A-41B illustrates graphs reflecting additional experimental results regarding the effectiveness of the splice-site prediction system 106 based on training of a neural network to generate tissue-specific conditional probabilities in accordance with one or more embodiments. FIG. 41A illustrates a first graph 4102 and a second graph 4104 comparing an implementation of the splice-site prediction system 106 that does not employ multi-task learning to an implementation that employs multi-task learning and a soft-weighted cross-entropy loss function. In particular, the tested models represented in the graphs include models that have been trained to differentiate between at least ten different tissues. Indeed, many of the immediately preceding graphs compare the performance of that have learned to differentiate between a signal pair of tissues (e.g., brain and liver). Thus, the graphs shown in FIG. 41A represent a more complex situation where splicing is determined for many more tissues. As noted, the graphs provide an average across forty-three tissue pairs.

The first graph 4102 shows the performance of the tested models with respect to an accuracy metric (e.g., the accuracy one or accuracy two metric mentioned above with reference to FIGS. 7-8), and the second graph 4104 shows the performance of the tested models with respect to the Spearman correlation metric. As shown by the graphs, multi-task learning with the implementation of the soft-weighted cross-entropy loss function significantly improves the performance of the splice-site prediction system 106 in terms of both accuracy and correlation. Notably, a comparison of the second graph 4104 to the second graph 3904 illustrated in FIG. 39A shows that correlation drops significantly when going from analysis involving a single tissue pair to analysis involving at least ten different tissues. While the implementation of the splice-site prediction system 106 that does not employ multi-task learning suffers significantly in terms of correlation when differentiating between ten different tissues, the implementation that does employ multi-task learning is able to provide a much stronger correlation.

FIG. 41B illustrates a first heat map 4106, a second heat map 4108, a third heat map 4110, and a fourth heat map 4112 that more compares the performance of each tested model represented in FIG. 41 in terms of specific tissue pairs. The first heat map 4106 and the second heat map 4108 provide the accuracy and correlation, respectively, of the implementation of the splice-site prediction system 106 that does not employ multi-task learning. The third heat map 4110 and the fourth heat map 4112 provide the accuracy and correlation, respectively, of the implementation that employs multi-task learning and a soft-weighed cross-entropy loss. As shown by the heat maps, the implementation of the splice-site prediction system 106 that employs multi-task learning and the soft-weighted cross-entropy loss shows significant improvement in terms of both accuracy and correlation across a significant number of tissue pairs.

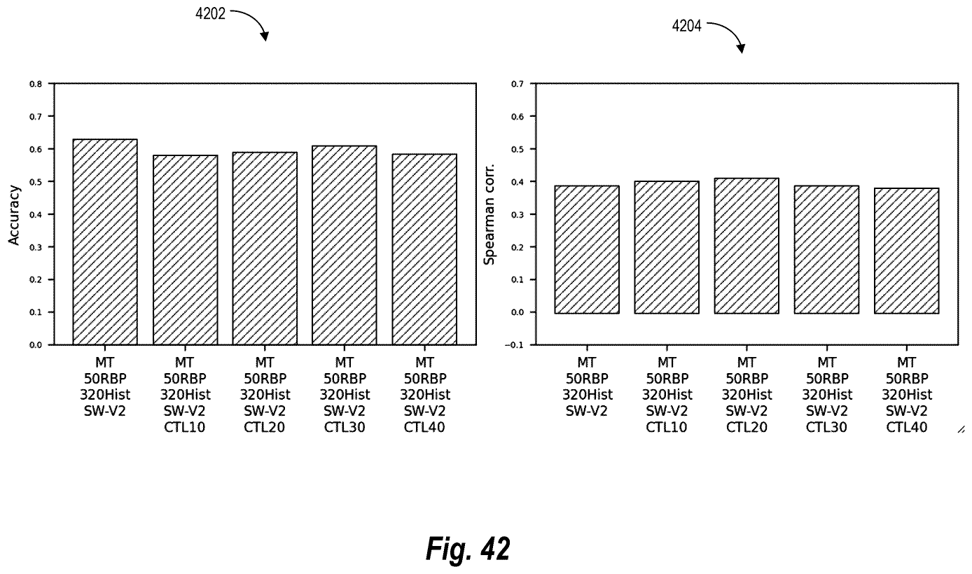

As mentioned above, in some embodiments, the splice-site prediction system 106 utilizes a cross-tissue loss function in training a neural network to generate tissue-specific conditional probabilities. FIG. 42 illustrates a graph reflecting further experimental results regarding the effectiveness of the splice-site prediction system 106 based on training of a neural network using a cross-tissue loss in accordance with one or more embodiments. FIG. 42 illustrates a first graph 4202 and a second graph 4204 comparing an implementation of the splice-site prediction system 106 that does not employ a cross-tissue loss function to various implementations that do employ a cross-tissue loss function (labeled “CT”). The weight used in the cross-tissue loss function (i.e., the value of K from function 15) is provided after the CT label. As shown in the graphs, the cross-tissue loss function provides some promising results. For instance, based on the weight provided to the cross-tissue loss function, the splice-site prediction system 106 can provide improved correlation with comparable accuracy. Thus, in some cases, the splice-site prediction system 106 selects a cross-tissue loss function weight that provides improvements for at least one of accuracy or correlation.

By modeling tissue-specific (or other context-specific) splicing, the splice-site prediction system 106 operates more flexible when compared to existing systems. In particular, the splice-site prediction system 106 uses a neural network that has been flexibly trained to recognize data patterns that provide nuance indicative of tissue-specific splicing. Further, as mentioned, the splice-site prediction system 106 unconventionally employs multi-task learning to train the neural network to learn tissue-specific splicing and genomic track signals for nucleotide sequences, allowing for tissue-specific splicing prediction that is unavailable under existing systems. The multi-task learning—as well as the use of soft-weighed cross-entropy loss and/or cross-tissue loss—improves the accuracy with which tissue-specific conditional probabilities are generated. Thus, the splice-site prediction system 106 offers more accurate, informative outputs that incorporate the data nuances within nucleotide sequences accounting for how those sequences are spliced in various tissues.

As previously mentioned, the splice-site prediction system 106 can leverage the probabilities (e.g., the conditional probabilities and/or the intron-retention probabilities) generated via the employed neural network for various purposes in some embodiments. FIGS. 42-46 illustrate the splice-site prediction system 106 leveraging probabilities generated using a neural network in accordance with one or more embodiments.

Indeed, as mentioned, in many instances, the splice-site prediction system 106 offers a more flexible and a more accurate approach to identifying splice sites within nucleotide sequences as well as their usage rates when compared to existing systems. Accordingly, the splice-site prediction system can more flexibly and accurately use splice-site predictions when compared to existing systems. For instance, the splice-site prediction system 106 can use its predictions to more accurately determine whether a variant within a nucleotide sequence is associated with alternative splicing of pre-mRNA relative to reference mRNA. The splice-site prediction system 106 can further use its predictions to more accurately determine treatments for the alternative splicing or its effects. Thus, the splice-site prediction system 106 utilizes improvements in predicting splice sites to provide better downstream applications of its predictions.

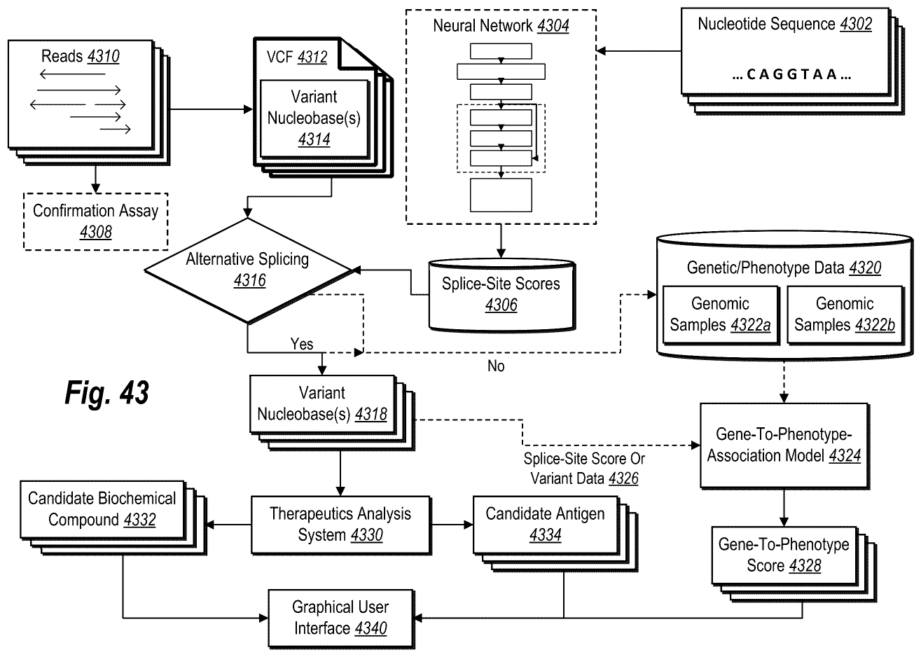

FIG. 43 illustrates an overview of the splice-site prediction system 106 leveraging conditional probabilities generated for a nucleotide sequence in accordance with one or more embodiments. As an overview of the process depicted in FIG. 43, the splice-site prediction system 106 identifies, for a genomic sample, a variant nucleobase at a target position within a nucleotide sequence 4302 corresponding to a genomic region. The splice-site prediction system 106 further accesses a splice-site score of the splice-site scores 4306 for the variant nucleobase based on one or more conditional probabilities that a flanking nucleobase from the nucleotide sequence 4302 is part of a matching donor or a matching acceptor corresponding to a conditioned nucleobase. Based on the splice-site score, the splice-site prediction system 106 determines that the variant nucleobase at the target position within the nucleotide sequence 4302 is associated with alternative splicing 4316 of pre-messenger ribonucleic acid (pre-mRNA) relative to a reference splicing of pre-mRNA.

Having determined that a variant nucleobase of variant nucleobase(s) 4318 is associated with alternative splicing 4316, as further depicted in FIG. 43, the splice-site prediction system 106 can determine a candidate biochemical compound 4332 or a candidate antigen 4334 that targets the variant nucleobase or one or more adjacent nucleobases flanking the variant nucleobase. Alternatively, the splice-site prediction system 106 can provide the splice-site score (and/or variant data) to a gene-to-phenotype-association model to determine a gene-to-phenotype score indicating a likelihood that a target gene is associated with a target phenotype.

The following paragraphs describe FIG. 43 in more detail. As shown in FIG. 43, the splice-site prediction system 106 uses a neural network 4304 to analyze a nucleotide sequence 4302. As previously discussed, the nucleotide sequence 4302 can include a conditioned nucleobase at a conditioned position and a plurality of flanking nucleobases. In some cases, the nucleotide sequence 4302 further includes a variant nucleobase. For instance, in some cases, the variant nucleobase is one of the flanking nucleobases. In some implementations, the nucleotide sequence 4302 corresponds to pre-mRNA for an organism that is a genomic host of the nucleotide sequence 4302. For instance, the nucleotide sequence 4302 can be part of a genomic sample extracted from the organism.

As further shown in FIG. 43, the splice-site prediction system 106 accesses one or more splice-site scores based on the analysis of the nucleotide sequence 4302 via the neural network 4304. Indeed, FIG. 43 illustrates adding one or more splice-site scores to a dataset of splice-site scores 4306. As used herein, the term “splice-site score” refers to a measure of splicing associated with a nucleotide sequence. In particular, a splice-site score can refer to a value or set of values that indicates splicing associated with a nucleotide sequence. Indeed, in one or more embodiments, the splice-site prediction system 106 determines the one or more splice-site scores based on the conditional probabilities generated by the neural network 4304 via its analysis of the nucleotide sequence 4302. For instance, in some embodiments, a splice-site score includes or is derived from one or more conditional probabilities generated from the analysis of the nucleotide sequence 4302.

Accordingly, in certain cases, the splice-site prediction system 106 accesses previously determined splice-site scores that have been derived (e.g., extracted or isolated from) from conditional probabilities determined by the neural network 4304, but the splice-site prediction system 106 need not itself determine the conditional probabilities. The splice-site scores 4306 may, therefore, be part of a database (e.g., lookup table of splice-site scores per variant nucleobase in a particular nucleotide sequence for a gene). Indeed, the splice-site scores may be available for download from a GitHub website or other accessible database.

To illustrate, in some cases, the splice-site prediction system 106 uses the neural network 4304 to generate conditional probabilities for the flanking nucleobases of the nucleotide sequence 4302 and determines a splice site-score for the nucleotide sequence 4302 based on one or more of those conditional probabilities. In some cases, the splice-site prediction system 106 uses the neural network 4304 to generate context-specific conditional probabilities for the flanking nucleobases with respect to a target context and determines the splice-site score for the nucleotide sequence 4302 based on one or more of those context-specific conditional probabilities. In some cases, the splice-site score includes a vector of the one or more (context-specific) conditional probabilities. In some cases, the splice-site score includes a sum, an average, or other mathematic derivation of the one or more (context-specific) conditional probabilities.

As previously mentioned, the splice-site prediction system 106 can generate context-specific conditional probabilities for a variety of contexts. Accordingly, the context-specific conditional probabilities used to determine one or more splice-site scores can include, but are not limited to, tissue-specific conditional probabilities determined with respect to one or more target tissues; disease-specific conditional probabilities determined with respect to one or more target diseases; cell-type-specific conditional probabilities determined with respect to one or more target cell types; cell-line-specific conditional probabilities determined with respect to one or more target cell lines; or assay-specific conditional probabilities determined with respect to one or more target assay types.

As mentioned, in some cases, the nucleotide sequence 4302 includes a variant nucleobase. As illustrated in FIG. 43, in one or more embodiments, the splice-site prediction system 106 determines or confirms that the nucleotide sequence 4302 includes a variant nucleobase via a confirmation assay 4308. For instance, as shown, via the confirmation assay 4308, the splice-site prediction system 106 obtains a plurality of nucleotide reads 4310 for the nucleotide sequence 4302. Based on the plurality of nucleotide reads 4310, the splice-site prediction system 106 generates a variant call file 4312. As indicated by FIG. 43, the variant call file 4312 can identify one or more variant nucleobases 4314 of the nucleotide sequence 4302 and variant nucleobases of one or more other nucleotide sequences.

In some cases, the splice-site prediction system 106 obtains the plurality of nucleotide reads 4310 via the confirmation assay 4308 based on one or more genomic samples. Thus, in some cases, the splice-site prediction system 106 identifies a variant nucleobase within the nucleotide sequence 4302 by identifying a set of genomic samples comprising the variant nucleobase within the nucleotide sequence 4302 based on a set of nucleotide reads for the set of genomic samples. In some instances, the splice-site prediction system 106 identifies the variant nucleobase by identifying a genomic sample (e.g., a single genomic sample) comprising the variant nucleobase within the nucleotide sequence 4302 based on nucleotide reads for the genomic sample.

As further shown in FIG. 43, the splice-site prediction system 106 determines the presence of alternative splicing 4316 using the one or more splice-site scores determined for the nucleotide sequence 4302 and the variant call file 4312 identifying one or more variant nucleobases within the nucleotide sequence 4302. In particular, the splice-site prediction system 106 determines whether the nucleotide sequence 4302 is associated with the alternative splicing 4316. To illustrate, in one or more embodiments, the splice-site prediction system 106 targets a variant nucleobase of the nucleotide sequence 4302 as identified by the variant call file 4312 (the variant nucleobase being located at a target position within the nucleotide sequence 4302). The splice-site prediction system 106 further accesses a splice-site score for the variant nucleobase (e.g., a splice-site score determined for the nucleotide sequence 4302). In some cases, the splice-site prediction system 106 accesses the splice-site score for the variant nucleobase by accessing one or more (context-specific) conditional probabilities from among the (context-specific) conditional probabilities generated for the nucleotide sequence 4302. In some cases, the splice-site prediction system 106 accesses the splice-site score for the variant nucleobase by accessing another value derived from one or more (context-specific) conditional probabilities.

In some embodiments, the splice-site prediction system 106 performs a minigene splicing assay (e.g., the confirmation assay 4308) on a genomic sample. As used herein, the term “minigene splicing assay” refers to an analysis of the splicing of a genomic sample. In particular, in some embodiments, a minigene splicing assay involves creating a “minigene” that includes a nucleotide sequence and analyzing how the minigene experiences splicing. By performing a minigene splicing assay, the splice-site prediction system 106 can identify alternative splicing events that may be associated with the genomic sample. Further, the splice-site prediction system 106 confirms, based on the minigene splicing assay, the variant nucleobase at the target position within the nucleotide sequence 4302 is associated with the alternative splicing 4316.

By determining whether the nucleotide sequence 4302 is associated with the alternative splicing 4316, the splice-site prediction system 106 can determine whether the variant nucleobase included in the nucleotide sequence 4302 is associated with the alternative splicing 4316. For instance, in some cases, the splice-site prediction system 106 can determine whether the variant nucleobase at least partially causes the alternative splicing 4316 (e.g., whether the presence of the variant nucleobase is at least one factor that causes the alternative splicing 4316). In some cases, where the nucleotide sequence 4302 includes multiple variant nucleobases, the splice-site prediction system 106 determines whether the multiple variant nucleobases are at least a partial cause of the alternative splicing 4316. Thus, as indicated in FIG. 43, upon determining that the alternative splicing 4316 is associated with the nucleotide sequence 4302, the splice-site prediction system 106 identifies a variant nucleobase of the variant nucleobase(s) 4318 of the nucleotide sequence 4302 that is associated with the alternative splicing 4316.

In some cases, the splice-site prediction system 106 determines, for a genomic sample, nucleotide reads corresponding to a genomic region. The splice-site prediction system 106 further compares the nucleotide reads corresponding to the genomic region with a reference genome. Based on comparing the nucleotide reads corresponding to the genomic region with the reference genome, the splice-site prediction system 106 can confirm the genomic sample comprises the variant nucleobase at a target position within a nucleotide sequence and that the variant nucleobase is associated with the alternative splicing.

In some cases, the splice-site prediction system 106 determines that a variant nucleobase at a target position within a nucleotide sequence is associated with alternative splicing of pre-mRNA by comparing the predicted splicing for the nucleotide sequence with a reference splicing. For instance, the splice-site prediction system 106 can determine that the variant nucleobase at the target position within the nucleotide sequence is: associated with splicing of pre-mRNA at a first matching donor or a first matching acceptor corresponding to the conditioned nucleobase in a first percentage of splicing events; and associated with splicing of pre-mRNA at a second matching donor or a second matching acceptor corresponding to the conditioned nucleobase in a second percentage of splicing events, the first percentage and the second percentage differing from percentages of splicing events in the reference splicing of pre-mRNA at the first matching donor or the first matching acceptor and the second matching donor or the second matching acceptor.

As further indicated, the splice-site prediction system 106 adds data to a dataset of genetic/phenotype data 4320. In particular, the splice-site prediction system 106 adds the data upon determining the alternative splicing 4316 is associated with the nucleotide sequence 4302. The splice-site prediction system 106 can also add the data upon determining the alternative splicing 4316 is not associated with the nucleotide sequence 4302.

As shown, the genetic/phenotype data 4320 includes genomic samples, such as the genomic samples 4322a-4322b. Thus, in some cases, the splice-site prediction system 106 adds the genomic sample associated with the nucleotide sequence 4302 (e.g., the genomic sample from which the nucleotide sequence 4302 was taken) to the genetic/phenotype data 4320. In some cases, the splice-site prediction system 106 labels the genomic sample added to the genetic/phenotype data 4320 as being associated with the alternative splicing 4316 or as being unassociated with the alternative splicing 4316. For instance, in some cases, the splice-site prediction system 106 updates the genetic/phenotype data 4320 to the include genomic sample and further updates the data to include a status indicating whether the genomic sample is associated with the alternative splicing 4316 (e.g., a single bit taking on one value to show an association or another value to show that the genomic sample is not associated with the alternative splicing 4316). Thus, the splice-site prediction system 106 can build a dataset that identifies which analyzed genomic samples represented therein are associated with alternative splicing and which are not. Further, as indicated, the splice-site prediction system 106 can associate the genomic samples included in the genetic/phenotype data 4320 with phenotypes. For instance, in some cases, the splice-site prediction system 106 creates a mapping that maps each of the genomic samples represented therein to one or more phenotypes.

As shown in FIG. 43, the splice-site prediction system 106 can perform one or more operations based on identifying variant nucleobases associated with the alternative splicing 4316. For instance, in one or more embodiments, the splice-site prediction system 106 determines whether a target gene is associated with a target phenotype. Indeed, as shown in FIG. 43, the splice-site prediction system 106 can provide a splice-site score for a variant nucleobase or corresponding variant data to a gene-to-phenotype-association model 4324 (as shown by the line 4326).

As used herein, the term “gene-to-phenotype-association model” refers to a computer-implemented model that associates genes and phenotypes. In particular, a gene-to-phenotype-association model can refer to a computer-implemented algorithm or set of computer-implemented algorithms that analyze data associated with a gene or genomic sample (e.g., a nucleotide sequence taken from a genomic sample) and determine a phenotype associated with the gene or genomic sample. In some cases, a gene-to-phenotype-association model includes a machine learning model, such as a neural network. In one or more embodiments, the splice-site prediction system 106 utilizes a gene-based burden test model as the gene-to-phenotype-association model 4324. For example, the splice-site prediction system 106 can input splice-site scores into a software model like Test Rare vAriants with Public Data (TRAPD) to perform gene-based burden testing, as described by Michael H. Guo, “Burden Testing of Rare Variants Identified through Exome Sequencing via Publicly Available Control Data,” 103 Am. J. Hum. Genet. 522-534 (2018), which is hereby incorporated by reference in its entirety.

Alternatively, in some implementations, the splice-site prediction system 106 uses a rare-variant collapsing model as the gene-to-phenotype-association model 4324. For example, the splice-site prediction system 106 can use splice-site scores (and optionally other scores) to identify rare variants and build a gene-by-individual indicator matrix for a rare-variant collapsing model, as described by Gundula Povysil et al., “Rare-variant Collapsing Analyses for Complex Traits: Guidelines and Applications,” 20 Nature Reviews Genetics 747-759 (2019), which is hereby incorporated by reference in its entirety. Additionally, or alternatively, in some embodiments, the splice-site prediction system 106 uses a probabilistic model as the gene-to-phenotype-association model 4324. For example, the splice-site prediction system 106 can input splice-site scores (and optionally other scores) into a probabilistic model to generate a probabilistic score that genetic variants have a causal link to a disease, such as Alzheimer's disease. In some cases, the probabilistic model takes the form of a Probabilistic Annotation INTegratOR (PAINTOR), as described by Jeremy Schwarzentruber et al., “Genome-wide Meta-analysis, Fine-Mapping and Integrative Prioritization Implicate New Alzheimer's Disease Risk Genes,” 53 Nature Genetics 392-402 (2021), which is hereby incorporated by reference in its entirety.

Indeed, as shown in FIG. 43, the splice-site prediction system 106 can utilize the gene-to-phenotype-association model 4324 to analyze the splice-site score for the variant nucleobase or the corresponding variant data along with data accessed from the genetic/phenotype data 4320. For instance, in some cases, the splice-site prediction system 106 uses the data accessed from the genetic/phenotype data 4320 to indicate the gene or genomic sample associated with the variant nucleobase (e.g., the gene or genomic sample associated with the nucleotide sequence 4302). In some cases, the splice-site prediction system 106 further uses the data accessed from the genetic/phenotype data 4320 as an indication of a target phenotype that may be associated with the gene or genetic sample. As shown in FIG. 43, the splice-site prediction system 106 uses the gene-to-phenotype-association model 4324 to generate a gene-to-phenotype score 4328 indicating a likelihood that the gene or genomic sample is associated with a phenotype.

To provide an example, in one or more embodiments, the splice-site prediction system 106 identifies (e.g., from the genetic/phenotype data 4320) a first set of genomic samples comprising the variant nucleobase at a target position within the nucleotide sequence 4302 based on a first set of nucleotide reads for the first set of genomic samples. The splice-site prediction system 106 further identifies (e.g., from the genetic/phenotype data 4320) a second set of genomic samples comprising a reference nucleobase at the target position within the nucleotide sequence 4302 based on a second set of nucleotide reads for the second set of genomic samples. Additionally, the splice-site prediction system 106 provides, to the gene-to-phenotype-association model 4324, the splice-site score for the variant nucleobase, an additional splice-site score for the reference nucleobase, and genetic data indicating a phenotype exhibited by the first set of genomic samples and the second set of genomic samples. Using the gene-to-phenotype-association model 4324, the splice-site prediction system 106 determines the gene-to-phenotype score 4328 comprising a value indicating a statistical likelihood that the target gene is associated with the target phenotype based on the splice-site score, the additional splice-site score, and the genetic data.

To provide another example, in some embodiments, the splice-site prediction system 106 provides the splice-site score for the variant nucleobase at the target position by identifying the variant nucleobase for the gene-to-phenotype-association model 4324 based on the splice-site score or inputting the splice-site score into the gene-to-phenotype-association model 4324. In some cases, the splice-site prediction system 106 determines the gene-to-phenotype score 4328 by generating, using the gene-to-phenotype-association model 4324 and based on a gene-by-individual matrix comprising data for the target gene, a value for the gene-to-phenotype score 4328 indicating the likelihood that the target gene is associated with the target phenotype. In some implementations, the splice-site prediction system 106 determines the gene-to-phenotype score 4328 by generating, utilizing the gene-to-phenotype-association model 4324 and based on the splice-site score, a probability for the gene-to-phenotype score 4328 indicating the likelihood that the target gene is associated with the target phenotype.

As further shown in FIG. 43, the splice-site prediction system 106 can employ a therapeutics analysis system 4330. In particular, the splice-site prediction system 106 can employ the therapeutics analysis system 4330 to determine one or more treatments for the alternative splicing 4316 associated with the variant nucleobase. Indeed, in one or more embodiments, the splice-site prediction system 106 determines that a variant nucleobase at a target position within a nucleotide sequence is associated with alternative splicing of pre-mRNA that causes a disease exhibited by an organism of a genomic sample. Thus, in some cases, the splice-site prediction system 106 uses the therapeutics analysis system 4330 to determine a treatment.

In some embodiments, the splice-site prediction system 106 uses the therapeutics analysis system 4330 to determine a candidate biochemical compound that targets the variant nucleobase or one or more adjacent nucleobases flanking the variant nucleobase to modify effects of the alternative splicing 4316 of the pre-mRNA for an organism of the corresponding genomic sample. For instance, as shown in FIG. 43, the splice-site prediction system 106 can employ the therapeutics analysis system 4330 to determine the candidate biochemical compound 4332 and/or the candidate antigen 4334.

To illustrate, in some embodiments, the splice-site prediction system 106 identifies (e.g., using an external database that provides a mapping between variants and treatments) one or more candidate antisense oligonucleotides that modify splicing of the pre-mRNA for the genomic sample relative to the alternative splicing of the pre-mRNA. In some instances, the splice-site prediction system 106 identifies one or more candidate pharmaceutical compounds that modify splicing of the pre-mRNA of the genomic sample relative to the alternative splicing 4316 of the pre-mRNA. In some implementations, the splice-site prediction system 106 identifies one or more candidate antigens that targets the variant nucleobase or the one or more adjacent nucleobases flanking the variant nucleobase.

In some embodiments, for example, the splice-site prediction system 106 can employ RESCUE-ESE developed by Massachusetts Institute of Technology and/or ESEfinder developed by the University of Minnesota, to identify candidate antisense oligonucleotides as the candidate biochemical compound 4332, as described by Jinkuk Kim et al., “Patient-Customized Oligonucleotide Therapy for a Rare Genetic Disease,” 381 N. Engl. J. Med. 1644-1652 (2019), which is hereby incorporated by reference in its entirety. Alternatively, as a further example, the splice-site prediction system 106 can identify a tumor antigen by using T lymphocyte-mediated recognition of tumor antigens and/or further molecular identification of different tumor antigen types, as described by Vid Leko & Steve A. Rosenberg, “Identifying and Targeting Human Tumor Antigens for T Cell-Based Immunotherapy of Solid Tumors,” 38 Cancer Cell 454-472 (2020), which is hereby incorporated by reference in its entirety.

As further shown in FIG. 43, the splice-site prediction system 106 provides one or more of the outputs (e.g., one or more of the candidate biochemical compound 4332, the candidate antigen 4334, and the gene-to-phenotype score 4328) to a graphical user interface 4340. In particular, the splice-site prediction system 106 provides one or more of the outputs to a client device for display within the graphical user interface 4340. Though not shown in FIG. 43, the splice-site prediction system 106 can provide other data for display within the graphical user interface 4340, such as the splice-site score.

In some cases, the splice-site prediction system 106 provides one or more of the outputs to the graphical user interface 4340 in response to user input received via the graphical user interface 4340. To illustrate, in one or more embodiments, the splice-site prediction system 106 receives, via the graphical user interface 4340, input indicating a nucleotide sequence or, more specifically, input indicating a variant (e.g., a nucleotide sequence including a variant or a code, indicator, or name of the variant) to be analyzed. Upon receiving the input, the splice-site prediction system 106 either (i) performs the process described above by executing a neural network to determine conditional probabilities (e.g., the relevant portions of the process) or (ii) identifies previously determined conditional probabilities and/or splice-site scores from a lookup table or database to obtain one or more outputs and provides the one or more outputs (or other data, such as a splice-site score) for display to the graphical user interface 4340. Thus, in some cases, the splice-site prediction system 106 provides interactive features by which a user can focus analysis on a targeted variant to determine a splice-site score for the variant, a gene-to-phenotype score for the variant, and/or one or more determined therapies for the variant.

As just mentioned, in some cases, the splice-site prediction system 106 determines whether variant nucleobases in target nucleotide sequences are associated with alternative splicing. In accordance with one or more embodiments, FIG. 44 illustrates examples of alternative splicing that can be identified or associated with variant nucleobases by the splice-site prediction system 106. For instance, FIG. 44 illustrates a nucleotide sequence that includes a pre-mRNA sequence 4402. As shown, the pre-mRNA sequence 4402 includes exons 4404a-4404d and introns 4406a-4406c between the exons 4404a-4404d.

FIG. 44 further illustrates a reference splicing example 4408 that shows constitutive splicing. As shown, the reference splicing example 4408 results in a sequence of the exons 4404a-4404d with the introns 4406a-4406c spliced out. In other words, in some cases, the reference splicing example 4408 shown in FIG. 44 is the result of splicing under normal conditions in which introns are taken out of the sequence and exons are maintained.

FIG. 44 further illustrates several alternative splicing examples 4410. For instance, FIG. 44 illustrates an exon skipping example 4412 in which the exon 4404b is spliced out. An intron retention example 4414 is also shown in which the intron 4406b between the exons 4404b-4404c is retained. FIG. 44 further shows an example 4416 and example 4418 in which alternative splice sites are used so that the exon 4404b is shortened or elongated, respectively, during the splicing process.

As previously mentioned, alternative splicing, such as that shown in FIG. 44, can cause disorders within a genomic host. For example, alternative splicing can disrupt the normal expression of proteins within the genomic host, resulting in one or more diseases or other disorders. Thus, by identifying variants that are associated with (e.g., at least partially cause) alternative splicing, in some embodiments, the splice-site prediction system 106 facilitates the treatment of these disorders.

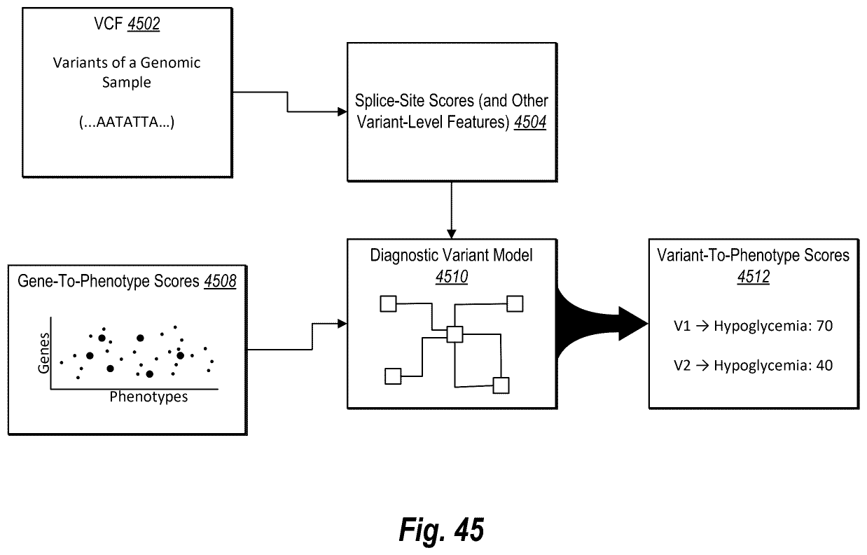

In addition or in the alternative to the gene-to-phenotype-association models described above, the splice-site prediction system 106 can leverage a splice-site score based on conditional probabilities to determine a link between a gene and a phenotype in different ways. In certain embodiments, the splice-site prediction system 106 generates variant-to-phenotype scores to determine whether a genomic sample includes a diagnostic variant. As used herein, the term “diagnostic variant” refers to a variant nucleotide that is diagnosed as corresponding to, or impacting the expression of, a particular phenotype. For example, a diagnostic variant includes a variant that causes or affects the expression of a certain genetic condition or disease within an organism. The splice-site prediction system 106 can utilize a diagnostic variant model to generate variant-to-phenotype scores for variant nucleobases of a genomic sample. In accordance with one or more embodiments, FIG. 45 illustrates an example diagram for generating variant-to-phenotype scores using a diagnostic variant model.

As indicated by FIG. 45, the splice-site prediction system 106 uses gene-to-phenotype scores 4508 for diagnostic purposes. The gene-to-phenotype scores 4508 may be generated by the gene-to-phenotype-association model 4324 depicted in FIG. 43 or by a gene embedding neural network, as described by Aashish Nath Adhikari et al., “Linking Human Genes to Clinical Phenotypes Using Graph Neural Networks,” U.S. Provisional Application No. 63/505,736 (filed Jun. 2, 2023) (hereinafter “Adhikari”), which is hereby incorporated by reference in its entirety. Such a gene embedding neural network can process gene embeddings encoding the relationships between genes in a latent space and generate the gene-to-phenotype scores 4508 linking genes to phenotypes (e.g., selected from a limited set of annotated phenotypes) based on the gene embeddings.

As shown in FIG. 45, the splice-site prediction system 106 accesses, for a set of genes from a genome associated with a genomic sample, the gene-to-phenotype scores 4508 indicating respective probabilities of the set of genes being associated with phenotypes determined for an organism of the genomic sample. The splice-site prediction system 106 also provides, to a diagnostic variant model 4510, splice-site scores 4504 (and/or other variant-level features) for a variant nucleobase at a target position and the gene-to-phenotype scores 4508. By using the diagnostic variant model 4510 to process the splice-site scores 4504 and the gene-to-phenotype scores 4508, the splice-site prediction system 106 determines variant-to-phenotype scores 4512. The variant-to-phenotype scores 4512 indicate a genomic sample comprises a diagnostic variant that is associated with a phenotype of the phenotypes determined for an organism.

The following paragraphs described FIG. 45 in more detail. As illustrated in FIG. 45, the splice-site prediction system 106 accesses a variant call file 4502. In particular, the splice-site prediction system 106 accesses, retrieves, or receives the variant call file 4502 that includes indications of variant nucleobases specific to a genomic sample of an organism (e.g., a human patient). In some cases, the variant call file 4502 also indicates other data, such as genotypes associated with genes of a genotype sample and/or phenotypes observed in or exhibited by an organism of a genomic sample.

As further shown in FIG. 45, in some cases, the splice-site prediction system 106 determines or accesses splice-site scores 4504 (or other variant-level features) for variant nucleotides, where, for example, a splice-site score indicates a probability of one or more of the variant nucleotides from the genomic sample as part of a splice site (e.g., a donor site or an acceptor site) or a non-splice site for pre-messenger RNA. For instance, in some cases, the splice-site prediction system 106 determines the splice-site scores 4504 based on (context-specific) conditional probabilities as discussed above with reference to FIG. 43.

As used herein, the term “variant-level feature” refers to a feature or a metric (or some other data) that measures, quantifies, or compares a variant nucleobase with respect to other variant nucleobases or reference nucleotides. For example, a variant-level feature includes a (i) pathogenicity score (e.g., from a PrimateAI model) indicating a degree to which a variant protein corresponding to a variant nucleotide is benign or pathogenic, (ii) a splice-site score indicating a conditional probability that the variant nucleobase is part of a splice site based on a conditioned position, (iii) an allele frequency corresponding to the variant nucleotides (e.g., from a gnomADg_AF model), (iv) a genotype for genes of a genomic sample, a loss-of-function status indicating that variant proteins reduce or destroy protein function, or (v) a de novo status of the variant nucleotides as private variants.

As further illustrated in FIG. 45, the splice-site prediction system 106 accesses or determines gene-to-phenotype scores 4508. For instance, the splice-site prediction system 106 can access a lookup table or some other form of data storage that indicates gene-to-phenotype scores for genes of a human genome. In some embodiments, by contrast, the splice-site prediction system 106 executes a gene embedding neural network, as described above and by Adhikari, to generate the gene-to-phenotype scores 4508. The splice-site prediction system 106 can use the gene-to-phenotype scores 4508 and the splice-site scores 4504 (or other variant-level features) to generate variant-to-phenotype scores 4512.

To elaborate, the splice-site prediction system 106 can use the splice-site scores 4504 and the gene-to-phenotype scores 4508 as input into the diagnostic variant model 4510. In turn, the diagnostic variant model 4510 processes the splice-site scores 4504 and the gene-to-phenotype scores 4508 to determine and encode relationships between the variant nucleobases of the variant call file 4502 and the phenotypes indicated by the gene-to-phenotype scores 4508. For example, the diagnostic variant model 4510 can generate probabilities (in the form of variant-to-phenotype scores 4512) that the variant nucleobases are associated with or impact the expression of one or more of the phenotypes (e.g., where a higher probability indicates a higher likelihood that the variant nucleobase is associated with or impacts the expression or indicates a stronger impact on the expression). Indeed, based on the encoded relationships between genes and phenotypes (as represented by the gene-to-phenotype scores 4508), the diagnostic variant model 4510 can link variant nucleobases among genes to various (observed) phenotypes as well (e.g., based on the variant level features).

Accordingly, the splice-site prediction system 106 generates the variant-to-phenotype scores 4512 that represent the links or relationships between variant nucleobases of a genomic sample and phenotypes observed in the genomic sample. As shown in FIG. 45, the splice-site prediction system 106 generates the variant-to-phenotype scores 4512 for at least two variants, where the score for variant V1 being associated with hypoglycemia is 70, and the score for variant V2 being associated with hypoglycemia is 40.

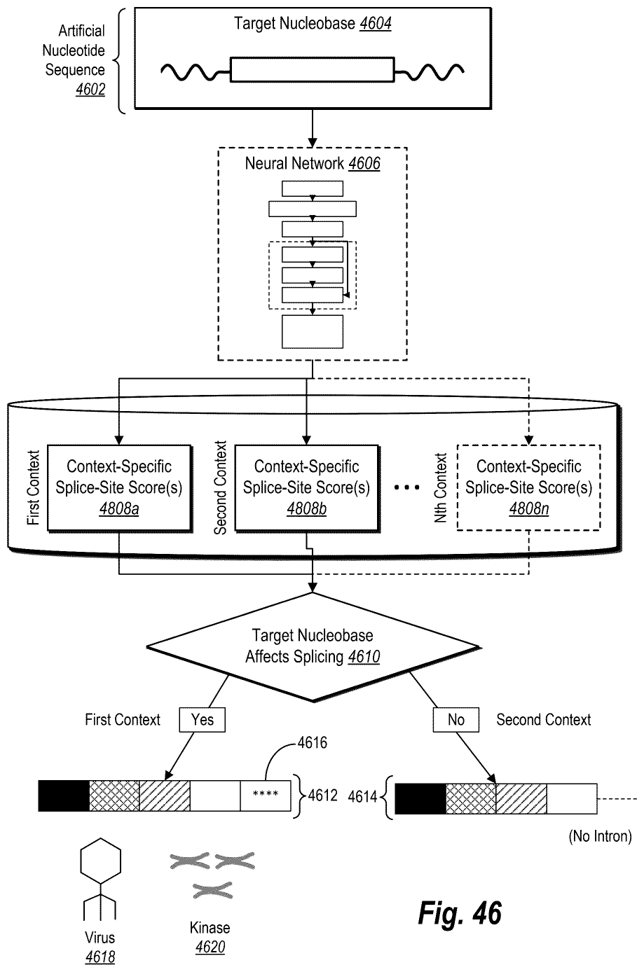

In addition or in the alternative to leveraging splice-site scores derived from conditional probabilities to link genes to phenotype or identify candidate biochemical compounds that target variant nucleobases or adjacent nucleobases, the splice-site prediction system 106 can leverage context-specific splice-site scores to determine whether a target nucleobase in an artificial nucleotide sequence is associated with splicing pre-mRNA. In accordance with one or more embodiments, FIG. 46 illustrates the splice-site prediction system 106 leveraging such context-specific conditional probabilities determined for an artificial nucleotide sequence.

By way of overview of FIG. 46, the splice-site prediction system 106 identifies, for a genomic region, an artificial nucleotide sequence 4602 comprising a target nucleobase 4604 at a target position. The splice-site prediction system 106 further accesses (i) a first context-specific splice-site score 4808a for the target nucleobase 4604 with respect to a first target context based on one or more first context-specific conditional probabilities and (ii) a second context-specific splice-site score 4808b for the target nucleobase 4604 with respect to a second target context differing from the first target context. Based on the first context-specific splice-site score 4808a and the second context-specific splice-site score 4808b—and optionally other context-specific splice-site scores 4808n for the target nucleobase 4604—the splice-site prediction system 106 determines that the target nucleobase 4604 at the target position within the artificial nucleotide sequence 4602 is associated with splicing of pre-mRNA in the first target context and not associated with splicing of pre-mRNA in the second target context.

Continuing the overview, when the target nucleobase 4604 is associated with pre-mRNA splicing in the first context (e.g., tumor or other cancer cells), the splice-site prediction system 106 can identify a synthetic intron, such as a synthetic intron encoding a virus 4618 or a kinase 4620, as part of the artificial nucleotide sequence 4602 for therapeutic purposes in the first context. When the target nucleobase 4604 is not associated with pre-mRNA splicing in the second context (e.g., non-tumor or other non-cancer cells), the splice-site prediction system 106 can ensure that the synthetic intron in the does is not activated or otherwise used for therapeutic purposes in the second context.

The following paragraphs describe FIG. 46 in more detail. FIG. 46 illustrates the artificial nucleotide sequence 4602 having the target nucleobase 4604 (e.g., a variant nucleobase or a reference nucleobase). The target nucleobase 4604 is located at a target position within the artificial nucleotide sequence 4602. In some implementations, the artificial nucleotide sequence 4602 includes an antisense oligonucleotide or a nucleotide sequence including a synthetic intron. In one or more embodiments, the synthetic intron encodes the kinase 4620 or the virus 4618. In some cases, the kinase 4620 or the virus 4618 encoded by the synthetic intron can include a kinase or virus designed to treat a disease.

For example, in some embodiments, the synthetic intron encodes herpes simplex virus-thymidine kinase (HSV-TK) as described by Khrystyna North et al., “Synthetic Introns Enable Splicing Factor Mutation-Dependent Targeting of Cancer Cells,” 40 Nature Biotechnology 1103-1113 (2022), which is hereby incorporated by reference in its entirety. In such embodiments, the splice-site prediction system 106 can identify the artificial nucleotide sequence 4602 as comprising the target nucleobase 4604 to facilitate (i) splicing a corresponding synthetic intron encoding HSV-TK in tumor cells but (ii) not splicing the corresponding synthetic intron in non-tumor cells, thereby selectively eliminating tumor cells.

As further shown in FIG. 46, in some embodiments, the splice-site prediction system 106 utilizes a neural network 4606 to analyze the artificial nucleotide sequence 4602. Based on the analysis, the splice-site prediction system 106 determines context-specific splice-site scores 4808a-4808n for the artificial nucleotide sequence 4602 with respect to various contexts (e.g., various instances of the same context). For instance, the splice-site prediction system 106 uses the neural network 4606 to generate multiple sets of context-specific conditional probabilities where each set of context-specific conditional probability corresponds to a context (e.g., an instance of a context). The splice-site prediction system 106 further determines the context-specific splice-site scores 4808a-4808n based on the sets of context-specific conditional probabilities where each context-specific splice-site score (or scores) corresponds to a context (e.g., an instance of a context).

As previously described, the splice-site prediction system 106 can operate in various contexts. Accordingly, the first context-specific splice-site score and the first target context and the second context-specific splice-site score and the second target context can include, but are not limited to, a first tissue-specific splice-site score for the target nucleobase with respect to a first tissue based on one or more first tissue-specific conditional probabilities and a second tissue-specific splice-site score for the target nucleobase with respect to a second tissue based on one or more second tissue-specific conditional probabilities; a first disease-specific splice-site score for the target nucleobase with respect to a first disease based on one or more first disease-specific conditional probabilities and a second disease-specific splice-site score for the target nucleobase with respect to a second disease based on one or more second disease-specific conditional probabilities; a first cell-type-specific splice-site score for the target nucleobase with respect to a first cell type based on one or more first cell-type-specific conditional probabilities and a second cell-type-specific splice-site score for the target nucleobase with respect to a second cell type based on one or more second cell-type-specific conditional probabilities; a first cell-line-specific splice-site score for the target nucleobase with respect to a first cell-line type based on one or more first cell-line-specific conditional probabilities and a second cell-line-specific splice-site score for the target nucleobase with respect to a second cell-line type based on one or more second cell-line-specific conditional probabilities; or a first assay-specific splice-site score for the target nucleobase with respect to a first assay type based on one or more first assay-specific conditional probabilities and a second assay-specific splice-site score for the target nucleobase with respect to a second assay type based on one or more second assay-specific conditional probabilities. For instance, in some cases, the first context-specific splice-site score and the first target context and the second context-specific splice-site score and the second target context comprise a first cell-type-specific splice-site score for the target nucleobase with respect to a first cell type comprising a target variant in a gene and a second cell-type-specific splice-site score for the target nucleobase with respect to a second cell type not comprising the target variant in the gene.

Based on the context-specific splice-site scores 4808a-4808n, the splice-site prediction system 106 can determine whether the target nucleobase 4604 affects splicing (e.g., is associated with alternative splicing) for the artificial nucleotide sequence 4602 (shown by box 4610). FIG. 46 shows that the splice-site prediction system 106 determines that the target nucleobase 4604 does affect splicing for the first context (e.g., the first instance of the context) but not for the second context (e.g., the second instance of the context). As indicated above, the first context may be tumor cells of an organism and the second context may be non-tumor cells of the organism. Indeed, comparing the resulting sequence 4612 for the first context with the resulting sequence 4614 for the second context reveals the difference in splicing results. In some embodiments, the splice-site prediction system 106 determines that the target nucleobase 4604 affects the splicing relative to a reference splicing of the artificial nucleotide sequence 4602.

In particular, FIG. 46 shows that the resulting sequence 4612 for the first context includes a synthetic intron 4616. As further shown, the resulting sequence 4612 includes (e.g., as encoded by the synthetic intron 4616), the virus 4618 or the kinase 4620. Thus, in some cases, the splice-site prediction system 106 uses the process described herein with reference to FIG. 46 to determine a virus or kinase that will remain after splicing with respect to a given context (e.g., a given instance of a context). In some cases, the virus 4618 or the kinase 4620 include those that can treat a disease or other disorder related to the splicing of the corresponding context. Thus, the splice-site prediction system 106 can utilize the context-specific conditional probabilities generated by the neural network 4606 (and the context-specific splice-site scores derived therefrom) to identify approaches to treating undesirable splicing.

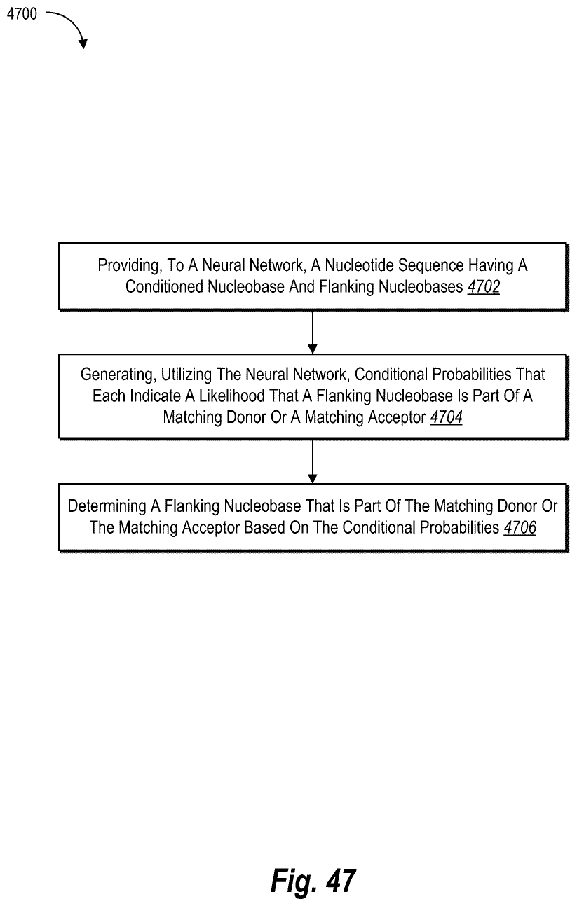

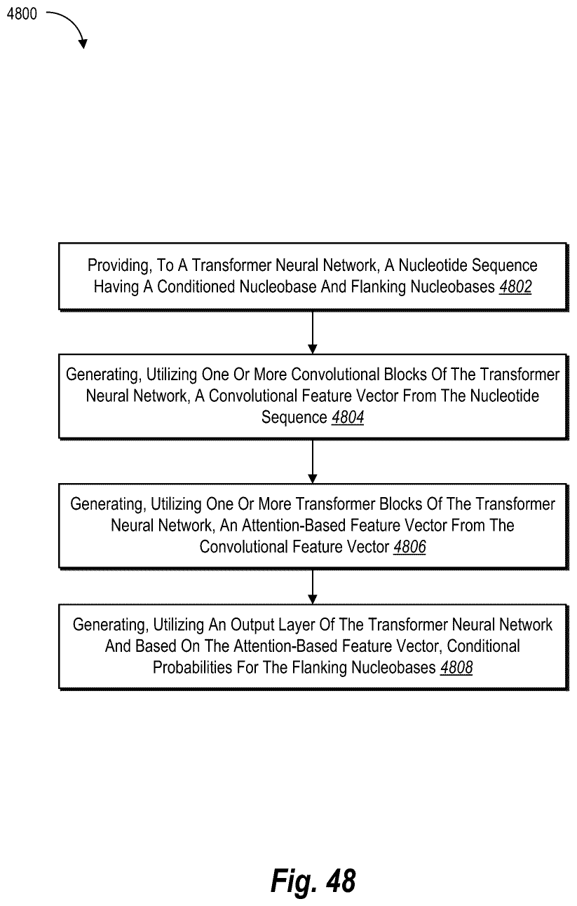

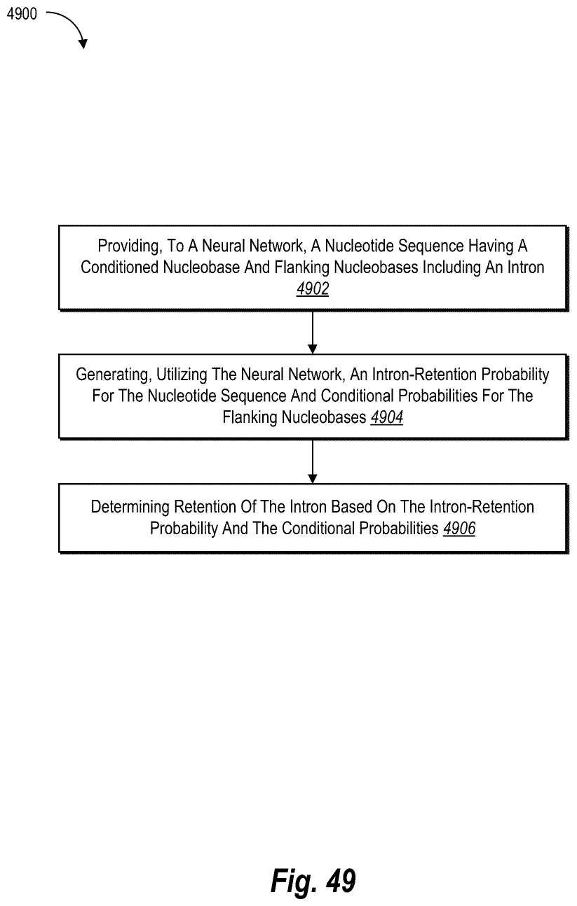

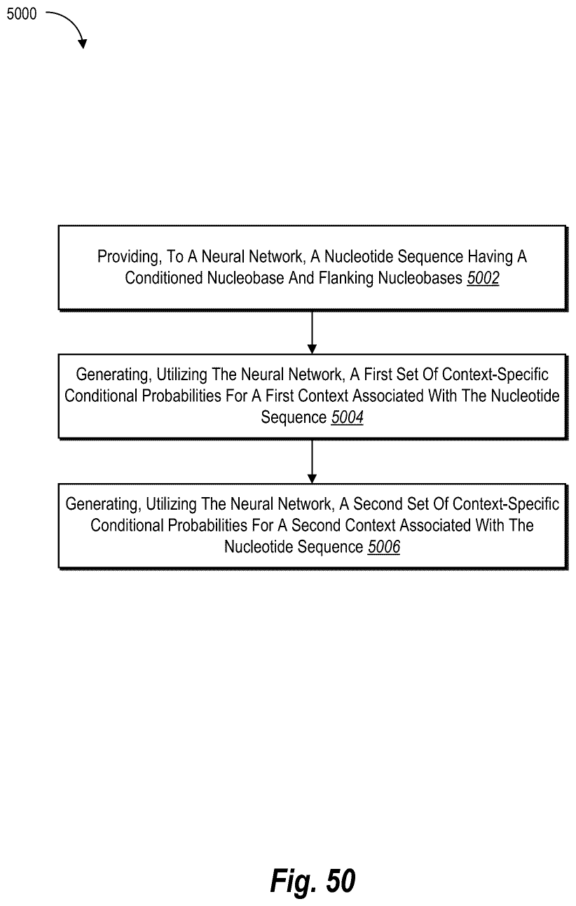

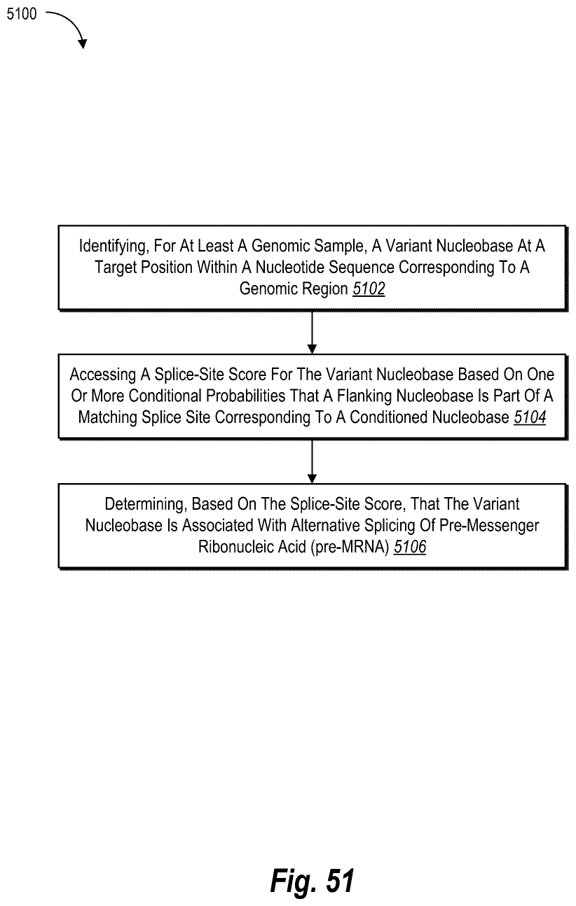

FIGS. 1-46, the corresponding text, and the examples provide a number of different methods, systems, devices, and non-transitory computer-readable media of the splice-site prediction system 106. In addition to the foregoing, one or more implementations can also be described in terms of flowcharts comprising acts for accomplishing a particular result, as shown in FIGS. 47-51. FIG. 47 illustrates a flowchart of a series of acts 4700 of generating conditional probabilities for nucleotide sequences indicating likelihoods of matching splice sites in accordance with one or more embodiments. FIG. 48 illustrates a flowchart of a series of acts 4800 of utilizing a transformer neural network to generate conditional probabilities for nucleotide sequences in accordance with one or more embodiments. FIG. 49 illustrates a flowchart of a series of acts 4900 of generating intron-retention probabilities and conditional probabilities for nucleotide sequences in accordance with one or more embodiments. FIG. 50 illustrates a flowchart of a series of acts 5000 of generating context-specific conditional probabilities for nucleotide sequences in accordance with one or more embodiments. FIG. 51 illustrates a flowchart of a series of acts 5100 of leveraging conditional probabilities for a nucleotide sequence to determine whether a variant nucleobase contained therein is associated is alternative splicing of pre-mRNA in accordance with one or more embodiments.

While FIGS. 47-51 illustrate acts according to one embodiment, alternative embodiments may omit, add to, reorder, and/or modify any of the acts shown in FIGS. 47-51. The acts of FIGS. 47-51 can be performed as part of a method. Alternatively, a non-transitory computer readable storage medium can comprise instructions that, when executed by one or more processors, cause a computing device or a system to perform the acts depicted in FIGS. 47-51. In still further embodiments, a system comprises at least one processor and a non-transitory computer readable medium comprising instructions that, when executed by the at least one processor, cause the system to perform the acts of FIGS. 47-51.

As shown in FIG. 47, the series of acts 4700 includes an act 4702 providing, to a neural network, a nucleotide sequence having a conditioned nucleobase and flanking nucleobases; an act 4704 of generating, utilizing the neural network, conditional probabilities that each indicate a likelihood that a flanking nucleobase is part of a matching donor or a matching acceptor; and an act 4706 of determining a flanking nucleobase that is part of the matching donor or the matching acceptor based on the conditional probabilities. For example, the series of acts 4700 can include acts to perform any of the operations described in the following clauses:

CLAUSE 1. A computer-implemented method comprising:
providing, to a neural network, a nucleotide sequence comprising a conditioned nucleobase at a conditioned position and flanking nucleobases within a threshold number of nucleobases of the conditioned nucleobase;
generating, utilizing the neural network and based on the conditioned nucleobase, conditional probabilities for the flanking nucleobases, each conditional probability indicating a likelihood that a flanking nucleobase is part of a matching donor or a matching acceptor corresponding to the conditioned nucleobase; and
determining, based on the conditional probabilities for the flanking nucleobases, at least one flanking nucleobase from the flanking nucleobases that is part of the matching donor or the matching acceptor for the conditioned nucleobase.

CLAUSE 2. The computer-implemented method of clause 1, wherein providing the nucleotide sequence comprising the conditioned nucleobase comprises providing the nucleotide sequence comprising a splice-junction nucleobase at the conditioned position representing a splice site.

CLAUSE 3. The computer-implemented method of clause 1, wherein:
providing the nucleotide sequence comprises providing the nucleotide sequence comprising the conditioned nucleobase representing a conditioned acceptor or a conditioned donor; and
generating the conditional probabilities for the flanking nucleobases comprises generating a probability that each flanking nucleobase is part of a matching donor or a matching acceptor respectively corresponding to the conditioned acceptor or the conditioned donor of a splice site.

CLAUSE 4. The computer-implemented method of clause 1, wherein generating the conditional probabilities for the flanking nucleobases comprises generating, for each flanking nucleobase corresponding to a conditioned donor represented by the conditioned nucleobase:
a first conditional probability that the flanking nucleobase is part of a downstream acceptor that corresponds to the conditioned donor; or
a second conditional probability that the flanking nucleobase is part of an upstream acceptor that corresponds to the conditioned donor.

CLAUSE 5. The computer-implemented method of clause 1, wherein generating the conditional probabilities for the flanking nucleobases comprises generating, for each flanking nucleobase corresponding to a conditioned acceptor represented by the conditioned nucleobase:
a first conditional probability that the flanking nucleobase is part of a downstream donor that corresponds to the conditioned acceptor; or
a second conditional probability that the flanking nucleobase is part of an upstream donor that corresponds to the conditioned acceptor.

CLAUSE 6. The computer-implemented method of clause 1, further comprising:
providing, to an input channel of the neural network, a conditioned position identifier indicating that the conditioned nucleobase is at the conditioned position; and
generating, utilizing the neural network, the conditional probabilities for the flanking nucleobases further based on the conditioned position identifier.

CLAUSE 7. The computer-implemented method of clause 1, further comprising:
providing, to an input channel of the neural network, a distance value for each flanking nucleobase that corresponds to a number of nucleobases between the flanking nucleobase and the conditioned nucleobase within the nucleotide sequence; and
generating, utilizing the neural network, the conditional probabilities for the flanking nucleobases further based on the distance value for each flanking nucleobase.

CLAUSE 8. The computer-implemented method of clause 7, wherein determining the distance value for each flanking nucleobase comprises:
determining an integer value for each flanking nucleobase that indicates a distance of the flanking nucleobase from the conditioned nucleobase based on a number of positions from the flanking nucleobase to the conditioned nucleobase within the nucleotide sequence; and
generating the distance value for each flanking nucleobase from the integer value using a logarithmic transform.

CLAUSE 9. The computer-implemented method of clause 1, wherein providing the nucleotide sequence comprises providing, for a genomic sample, a pre-messenger ribonucleic acid (pre-mRNA) sequence comprising a variant nucleobase.

CLAUSE 10. The computer-implemented method of clause 1, further comprising:
providing, to the neural network, an additional nucleotide sequence comprising an additional conditioned nucleobase at an additional conditioned position and additional flanking nucleobases;
generating, utilizing the neural network and based on the additional conditioned nucleobase, additional conditional probabilities for the additional flanking nucleobases; and
determining, based on the additional conditional probabilities, that the additional nucleotide sequence excludes a matching donor or a matching acceptor for the additional conditioned nucleobase.

CLAUSE 11. The computer-implemented method of clause 1, wherein:
providing the nucleotide sequence comprises providing a ribonucleic acid (RNA) sequence or a deoxyribonucleic acid (DNA) sequence based on long nucleotide reads of a threshold number of nucleobases; and
generating the conditional probabilities comprises generating:
   a first set of conditional probabilities that flanking nucleobases are part of an upstream acceptor that corresponds to a conditioned donor; or
   a second set of conditional probabilities that flanking nucleobases are part of a downstream donor that corresponds to a conditioned acceptor.

CLAUSE 12. The computer-implemented method of clause 1,
wherein providing the nucleotide sequence comprises providing a reference-sequence vector representing a reference nucleotide sequence comprising the flanking nucleobases; the computer-implemented method further comprising:
comparing the conditional probabilities for the flanking nucleobases to values within a ground-truth-label vector comprising a non-zero value for at least one flanking nucleobase that is part of the matching donor or the matching acceptor and at least two zero values for other flanking nucleobases that are not part of the matching donor or the matching acceptor; and
adjusting parameters for the neural network based on comparing the conditional probabilities for the flanking nucleobases to the values within the ground-truth-label vector.

As shown in FIG. 48, the series of acts 4800 includes an act 4802 of providing, to a transformer neural network, a nucleotide sequence having a conditioned nucleobase and flanking nucleobases; an act 4804 of generating, utilizing one or more convolutional blocks of the transformer neural network, a convolutional feature vector from the nucleotide sequence; an act 4806 of generating, utilizing one or more transformer blocks of the transformer neural network, an attention-based feature vector from the convolutional feature vector; and act 4808 of generating, utilizing an output layer of the transformer neural network and based on the attention-based feature vector, conditional probabilities for the flanking nucleobases. For example, the series of acts 4800 can include acts to perform any of the operations described in the following clauses:

CLAUSE 1. A computer-implemented method comprising:
providing, to a transformer neural network, a nucleotide sequence comprising a conditioned nucleobase at a conditioned position and flanking nucleobases within a threshold number of nucleobases of the conditioned nucleobase;
generating, utilizing one or more convolutional blocks of the transformer neural network, a convolutional feature vector representing the nucleotide sequence;
generating, utilizing one or more transformer blocks of the transformer neural network and from the convolutional feature vector, an attention-based feature vector that captures sequence patterns across the nucleotide sequence; and
generating, utilizing an output layer of the transformer neural network and based on the attention-based feature vector, conditional probabilities for the flanking nucleobases, each conditional probability indicating a likelihood that a flanking nucleobase is part of a matching donor or a matching acceptor corresponding to the conditioned nucleobase at the conditioned position.

CLAUSE 2. The computer-implemented method of clause 1, wherein generating the attention-based feature vector utilizing the one or more transformer blocks of the transformer neural network comprises generating the attention-based feature vector utilizing one or more fast-linear-attention-with-a-single-head (FLASH) blocks of the transformer neural network.

CLAUSE 3. The computer-implemented method of clause 1, wherein generating the attention-based feature vector comprises generating the attention-based feature vector utilizing at least one transformer block that includes an attention unit with a normalization layer positioned to accept at least one of a linear-attention output or quadratic-attention output of an internal layer of the attention unit.

CLAUSE 4. The computer-implemented method of clause 1, wherein generating the attention-based feature vector comprises:
generating, from the convolutional feature vector, at least one feature tensor that is internal to an attention unit;
reshaping the at least one feature tensor according to a reshaping factor and along a sequence dimension corresponding to the nucleotide sequence; and
generating the attention-based feature vector from the at least one reshaped feature tensor utilizing the attention unit.

CLAUSE 5. The computer-implemented method of clause 4, wherein reshaping the at least one feature tensor according to the reshaping factor comprises applying the reshaping factor to a query feature tensor, a key feature tensor, and a value feature tensor along the sequence dimension.

CLAUSE 6. The computer-implemented method of clause 1, wherein generating the convolutional feature vector utilizing the one or more convolutional blocks of the transformer neural network comprises generating the convolutional feature vector utilizing three or four convolutional blocks.

CLAUSE 7. The computer-implemented method of clause 1, wherein generating the attention-based feature vector utilizing the one or more transformer blocks of the transformer neural network comprises:
generating a first attention-based feature vector from the convolutional feature vector utilizing a first transformer block of the transformer neural network, the first transformer block comprising a first normalization layer, a first attention unit, and a first concatenation layer;
providing the first attention-based feature vector to a second transformer block of the transformer neural network, the second transformer block comprising a second normalization layer, a second attention unit, and a second concatenation layer; and
generating a second attention-based feature vector from the first attention-based feature vector utilizing the second transformer block of the transformer neural network.

CLAUSE 8. The computer-implemented method of clause 1, wherein providing the nucleotide sequence to the transformer neural network comprises providing the nucleotide sequence having a sequence length of between one thousand nucleobases to thirty-two thousand nucleobases to the transformer neural network.

CLAUSE 9. The computer-implemented method of clause 1, further comprising determining parameters for the transformer neural network using a stepwise learning rate decay.

CLAUSE 10. The computer-implemented method of clause 9, wherein determining the parameters for the transformer neural network using the stepwise learning rate decay comprises:
updating the parameters of the transformer neural network utilizing an initial learning rate that incrementally increases up to between 1e-4 and 5e-3;
updating the parameters of the transformer neural network utilizing a first fixed learning rate of between 1e-4 and 5e-3 during a first set of update steps that ends after a threshold number of update steps between one thousand update steps and two thousand update steps; and
updating the parameters of the transformer neural network utilizing a second fixed learning rate that is lower than 1e-4 during a second set of update steps that begins after the threshold number of update steps.

CLAUSE 11. The computer-implemented method of clause 1, further comprising determining parameters for the transformer neural network based on comparing, via a cross-entropy loss function, predicted conditional probabilities generated from training nucleotide sequences to corresponding ground truth probabilities of the flanking nucleobases being part of the matching donor or the matching acceptor.

As shown in FIG. 49, the series of acts 4900 includes an act 4902 of providing, to a neural network, a nucleotide sequence having a conditioned nucleobase and flanking nucleobases including an intron; an act 4904 of generating, utilizing the neural network, an intron-retention probability for the nucleotide sequence and conditional probabilities for the flanking nucleobases; and an act 4906 of determining retention of the intron based on the intron-retention probability and the conditional probabilities. For example, the series of acts 4900 can include acts to perform any of the operations described in the following clauses:

CLAUSE 1. A computer-implemented method comprising:
providing, to a neural network, a nucleotide sequence comprising a conditioned nucleobase at a conditioned position and a series of flanking nucleobases including an intron of the nucleotide sequence;
generating, utilizing the neural network, an intron-retention probability indicating a likelihood that the intron is retained in messenger ribonucleic acid (mRNA) corresponding to the nucleotide sequence and a set of conditional probabilities that each indicate a likelihood that a flanking nucleobase is part of one or more matching acceptors or one or more matching donors for the conditioned nucleobase; and
determining, based on the intron-retention probability and the set of conditional probabilities, that the mRNA retains the intron.

CLAUSE 2. The computer-implemented method of clause 1, wherein providing the nucleotide sequence comprising the conditioned nucleobase and the series of flanking nucleobases comprises:
providing the nucleotide sequence comprising a conditioned donor and a series of downstream nucleobases that includes the intron followed by a matching acceptor; or
providing the nucleotide sequence comprising a conditioned acceptor and a series of upstream nucleobases that includes the intron preceded by a matching donor.

CLAUSE 3. The computer-implemented method of clause 1, wherein generating the intron-retention probability and the set of conditional probabilities comprises generating an output vector comprising the intron-retention probability and the set of conditional probabilities, one or more positions of the output vector corresponding to positions of flanking nucleobases from the series of flanking nucleobases within the nucleotide sequence.

CLAUSE 4. The computer-implemented method of clause 3,
wherein generating the intron-retention probability comprises generating, from the nucleotide sequence, the intron-retention probability; the computer-implemented method further comprising:
generating, from the nucleotide sequence, a vector comprising the set of conditional probabilities, each position of the vector corresponding to a position of a flanking nucleobase from the series of flanking nucleobases; and
generating the output vector by combining the intron-retention probability and the vector comprising the set of conditional probabilities.

CLAUSE 5. The computer-implemented method of clause 1, wherein generating the intron-retention probability comprises generating the intron-retention probability for a position of the nucleotide sequence that is within a threshold number of positions from the conditioned position.

CLAUSE 6. The computer-implemented method of clause 1, generating the intron-retention probability and the set of conditional probabilities comprises generating a plurality of probabilities that sum to a value of one.

CLAUSE 7. The computer-implemented method of clause 1, further comprising generating, for display within a graphical user interface, an intron-retention indicator that a gene of a genomic sample associated with the nucleotide sequence comprises a variant associated with the mRNA retaining the intron after splicing.

CLAUSE 8. The computer-implemented method of clause 1, wherein providing the nucleotide sequence to the neural network comprises providing the nucleotide sequence comprising a pre-messenger ribonucleic acid (pre-mRNA) sequence comprising a variant nucleobase.

CLAUSE 9. The computer-implemented method of clause 1, further comprising:
providing, to the neural network, an additional nucleotide sequence comprising an additional conditioned nucleobase at an additional conditioned position and an additional series of flanking nucleobases including an additional intron of the additional nucleotide sequence; and
generating, utilizing the neural network, an additional intron-retention probability having a zero value or a value below a threshold value that indicates that the intron is spliced out of mRNA corresponding to the additional nucleotide sequence during splicing.

CLAUSE 10. The computer-implemented method of clause 1, wherein generating, utilizing the neural network, the intron-retention probability and the set of conditional probabilities comprises generating the intron-retention probability and the set of conditional probabilities utilizing a transformer neural network.

CLAUSE 11. A computer-implemented method comprising:
providing, to a neural network, a training nucleotide sequence comprising a conditioned nucleobase at a conditioned position and a series of flanking nucleobases including an intron of the training nucleotide sequence;
generating, utilizing the neural network, a predicted intron-retention probability indicating a likelihood that at least a portion of the intron is retained in messenger ribonucleic acid (mRNA) corresponding to the training nucleotide sequence and a predicted set of conditional probabilities that each indicate a likelihood that a flanking nucleobase is part of one or more matching acceptors or one or more matching donors for the conditioned nucleobase;
comparing, to determine a loss, the predicted intron-retention probability to a ground truth indicator of intron retention and the predicted set of conditional probabilities to ground truth indicators for the one or more matching acceptors or the one or more matching donors generated for the training nucleotide sequence using an exon-intron boundary count associated with the training nucleotide sequence; and
adjusting parameters of the neural network based on the determined loss.

CLAUSE 12. The computer-implemented method of clause 11, wherein comparing the predicted intron-retention probability to the ground truth indicator of intron retention using the exon-intron boundary count associated with the training nucleotide sequence comprises comparing the predicted intron-retention probability to a ground truth intron-retention probability derived using the exon-intron boundary count.

CLAUSE 13. The computer-implemented method of clause 11, further comprising determining the exon-intron boundary count associated with the training nucleotide sequence by determining, for a genomic sample associated with the training nucleotide sequence, a number of nucleotide reads that cover a boundary of the intron.

CLAUSE 14. The computer-implemented method of clause 11, further comprising determining the exon-intron boundary count associated with the training nucleotide sequence by:
identifying a set of nucleotide reads of a genomic sample associated with the training nucleotide sequence; and
determining, from among the set of nucleotide reads, a first number of nucleotide reads that cover a threshold number of nucleobases upstream and downstream from an exon-intron boundary between an exon and an adjacent intron.

CLAUSE 15. The computer-implemented method of clause 14, wherein determining the exon-intron boundary count associated with the training nucleotide sequence comprises:
determining, from among the set of nucleotide reads, a second number of nucleotide reads that cover a threshold number of nucleobases within an exon of the exon-intron boundary; and
determining the exon-intron boundary count for the training nucleotide sequence using a ratio of the first number of nucleotide reads and the second number of nucleotide reads.

CLAUSE 16. The computer-implemented method of clause 15, wherein determining the exon-intron boundary count for the training nucleotide sequence using the ratio of the first number of nucleotide reads and the second number of nucleotide reads comprises determining a normalized exon-intron boundary count using the ratio and a third number of nucleotide reads from the set of nucleotide reads that represents a junction count associated with the training nucleotide sequence.

CLAUSE 17. The computer-implemented method of clause 11, further comprising:
determining exon-intron boundary counts for a training dataset used in learning parameters for the neural network, the training dataset comprising the training nucleotide sequence; and
up-sampling the exon-intron boundary counts to increase representation of the exon-intron boundary counts within the training dataset.

CLAUSE 18. The computer-implemented method of clause 11, further comprising determining exon-intron boundary counts for a training dataset used in learning parameters for the neural network by:
determining a first set of counts based on nucleotide reads for the training dataset that cover boundaries between exons and adjacent introns represented in the training dataset; and
determining a second set of counts based on exon-intron boundary proxies that include positions within one or more exons represented in the training dataset.

CLAUSE 19. The computer-implemented method of clause 11, further comprising determining exon-intron boundary counts for a training dataset used in learning parameters for the neural network by:
determining raw counts using nucleotide reads of genomic samples associated with training nucleotide sequences included in the training dataset;
generating transformed counts by applying an arcsinh transformation to the raw counts; and
determining the exon-intron boundary counts for the training dataset using the transformed counts.

CLAUSE 20. The computer-implemented method of clause 11, further comprising determine the loss using at least one of:
a focal loss function that modifies a weight for nucleobase positions based on an associated probability; or
a weighted loss function that increases a weight for exon-intron boundary counts.

As shown in FIG. 50, the series of acts 5000 includes an act 5002 of providing, to a neural network, a nucleotide sequence having a conditioned nucleobase and flanking nucleobases; an act 5004 of generating, utilizing the neural network, a first set of context-specific conditional probabilities for a first context associated with the nucleotide sequence; and an act 5006 of generating, utilizing the neural network, a second set of context-specific conditional probabilities for a second context associated with the nucleotide sequence. For example, the series of acts 5000 can include acts to perform any of the operations described in the following clauses:

CLAUSE 1. A computer-implemented method comprising:
providing, to a neural network, a nucleotide sequence comprising a conditioned nucleobase at a conditioned position and a series of flanking nucleobases;
generating, from the nucleotide sequence utilizing the neural network, a first set of context-specific conditional probabilities for a first context associated with the nucleotide sequence, each conditional probability from the first set of context-specific conditional probabilities indicating a likelihood that a corresponding flanking nucleobase is part of one or more matching acceptors or one or more matching donors for the conditioned nucleobase with respect to the first context; and
generating, from the nucleotide sequence utilizing the neural network, a second set of context-specific conditional probabilities for a second context associated with the nucleotide sequence, each conditional probability from the second set of context-specific conditional probabilities indicating an additional likelihood that a corresponding flanking nucleobase is part of the one or more matching acceptors or the one or more matching donors for the conditioned nucleobase with respect to the second context.

CLAUSE 2. The computer-implemented method of clause 1, further comprising generating, utilizing the neural network, one or more genomic track signals that indicate one or more properties of the nucleotide sequence.

CLAUSE 3. The computer-implemented method of clause 2, wherein:
generating the first set of context-specific conditional probabilities and the second set of context-specific conditional probabilities utilizing the neural network comprises generating the first set of context-specific conditional probabilities and the second set of context-specific conditional probabilities utilizing a softmax layer of the neural network; and
generating the one or more genomic track signals utilizing the neural network comprises generating the one or more genomic track signals utilizing a feedforward layer of the neural network.

CLAUSE 4. The computer-implemented method of clause 2, wherein generating the one or more genomic track signals comprises generating ribonucleic acid (RNA) binding protein signals that indicate probabilities that positions within the nucleotide sequence correspond to a binding site for an RNA binding protein.

CLAUSE 5. The computer-implemented method of clause 2, wherein generating the one or more genomic track signals comprises generating histone mark signals that indicate probabilities that positions within the nucleotide sequence correspond to a histone mark.

CLAUSE 6. The computer-implemented method of clause 1, wherein the first context comprises a first tissue and the second context comprises a second tissue, the first tissue and the second tissue comprising two of brain tissue, liver tissue, adipose tissue, skin tissue, muscle tissue, heart tissue, pancreas tissue, kidney tissue, artery tissue, or a whole blood sample.

CLAUSE 7. The computer-implemented method of clause 1, further comprising generating, from the nucleotide sequence utilizing the neural network, one or more additional sets of context-specific conditional probabilities for one or more additional contexts associated with the nucleotide sequence.

CLAUSE 8. The computer-implemented method of clause 1, further comprising wherein generating the first set of context-specific conditional probabilities for the first context and the second set of context-specific conditional probabilities for the second context comprises generating:
a first set of tissue-specific conditional probabilities for a first tissue and a second set of tissue-specific conditional probabilities for a second tissue;
a first set of disease-specific conditional probabilities for a first disease and a second set of disease-specific conditional probabilities for a second disease;
a first set of cell-type-specific conditional probabilities for a first cell type and a second set of cell-type-specific conditional probabilities for a second cell type;
a first set of cell-line-specific conditional probabilities for a first cell line and a second set of cell-line-specific conditional probabilities for a second cell line; or
a first set of assay-specific conditional probabilities for a first assay type and a second set of assay-specific conditional probabilities for a second assay type.

CLAUSE 9. The computer-implemented method of clause 1, further comprising determining one or more context-specific spliced isoforms of a messenger ribonucleic acid (mRNA) sequence corresponding to the nucleotide sequence using the first set of context-specific conditional probabilities and the second set of context-specific conditional probabilities.

CLAUSE 10. The computer-implemented method of clause 1, further comprising:
generating a first set of logits from the first set of context-specific conditional probabilities and a second set of logits from the second set of context-specific conditional probabilities;
determining logit differences by comparing the first set of logits and the second set of logits; and
determining relative usage rates for the one or more matching acceptors or the one or more matching donors with respect to the first context and the second context based on the logit differences.

CLAUSE 11. The computer-implemented method of clause 10, further comprising generating, utilizing the logit differences, a performance matrix indicating mean pair-wise context-specific performance of the neural network.

CLAUSE 12. The computer-implemented method of clause 1, further comprising:
determining, for the first context and based on the first set of context-specific conditional probabilities, a first flanking nucleobase from the series of flanking nucleobases having a first highest likelihood of being a matching acceptor or a matching donor for the conditioned nucleobase; and
determining, for the second context and based on the second set of context-specific conditional probabilities, a second flanking nucleobase from the series of flanking nucleobases having a second highest likelihood of being the matching acceptor or the matching donor for the conditioned nucleobase.

CLAUSE 13. A computer-implemented method comprising:
providing, to a neural network, a training nucleotide sequence comprising a conditioned nucleobase at a conditioned position and a series of flanking nucleobases;
generating, from the training nucleotide sequence utilizing the neural network, a first set of predicted context-specific conditional probabilities for a first context associated with the training nucleotide sequence, each conditional probability from the first set of predicted context-specific conditional probabilities indicating a likelihood that a corresponding flanking nucleobase is part of one or more matching acceptors or one or more matching donors for the conditioned nucleobase with respect to the first context;
generating, from the training nucleotide sequence utilizing the neural network, a second set of predicted context-specific conditional probabilities for a second context associated with the training nucleotide sequence, each conditional probability from the second set of predicted context-specific conditional probabilities indicating a likelihood that a corresponding flanking nucleobase is part of one or more matching acceptors or one or more matching donors for the conditioned nucleobase with respect to the second context;
comparing, to determine a loss, the first set of predicted context-specific conditional probabilities and the second set of predicted context-specific conditional probabilities to corresponding context-specific ground truth probabilities for the training nucleotide sequence; and
adjusting parameters of the neural network based on the determined loss.

CLAUSE 14. The computer-implemented method of clause 13, wherein comparing, to determine the loss, the first set of predicted context-specific conditional probabilities and the second set of predicted context-specific conditional probabilities to the corresponding context-specific ground truth probabilities comprises:
generating a first set of predicted logits from the first set of predicted context-specific conditional probabilities and a second set of predicted logits from the second set of predicted context-specific conditional probabilities; and
determining the loss by, via a loss function, comparing the predicted logits to true logits derived from the corresponding context-specific ground truth probabilities.

CLAUSE 15. The computer-implemented method of clause 13, wherein comparing the first set of predicted context-specific conditional probabilities and the second set of predicted context-specific conditional probabilities to the corresponding context-specific ground truth probabilities comprises using a cross-context loss function that adjusts parameters to improve a prediction accuracy across a context dimension.

CLAUSE 16. The computer-implemented method of clause 13, wherein comparing the first set of predicted context-specific conditional probabilities and the second set of predicted context-specific conditional probabilities to the corresponding context-specific ground truth probabilities comprises using a soft-weighted cross-entropy loss function that increases a weight for context-specific training nucleotide sequences.

CLAUSE 17. The computer-implemented method of clause 13, further comprising:
generating, from the training nucleotide sequence utilizing the neural network, one or more predicted genomic track signals that indicate one or more properties of the training nucleotide sequence;
comparing, to determine an additional loss, the one or more predicted genomic track signals to one or more corresponding ground truth genomic track signals; and
adjusting the parameters of the neural network based on the determined loss and the determined additional loss.

CLAUSE 18. The computer-implemented method of clause 17, wherein comparing the one or more predicted genomic track signals to one or more corresponding ground truth genomic track signals comprises comparing the one or more predicted genomic track signals to one or more corresponding ground truth genomic track signals via a mean-squared-error loss function.

CLAUSE 19. The computer-implemented method of clause 18, further comprising:
generating one or more transformed ground truth genomic track signals by applying a logarithmic transformation to the one or more corresponding ground truth genomic track signals; and
comparing the one or more predicted genomic track signals to the one or more corresponding ground truth genomic track signals by comparing the one or more predicted genomic track signals to the one or more transformed ground truth genomic track signals.

CLAUSE 20. The computer-implemented method of clause 17, further comprising wherein generating the one or more predicted genomic track signals comprises:
generating ribonucleic acid (RNA) binding protein signals that indicate probabilities that positions within the training nucleotide sequence correspond to a binding site for an RNA binding protein; and
generating histone mark signals that indicate probabilities that positions within the training nucleotide sequence correspond to a histone mark.

CLAUSE 21. The computer-implemented method of clause 17, wherein generating the first set of context-specific conditional probabilities for the first context and the second set of context-specific conditional probabilities for the second context comprises generating:
a first set of tissue-specific conditional probabilities for a first tissue and a second set of tissue-specific conditional probabilities for a second tissue;
a first set of disease-specific conditional probabilities for a first disease and a second set of disease-specific conditional probabilities for a second disease;
a first set of cell-type-specific conditional probabilities for a first cell type and a second set of cell-type-specific conditional probabilities for a second cell type;
a first set of cell-line-specific conditional probabilities for a first cell line and a second set of cell-line-specific conditional probabilities for a second cell line; or
a first set of assay-specific conditional probabilities for a first assay type and a second set of assay-specific conditional probabilities for a second assay type.

As shown in FIG. 51, the series of acts 5100 includes an act 5102 of identifying, for at least a genomic sample, a variant nucleobase at a target position within a nucleotide sequence corresponding to a genomic region; an act 5104 of accessing a splice-site score for the variant nucleobase based on one or more conditional probabilities that a flanking nucleobase is part of a matching splice site corresponding to a conditioned nucleobase; and an act 5106 of determining, based on the splice-site score, that the variant nucleobase is associated with alternative splicing of pre-messenger ribonucleic acid (pre-mRNA).

CLAUSE 1. A computer-implemented method comprising:
identifying, for at least a genomic sample, a variant nucleobase at a target position within a nucleotide sequence corresponding to a genomic region;
accessing a splice-site score for the variant nucleobase based on one or more conditional probabilities that a flanking nucleobase from the nucleotide sequence is part of a matching donor or a matching acceptor corresponding to a conditioned nucleobase at a conditioned position within the nucleotide sequence; and
determining, based on the splice-site score, that the variant nucleobase at the target position within the nucleotide sequence is associated with alternative splicing of pre-messenger ribonucleic acid (pre-mRNA) relative to a reference splicing of pre-mRNA.

CLAUSE 2. The computer-implemented method of clause 1, wherein accessing the splice-site score for the variant nucleobase based on the one or more conditional probabilities comprises accessing the one or more conditional probabilities from among conditional probabilities determined for the nucleotide sequence comprising the conditioned nucleobase at the conditioned position and flanking nucleobases within a threshold number of nucleobases of the conditioned nucleobase, each conditional probability from the conditional probabilities indicating a likelihood that a corresponding flanking nucleobase is part of a matching donor or a matching acceptor corresponding to the conditioned nucleobase.

CLAUSE 3. The computer-implemented method of clause 1, wherein accessing the splice-site score for the variant nucleobase based on the one or more conditional probabilities comprises accessing one or more context-specific conditional probabilities from among context-specific conditional probabilities determined, with respect to a target context, for the nucleotide sequence comprising the conditioned nucleobase at the conditioned position and flanking nucleobases within a threshold number of nucleobases of the conditioned nucleobase, each context-specific conditional probability from the context-specific conditional probabilities indicating a likelihood that a corresponding flanking nucleobase is part of a matching acceptor or a matching donor for the conditioned nucleobase with respect to the target context.

CLAUSE 4. The computer-implemented method of clause 3, wherein accessing the one or more context-specific conditional probabilities from among the context-specific conditional probabilities determined for the nucleotide sequence with respect to the target context comprises accessing:
one or more tissue-specific conditional probabilities from among tissue-specific conditional probabilities determined for the nucleotide sequence with respect to a target tissue;
one or more disease-specific conditional probabilities from among disease-specific conditional probabilities determined for the nucleotide sequence with respect to a target disease;
one or more cell-type-specific conditional probabilities from among cell-type-specific conditional probabilities determined for the nucleotide sequence with respect to a target cell type;
one or more cell-line-specific conditional probabilities from among cell-line-specific conditional probabilities determined for the nucleotide sequence with respect to a target cell line; or
one or more assay-specific conditional probabilities from among assay-specific conditional probabilities determined for the nucleotide sequence with respect to a target assay type.

CLAUSE 5. The computer-implemented method of clause 1, wherein identifying the variant nucleobase comprises:
identifying a set of genomic samples comprising the variant nucleobase at the target position within the nucleotide sequence based on a set of nucleotide reads for the set of genomic samples; or
identifying a genomic sample comprising the variant nucleobase at the target position within the nucleotide sequence based on nucleotide reads for the genomic sample.

CLAUSE 6. The computer-implemented method of clause 1, further comprising:
determining, for at least the genomic sample, nucleotide reads corresponding to the genomic region;
comparing the nucleotide reads corresponding to the genomic region with a reference genome; and
confirming at least the genomic sample comprises the variant nucleobase at the target position within the nucleotide sequence and that the variant nucleobase is associated with the alternative splicing based on comparing the nucleotide reads corresponding to the genomic region with the reference genome.

CLAUSE 7. The computer-implemented method of clause 1, wherein determining that the variant nucleobase at the target position within the nucleotide sequence is associated with the alternative splicing of pre-mRNA comprises determining that the variant nucleobase at the target position within the nucleotide sequence is:
associated with splicing of pre-mRNA at a first matching donor or a first matching acceptor corresponding to the conditioned nucleobase in a first percentage of splicing events; and
associated with splicing of pre-mRNA at a second matching donor or a second matching acceptor corresponding to the conditioned nucleobase in a second percentage of splicing events, the first percentage and the second percentage differing from percentages of splicing events in the reference splicing of pre-mRNA at the first matching donor or the first matching acceptor and the second matching donor or the second matching acceptor.

CLAUSE 8. The computer-implemented method of clause 1, wherein determining that the variant nucleobase at the target position within the nucleotide sequence is associated with the alternative splicing of the pre-mRNA comprises determining that the variant nucleobase at the target position within the nucleotide sequence is associated with the alternative splicing of the pre-mRNA that causes a disease exhibited by an organism of at least the genomic sample.

CLAUSE 9. The computer-implemented method of clause 8, further comprising:
performing a minigene splicing assay on at least the genomic sample; and
confirming, based on the minigene splicing assay, the variant nucleobase at the target position within the nucleotide sequence is associated with the alternative splicing.

CLAUSE 10. The computer-implemented method of clause 1, further comprising determining at least a candidate biochemical compound that targets the variant nucleobase or one or more adjacent nucleobases flanking the variant nucleobase to modify effects of the alternative splicing of the pre-mRNA for an organism of at least the genomic sample.

CLAUSE 11. The computer-implemented method of clause 10, wherein determining at least the candidate biochemical compound comprises identifying one or more candidate antisense oligonucleotides that modify splicing of the pre-mRNA for at least the genomic sample relative to the alternative splicing of the pre-mRNA.

CLAUSE 12. The computer-implemented method of clause 10, wherein determining at least the candidate biochemical compound comprises identifying one or more candidate pharmaceutical compounds that modify splicing of the pre-mRNA of at least the genomic sample relative to the alternative splicing of the pre-mRNA.

CLAUSE 13. The computer-implemented method of clause 10, wherein determining at least the candidate biochemical compound comprises identifying one or more candidate antigens that targets the variant nucleobase or the one or more adjacent nucleobases flanking the variant nucleobase.

CLAUSE 14. The computer-implemented method of clause 1, wherein determining, based on the splice-site score, that the variant nucleobase at the target position within the nucleotide sequence is associated with the alternative splicing corresponding to a target phenotype comprises:
providing, to a gene-to-phenotype-association model, the splice-site score for the variant nucleobase at the target position within the nucleotide sequence corresponding to a target gene; and
determining, utilizing the gene-to-phenotype-association model, a gene-to-phenotype score indicating a likelihood that the target gene is associated with the target phenotype.

CLAUSE 15. The computer-implemented method of clause 14, wherein determining the gene-to-phenotype score indicating the likelihood that the target gene is associated with the target phenotype utilizing a gene-based burden-test model as the gene-to-phenotype-association model comprises:
identifying a first set of genomic samples comprising the variant nucleobase at the target position within the nucleotide sequence based on a first set of nucleotide reads for the first set of genomic samples;
identifying a second set of genomic samples comprising a reference nucleobase at the target position within the nucleotide sequence based on a second set of nucleotide reads for the second set of genomic samples;
providing, to the gene-based burden-test model, the splice-site score for the variant nucleobase at the target position, an additional splice-site score for the reference nucleobase at the target position within the nucleotide sequence, and genetic data indicating a phenotype exhibited by the first set of genomic samples and the second set of genomic samples; and
determining, utilizing the gene-based burden-test model, the gene-to-phenotype score comprising a value indicating a statistical likelihood that the target gene is associated with the target phenotype based on the splice-site score, the additional splice-site score, and the genetic data.

CLAUSE 16. The computer-implemented method of clause 14, wherein:
providing the splice-site score for the variant nucleobase at the target position comprises identifying the variant nucleobase for the gene-to-phenotype-association model based on the splice-site score or inputting the splice-site score into the gene-to-phenotype-association model; and
determining the gene-to-phenotype score comprises:
   generating, utilizing a rare-variant collapsing model as the gene-to-phenotype-association model and based on a gene-by-individual matrix comprising data for the target gene, a value for the gene-to-phenotype score indicating the likelihood that the target gene is associated with the target phenotype; or
   generating, utilizing a probabilistic model as the gene-to-phenotype-association model and based on the splice-site score, a probability for the gene-to-phenotype score indicating the likelihood that the target gene is associated with the target phenotype.

CLAUSE 17. The computer-implemented method of clause 1, wherein determining, based on the splice-site score, that the variant nucleobase at the target position within the nucleotide sequence is associated with the alternative splicing comprises:
accessing, for a set of genes from a genome associated with at least the genomic sample and processed by a gene embedding neural network, gene-to-phenotype scores indicating respective probabilities of the set of genes being associated with phenotypes determined for an organism of the genomic sample;
providing, to a diagnostic variant model, the splice-site score for the variant nucleobase at the target position and the gene-to-phenotype scores; and
determining, utilizing the diagnostic variant model and based on the splice-site score and the gene-to-phenotype scores, the genomic sample comprises a diagnostic variant that is associated with a phenotype of the phenotypes determined for the organism.

CLAUSE 18. A computer-implemented method comprising:
identifying, for a genomic region, an artificial nucleotide sequence comprising a target nucleobase at a target position;
accessing a first context-specific splice-site score for the target nucleobase with respect to a first target context based on one or more first context-specific conditional probabilities that one or more flanking nucleobases from the artificial nucleotide sequence are part of one or more matching acceptors or one or more matching donors for a conditioned nucleobase at a conditioned position within the artificial nucleotide sequence with respect to the first target context;
accessing a second context-specific splice-site score for the target nucleobase with respect to a second target context differing from the first target context based on one or more second context-specific conditional probabilities that the one or more flanking nucleobases are part of the one or more matching acceptors or the one or more matching donors for the conditioned nucleobase at the conditioned position within the artificial nucleotide sequence with respect to the second target context; and
determining, based on the first context-specific splice-site score and the second context-specific splice-site score, that the target nucleobase at the target position within the artificial nucleotide sequence is associated with splicing of pre-messenger ribonucleic acid (pre-mRNA) in the first target context and not associated with splicing of pre-mRNA in the second target context.

CLAUSE 19. The computer-implemented method of clause 18, wherein the first context-specific splice-site score and the first target context and the second context-specific splice-site score and the second target context comprise:
a first tissue-specific splice-site score for the target nucleobase with respect to a first tissue based on one or more first tissue-specific conditional probabilities and a second tissue-specific splice-site score for the target nucleobase with respect to a second tissue based on one or more second tissue-specific conditional probabilities;
a first disease-specific splice-site score for the target nucleobase with respect to a first disease based on one or more first disease-specific conditional probabilities and a second disease-specific splice-site score for the target nucleobase with respect to a second disease based on one or more second disease-specific conditional probabilities;
a first cell-type-specific splice-site score for the target nucleobase with respect to a first cell type based on one or more first cell-type-specific conditional probabilities and a second cell-type-specific splice-site score for the target nucleobase with respect to a second cell type based on one or more second cell-type-specific conditional probabilities;
a first cell-line-specific splice-site score for the target nucleobase with respect to a first cell-line type based on one or more first cell-line-specific conditional probabilities and a second cell-line-specific splice-site score for the target nucleobase with respect to a second cell-line type based on one or more second cell-line-specific conditional probabilities; or
a first assay-specific splice-site score for the target nucleobase with respect to a first assay type based on one or more first assay-specific conditional probabilities and a second assay-specific splice-site score for the target nucleobase with respect to a second assay type based on one or more second assay-specific conditional probabilities.

CLAUSE 20. The computer-implemented method of clause 18, wherein the first context-specific splice-site score and the first target context and the second context-specific splice-site score and the second target context comprise a first cell-type-specific splice-site score for the target nucleobase with respect to a first cell type comprising a target variant in a gene and a second cell-type-specific splice-site score for the target nucleobase with respect to a second cell type not comprising the target variant in the gene.

CLAUSE 21. The computer-implemented method of clause 18, wherein determining that the target nucleobase at the target position within the artificial nucleotide sequence is associated with alternative splicing of the pre-mRNA in the first target context relative to a reference splicing of the pre-mRNA and not associated with the alternative splicing of the pre-mRNA in the second target context.

CLAUSE 22. The computer-implemented method of clause 22, further comprising determining that the target nucleobase at the target position within the artificial nucleotide sequence is associated with alternative splicing of pre-mRNA in the first target context and not associated with the alternative splicing of the pre-mRNA in the second target context.

CLAUSE 23. The computer-implemented method of clause 18, wherein the artificial nucleotide sequence comprises an antisense oligonucleotide or a nucleotide sequence including a synthetic intron.

CLAUSE 24. The computer-implemented method of clause 23, wherein the synthetic intron encodes a kinase or a virus designed to treat a disease.

CLAUSE 25. The computer-implemented method of clause 18, wherein the target nucleobase comprises a reference nucleobase or a variant nucleobase.

The components of the splice-site prediction system 106 can include software, hardware, or both. For example, the components of the splice-site prediction system 106 can include one or more instructions stored on a computer-readable storage medium and executable by processors of one or more computing devices (e.g., the server device(s) 102, the client device 110, and/or the therapeutics analysis device(s) 114). When executed by the one or more processors, the computer-executable instructions of the splice-site prediction system 106 can cause the computing devices to perform the splice site detection methods described herein. Alternatively, the components of the splice-site prediction system 106 can comprise hardware, such as special purpose processing devices to perform a certain function or group of functions. Additionally, or alternatively, the components of the splice-site prediction system 106 can include a combination of computer-executable instructions and hardware.

Furthermore, the components of the splice-site prediction system 106 performing the functions described herein with respect to the splice-site prediction system 106 may, for example, be implemented as part of a stand-alone application, as a module of an application, as a plug-in for applications, as a library function or functions that may be called by other applications, and/or as a cloud-computing model. Thus, components of the splice-site prediction system 106 may be implemented as part of a stand-alone application on a personal computing device or a mobile device. Additionally, or alternatively, the components of the splice-site prediction system 106 may be implemented in any application that provides sequencing services including, but not limited to Illumina SpliceAI, Illumina PrimateAI, Illumina PrimateAI1D, Illumina PrimateAI2D, Illumina PrimateAI3D, or Illumina TruSight. “Illumina,” “SpliceAI,” “PrimateAI,” “PrimateAI1D,” “PrimateAI2D,” “PrimateAI3D,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 52 illustrates a block diagram of a computing device 5200 that may be configured to perform one or more of the processes described above. One will appreciate that one or more computing devices such as the computing device 5200 may implement the splice-site prediction system 106. As shown by FIG. 52, the computing device 5200 can comprise a processor 5202, a memory 5204, a storage device 5206, an I/O interface 5208, and a communication interface 5210, which may be communicatively coupled by way of a communication infrastructure 5212. In certain embodiments, the computing device 5200 can include fewer or more components than those shown in FIG. 52. The following paragraphs describe components of the computing device 5200 shown in FIG. 52 in additional detail.

In one or more embodiments, the processor 5202 includes hardware for executing instructions, such as those making up a computer program. As an example, and not by way of limitation, to execute instructions for dynamically modifying workflows, the processor 5202 may retrieve (or fetch) the instructions from an internal register, an internal cache, the memory 5204, or the storage device 5206 and decode and execute them. The memory 5204 may be a volatile or non-volatile memory used for storing data, metadata, and programs for execution by the processor(s). The storage device 5206 includes storage, such as a hard disk, flash disk drive, or other digital storage device, for storing data or instructions for performing the methods described herein.

The I/O interface 5208 allows a user to provide input to, receive output from, and otherwise transfer data to and receive data from computing device 5200. The I/O interface 5208 may include a mouse, a keypad or a keyboard, a touch screen, a camera, an optical scanner, network interface, modem, other known I/O devices or a combination of such I/O interfaces. The I/O interface 5208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 5208 is configured to provide graphical data to a display for presentation to a user. The graphical data may be representative of one or more graphical user interfaces and/or any other graphical content as may serve a particular implementation.

The communication interface 5210 can include hardware, software, or both. In any event, the communication interface 5210 can provide one or more interfaces for communication (such as, for example, packet-based communication) between the computing device 5200 and one or more other computing devices or networks. As an example, and not by way of limitation, the communication interface 5210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 5210 may facilitate communications with various types of wired or wireless networks. The communication interface 5210 may also facilitate communications using various communication protocols. The communication infrastructure 5212 may also include hardware, software, or both that couples components of the computing device 5200 to each other. For example, the communication interface 5210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
provide, to a neural network, a nucleotide sequence comprising a conditioned nucleobase at a conditioned position and flanking nucleobases within a threshold number of nucleobases of the conditioned nucleobase;
generate, utilizing the neural network and based on the conditioned nucleobase, conditional probabilities for the flanking nucleobases, each conditional probability indicating a likelihood that a flanking nucleobase is part of a matching donor or a matching acceptor corresponding to the conditioned nucleobase; and
determine, based on the conditional probabilities for the flanking nucleobases, at least one flanking nucleobase from the flanking nucleobases that is part of the matching donor or the matching acceptor for the conditioned nucleobase.

2. The system of claim 1, further comprising instructions, that when executed by the at least one processor, cause the system to provide the nucleotide sequence comprising the conditioned nucleobase by providing the nucleotide sequence comprising a splice-junction nucleobase at the conditioned position representing a splice site.

3. The system of claim 1, further comprising instructions that, when executed by the at least one processor, cause the system to:
provide the nucleotide sequence by providing the nucleotide sequence comprising the conditioned nucleobase representing a conditioned acceptor or a conditioned donor; and
generate the conditional probabilities for the flanking nucleobases by generating a probability that each flanking nucleobase is part of a matching donor or a matching acceptor respectively corresponding to the conditioned acceptor or the conditioned donor of a splice site.

4. The system of claim 1, further comprising instructions that, when executed by the at least one processor, cause the system to generate the conditional probabilities for the flanking nucleobases by generating, for each flanking nucleobase corresponding to a conditioned donor represented by the conditioned nucleobase:
a first conditional probability that the flanking nucleobase is part of a downstream acceptor that corresponds to the conditioned donor; or
a second conditional probability that the flanking nucleobase is part of an upstream acceptor that corresponds to the conditioned donor.

5. The system of claim 1, further comprising instructions that, when executed by the at least one processor, cause the system to generate the conditional probabilities for the flanking nucleobases by generating, for each flanking nucleobase corresponding to a conditioned acceptor represented by the conditioned nucleobase:
a first conditional probability that the flanking nucleobase is part of a downstream donor that corresponds to the conditioned acceptor; or
a second conditional probability that the flanking nucleobase is part of an upstream donor that corresponds to the conditioned acceptor.

6. The system of claim 1, further comprising instructions that, when executed by the at least one processor, cause the system to:
provide, to an input channel of the neural network, a conditioned position identifier indicating that the conditioned nucleobase is at the conditioned position; and
generate, utilizing the neural network, the conditional probabilities for the flanking nucleobases further based on the conditioned position identifier.

7. The system of claim 1, further comprising instructions that, when executed by the at least one processor, cause the system to:
provide, to an input channel of the neural network, a distance value for each flanking nucleobase that corresponds to a number of nucleobases between the flanking nucleobase and the conditioned nucleobase within the nucleotide sequence; and
generate, utilizing the neural network, the conditional probabilities for the flanking nucleobases further based on the distance value for each flanking nucleobase.

8. The system of claim 7, further comprising instructions that, when executed by the at least one processor, cause the system to determine the distance value for each flanking nucleobase by:
determining an integer value for each flanking nucleobase that indicates a distance of the flanking nucleobase from the conditioned nucleobase based on a number of positions from the flanking nucleobase to the conditioned nucleobase within the nucleotide sequence; and
generating the distance value for each flanking nucleobase from the integer value using a logarithmic transform.

9. The system of claim 1, further comprising instructions that, when executed by the at least one processor, cause the system to provide the nucleotide sequence by providing, for a genomic sample, a pre-messenger ribonucleic acid (pre-mRNA) sequence comprising a variant nucleobase.

10. The system of claim 1, further comprising instructions that, when executed by the at least one processor, cause the system to:
provide, to the neural network, an additional nucleotide sequence comprising an additional conditioned nucleobase at an additional conditioned position and additional flanking nucleobases;
generate, utilizing the neural network and based on the additional conditioned nucleobase, additional conditional probabilities for the additional flanking nucleobases; and
determining, based on the additional conditional probabilities, that the additional nucleotide sequence excludes a matching donor or a matching acceptor for the additional conditioned nucleobase.

11. The system of claim 1, further comprising instructions that, when executed by the at least one processor, cause the system to:
provide the nucleotide sequence by providing a ribonucleic acid (RNA) sequence or a deoxyribonucleic acid (DNA) sequence based on long nucleotide reads of a threshold number of nucleobases; and
generate the conditional probabilities by generating:
a first set of conditional probabilities that flanking nucleobases are part of an upstream acceptor that corresponds to a conditioned donor; or
a second set of conditional probabilities that flanking nucleobases are part of a downstream donor that corresponds to a conditioned acceptor.

12. The system of claim 1, further comprising instructions that, when executed by the at least one processor, cause the system to:
provide the nucleotide sequence by providing a reference-sequence vector representing a reference nucleotide sequence comprising the flanking nucleobases;
compare the conditional probabilities for the flanking nucleobases to values within a ground-truth-label vector comprising a non-zero value for at least one flanking nucleobase that is part of the matching donor or the matching acceptor and at least two zero values for other flanking nucleobases that are not part of the matching donor or the matching acceptor; and
adjust parameters for the neural network based on comparing the conditional probabilities for the flanking nucleobases to the values within the ground-truth-label vector.

13. A system comprising:
at least one processor; and
a non-transitory computer readable medium comprising instructions that, when executed by the at least one processor, cause the system to:
provide, to a neural network, a nucleotide sequence comprising a conditioned nucleobase at a conditioned position and a series of flanking nucleobases;
generate, from the nucleotide sequence utilizing the neural network, a first set of context-specific conditional probabilities for a first context associated with the nucleotide sequence, each conditional probability from the first set of context-specific conditional probabilities indicating a likelihood that a corresponding flanking nucleobase is part of one or more matching acceptors or one or more matching donors for the conditioned nucleobase with respect to the first context; and
generate, from the nucleotide sequence utilizing the neural network, a second set of context-specific conditional probabilities for a second context associated with the nucleotide sequence, each conditional probability from the second set of context-specific conditional probabilities indicating an additional likelihood that a corresponding flanking nucleobase is part of the one or more matching acceptors or the one or more matching donors for the conditioned nucleobase with respect to the second context.

14. The system of claim 13, further comprising instructions that, when executed by the at least one processor, cause the system to generate, utilizing the neural network, one or more genomic track signals that indicate one or more properties of the nucleotide sequence.

15. The system of claim 14, further comprising instructions that, when executed by the at least one processor, cause the system to:
generate the first set of context-specific conditional probabilities and the second set of context-specific conditional probabilities utilizing the neural network by generating the first set of context-specific conditional probabilities and the second set of context-specific conditional probabilities utilizing a softmax layer of the neural network; and
generate the one or more genomic track signals utilizing the neural network by generating the one or more genomic track signals utilizing a feedforward layer of the neural network.

16. The system of claim 14, further comprising instructions that, when executed by the at least one processor, cause the system to generate the one or more genomic track signals by generating ribonucleic acid (RNA) binding protein signals that indicate probabilities that positions within the nucleotide sequence correspond to a binding site for an RNA binding protein.

17. The system of claim 14, further comprising instructions that, when executed by the at least one processor, cause the system to generate the one or more genomic track signals by generating histone mark signals that indicate probabilities that positions within the nucleotide sequence correspond to a histone mark.

18. The system of claim 13, wherein the first context comprises a first tissue and the second context comprises a second tissue, the first tissue and the second tissue comprising two of brain tissue, liver tissue, adipose tissue, skin tissue, muscle tissue, heart tissue, pancreas tissue, kidney tissue, artery tissue, or a whole blood sample.

19. The system of claim 13, further comprising instructions that, when executed by the at least one processor, cause the system to generate the first set of context-specific conditional probabilities for the first context and the second set of context-specific conditional probabilities for the second context by generating:
a first set of tissue-specific conditional probabilities for a first tissue and a second set of tissue-specific conditional probabilities for a second tissue;
a first set of disease-specific conditional probabilities for a first disease and a second set of disease-specific conditional probabilities for a second disease;
a first set of cell-type-specific conditional probabilities for a first cell type and a second set of cell-type-specific conditional probabilities for a second cell type;
a first set of cell-line-specific conditional probabilities for a first cell line and a second set of cell-line-specific conditional probabilities for a second cell line; or
a first set of assay-specific conditional probabilities for a first assay type and a second set of assay-specific conditional probabilities for a second assay type.

20. The system of claim 13, further comprising instructions that, when executed by the at least one processor, cause the system to generate, from the nucleotide sequence utilizing the neural network, one or more additional sets of context-specific conditional probabilities for one or more additional contexts associated with the nucleotide sequence.

21. The system of claim 13, further comprising instructions that, when executed by the at least one processor, cause the system to determine one or more context-specific spliced isoforms of a messenger ribonucleic acid (mRNA) sequence corresponding to the nucleotide sequence using the first set of context-specific conditional probabilities and the second set of context-specific conditional probabilities.

22. The system of claim 13, further comprising instructions that, when executed by the at least one processor, cause the system to:
generate a first set of logits from the first set of context-specific conditional probabilities and a second set of logits from the second set of context-specific conditional probabilities;
determine logit differences by comparing the first set of logits and the second set of logits; and
determine relative usage rates for the one or more matching acceptors or the one or more matching donors with respect to the first context and the second context based on the logit differences.

23. The system of claim 22, further comprising instructions that, when executed by the at least one processor, cause the system to generate, utilizing the logit differences, a performance matrix indicating mean pair-wise context-specific performance of the neural network.

24. The system of claim 13, further comprising instructions that, when executed by the at least one processor, cause the system to:
determine, for the first context and based on the first set of context-specific conditional probabilities, a first flanking nucleobase from the series of flanking nucleobases having a first highest likelihood of being a matching acceptor or a matching donor for the conditioned nucleobase; and
determine, for the second context and based on the second set of context-specific conditional probabilities, a second flanking nucleobase from the series of flanking nucleobases having a second highest likelihood of being the matching acceptor or the matching donor for the conditioned nucleobase.

25. A system comprising:
at least one processor; and
a non-transitory computer readable medium comprising instructions that, when executed by the at least one processor, cause the system to:
provide, to a neural network, a training nucleotide sequence comprising a conditioned nucleobase at a conditioned position and a series of flanking nucleobases;
generate, from the training nucleotide sequence utilizing the neural network, a first set of predicted context-specific conditional probabilities for a first context associated with the training nucleotide sequence, each conditional probability from the first set of predicted context-specific conditional probabilities indicating a likelihood that a corresponding flanking nucleobase is part of one or more matching acceptors or one or more matching donors for the conditioned nucleobase with respect to the first context;
generate, from the training nucleotide sequence utilizing the neural network, a second set of predicted context-specific conditional probabilities for a second context associated with the training nucleotide sequence, each conditional probability from the second set of predicted context-specific conditional probabilities indicating a likelihood that a corresponding flanking nucleobase is part of one or more matching acceptors or one or more matching donors for the conditioned nucleobase with respect to the second context;
compare, to determine a loss, the first set of predicted context-specific conditional probabilities and the second set of predicted context-specific conditional probabilities to corresponding context-specific ground truth probabilities for the training nucleotide sequence; and
adjust parameters of the neural network based on the determined loss.

26. The system of claim 25, further comprising instructions that, when executed by the at least one processor, cause the system to compare, to determine the loss, the first set of predicted context-specific conditional probabilities and the second set of predicted context-specific conditional probabilities to the corresponding context-specific ground truth probabilities by:
generate a first set of predicted logits from the first set of predicted context-specific conditional probabilities and a second set of predicted logits from the second set of predicted context-specific conditional probabilities; and
determine the loss by, via a loss function, comparing the predicted logits to true logits derived from the corresponding context-specific ground truth probabilities.

27. The system of claim 25, further comprising instructions that, when executed by the at least one processor, cause the system to compare the first set of predicted context-specific conditional probabilities and the second set of predicted context-specific conditional probabilities to the corresponding context-specific ground truth probabilities by using a cross-context loss function that adjusts parameters to improve a prediction accuracy across a context dimension.

28. The system of claim 25, further comprising instructions that, when executed by the at least one processor, cause the system to compare the first set of predicted context-specific conditional probabilities and the second set of predicted context-specific conditional probabilities to the corresponding context-specific ground truth probabilities by using a soft-weighted cross-entropy loss function that increases a weight for context-specific training nucleotide sequences.

29. The system of claim 25, further comprising instructions that, when executed by the at least one processor, cause the system to:
generate, from the training nucleotide sequence utilizing the neural network, one or more predicted genomic track signals that indicate one or more properties of the training nucleotide sequence;
compare, to determine an additional loss, the one or more predicted genomic track signals to one or more corresponding ground truth genomic track signals; and
adjust the parameters of the neural network based on the determined loss and the determined additional loss.

30. The system of claim 29, further comprising instructions that, when executed by the at least one processor, cause the system to compare the one or more predicted genomic track signals to one or more corresponding ground truth genomic track signals by comparing the one or more predicted genomic track signals to one or more corresponding ground truth genomic track signals via a mean-squared-error loss function.

31. The system of claim 29, further comprising instructions that, when executed by the at least one processor, cause the system to:
generate one or more transformed ground truth genomic track signals by applying a logarithmic transformation to the one or more corresponding ground truth genomic track signals; and
compare the one or more predicted genomic track signals to the one or more corresponding ground truth genomic track signals by comparing the one or more predicted genomic track signals to the one or more transformed ground truth genomic track signals.

32. The system of claim 29, further comprising instructions that, when executed by the at least one processor, cause the system to generate the one or more predicted genomic track signals by:
generating ribonucleic acid (RNA) binding protein signals that indicate probabilities that positions within the training nucleotide sequence correspond to a binding site for an RNA binding protein; and
generating histone mark signals that indicate probabilities that positions within the training nucleotide sequence correspond to a histone mark.

33. A system comprising:
at least one processor; and
a non-transitory computer readable medium comprising instructions that, when executed by the at least one processor, cause the system to:
provide, to a neural network, a nucleotide sequence comprising a conditioned nucleobase at a conditioned position and a series of flanking nucleobases including an intron of the nucleotide sequence;
generate, utilizing the neural network, an intron-retention probability indicating a likelihood that the intron is retained in messenger ribonucleic acid (mRNA) corresponding to the nucleotide sequence and a set of conditional probabilities that each indicate a likelihood that a flanking nucleobase is part of one or more matching acceptors or one or more matching donors for the conditioned nucleobase; and
determine, based on the intron-retention probability and the set of conditional probabilities, that the mRNA retains the intron.

34. The system of claim 33, further comprising instructions that, when executed by the at least one processor, cause the system to provide the nucleotide sequence comprising the conditioned nucleobase and the series of flanking nucleobases by:
providing the nucleotide sequence comprising a conditioned donor and a series of downstream nucleobases that includes the intron followed by a matching acceptor; or
providing the nucleotide sequence comprising a conditioned acceptor and a series of upstream nucleobases that includes the intron preceded by a matching donor.

35. The system of claim 33, further comprising instructions that, when executed by the at least one processor, cause the system to generate the intron-retention probability and the set of conditional probabilities by generating an output vector comprising the intron-retention probability and the set of conditional probabilities, one or more positions of the output vector corresponding to positions of flanking nucleobases from the series of flanking nucleobases within the nucleotide sequence.

36. The system of claim 35, further comprising instructions that, when executed by the at least one processor, cause the system to:
generate, from the nucleotide sequence, the intron-retention probability;
generate, from the nucleotide sequence, a vector comprising the set of conditional probabilities, each position of the vector corresponding to a position of a flanking nucleobase from the series of flanking nucleobases; and
generate the output vector by combining the intron-retention probability and the vector comprising the set of conditional probabilities.

37. The system of claim 33, further comprising instructions that, when executed by the at least one processor, cause the system to generate the intron-retention probability by generating the intron-retention probability for a position of the nucleotide sequence that is within a threshold number of positions from the conditioned position.

38. The system of claim 33, further comprising instructions that, when executed by the at least one processor, cause the system to generate the intron-retention probability and the set of conditional probabilities by generating a plurality of probabilities that sum to a value of one.

39. The system of claim 33, further comprising instructions that, when executed by the at least one processor, cause the system to generate, for display within a graphical user interface, an intron-retention indicator that a gene of a genomic sample associated with the nucleotide sequence comprises a variant associated with the mRNA retaining the intron after splicing.

40. The system of claim 33, further comprising instructions that, when executed by the at least one processor, cause the system to provide the nucleotide sequence to the neural network by providing the nucleotide sequence comprising a pre-messenger ribonucleic acid (pre-mRNA) sequence comprising a variant nucleobase.

41. The system of claim 33, further comprising instructions that, when executed by at least one processor, cause the system to:
provide, to the neural network, an additional nucleotide sequence comprising an additional conditioned nucleobase at an additional conditioned position and an additional series of flanking nucleobases including an additional intron of the additional nucleotide sequence; and
generate, utilizing the neural network, an additional intron-retention probability having a zero value or a value below a threshold value that indicates that the intron is spliced out of mRNA corresponding to the additional nucleotide sequence during splicing.

42. The system of claim 33, further comprising instructions that, when executed by the at least one processor, cause the system to generate, utilizing the neural network, the intron-retention probability and the set of conditional probabilities by generating the intron-retention probability and the set of conditional probabilities utilizing a transformer neural network.

43. A system comprising:
at least one processor; and
a non-transitory computer readable medium comprising instructions that, when executed by the at least one processor, cause the system to:
provide, to a neural network, a training nucleotide sequence comprising a conditioned nucleobase at a conditioned position and a series of flanking nucleobases including an intron of the training nucleotide sequence;
generate, utilizing the neural network, a predicted intron-retention probability indicating a likelihood that at least a portion of the intron is retained in messenger ribonucleic acid (mRNA) corresponding to the training nucleotide sequence and a predicted set of conditional probabilities that each indicate a likelihood that a flanking nucleobase is part of one or more matching acceptors or one or more matching donors for the conditioned nucleobase;
compare, to determine a loss, the predicted intron-retention probability to a ground truth indicator of intron retention and the predicted set of conditional probabilities to ground truth indicators for the one or more matching acceptors or the one or more matching donors generated for the training nucleotide sequence using an exon-intron boundary count associated with the training nucleotide sequence; and
adjust parameters of the neural network based on the determined loss.

44. The system of claim 43, further comprising instructions that, when executed by the at least one processor, cause the system to compare the predicted intron-retention probability to the ground truth indicator of intron retention using the exon-intron boundary count associated with the training nucleotide sequence by comparing the predicted intron-retention probability to a ground truth intron-retention probability derived using the exon-intron boundary count.

45. The system of claim 43, further comprising instructions that, when executed by the at least one processor, cause the system to determine the exon-intron boundary count associated with the training nucleotide sequence by determining, for a genomic sample associated with the training nucleotide sequence, a number of nucleotide reads that cover a boundary of the intron.

46. The system of claim 43, further comprising instructions that, when executed by the at least one processor, cause the system to determine the exon-intron boundary count associated with the training nucleotide sequence by:
identifying a set of nucleotide reads of a genomic sample associated with the training nucleotide sequence; and
determining, from among the set of nucleotide reads, a first number of nucleotide reads that cover a threshold number of nucleobases upstream and downstream from an exon-intron boundary between an exon and an adjacent intron.

47. The system of claim 46, further comprising instructions that, when executed by the at least one processor, cause the system to determine the exon-intron boundary count associated with the training nucleotide sequence by:
determining, from among the set of nucleotide reads, a second number of nucleotide reads that cover a threshold number of nucleobases within an exon of the exon-intron boundary; and
determining the exon-intron boundary count for the training nucleotide sequence using a ratio of the first number of nucleotide reads and the second number of nucleotide reads.

48. The system of claim 47, further comprising instructions that, when executed by the at least one processor, cause the system to determine the exon-intron boundary count for the training nucleotide sequence using the ratio of the first number of nucleotide reads and the second number of nucleotide reads by determining a normalized exon-intron boundary count using the ratio and a third number of nucleotide reads from the set of nucleotide reads that represents a junction count associated with the training nucleotide sequence.

49. The system of claim 43, further comprising instructions that, when executed by the at least one processor, cause the system to:
determine exon-intron boundary counts for a training dataset used in learning parameters for the neural network, the training dataset comprising the training nucleotide sequence; and
up-sample the exon-intron boundary counts to increase representation of the exon-intron boundary counts within the training dataset.

50. The system of claim 43, further comprising instructions that, when executed by the at least one processor, cause the system to determine exon-intron boundary counts for a training dataset used in learning parameters for the neural network by:
determining a first set of counts based on nucleotide reads for the training dataset that cover boundaries between exons and adjacent introns represented in the training dataset; and
determining a second set of counts based on exon-intron boundary proxies that include positions within one or more exons represented in the training dataset.

51. The system of claim 43, further comprising instructions that, when executed by the at least one processor, cause the system to determine exon-intron boundary counts for a training dataset used in learning parameters for the neural network by:
determining raw counts using nucleotide reads of genomic samples associated with training nucleotide sequences included in the training dataset;
generating transformed counts by applying an arcsinh transformation to the raw counts; and
determining the exon-intron boundary counts for the training dataset using the transformed counts.

52. The system of claim 43, further comprising instructions that, when executed by the at least one processor, cause the system to determine the loss using at least one of:
a focal loss function that modifies a weight for nucleobase positions based on an associated probability; or
a weighted loss function that increases a weight for exon-intron boundary counts.

53. A system comprising:
at least one processor; and
a non-transitory computer readable medium comprising instructions that, when executed by the at least one processor, cause the system to:
provide, to a transformer neural network, a nucleotide sequence comprising a conditioned nucleobase at a conditioned position and flanking nucleobases within a threshold number of nucleobases of the conditioned nucleobase;
generate, utilizing one or more convolutional blocks of the transformer neural network, a convolutional feature vector representing the nucleotide sequence;
generate, utilizing one or more transformer blocks of the transformer neural network and from the convolutional feature vector, an attention-based feature vector that captures sequence patterns across the nucleotide sequence; and
generate, utilizing an output layer of the transformer neural network and based on the attention-based feature vector, conditional probabilities for the flanking nucleobases, each conditional probability indicating a likelihood that a flanking nucleobase is part of a matching donor or a matching acceptor corresponding to the conditioned nucleobase at the conditioned position.

54. The system of claim 53, further comprising instructions that, when executed by the at least one processor, cause the system to generate the attention-based feature vector utilizing the one or more transformer blocks of the transformer neural network by generating the attention-based feature vector utilizing one or more fast-linear-attention-with-a-single-head (FLASH) blocks of the transformer neural network.

55. The system of claim 53, further comprising instructions that, when executed by the at least one processor, cause the system to generate the attention-based feature vector by generating the attention-based feature vector utilizing at least one transformer block that includes an attention unit with a normalization layer positioned to accept at least one of a linear-attention output or quadratic-attention output of an internal layer of the attention unit.

56. The system of claim 53, further comprising instructions that, when executed by the at least one processor, cause the system to generate the attention-based feature vector by:
generating, from the convolutional feature vector, at least one feature tensor that is internal to an attention unit;
reshaping the at least one feature tensor according to a reshaping factor and along a sequence dimension corresponding to the nucleotide sequence; and
generating the attention-based feature vector from the at least one reshaped feature tensor utilizing the attention unit.

57. The system of claim 56, further comprising instructions that, when executed by the at least one processor, cause the system to reshape the at least one feature tensor according to the reshaping factor by applying the reshaping factor to a query feature tensor, a key feature tensor, and a value feature tensor along the sequence dimension.

58. The system of claim 53, further comprising instructions that, when executed by the at least one processor, cause the system to generate the convolutional feature vector utilizing the one or more convolutional blocks of the transformer neural network by generating the convolutional feature vector utilizing three or four convolutional blocks.

59. The system of claim 53, further comprising instructions that, when executed by the at least one processor, cause the system to generate the attention-based feature vector utilizing the one or more transformer blocks of the transformer neural network by:
generating a first attention-based feature vector from the convolutional feature vector utilizing a first transformer block of the transformer neural network, the first transformer block comprising a first normalization layer, a first attention unit, and a first concatenation layer;
providing the first attention-based feature vector to a second transformer block of the transformer neural network, the second transformer block comprising a second normalization layer, a second attention unit, and a second concatenation layer; and
generating a second attention-based feature vector from the first attention-based feature vector utilizing the second transformer block of the transformer neural network.

60. The system of claim 53, further comprising instructions that, when executed by the at least one processor, cause the system to provide the nucleotide sequence to the transformer neural network by providing the nucleotide sequence having a sequence length of between one thousand nucleobases to thirty-two thousand nucleobases to the transformer neural network.

61. The system of claim 53, further comprising instructions that, when executed by the at least one processor, cause the system to determine parameters for the transformer neural network using a stepwise learning rate decay.

62. The system of claim 61, further comprising instructions that, when executed by the at least one processor, cause the system to determine the parameters for the transformer neural network using the stepwise learning rate decay by:
updating the parameters of the transformer neural network utilizing an initial learning rate that incrementally increases up to between 1e-4 and 5e-3;
updating the parameters of the transformer neural network utilizing a first fixed learning rate of between 1e-4 and 5e-3 during a first set of update steps that ends after a threshold number of update steps between one thousand update steps and two thousand update steps; and
updating the parameters of the transformer neural network utilizing a second fixed learning rate that is lower than 1e-4 during a second set of update steps that begins after the threshold number of update steps.

63. The system of claim 53, further comprising instructions that, when executed by at least one processor, cause the system to determine parameters for the transformer neural network based on comparing, via a cross-entropy loss function, predicted conditional probabilities generated from training nucleotide sequences to corresponding ground truth probabilities of the flanking nucleobases being part of the matching donor or the matching acceptor.

64. A system comprising:
at least one processor; and
a non-transitory computer readable medium comprising instructions that, when executed by the at least one processor, cause the system to:
identify, for at least a genomic sample, a variant nucleobase at a target position within a nucleotide sequence corresponding to a genomic region;
access a splice-site score for the variant nucleobase based on one or more conditional probabilities that a flanking nucleobase from the nucleotide sequence is part of a matching donor or a matching acceptor corresponding to a conditioned nucleobase at a conditioned position within the nucleotide sequence; and
determine, based on the splice-site score, that the variant nucleobase at the target position within the nucleotide sequence is associated with alternative splicing of pre-messenger ribonucleic acid (pre-mRNA) relative to a reference splicing of pre-mRNA.

65. The system of claim 64, further comprising instructions that, when executed by the at least one processor, cause the system to access the splice-site score for the variant nucleobase based on the one or more conditional probabilities by accessing the one or more conditional probabilities from among conditional probabilities determined for the nucleotide sequence comprising the conditioned nucleobase at the conditioned position and flanking nucleobases within a threshold number of nucleobases of the conditioned nucleobase, each conditional probability from the conditional probabilities indicating a likelihood that a corresponding flanking nucleobase is part of a matching donor or a matching acceptor corresponding to the conditioned nucleobase.

66. The system of claim 64, further comprising instructions that, when executed by the at least one processor, cause the system to access the splice-site score for the variant nucleobase based on the one or more conditional probabilities by accessing one or more context-specific conditional probabilities from among context-specific conditional probabilities determined, with respect to a target context, for the nucleotide sequence comprising the conditioned nucleobase at the conditioned position and flanking nucleobases within a threshold number of nucleobases of the conditioned nucleobase, each context-specific conditional probability from the context-specific conditional probabilities indicating a likelihood that a corresponding flanking nucleobase is part of a matching acceptor or a matching donor for the conditioned nucleobase with respect to the target context.

67. The system of claim 66, further comprising instructions that, when executed by the at least one processor, cause the system to access the one or more context-specific conditional probabilities from among the context-specific conditional probabilities determined for the nucleotide sequence with respect to the target context by accessing:
one or more tissue-specific conditional probabilities from among tissue-specific conditional probabilities determined for the nucleotide sequence with respect to a target tissue;
one or more disease-specific conditional probabilities from among disease-specific conditional probabilities determined for the nucleotide sequence with respect to a target disease;
one or more cell-type-specific conditional probabilities from among cell-type-specific conditional probabilities determined for the nucleotide sequence with respect to a target cell type;
one or more cell-line-specific conditional probabilities from among cell-line-specific conditional probabilities determined for the nucleotide sequence with respect to a target cell line;
or
one or more assay-specific conditional probabilities from among assay-specific conditional probabilities determined for the nucleotide sequence with respect to a target assay type.

68. The system of claim 64, further comprising instructions that, when executed by the at least one processor, cause the system to identify the variant nucleobase by:
identifying a set of genomic samples comprising the variant nucleobase at the target position within the nucleotide sequence based on a set of nucleotide reads for the set of genomic samples; or
identifying a genomic sample comprising the variant nucleobase at the target position within the nucleotide sequence based on nucleotide reads for the genomic sample.

69. The system of claim 64, further comprising instructions that, when executed by the at least one processor, cause the system to:
determine, for at least the genomic sample, nucleotide reads corresponding to the genomic region;
compare the nucleotide reads corresponding to the genomic region with a reference genome; and
confirm at least the genomic sample comprises the variant nucleobase at the target position within the nucleotide sequence and that the variant nucleobase is associated with the alternative splicing based on comparing the nucleotide reads corresponding to the genomic region with the reference genome.

70. The system of claim 64, further comprising instructions that, when executed by the at least one processor, cause the system to determine that the variant nucleobase at the target position within the nucleotide sequence is associated with the alternative splicing of pre-mRNA by determining that the variant nucleobase at the target position within the nucleotide sequence is:
associated with splicing of pre-mRNA at a first matching donor or a first matching acceptor corresponding to the conditioned nucleobase in a first percentage of splicing events; and
associated with splicing of pre-mRNA at a second matching donor or a second matching acceptor corresponding to the conditioned nucleobase in a second percentage of splicing events, the first percentage and the second percentage differing from percentages of splicing events in the reference splicing of pre-mRNA at the first matching donor or the first matching acceptor and the second matching donor or the second matching acceptor.

71. The system of claim 64, further comprising instructions that, when executed by the at least one processor, cause the system to determine that the variant nucleobase at the target position within the nucleotide sequence is associated with the alternative splicing of the pre-mRNA by determining that the variant nucleobase at the target position within the nucleotide sequence is associated with the alternative splicing of the pre-mRNA that causes a disease exhibited by an organism of at least the genomic sample.

72. The system of claim 71, further comprising instructions that, when executed by the at least one processor, cause the system to:
perform a minigene splicing assay on at least the genomic sample; and
confirm, based on the minigene splicing assay, the variant nucleobase at the target position within the nucleotide sequence is associated with the alternative splicing.

73. The system of claim 64, further comprising instructions that, when executed by the at least one processor, cause the system to determine at least a candidate biochemical compound that targets the variant nucleobase or one or more adjacent nucleobases flanking the variant nucleobase to modify effects of the alternative splicing of the pre-mRNA for an organism of at least the genomic sample.

74. The system of claim 73, further comprising instructions that, when executed by the at least one processor, cause the system to determine at least the candidate biochemical compound by identifying one or more candidate antisense oligonucleotides that modify splicing of the pre-mRNA for at least the genomic sample relative to the alternative splicing of the pre-mRNA.

75. The system of claim 73, further comprising instructions that, when executed by the at least one processor, cause the system to determine at least the candidate biochemical compound by identifying one or more candidate pharmaceutical compounds that modify splicing of the pre-mRNA of at least the genomic sample relative to the alternative splicing of the pre-mRNA.

76. The system of claim 73, further comprising instructions that, when executed by the at least one processor, cause the system to determine at least the candidate biochemical compound by identifying one or more candidate antigens that targets the variant nucleobase or the one or more adjacent nucleobases flanking the variant nucleobase.

77. The system of claim 64, further comprising instructions that, when executed by the at least one processor, cause the system to determine, based on the splice-site score, that the variant nucleobase at the target position within the nucleotide sequence is associated with the alternative splicing corresponding to a target phenotype by:
providing, to a gene-to-phenotype-association model, the splice-site score for the variant nucleobase at the target position within the nucleotide sequence corresponding to a target gene; and
determining, utilizing the gene-to-phenotype-association model, a gene-to-phenotype score indicating a likelihood that the target gene is associated with the target phenotype.

78. The system of claim 77, further comprising instructions that, when executed by the at least one processor, cause the system to determine the gene-to-phenotype score indicating the likelihood that the target gene is associated with the target phenotype utilizing a gene-based burden-test model as the gene-to-phenotype-association model by:
identifying a first set of genomic samples comprising the variant nucleobase at the target position within the nucleotide sequence based on a first set of nucleotide reads for the first set of genomic samples;
identifying a second set of genomic samples comprising a reference nucleobase at the target position within the nucleotide sequence based on a second set of nucleotide reads for the second set of genomic samples;
providing, to the gene-based burden-test model, the splice-site score for the variant nucleobase at the target position, an additional splice-site score for the reference nucleobase at the target position within the nucleotide sequence, and genetic data indicating a phenotype exhibited by the first set of genomic samples and the second set of genomic samples; and
determining, utilizing the gene-based burden-test model, the gene-to-phenotype score comprising a value indicating a statistical likelihood that the target gene is associated with the target phenotype based on the splice-site score, the additional splice-site score, and the genetic data.

79. The system of claim 77, further comprising instructions that, when executed by the at least one processor, cause the system to:
provide the splice-site score for the variant nucleobase at the target position by identifying the variant nucleobase for the gene-to-phenotype-association model based on the splice-site score or inputting the splice-site score into the gene-to-phenotype-association model; and
determine the gene-to-phenotype score by:
generating, utilizing a rare-variant collapsing model as the gene-to-phenotype-association model and based on a gene-by-individual matrix comprising data for the target gene, a value for the gene-to-phenotype score indicating the likelihood that the target gene is associated with the target phenotype; or
generating, utilizing a probabilistic model as the gene-to-phenotype-association model and based on the splice-site score, a probability for the gene-to-phenotype score indicating the likelihood that the target gene is associated with the target phenotype.

80. The system of claim 64, further comprising instructions that, when executed by the at least one processor, cause the system to determine, based on the splice-site score, that the variant nucleobase at the target position within the nucleotide sequence is associated with the alternative splicing by:
accessing, for a set of genes from a genome associated with at least the genomic sample and processed by a gene embedding neural network, gene-to-phenotype scores indicating respective probabilities of the set of genes being associated with phenotypes determined for an organism of the genomic sample;
providing, to a diagnostic variant model, the splice-site score for the variant nucleobase at the target position and the gene-to-phenotype scores; and
determining, utilizing the diagnostic variant model and based on the splice-site score and the gene-to-phenotype scores, the genomic sample comprises a diagnostic variant that is associated with a phenotype of the phenotypes determined for the organism.

81. A system comprising:
at least one processor; and
a non-transitory computer readable medium comprising instructions that, when executed by the at least one processor, cause the system to:
identify, for a genomic region, an artificial nucleotide sequence comprising a target nucleobase at a target position;
access a first context-specific splice-site score for the target nucleobase with respect to a first target context based on one or more first context-specific conditional probabilities that one or more flanking nucleobases from the artificial nucleotide sequence are part of one or more matching acceptors or one or more matching donors for a conditioned nucleobase at a conditioned position within the artificial nucleotide sequence with respect to the first target context;
access a second context-specific splice-site score for the target nucleobase with respect to a second target context differing from the first target context based on one or more second context-specific conditional probabilities that the one or more flanking nucleobases are part of the one or more matching acceptors or the one or more matching donors for the conditioned nucleobase at the conditioned position within the artificial nucleotide sequence with respect to the second target context; and
determine, based on the first context-specific splice-site score and the second context-specific splice-site score, that the target nucleobase at the target position within the artificial nucleotide sequence is associated with splicing of pre-messenger ribonucleic acid (pre-mRNA) in the first target context and not associated with splicing of pre-mRNA in the second target context.

82. The system of claim 81, wherein the first context-specific splice-site score and the first target context and the second context-specific splice-site score and the second target context comprise:
a first tissue-specific splice-site score for the target nucleobase with respect to a first tissue based on one or more first tissue-specific conditional probabilities and a second tissue-specific splice-site score for the target nucleobase with respect to a second tissue based on one or more second tissue-specific conditional probabilities;
a first disease-specific splice-site score for the target nucleobase with respect to a first disease based on one or more first disease-specific conditional probabilities and a second disease-specific splice-site score for the target nucleobase with respect to a second disease based on one or more second disease-specific conditional probabilities;
a first cell-type-specific splice-site score for the target nucleobase with respect to a first cell type based on one or more first cell-type-specific conditional probabilities and a second cell-type-specific splice-site score for the target nucleobase with respect to a second cell type based on one or more second cell-type-specific conditional probabilities;
a first cell-line-specific splice-site score for the target nucleobase with respect to a first cell-line type based on one or more first cell-line-specific conditional probabilities and a second cell-line-specific splice-site score for the target nucleobase with respect to a second cell-line type based on one or more second cell-line-specific conditional probabilities; or
a first assay-specific splice-site score for the target nucleobase with respect to a first assay type based on one or more first assay-specific conditional probabilities and a second assay-specific splice-site score for the target nucleobase with respect to a second assay type based on one or more second assay-specific conditional probabilities.

83. The system of claim 81, wherein the first context-specific splice-site score and the first target context and the second context-specific splice-site score and the second target context comprise a first cell-type-specific splice-site score for the target nucleobase with respect to a first cell type comprising a target variant in a gene and a second cell-type-specific splice-site score for the target nucleobase with respect to a second cell type not comprising the target variant in the gene.

84. The system of claim 81, further comprising instructions that, when executed by the at least one processor, cause the system to determine that the target nucleobase at the target position within the artificial nucleotide sequence is associated with alternative splicing of the pre-mRNA in the first target context relative to a reference splicing of the pre-mRNA and not associated with the alternative splicing of the pre-mRNA in the second target context.

85. The system of claim 81, further comprising instructions that, when executed by the at least one processor, cause the system to determine that the target nucleobase at the target position within the artificial nucleotide sequence is associated with alternative splicing of pre-mRNA in the first target context and not associated with the alternative splicing of the pre-mRNA in the second target context.

86. The system of claim 81, wherein the artificial nucleotide sequence comprises an antisense oligonucleotide or a nucleotide sequence including a synthetic intron.

87. The system of claim 86, wherein the synthetic intron encodes a kinase or a virus designed to treat a disease.

88. The system of claim 81, wherein the target nucleobase comprises a reference nucleobase or a variant nucleobase.
